(19) United States
(12) Patent Application Publication
(10) Pub. No.: US 2024/0228588 A1
(43) Pub. Date: Jul. 11, 2024

(54) VARIANT CH1 DOMAINS AND VARIANT CL DOMAINS ENGINEERED FOR PREFERENTIAL CHAIN PAIRING AND MULTI-SPECIFIC ANTIBODIES COMPRISING THE SAME

(71) Applicant: ADIMAB, LLC, Lebanon, NH (US)

(72) Inventors: Kyle BARLOW, Lebanon, NH (US); Arvind SIVASUBRAMANIAN, Lebanon, NH (US); Michael Benjamin BATTLES, Lebanon, NH (US)

(21) Appl. No.: 18/271,486

(22) PCT Filed: Jan. 11, 2022
(86) PCT No.: PCT/US2022/012044

§ 371 Date: Jul. 10, 2023

Related U.S. Application Data
(60) Provisional application No. 63/136,091, filed on Jan. 11, 2021.

(51) Int. Cl. C07K 16/00

(52) U.S. Cl. CPC C07K 16/005; C07K 2317/31; C07K 2317/522

(57) ABSTRACT

Variant CH1 domains, variant CL domains, and variant CH1-CL domain sets which contain at least one amino acid substitution that promotes preferential CH1-CL pairing are provided. Also provided are polypeptides, molecules, and multi-specific antibodies comprising such variants; and compositions comprising any of the foregoing. Methods of generating a variant CH1 and/or variant CL domain library and methods of using same to identify one or more variant CH1 and/or variant CL domains and/or variant CH1-CL domain sets are also provided. Further provided are methods of screening for a combination of CH1-CL sets suited for multi-specific antibodies and/or antigen-binding antibody fragments.

### RELATED APPLICATIONS

This application claims priority to U.S. Provisional Application No. 63/136,091 filed on Jan. 11, 2021, entitled “CH1 AND KAPPA CL DOMAIN VARIANTS ENGINEERED FOR PREFERENTIAL CHAIN PAIRING AND MULTI-SPECIFIC ANTIBODIES COMPRISING THE SAME”, the contents of which are incorporated by reference in their entirety herein.

### FIELD OF THE INVENTION

The present invention relates to variant CH1 domain and variant CL domain polypeptides, which variants contain at least one amino acid substitution that promotes preferential chain pairing between a heavy chain containing said variant CH1 domain and a light chain containing said variant CL domain; polypeptides, molecules, and multi-specific antibodies or antigen-binding antibody fragments comprising such variants; and compositions comprising any of the foregoing. The present invention further relates to: polynucleotides encoding such variant CH1 and/or CL domain polypeptides; molecules, multi-specific antibodies or antigen-binding antibody fragments comprising said variant CH1 and/or CL domain polypeptides; and compositions and libraries comprising any of the foregoing. The present invention further relates to methods of generating a variant CH1 and/or CL domain library and methods of using same to identify one or more variant CH1 and/or CL domains and libraries and methods for identifying two polypeptides which preferentially pair with each other.

### BACKGROUND OF THE INVENTION

There are ongoing efforts to develop antibody therapeutics that have more than one antigen binding specificity, e.g., bispecific antibodies. Bispecific antibodies can be used to interfere with multiple surface receptors associated with cancer, autoimmune diseases, inflammation, or other diseases and conditions. Bispecific antibodies can also be used to place targets into close proximity and modulate protein complex formation or drive contact between cells. Production of bispecific antibodies was first reported in the early 1960s (Nisonoff et al., Arch Biochem Biophys 1961 93(2): 460-462) and the first monoclonal bispecific antibodies were generated using hybridoma technology in the 1980s (Milstein et al., Nature 1983 305(5934): 537-540). Interest in bispecific antibodies has increased significantly in the last decade due to their therapeutic potential and bispecific antibodies are now used in the clinic, e.g., blinatumomab and emicizumab have been approved for treatment of particular cancers (see Sedykh et al., Drug Des Devel Ther 12:195-208 (2018) and Labrijn et al. Nature Reviews Drug Discovery 18:585-608 (2019), for recent reviews of bispecific antibody production methods and features of bispecific antibodies approved for medical use).

While bispecific antibodies have shown considerable benefits over monospecific antibodies, broad commercial application of bispecific antibodies has been hampered by the lack of efficient/low-cost production methods, the lack of stability of bispecific antibodies, and the lack of long half-lives in humans. A bispecific antibody can be formed by co-expressing two different heavy chains and two different light chains. However, because heavy chains bind light chains in a relatively promiscuous manner, co-expression of two heavy chains and two light chains can lead to a mixture of sixteen possible combinations, representing ten different antibodies only one of which corresponds with the desired bispecific antibody (maximal yield 12.5% in the mixture if there is perfect promiscuity). This mispairing (also referred to as the chain-association issue) pauses a major challenge in manufacturing bispecific antibodies, and a variety of technologies have been developed to address the issue.

One strategy used to alleviate such chain mispairing is to design a bispecific antibody having common light chains, i.e., two different heavy chains and two identical light chains (see e.g., Merchant et al., Nat. Biotech. 16:677-681 (1998)). However, this strategy requires identifying two antibodies having different specificity but the same light chain, i.e., only differing in the heavy chain, which is difficult and tends to compromise the specificity of each binding arm and substantially reduces diversity (see, e.g., Wang et al., MABS 10(8):1226-1235 (2018)).

Another strategy is to modify the heavy chain constant region 1 (“CH1”) domain or the CH1 and the light chain constant region (“CL”) domain to promote CH1 pairing with a light chain of a particular isotype (kappa (“κ”) or lambda (“λ”)). For example, a kappa CL domain (“CLκ”)-preferring CH1 domain (may be referred to as “CH1κ”) would preferentially pair with a CLκ domain (or a variant CLκ domain) rather than with a CLλ domain (or a variant CLλ domain), and a lambda CL domain (“CLλ”)-preferring CH1 domain (may be referred to as “CH1λ”) would preferentially pair with a CLλ domain (or a variant CLλ domain) rather than with a CLκ domain (or a variant CLκ domain). Many engineering efforts have been made in the combination of CH1 and CLκ domains to facilitate proper heavy-light chain pairing. Although some CH1 and/or CLκ domain modifications have been reported which allegedly increase the propensity to result in preferential pairing between given CH1 and CLκ domains rather than pairing of a variant CH1 domain with another CL domain and/or pairing of a variant CLκ domain with another CH1 domain, the previous technologies appear to have a shortcoming(s) such as: not being universally applicable to multi-specific antibodies of different specificity combinations; not achieving sufficient preferential CH1-CLκ pairing; needing to incorporate numerous substitutions in the CH1 and/or CLκ domains; and/or needing to incorporate a substitution(s) additionally to the variable region(s) to achieve high preferential CH1-CLκ pairing. Therefore, notwithstanding the foregoing, there is still the need for improvement, particularly given the recent clinical focus on producing multi-specific, e.g., bispecific antibodies or antigen-binding antibody fragments for use in human therapies.

### SUMMARY OF THE INVENTION

An object of the present invention is to provide engineered variant CH1 domain polypeptides, or heavy chains comprising such a variant CH1 domain polypeptide, that may preferentially pair with a given CL domain or variant CL domain polypeptide, or with a light chain comprising such a CL domain or variant CL domain polypeptide. A variant CH1 domain polypeptide according to the present invention may be incorporated in a polypeptide(s), a molecule, or an antibody or antigen-binding antibody fragment such as a multi-specific (such as bispecific) antibody or antigen-binding antibody fragment.

In one aspect, provided herein are variant immunoglobulin heavy chain constant region 1 (“CH1”) domain polypeptides (also referred to herein as variant CH1 domains), and also provided herein are heavy chain polypeptides comprising such a variant CH1 domain polypeptide.

In some embodiments, such a variant CH1 domain polypeptide or a heavy chain polypeptide comprising such a variant CH1 domain polypeptide may preferentially pair with a variant CLκ or CLλ domain polypeptide rather than another given CL domain polypeptide (such as a WT CLλ or CLλ domain or another variant CLκ or CLλ domain polypeptide) or a light chain comprising such a variant CL domain polypeptide.

In some embodiments, the variant CH1 domain polypeptide contain at least one amino acid substitution (relative to a parent, e.g., wild-type, sequence, such as SEQ ID NO: 1 or allelic variants thereof such as but not limited to SEQ ID NO: 3).

In some embodiments, the variant CH1 domain polypeptide may comprise an amino acid substitution(s), and the amino acid substitution(s) may comprise or consist of an amino acid substitution(s) at one or more of the following CH1 amino acid positions: 145, 147, 181, 128, 124, 139, 141, 148, 166, 168, 175, 185, and 187, according to EU numbering. (Also, in each instance in this application when Applicant refers to a specific position in an immunoglobulin polypeptide the position is according to EU numbering unless specified otherwise). Optionally, the variant CH1 domain polypeptide is a variant of a CH1 domain of a human IgG, further optionally a human IgG1, human IgG2, or human IgG4.

In some embodiments, the variant CH1 domain polypeptide may comprise one or more additional amino acid substitutions at a CH1 position(s) outside of positions: 124, 128, 139, 141, 145, 147, 148, 166, 168, 175, 181, 185, and/or 187. In some instances, such additional position(s) may be optionally selected from the CH1 positions listed in Table 1.

In some embodiments, such a variant CH1 domain polypeptide or a heavy chain polypeptide comprising such a variant CH1 domain polypeptide may preferentially pair with an variant immunoglobulin kappa light chain constant region (CLκ) or lambda light chain constant region (CLλ) domain polypeptide or with a light chain polypeptide comprising the variant CLκ or CLλ domain, rather than with another given immunoglobulin light chain constant region (CL) domain or variant CL domain polypeptide (such as a wildtype (WT) CLκ or CLλ domain polypeptide or another variant CLκ or CLλ domain polypeptide) or rather than with a light chain polypeptide comprising a wild-type or another given variant CL domain polypeptide.

In some embodiments, the variant CLκ or CLλ domain polypeptide or a light chain comprising such a variant CL domain polypeptide with which such a variant CH1 domain polypeptide or a heavy chain polypeptide comprising the variant CH1 domain polypeptide preferentially pairs may comprise at least one amino acid substitution, which may comprise of consist of an amino acid substitution(s) at one or more of the following CLκ or CLλ amino acid positions: 114, 120, 124, 127, 129, 133, 135, 137, 138, 178, and/or 180, according to EU numbering.

In some embodiments, such a variant CH1 domain polypeptide is a variant of a CH1 domain of a human IgG, further optionally a human IgG1, human IgG2, or IgG4.

Such a variant CH1 domain polypeptide may not be part of a pre-existing CH1-CL set listed in Table 1.

In some embodiments, the amino acid substitution(s) of the variant CH1 domain polypeptide may comprise or consist of an amino acid substitution(s) at (I) position(s) 185 and/or 187; (II) position(s) 145, 147, and/or 148; (III) position(s) 147 or 148; (IV) position 145; (V) position(s) 166 and/or 187; (VI) position(s) 145 and/or 147; or (VII) position(s) 124 and/or 147.

In further embodiments, such a variant CH1 domain polypeptide or a heavy chain polypeptide comprising such a variant CH1 domain polypeptide may preferentially pair with a variant CLκ or CLλ domain polypeptide or a light chain polypeptide comprising such a variant CL domain polypeptide, and the variant CL domain polypeptide may comprise at least one amino acid substitution, and the amino acid substitution position(s) in the variant CL (CLκ or CLλ) domain polypeptide may comprise or consist of an amino acid substitution(s) at (I) position 135; (II) position 124; (III) position 129; (IV) position 133; (V) position(s) 137 and/or 138; (VI) position(s) 178 and/or 180; or (VI) position 127.

In some embodiments, the substitution position combination of the CH1-CL set may be according to the substitution position combination of any one of the CH1-CLκ sets in Table 2 or any one of the CH1-CLλ sets in Table 28.

In some embodiments, the amino acid substitution(s) of the variant CH1 domain polypeptide may comprise or consist of an amino acid substitution(s) at any of the following position combinations: (i) positions 145, 147, and 181; (ii) positions 128 and 147; (iii) positions 168, 185, and 187; (iv) positions 147 and 185; (v) position 148; (vi) positions 139, 141, and 187; (vii) positions 166 and 187; (viii) positions 168 and 185; (ix) positions 124 and 147; (x) positions 147 and 148; (xi) position 145; (xii) positions 145 and 181; (xiii) positions 124, 145, and 147; (xiv) positions 166 and 187; (xv) positions 147 and 175 (xvi) positions 147, 175, and 181; (xvii) positions 145 and 147; or (xviii) positions 147 and 185.

In further embodiments, such a variant CH1 domain polypeptide or a heavy chain polypeptide comprising such a variant CH1 domain polypeptide may preferentially pair with a variant CLκ domain polypeptide or a light chain polypeptide comprising such a variant CLκ domain polypeptide and, optionally, the amino acid substitution position(s) in such a variant CLκ domain polypeptide may comprise or consist of: (i) positions 129, 178, and 180; (ii) positions 124, 133, and 178; (iii) at position 135; (iv) positions 135 and 178; (v) positions 124 and 129; (vi) positions 114, 135, and 138; (vii) positions 137 and 138; (viii) position 135; (ix) positions 127 and 129; (x) positions 127 and 129; (xi) position 133 or positions 124 and 133; (xii) position 133 or positions 120, 178, and 180; (xiii) positions 127, 129, and 178; (xiv) positions 114, 137, and 138; (xv) positions 129, 178, and 180; (xvi) positions 129 and 180; (xvii) positions 133 and 180; or (xviii) positions 129 and 180.

In further embodiments, such a variant CH1 domain polypeptide or a heavy chain polypeptide comprising such a variant CH1 domain polypeptide may preferentially pair with a variant CLλ domain polypeptide or a light chain polypeptide comprising such a variant CLλ domain polypeptide, and optionally the amino acid substitution position(s) in such a variant CLλ domain polypeptide may comprise or consist of: (i) positions 129, 178, and 180; (ii) positions 133 and 178; (iii) at position 135; (iv) positions 135 and 178; (v) positions 124 and 129; (vi) positions 114, 135, and 138; (vii) positions 138; (viii) position 135; (ix) positions 127 and 129; (x) positions 127 and 129; (xi) position 133; (xii) position 133 or positions 120, 178, and 180; (xiii) positions 127, 129, and 178; (xiv) positions 114, 137, and 138; (xv) positions 129, 178, and 180; (xvi) positions 129 and 180; (xvii) positions 133 and 180; or (xviii) position 129.

In yet further embodiments, such a variant CH1 domain polypeptide may comprise one or more of the following amino acid substitutions: 124R, 128R, 139R, 141Q, 145Q, 145S, 147E, 147H, 147N, 147Q, 147R, 147T, 148E, 148R, 166K, 168R, 168S, 175D, 175E, 181E, 181Q, 185E, 185Q, 185S, 185Y, 187D, 187K, and/or 187Q.

In certain embodiments, the amino acid substitution(s) of such a variant CH1 domain polypeptide may comprise or consist of (i) 145Q, 147E, and 181E; (ii) 128R and 147R; (iii) 168S, 185S, and 187D; (iv) 147T and 185Q; (v) 148R; (vi) 139R, 141Q, and 187Q; (vii) 166K and 187K; (viii) 168R and 185E; (ix) 124R and 147R; (x) 147H and 148E; (xi) 145S; (xii) 145S and 181Q; (xiii) 145S; (xiv) 145Q and 181E; (xv) 124R, 145S, and 147Q; (xvi) 166K and 187K; (xvii) 147R and 175D; (xviii) 147R, 175E, and 181Q; (xix) 145S and 147N; or (xx) 147N and 185Y.

In certain embodiments, the variant CH1 domain polypeptide or a heavy chain polypeptide comprising such a variant CH1 domain polypeptide preferentially pairs with a variant CLκ domain polypeptide or a light chain polypeptide comprising such a variant CLκ domain polypeptide, and optionally the amino acid substitution(s) in the variant CLκ domain polypeptide may comprise or consist of (i) 129R, 178R, and 180Q; (ii) 124E, 133Q, and 178E; (iii) 135R; (iv) 135S and 178R; (v) 124S and 129E; (vi) 114D, 135S, and 138R; (vii) 137S and 138E; (viii) 135S; (ix) 127D and 129E; (x) 127R and 129R; (xi) 133Y; or 124E and 133Y; (xii) 133Y; (xiii) 120S, 178H, and 180Q; (xiv) 127T, 129D, and 178R; (xv) 114Q, 137T, and 138E; (xvi) 129D, 178R, and 180H; (xvii) 129D and 180Q; (xviii) 133Y and 180R; or (xix) 129R and 180S.

In certain embodiments, the variant CH1 domain polypeptide or a heavy chain polypeptide comprising such a variant CH1 domain polypeptide preferentially pairs with a variant CLλ domain polypeptide or a light chain polypeptide comprising such a variant CLλ domain polypeptide, and optionally the amino acid substitution(s) in the variant CLλ domain polypeptide may comprise or consist of (i) 129R, 178R, and 180Q; (ii) 133Q and 178E; (iii) 135R; (iv) 135S and 178R; (v) 124S and 129E; (vi) 114D, 135S, and 138R; (vii) 138E; (viii) 135S; (ix) 127D and 129E; (x) 127R and 129R; (xi) 133Y; (xii) 133Y; (xiii) 120S, 178H, and 180Q; (xiv) 127T, 129D, and 178R; (xv) 114Q, 137T, and 138E; (xvi) 129D, 178R, and 180H; (xvii) 129D and 180Q; (xviii) 133Y and 180R; or (xix) 129R.

In certain embodiments, the amino acid substitution(s) of such a variant CH1 domain polypeptide may comprise or consist of (i) 145Q, 147E, and 181E; (ii) 128R and 147R; (iii) 168S, 185S, and 187D; or (iv) 147T and 185Q.

In certain embodiments, the variant CH1 domain polypeptide or a heavy chain polypeptide comprising such a variant CH1 domain polypeptide preferentially pairs with a variant CLκ domain polypeptide or a light chain polypeptide comprising such a variant CLκ domain polypeptide, and optionally the amino acid substitution(s) in the variant CLκ domain polypeptide may comprise or consist of (i) 129R, 178R, and 180Q, (ii) 124E, 133Q, and 178E; (iii) 135R; or (iv) 135S and 178R.

In certain embodiments, the variant CH1 domain polypeptide or a heavy chain polypeptide comprising such a variant CH1 domain polypeptide may preferentially pair with a variant CLλ domain polypeptide or a light chain polypeptide comprising such a variant CLλ domain polypeptide, the amino acid substitution(s) in the variant CLλ domain polypeptide may comprise or consist of (i) 129R, 178R, and 180Q, (ii) 133Q and 178E; (iii) 135R; or (iv) 135S and 178R.

In particular embodiments, the variant CH1 domain polypeptide may comprise the amino acid sequence according to any one of SEQ ID NOS: 31, 21, 11, 41, 51, 61, 71, 81, 91, 101, 111, 121, 131, 141, 151, 161, 171, 181, 191, or 201.

In some preferred embodiments, the variant CH1 domain polypeptide may comprise the amino acid sequence according to SEQ ID NOS: 31, 21, 11, or 41.

In some embodiments, the heavy chain polypeptides according to the present disclosure may comprise any of the variant CH1 domain polypeptides described above.

Another object of the present invention is to provide engineered variant CL domain (e.g., variant CLκ or CLλ domain) polypeptides, or light chains comprising such a variant CL domain polypeptide, that may preferentially pair with a given CH1 domain or variant CH1 domain polypeptide or with a heavy chain comprising such a CH1 domain or variant CH1 domain polypeptide. A variant CLκ or CLλ domain polypeptide according to the present invention may be incorporated in a polypeptide, a molecule, or an antibody or antigen-binding antibody fragment such as a multi-specific (such as bispecific) antibody or antigen-binding antibody fragment.

In one aspect, provided herein are variant immunoglobulin CLκ or CLλ domain polypeptides (also referred to herein as variant CLκ or CLλ domain polypeptides, variant CLκ or variant CLλ, or the like), and also provided herein are light chain polypeptides comprising such a variant CL domain polypeptide.

In some embodiments, the variant CLκ or CLλ domain polypeptides or light chains comprising such a variant CLκ or CLλ domain polypeptide may preferentially pair with a variant CH1 domain polypeptide rather than with another given CH1 domain (such as a WT CH1 domain polypeptide or another variant CH1 domain polypeptide) and/or may preferentially pair with a heavy chain polypeptide comprising a variant CH1 domain polypeptide rather than with another heavy chain polypeptide comprising a wild-type or another given variant CH1 domain polypeptide.

In some embodiments, the variant CLκ or CLλ domain polypeptides may contain at least one amino acid substitution (relative to a parent, e.g., wild-type, sequence, such as SEQ ID NO: 2 or 9).

In some embodiments, the variant CLκ or CLλ domain polypeptide may comprise at least one amino acid substitution, which may comprise or consist of an amino acid substitution(s) at one or more of the following amino acid positions (CL positions): 114, 120, 124, 127, 129, 133, 135, 137, 138, 178, and/or 180, according to EU numbering.

In some embodiments, the variant CLκ or CLλ domain polypeptide may comprise one or more additional amino acid substitutions at a CLκ position(s) outside of positions: 114, 120, 124, 127, 129, 133, 135, 137, 138, 178, and/or 180. In some instances, such additional position(s) may be optionally selected from the CLκ or CLλ positions listed in Table 1.

In some embodiments, the variant CLκ or CLλ domain polypeptide or a light chain comprising such a variant CLκ or CLλ domain polypeptide may optionally preferentially pairs with a variant CH1 domain polypeptide or a heavy chain comprising a variant CH1 domain polypeptide. In such embodiment, the variant CH1 domain polypeptide or a light chain comprising such a variant CLκ or CLλ domain polypeptide with which the variant CLκ or CLλ domain polypeptide or a heavy chain comprising a CH1 domain polypeptide preferentially pairs may comprise at least one amino acid substitution, which may comprise or consist of an amino acid substitution(s) at one or more of the following positions: 124, 128, 139, 141, 145, 147, 148, 166, 168, 175, 181, 185, and 187, according to EU numbering, with the proviso that such a variant CLκ domain polypeptide may not be part of a pre-existing CH1-CLκ set listed in Table 1 (i.e., sets other than CTL31), and such a variant CLλ domain polypeptide may not be part of a pre-existing CH1-CLλ set listed in Table t (i.e., the CTL3l set).

In some embodiments, the amino acid substitution(s) of the variant CLκ or CLλ domain polypeptide may comprise or consist of amino acid substitution(s) at: (I) position 135; (II) position 124; (III) position 129; (IV) position 133; (V) position(s) 137 and/or 138; (VI) position(s) 178 and/or 180; or (VII) position 127.

In some embodiments, the variant CLκ or CLλ domain polypeptide or a light chain comprising such a variant CLκ or CLλ domain polypeptide may preferentially pair with a variant CH1 domain polypeptide or a heavy chain comprising a variant CH1 domain polypeptide. In such embodiments, the amino acid substitution(s) in the variant CH1 domain polypeptide may comprise or consist of an amino acid substitution(s) at: (I) position(s) 185 and/or 187; (II) position(s) 145, 147, and/or 148; (III) position(s) 147 or 148; (IV) position 145; (V) position(s) 166 and/or 187; (VI) position(s) 145 and/or 147; or (VII) position(s) 124 and/or 147.

In some embodiments, the substitution position combination of the CH1-CL set may comprise any one of the CH1-CLκ sets in Table 2 and/or any one of the CH1-CLλ sets in Table 28.

In some embodiments, the amino acid substitution(s) of the variant CLκ or CLλ domain polypeptide may comprise or consist of amino acid substitution(s) at: (i) positions 129, 178, and 180; (ii) positions 124, 133, and 178; or positions 133 and 178; (iii) position 135; (iv) positions 135 and 178; (v) positions 124 and 129; (vi) positions 114, 135, and 138; (vii) positions 137 and 138; or position 138; (viii) positions 127 and 129; (ix) position 133; (x) positions 124 and 133; (xi) positions 120, 178, and 180; (xii) positions 127, 129, and 178; (xiii) positions 114, 137, and 138; (xiv) positions 129 and 180; (xv) positions 133 and 180; or (xvi) position 129.

In some embodiments, the variant CLκ or CLλ domain polypeptide or a light chain comprising such a variant CLκ or CLλ domain polypeptide may preferentially pair with a variant CH1 domain polypeptide or a heavy chain comprising a variant CH1 domain polypeptide. In such embodiments, the amino acid substitution(s) in the variant CH1 domain polypeptide may comprise at least one amino acid substitution(s) which comprises or consists of an amino acid substitution(s) at: (i) positions 145, 147, and 181 or positions 147 and 175; (ii) positions 128 and 147; (iii) positions 168 and 185 or positions 168, 185, and 187; (iv) positions 147 and 185; (v) position 148; (vi) positions 139, 141, and 187; (vii) positions 166 and 187; (viii) positions 124 and 147 or positions 147 and 148; (ix) position 145 or positions 145 and 181; (x) position 145; (xi) positions 145 and 181; (xii) positions 124, 145, and 147; (xiii) positions 166 and 187; (xiv) positions 147 and 185 or positions 147, 175, and 181; (xv) positions 145 and 147; or (xvi) positions 147 and 185.

In further embodiments, the variant CLκ or CLλ domain polypeptide may comprise one or more of the following amino acid substitutions: 114D, 114Q, 120S, 124E, 124S, 127D, 127R, 127T, 129D, 129E, 129R, 133Q, 133Y, 135R, 135S, 137S, 137T, 138E, 138R, 178E, 178H, 178R, and 180H, 180Q, 180R, and/or 180S.

In yet further embodiments, the amino acid substitution(s) of the variant CLκ or CLλ domain polypeptide may comprise or consist of: (i) 129R, 178R, and 180Q; (ii) 124E, 133Q, and 178E; or 133Q and 178E; (iii) 135R; (iv) 135S and 178R; (v) 124S and 129E; (vi) 114D, 135S, and 138R; (vii) 137S and 138E; or 138E; (viii) 135S; (ix) 127D and 129E; (x) 127R and 129R; (xi) 133Y; (xii) 133Y; (xiii) 124E and 133Y; or 133Y; (xiv) 120S, 178H, and 180Q; (xv) 127T, 129D, and 178R; (xvi) 114Q, 137T, and 138E; (xvii) 129D, 178R, and 180H; (xviii) 129D and 180Q; (xix) 133Y and 180R; or (xx) 129R and 180S; or 129R.

In certain embodiments, the variant CLκ or CLλ domain polypeptide or a light chain polypeptide comprising such a variant CLκ or CLλ domain polypeptide may preferentially pair with a variant CH1 domain polypeptide or a heavy chain polypeptide comprising a variant CH1 domain polypeptide. In such embodiments, the amino acid substitution(s) in such a variant CH1 domain polypeptide may comprise or consist of (i) 168S, 185S, and 187D; (ii) 128R and 147R; (iii) 145Q, 147E, and 181E; (iv) 147T and 185Q; (v) 148R; (vi) 139R, 141Q, and 187Q; (vii) 166K and 187K; (viii) 168R and 185E; (ix) 124R and 147R; (x) 147H and 148E; (xi) 145S; (xii) 145S and 181Q; (xiii) 145S; (xiv) 145Q and 181E; (xv) 124R, 145S, and 147Q; (xvi) 166K and 187K; (xvii) 147R and 175D; (xviii) 147R, 175E, and 181Q; (xix) 145S and 147N; or (xx) 147N and 185Y.

In some preferred embodiments, the amino acid substitution(s) in the variant CLκ or CLλ domain polypeptide may consist of (i) 129R, 178R, and 180Q; (ii) 124E, 133Q, and 178E; 133Q and 178E (iii) 135R; or (iv) 135S and 178R.

In some embodiments, the variant CLκ or CLλ domain polypeptide or a light chain polypeptide comprising such a variant CLκ or CLλ domain polypeptide preferentially pairs with a variant CH1 domain polypeptide or a heavy chain polypeptide comprising a variant CH1 domain polypeptide. In such instances, the amino acid substitution(s) in the variant CH1 domain polypeptide may comprise or consist of: (i) 168S, 185S, and 187D; (ii) 128R and 147R; (iii) 145Q, 147E, and 181E; or (iv) 147T and 185Q.

In particular embodiments, the variant CLκ or CLλ domain polypeptide may comprise an amino acid sequence selected from one of SEQ ID NOS: 32, 22, 12, 42, 52, 62, 72, 82, 92, 102, 112, 122, 132, 142, 152, 162, 172, 182, 192, or 202 or any one of SEQ ID NOS: 59, 99, 39, 199, 89, 49, 29, 19, 69, 79, 109, 119, 129, 139, 149, 159, 169, 179, 189, or 209.

In some preferred embodiments, the variant CLκ or CLλ domain polypeptide may comprise an amino acid sequence selected from one of SEQ ID NOS: 12, 22, 32, 42 or any one of SEQ ID NOS: 59, 99, 39, 199, 89, 49, or 29. In some embodiments, the light chain polypeptides according to the present disclosure may comprise any of the variant CL domain polypeptides described above.

Another object of the present invention is to provide sets of a variant CH1 domain polypeptide and a variant CLκ or CLλ domain polypeptide which preferentially pair with each other (such a set is a “variant CH1-CL set”, a “CH1-CL variant set”, “CH1-CL design”, “design CH1-CL”, “network” or the like). One or more CH1-CL sets according to the present invention may be incorporated in a polypeptide, a molecule, or a multi-specific (such as bispecific) antibody or antigen-binding antibody fragment.

In one aspect, provided herein are CH1-CL sets (which may be a kit comprising a CH1 domain polypeptide and a CL domain polypeptide), which may comprise a variant CH1 domain polypeptide and/or a variant CLκ or CLλ domain polypeptide.

In some embodiments, a CH1-CL set according to the present invention may comprise any of the variant CH1 domain polypeptide as described above and/or any of the variant CLκ or CLλ domain polypeptide as described above.

In some embodiments, the CH1-CL sets may be any of the CH1-CLκ sets listed in Table 2 or any of the CH1-CLλ sets listed in Table 28.

In certain embodiments, the variant CH1 domain polypeptide and the variant CLκ or CLλ domain polypeptide of the CH1-CL sets may comprise the amino acid sequence of SEQ ID NOS: 31 and 32, respectively; SEQ ID NOS: 21 and 22, respectively; SEQ ID NOS: 11 and 12, respectively SEQ ID NOS: 41 and 42, respectively; SEQ ID NOS: 51 and 52, respectively; SEQ ID NOS: 61 and 62, respectively; SEQ ID NOS: 71 and 72, respectively; SEQ ID NOS: 81 and 82, respectively; SEQ ID NOS: 91 and 92, respectively; SEQ ID NOS: 101 and 102, respectively; SEQ ID NOS: 111 and 112, respectively; SEQ ID NOS: 121 and 122, respectively; SEQ ID NOS: 131 and 132, respectively; SEQ ID NOS: 141 and 142, respectively; SEQ ID NOS: 151 and 152, respectively; SEQ ID NOS: 161 and 162, respectively; SEQ ID NOS: 171 and 172, respectively; SEQ ID NOS: 181 and 182, respectively; SEQ ID NOS: 191 and 192, respectively; or SEQ ID NOS: 201 and 202, respectively; SEQ ID NOS: 51 and 59, respectively; SEQ ID NOS: 91 and 99, respectively; SEQ ID NOS: 31 and 39, respectively; SEQ ID NOS: 191 and 199, respectively; respectively; SEQ ID NOS: 81 and 89, respectively; SEQ ID NOS: 21 and 29, respectively; SEQ ID NOS: 41 and 49, respectively; SEQ ID NOS: 11 and 19, respectively; SEQ ID NOS: 61 and 69, respectively; SEQ ID NOS: 71 and 79, SEQ ID NOS: 101 and 109, respectively; SEQ ID NOS: 111 and 119, respectively; SEQ ID NOS: 121 and 129, respectively; SEQ ID NOS: 131 and 139, respectively; SEQ ID NOS: 141 and 149, respectively; SEQ ID NOS: 151 and 159, respectively; SEQ ID NOS: 161 and 169, respectively; SEQ ID NOS: 171 and 179, respectively; SEQ ID NOS: 181 and 189, respectively; or SEQ ID NOS: 201 and 209, respectively.

In particular embodiments, the variant CH1 domain polypeptide and the variant CL domain polypeptide of the CH1-CL sets may comprise the amino acid sequence of: SEQ ID NOS: 31 and 32, respectively; SEQ ID NOS: 21 and 22, respectively; SEQ ID NOS: 11 and 12, respectively; SEQ ID NOS: 41 and 42, respectively; SEQ ID NOS: 51 and 59, respectively; SEQ ID NOS: 91 and 99, respectively; SEQ ID NOS: 31 and 39, respectively; SEQ ID NOS: 191 and 199, respectively; respectively; SEQ ID NOS: 81 and 89, respectively; SEQ ID NOS: 21 and 29, respectively; or SEQ ID NOS: 41 and 49, respectively.

In another aspect, provided herein are immunoglobulin polypeptides comprising (i) at least one variant CH1 domain polypeptide or at least one heavy chain polypeptide comprising a variant CH1 domain polypeptide and/or (ii) at least one variant CLκ or CLλ domain polypeptide or a light chain polypeptide comprising a variant CLκ or CLλ domain polypeptide.

In some embodiments, the immunoglobulin polypeptide may comprise at least one variant CH1 domain polypeptide or heavy chain polypeptide comprising a variant CH1 domain polypeptide, and the variant CH1 domain polypeptide may be any of the variant CH1 domain polypeptides described above.

In some embodiments, when the immunoglobulin polypeptide may comprise at least one variant CLκ or CLλ domain polypeptide or light chain polypeptide comprising a variant CLκ or CLλ domain polypeptide, and the variant CLκ or CLλ domain polypeptide may be any of the variant CLκ or CLλ domain polypeptides described above.

In some embodiments, an immunoglobulin polypeptide according to the present invention may comprise one or more of: (i) an antigen-binding domain; (ii) a CH1 domain or variant CH1 domain polypeptide; (iii) an immunoglobulin heavy chain constant region 2 (“CH2”) domain or variant CH2 domain polypeptide; (iv) an immunoglobulin heavy chain constant region 3 (“CH3”) domain or variant CH3 domain polypeptide; and/or (v) a light chain constant region (CL) domain or variant CL (e.g., variant CLκ or CLλ) domain polypeptide.

In certain embodiments, the antigen-binding domain may comprise an immunoglobulin heavy chain variable region (“VH”) domain, an immunoglobulin light chain variable region (“VL”) domain, a single chain fragment variable (“scFv”), an antigen-binding fragment (Fab), a F(ab′), a F(ab′)₂, F(ab′)2, or a combination thereof. In certain embodiments, the CH1 domain may comprise a wild-type CH1 amino acid sequence or comprises one or more amino acid substitutions relative to a wild-type CH1 amino acid sequence. In certain embodiments, the CH2 domain may comprise a wild-type CH2 amino acid sequence or comprises one or more amino acid substitutions relative to a wild-type CH2 amino acid sequence. In certain embodiments, the CH3 domain may comprise a wild-type CH3 amino acid sequence or comprises one or more amino acid substitutions relative to a wild-type CH3 amino acid sequence. In certain embodiments, the CL domain may comprise a wild-type CL amino acid sequence or comprises one or more amino acid substitutions relative to a wild-type CL amino acid sequence.

In certain embodiments, the immunoglobulin polypeptide may comprise a VH domain and may be bound to or paired with another polypeptide comprising a VL domain, wherein the VH domain and the VL domain may form an antigen-binding site. In certain embodiments, the polypeptide may comprise a VL domain and may be bound to or paired with another polypeptide comprising a VH domain, wherein the VL domain and the VH domain may form an antigen-binding site.

In another aspect, provided herein are molecules comprising at least a first polypeptide comprising at least one variant CH1 domain polypeptide or heavy chain polypeptide comprising a variant CH1 domain polypeptide and a second polypeptide comprising at least one variant CLκ or CLλ domain polypeptide or light chain polypeptide comprising a variant CLκ or CLλ domain polypeptide.

In some embodiments, the first polypeptide and the second polypeptide of such a molecule may be bound to or paired with each other, optionally via a disulfide bond(s).

In some embodiments, the variant CH1 domain polypeptide of such a molecule may be any of the variant CH1 domain polypeptides according to the present invention.

In some embodiments, the variant CLκ or CLλ domain polypeptide of such a molecule may be any of the variant CLκ or CLλ domain polypeptides according to the present invention.

In some embodiments, the first polypeptide and the second polypeptide may be any of the variant CH1 domain-containing polypeptides described above and any of the variant CLκ or CLλ domain-containing polypeptides described above, respectively.

In certain embodiments, the first polypeptide comprises an antigen-binding domain and/or the second polypeptide comprises an antigen-binding domain.

In some instances, the antigen-binding domain of the first polypeptide and the antigen-binding domain of the second polypeptide of such a molecule may optionally comprise a VH and a VL, respectively, or a VL and a VH, respectively, further optionally forming an antigen binding site specific for a first epitope In some instances, the antigen-binding domain of the first polypeptide may optionally comprise a scFv or nanobody specific for a first epitope and/or the antigen-binding domain of the second polypeptide may comprise a scFv or nanobody specific for a second the, respectively, further optionally wherein the first epitope is the same as or is different than the second epitope.

In other embodiments, the molecule may further comprise a third polypeptide comprising at least one variant CH1 domain polypeptide or heavy chain polypeptide comprising a variant CH1 domain polypeptide and a fourth polypeptide comprising at least one variant CLκ or CLλ domain polypeptide or light chain polypeptide comprising a variant CLκ or CLλ domain polypeptide. In such embodiments, the variant CH1 domain polypeptide may be any of the variant CH1 domain polypeptide according to the present invention and/or the variant CLκ or CLλ domain polypeptide may be any of the variant CLκ or CLλ domain polypeptide according to the present invention.

In certain embodiments, the third polypeptide and the fourth polypeptide may be bound to or paired with each other, optionally via a disulfide bond(s).

In some embodiments, the variant CH1 domain polypeptide of the third polypeptide may be the same as or different than the variant CH1 domain polypeptide of the first polypeptide; and/or the variant CLκ or CLλ domain polypeptide of the fourth polypeptide may be the same as or different than the variant CLκ or CLλ domain polypeptide of the second polypeptide.

In some embodiments, the third polypeptide and the fourth polypeptide may be any of the variant CH1 domain-containing polypeptides described above and any of the variant CLκ or CLλ domain-containing polypeptides described above, respectively.

In some embodiments, the third polypeptide may comprise an antigen-binding domain and/or the fourth polypeptide may comprise an antigen-binding domain.

In some instances, the antigen-binding domain of the third polypeptide and the antigen-binding domain of the fourth polypeptide may comprise a VH and a VL, respectively, or a VL and a VH, respectively, optionally forming an antigen-binding site specific for a third epitope, further optionally wherein the third epitope may be the same as or different than the first and/or second epitope. In some instances, the antigen-binding domain of the third polypeptide may comprise a scFv or nanobody specific for a third epitope and/or the antigen-binding domain of the fourth polypeptide may comprise a scFv or nanobody specific for a fourth epitope, respectively, optionally wherein the third epitope is the same as or is different than the fourth epitope, further optionally wherein the third and/or fourth epitopes may be same as or different from the first and/or second epitope.

In certain embodiments, the molecule according to the present disclosure may be a multi-specific antibody or antigen-binding antibody fragment, optionally a bispecific, tri-specific, tetra-specific, penta-specific, or hexa-specific antibody or antigen-binding antibody fragment.

In further embodiments, the molecule may optionally comprise a structure as depicted in any one of FIGS. 2-7.

In further embodiments, the molecule may optionally comprise an IgG, still further optionally an IgG1, IgG2, IgG3 or IgG4.

In certain embodiments, in such molecules, the variant CH1 domain polypeptide of the third polypeptide may be different from the variant CH1 domain polypeptide of the first polypeptide; and/or the variant CLκ or CLλ domain polypeptide of the fourth polypeptide may be different from the variant CLκ or CLλ domain polypeptide of the second polypeptide. In such embodiments, the CH1 and variant CLκ or CLλ domain polypeptides of the first and second polypeptides may be referred to as the first CH1-CL set and the CH1 and variant CLκ or CLλ domain polypeptides of the third and fourth polypeptides may be referred to as the second CH1-CL set.

In particular embodiments, the first CH1-CL set and the second CH1-CL set may be individually selected from the CH1-CLκ sets listed in Table 2 and the CH1-CLλ sets listed in Table 28.

In some preferred embodiments, the first CH1-CL set and the second CH1-CL set may be two CH1-CLκ sets of Network 1443 and Network 1993, respectively; Network 1039 and Network 1993, respectively; Network 1443 and Network 964, respectively; Network 1443 and Network 1039, respectively; Network 1443 and Network 367, respectively; Network 1443 and Network 2366, respectively; Network 1039 and Network 367, respectively; Network 1039 and Network 2529, respectively; Network 1039 and Network 742, respectively; Network 1039 and Network 2366, respectively; Network 1993 and Network 1443, respectively; Network 1993 and Network 1039, respectively; Network 964 and Network 1443, respectively; Network 1039 and Network 1443, respectively; Network 367 and Network 1443, respectively; Network 2366 and Network 1443, respectively; Network 367 and Network 1039, respectively; Network 2529 and Network 1039, respectively; Network 742 and Network 1039, respectively; or Network 2366 and Network 1039, respectively.

In some exemplary embodiments, the first CH1-CL set and the second CH1-CL set may be two CH1-CLλ sets of Network 367 and Network 1621, respectively; Network 964 and Network 1443, respectively; Network 367 and Network 2529, respectively; Network 964 and Network 1621, respectively; Network 367 and Network 1443, respectively; Network 964 and Network 2529, respectively; or Network 1443 and Network 1993, respectively.

In some specific embodiments, the first CH1-CLκ set and the second CH1-CLκ set may be two CH1-CLκ sets of Network 1443 and Network 1993, respectively or Network 1993 and Network 1443, respectively.

In some specific embodiments, the first CH1-CL set and the second CH1-CL set may be two CH1-CLλ sets of Network 367 and Network 1621, respectively; or Network 964 and Network 1443, respectively.

In some specific embodiments, the amino acid substitutions in the variant CH1 domain of the first polypeptide may comprise or consist of 145Q, 147E, and 181, the amino acid substitutions in the variant CLκ domain of the second polypeptide may comprise or consist of 129R, 178R, and 180Q, the amino acid substitutions in the variant CH1 domain of the third polypeptide may comprise or consist of 128R and 147R, and the amino acid substitutions in the variant CLκ domain of the fourth polypeptide may comprise or consist of 124E, 133Q, and 178E.

In some specific embodiments, the amino acid substitutions in the variant CH1 domain of the first polypeptide may comprise or consist of 128R and 147R, the amino acid substitutions in the variant CLκ domain of the second polypeptide may comprise or consist of 124E, 133Q, and 178E, the amino acid substitutions in the variant CH1 domain of the third polypeptide may comprise or consist of 145Q, 147E, and 181E, and the amino acid substitutions in the variant CLκ domain of the fourth polypeptide may comprise or consist of 129R, 178R, and 180Q.

In some specific embodiments, the amino acid substitutions in the variant CH1 domain of the first polypeptide may comprise or consist of 148R, the amino acid substitutions in the variant CLλ domain of the second polypeptide may comprise or consist of 124S and 129E, the amino acid substitutions in the variant CH1 domain of the third polypeptide may comprise or consist of 145S and 147N, and the amino acid substitutions in the variant CLλ domain of the fourth polypeptide may comprise or consist of 133Y and 180R.

In some specific embodiments, the amino acid substitutions in the variant CH1 domain of the first polypeptide may comprise or consist of 145S and 147N, the amino acid substitutions in the variant CLλ domain of the second polypeptide may comprise or consist of 133Y and 180R, the amino acid substitutions in the variant CH1 domain of the third polypeptide may comprise or consist of 148R, and the amino acid substitutions in the variant CLλ domain of the fourth polypeptide may comprise or consist of 124S and 129E.

In some specific embodiments, the amino acid substitutions in the variant CH1 domain of the first polypeptide may comprise or consist of 124R and 147R, the amino acid substitutions in the variant CLλ domain of the second polypeptide may comprise or consist of 127D and 129E, and the amino acid substitutions in the variant CH1 domain of the third polypeptide may comprise or consist of 145Q, 147E, and 181E, and the amino acid substitutions in the variant CLλ domain of the fourth polypeptide may comprise or consist of 129R, 178R, and 180Q.

In some specific embodiments, the amino acid substitutions in the variant CH1 domain of the first polypeptide may comprise or consist of 145Q, 147E, and 181E, the amino acid substitutions in the variant CLλ domain of the second polypeptide may comprise or consist of 129R, 178R, and 180Q, and the amino acid substitutions in the variant CH1 domain of the third polypeptide may comprise or consist of 124R and 147R, and the amino acid substitutions in the variant CLλ domain of the fourth polypeptide may comprise or consist of 127D and 129E.

In some specific embodiments of the molecule, the variant CH1 domain of the first polypeptide, the variant CL domain of the second polypeptide, the variant CH1 domain of the third polypeptide, and the variant CL domain of the fourth polypeptide comprise the amino acid sequence of (A) SEQ ID NOS: 31, 32, 21, and 22, respectively; (B) SEQ ID NOS: 21, 22, 31, and 32; (C) SEQ ID NOS: 51, 59, 191, and 199, respectively; (D) SEQ ID NOS: 191, 199, 51, and 59, respectively; (E) SEQ ID NOS: 91, 99, 31, and 39, respectively; or (F) SEQ ID NOS: 31, 39, 91, and 99, respectively, respectively.

In some embodiments, when such a molecule is a multi-specific antibody or fragment thereof, the molecule may be specific for two different antigens. In yet another aspect, provided herein are polynucleotides.

In some embodiments, a polynucleotide or polynucleotides according to the present invention may encode: (i) any of the variant CH1 domain polypeptides described above or any heavy chain polypeptides comprising any of the variant CH1 domains described above, (ii) any of the variant CLκ or CLλ domain polypeptides or any light chain polypeptides comprising any of the variant CLκ or CLλ domains described above; (iii) any of the polypeptides described above; and/or (iv) any of the molecules described above or vectors containing.

In some embodiments, a vector or vectors according to the present invention may comprise one or more of the polynucleotide(s) described above.

In yet another aspect, provided herein are cells which comprise (i) any of the variant CH1 domain polypeptides described above or any heavy chain polypeptides comprising any of the variant CH1 domains described above, (ii) any of the variant CLκ or CLλ domain polypeptides described above or any light chain polypeptides comprising any of the variant CLκ or CLλ domains; (iii) any of the immunoglobulin polypeptides described above; (iv) any of the molecules described above; (v) any of the polynucleotides described above; and/or (vi) any of the vectors described above.

In some embodiments, such a cell is a mammalian cell, optionally a Chinese hamster ovary (CHO) cell or a human embryonic kidney (HEK) cells such as HEK293 cells. In some embodiments, such a cell is a yeast cell.

In yet another aspect, provided herein are compositions which comprise: (I) (i) any of the variant CH1 domain polypeptides described above or any heavy chain polypeptides comprising any of the variant CH1 domains described above, (ii) any of the variant CLκ or CLλ domain polypeptides described above or any light chain polypeptides comprising any of the variant CLκ or CLλ domains; (iii) any of the immunoglobulin polypeptides described above; (iv) any of the molecules described above; (v) any of the polynucleotides described above; and/or (vi) any of the vectors described above; and/or (vii) any of the cells described above; and (II) a pharmaceutically or diagnostically acceptable carrier.

In yet another aspect, provided herein are methods of generating a CH1 domain library. Such a library may be a CH1 domain-encoding polynucleotide library or a CH1 domain polypeptide library.

In some embodiments, such a method of generating a CH1 domain-encoding polynucleotide library may comprise in silico or in vitro incorporating a mutation at or randomizing the nucleic acid at one or more pre-determined nucleotide positions in a plurality of CH1 domain-encoding polynucleotides, wherein at least one of the one or more pre-determined nucleotide positions may be within the codon(s) encoding the amino acid at one or more of pre-determined CH1 domain amino acid positions.

In certain embodiments, the one or more of pre-determined CH1 domain amino acid positions may be present in or proximate to the interface of a CH1 domain and a CL domain.

In certain embodiments, the one or more of pre-determined CH1 domain amino acid positions may be predicted to affect CH1-CL interdomain interaction. In some cases, the interaction may be hydrogen bond-mediated interaction. In some cases, the prediction may be performed in silico or in vitro. In particular cases, the prediction may be performed in silico using Rosetta Monte Carlo (MC) Hydrogen Bond Network (HBNet).

In certain embodiments, at least one of the one or more pre-determined nucleotide positions may be within the codon(s) encoding the amino acid at one or more of pre-determined CH1 domain amino acid positions selected from positions 145, 147, 181, 128, 124, 128, 139, 141, 145, 147, 148, 166, 168, 175, 181, 185, and 187 according to EU numbering.

In certain embodiments, incorporating a mutation and/or randomizing the nucleic acid may use a degenerate codon, optionally a degenerate RMW codon representing six naturally occurring amino acids (D, T, A, E, K, and N) or a degenerate NNK codon representing all 20 naturally occurring amino acid residues.

In certain embodiments, such a variant CH1 domain library may be for identifying one or more variant CH1 domain polypeptides which preferentially pairs with a given CL (CLκ or CLλ) domain or a variant CL (CLκ or CLλ) domain polypeptide rather than with a wild-type CL (CLκ or CLλ) domain polypeptide or rather than with another given variant CL (CLκ or CLλ) domain polypeptide.

CH1 domain-encoding polynucleotide libraries generated using such a method are also provided.

In some embodiments, such a method of generating a CH1 domain polypeptide library may comprise in silico or in vitro obtaining a plurality of CH1 domain polypeptides corresponding to a plurality of CH1 domain-encoding polynucleotides contained in such a CH1 domain-encoding polynucleotide library.

Alternatively, in some embodiments, a method of generating a CH1 domain polypeptide library may comprise in silico or in vitro incorporating a substitution at one or more pre-determined CH1 domain amino acid positions in a plurality of CH1 domain polypeptides.

In certain embodiments, wherein one or more of the one or more pre-determined CH1 domain amino acid position(s) may be: (i) present in or proximate to the interface of a CH1 domain and a CL domain; (ii) predicted to affect CH1-CL interdomain interaction, optionally hydrogen bond-mediated interaction, optionally wherein the prediction is performed in silico or in vitro, further optionally wherein the prediction is performed in silico using Rosetta MC HBNet; and/or (iii) selected from positions 145, 147, 181, 128, 124, 139, 141, 148, 166, 168, 175, 185, and 187, according to EU numbering.

In some embodiments, such a CH1 domain polypeptide library may be for identifying one or more variant CH1 domain polypeptides which preferentially pairs with a given or variant CL domain polypeptide rather than with a wild-type or another given variant CL domain polypeptide.

In some embodiments, such a CH1 domain polypeptide library may comprise a pre-determined number of CH1 substitution positions, optionally wherein the pre-determined number is 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

CH1 domain polypeptide libraries generated using such a method are also provided.

In yet another aspect, provided herein are methods of generating a CLκ and/or CLλ domain library. Such a library may be a CLκ and/or CLλ domain-encoding polynucleotide library or a CLκ and/or CLλ domain polypeptide library.

In some embodiments, such a method of generating a CLκ and/or CLλ domain-encoding polynucleotide library may comprise in silico or in vitro incorporating a mutation at or randomizing the nucleic acid at one or more pre-determined nucleotide positions in a plurality of CLκ and/or CLλ domain-encoding polynucleotides, wherein at least one of the one or more pre-determined nucleotide positions is within the codon(s) encoding the amino acid at one or more of pre-determined CLκ and/or CLλ domain amino acid positions.

In certain embodiments, the one or more of pre-determined CLκ and/or CLλ domain amino acid positions may be present in or proximate to the interface of a CH1 domain and a CLκ and/or CLλ domain.

In certain embodiments, the one or more of pre-determined CLκ and/or CLλ domain amino acid positions may be predicted to affect CH1-CL interdomain interaction. In some cases the interaction may be hydrogen bond-mediated interaction. In some cases, the prediction may be performed in silico or in vitro. In particular cases, the prediction may be performed in silico using Rosetta Monte Carlo (MC) Hydrogen Bond Network (HBNet).

In certain embodiments, at least one of the one or more pre-determined nucleotide positions may be within the codon(s) encoding the amino acid at one or more of pre-determined CLκ and/or CLλ domain amino acid positions selected from positions 129, 178, 180, 124, 133, 114, 120, 124, 127, 129, 133, 135, 137, and 138, 178, and 180, according to EU numbering.

In certain embodiments, incorporating a mutation and/or randomizing the nucleic acid may use a degenerate codon, optionally a degenerate RMW codon representing six naturally occurring amino acids (D, T, A, E, K, and N) or a degenerate NNK codon representing all 20 naturally occurring amino acid residues.

In certain embodiments, the variant CLκ and/or CLλ domain library may comprise CL domains of κ isotype only, CL domains of λ isotype only, or at least one CL domain of κ isotype and at least one CL domain of λ isotype.

In certain embodiments, such a variant CLκ and/or CLλ domain library may be for identifying one or more variant CLκ and/or CLλ domain polypeptides which preferentially pairs with a given or variant CH1 domain polypeptide rather than with a wild-type CH1 domain polypeptide or another given variant CH1 domain polypeptide.

In yet another aspect, provided herein are variant CLκ and/or CLλ domain libraries.

CLκ and/or CLλ domain-encoding polynucleotide libraries generated using the method described above are further provided.

In some embodiments, such a method of generating a CLκ and/or CLλ domain polypeptide library may comprise in silico or in vitro obtaining a plurality of CLκ and/or CLλ domain polypeptides corresponding to a plurality of CLκ and/or CLλ domain-encoding polynucleotides contained in the CLκ and/or CLλ domain-encoding polynucleotide library described above.

Alternatively, in some embodiments, such a method of generating a CLκ and/or CLλ domain polypeptide library may comprise in silico or in vitro incorporating a substitution at one or more pre-determined CLκ and/or CLλ domain amino acid positions in a plurality of CLκ and/or CLλ domain polypeptides.

In certain embodiments, the one or more of the one or more pre-determined CLκ and/or CLλ domain amino acid position(s) may be present in or proximate to the interface of a CH1 domain and a CL domain,

In certain embodiments, the one or more of the one or more pre-determined CLκ and/or CLλ domain amino acid position(s) may be predicted to affect CH1-CL interdomain interaction, optionally hydrogen bond-mediated interaction, optionally wherein the prediction is performed in silico or in vitro, further optionally wherein the prediction is performed in silico using Rosetta MC HBNet.

In certain embodiments, the one or more of the one or more pre-determined CLκ and/or CLλ domain amino acid position(s) may be selected from positions 129, 178, 180, 124, 133, 114, 120, 127, 135, 137, and 138, according to EU numbering.

In some embodiments, the library may be for identifying one or more variant CLκ and/or CLλ domain polypeptides which preferentially pairs with a given or variant CH1 domain polypeptide rather than with a wild-type or another given variant CH1 domain polypeptide.

In some embodiments, the CLκ and/or CLλ domain polypeptides of the library may comprise a pre-determined number of CLκ and/or CLλ substitution positions. In particular embodiments, the pre-determined number may be 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

CLκ and/or CLλ domain polypeptide library generated using the method described above are further provided herein.

In yet another aspect, provided herein are methods of generating a CH1-CL domain set library. Such a library may be a CH1-CL domain-encoding polynucleotide set library or a CH1-CL domain polypeptide set library.

In some embodiments, such a method of generating a CH1-CL domain-encoding polynucleotide set library may comprise in silico or in vitro incorporating a mutation at or randomizing the nucleic acid at one or more pre-determined nucleotide positions in a plurality of CH1-CL domain-encoding polynucleotide sets, wherein at least one of the one or more pre-determined nucleotide positions may be within the codon(s) encoding the amino acid at one or more of pre-determined CH1 and/or CL domain amino acid positions.

In certain embodiments, the one or more of pre-determined CH1 and/or CL domain amino acid positions may be present in or proximate to the interface of a CH1 domain and a CL domain;

In certain embodiments, the one or more of pre-determined CH1 and/or CL domain amino acid positions may be predicted to affect CH1-CL interdomain interaction. In some cases, the interaction may be hydrogen bond-mediated interaction. In some cases, the prediction may be performed in silico or in vitro. In particular cases, the prediction may be performed in silico using Rosetta Monte Carlo (MC) Hydrogen Bond Network (HBNet);

In certain embodiments, the one or more of pre-determined CH1 domain amino acid positions may be selected from CH1 positions 145, 147, 181, 128, 124, 139, 141, 148, 166, 168, 175, 185, and 187, according to EU numbering; and/or

In certain embodiments, the one or more of pre-determined CL domain amino acid positions may be selected from CL positions 129, 178, 180, 124, 133, 114, 120, 127, 135, 137, and 138, according to EU numbering,

In some embodiments, the one or more mutations may be generated via a degenerate codon, optionally a degenerate RMW codon representing six naturally occurring amino acids (D, T, A, E, K, and N) or a degenerate NNK codon representing all 20 naturally occurring amino acid residues.

In some embodiments, the library may be for identifying one or more variant CL domain polypeptides which preferentially pairs with a given or variant CH1 domain rather than or with a wild-type or another given variant CH1 domain polypeptide and/or for identifying one or more variant CH1 domain polypeptides which preferentially pairs with a given or variant CL domain rather than with a wild-type or another given variant CL domain polypeptide, or for identifying one or more sets of a variant CH1 domain and a variant CL domain that preferentially pair with each other.

In some embodiments, the CL domains encoded in the CH1-CL domain-encoding polynucleotide set library may comprise a CLκ domain(s) and/or a CLλ domain(s).

CH1-CL domain-encoding polynucleotide set libraries generated using such a method are also provided herein.

In some embodiments, such a method of generating a CH1-CL domain polypeptide set library may comprise in silico or in vitro obtaining a plurality of CH1-CL domain polypeptide sets corresponding to a plurality of CH1-CL domain-encoding polynucleotide sets contained in the CH1-CL domain-encoding polynucleotide set library described above.

Alternatively, in some embodiments, such a method of generating a CH1-CL domain polypeptide set library may comprise in silico or in vitro incorporating a substitution at one or more pre-determined CH1 and/or CL domain amino acid positions in a plurality of CH1-CL domain polypeptide sets.

In certain embodiments, the one or more of the one or more pre-determined CH1 and/or CL domain amino acid position(s) may be present in or proximate to the interface of a CH1 domain and a CL domain.

In certain embodiments, the one or more of the one or more pre-determined CH1 and/or CL domain amino acid position(s) may be predicted to affect CH1-CL interdomain interaction, optionally hydrogen bond-mediated interaction, optionally wherein the prediction is performed in silico or in vitro, further optionally wherein the prediction is performed in silico using Rosetta Monte Carlo (MC) Hydrogen Bond Network (HBNet).

In certain embodiments, the one or more of the one or more pre-determined CH1 and/or CL domain amino acid position(s) may be selected from CH1 domain amino acid positions 145, 147, 181, 128, 124, 139, 141, 148, 166, 168, 175, 185, and 187, according to EU numbering; and/or selected from CL domain amino acid positions 129, 178, 180, 124, 133, 114, 120, 127, 135, 137, and 138, according to EU numbering.

In some embodiments, the library may be for identifying one or more variant CL domain polypeptides which preferentially pairs with a given or variant CH1 domain rather than with a wild-type or another given variant CH1 domain polypeptide and/or for identifying one or more variant CH1 domain polypeptides which preferentially pairs with a given or variant CL domain rather than with a wild-type or another given variant CL domain polypeptide, or for identifying one or more sets of a variant CH1 domain and a variant CL domain that preferentially pair with each other.

In some embodiments, the CL domains encoded in the CH1-CL domain-encoding polynucleotide set library may comprise a CLκ domain(s) and/or a CLλ domain(s).

In some embodiments, the CH1 domain polypeptides of the CH1-CL domain polypeptide set library may comprise a pre-determined number of CH1 substitution positions, optionally wherein the pre-determined number is 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

In some embodiments, the CL domain polypeptides of the CH1-CL domain polypeptide set library comprises a pre-determined number of CL substitution positions, optionally wherein the pre-determined number is 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

In some embodiments, a method of generating a CH1-CL domain polypeptide set library may comprise: a first step of providing a plurality of CH1-CL domain polypeptide sets; a second step of calculating the CH1-CL interdomain interaction strength for one or more of the a plurality of CH1-CL domain polypeptide sets, optionally wherein the calculating is (a) in silico or in vitro, optionally in silico using Rosetta Monte Carlo (MC) Hydrogen Bond Network (HBNet) and/or (b) based on the strength of CH1-CL interdomain hydrogen bond(s) and/or of CH1-CL interdomain binding energy; a third step of selecting one or more CH1-CL domain polypeptide sets calculated to have stronger CH1-CL interdomain interaction compared to (a) a reference CH1-CL domain polypeptide set, which is optionally a WT CH1-CL domain polypeptide set or a known CH1-CL domain polypeptide set or (b) a reference CH1-CL interdomain interaction strength, which is optionally a the CH1-CL interdomain interaction strength of a WT CH1-CL domain set or of a known CH1-CL domain polypeptide set.

In some embodiments, the CH1-CL domain polypeptide set library may be for identifying one or more variant CL domain polypeptides which preferentially pairs with a variant CH1 domain polypeptide rather than with a wild-type or another given variant CH1 domain polypeptide.

In some embodiments, the CL domains in the CH1-CL domain polypeptide set library may comprise a CLκ domain(s) and/or a CLλ domain(s).

In some embodiments, the CH1 domain polypeptides of the CH1-CL domain polypeptide set library comprises a pre-determined number of CH1 substitution positions, optionally wherein the pre-determined number is 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5; and/or

In some embodiments, the CL domain polypeptides of the CH1-CL domain polypeptide set library comprises a pre-determined number of CL substitution positions, optionally wherein the pre-determined number is 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

In yet another aspect, provided herein CH1-CL domain polypeptide set libraries.

In some embodiments, such a library may be produced by any of the methods of generating a CH1-CL domain polypeptide set library described herein.

In some embodiments, the CH1-CL domain set library may be a CH1-CLκ domain set library, CH1-CLλ domain set library, or a CH1-CL domain set library in which the CL domains of the library comprise one or more CLκ domains and one or more CH1-CLλ domains.

In another aspect, provided herein are methods of identifying one or more sets of a variant CH1 domain polypeptide and a variant CLκ and/or CLλ domain polypeptide, wherein the variant CH1 domain polypeptide and the variant CLκ or CLλ domain polypeptide preferentially pair with each other.

In some embodiments, such a method may comprise three steps (steps (a) through (c)).

In some instances, the step (a) may comprise providing (a-1) a first polypeptide comprising a wild-type or a variant CH1 domain polypeptide and (a-2) a second polypeptide comprising a wild-type or variant CLκ or CLλ domain polypeptide. Optionally, the multiple sets of (a-1) and (a-2) are provided in silico or in vitro.

In particular instances, (i) said first polypeptide in step (a) may be derived from any CH1 domain polypeptide library described herein or expressed from any variant CH1 domain-encoding polynucleotide library described herein.

In particular instances, (ii) said second polypeptide in step (b) may be derived from any CLκ and/or CLλ domain polypeptide library or expressed from any CLκ and/or CLλ domain-encoding polynucleotide library described herein.

In particular instances, (iii) said first polypeptide in step (a) and said second polypeptide in step (b) may be derived from any CH1-CL domain polypeptide set library described herein or expressed from any CH1-CL domain-encoding polynucleotide set library described herein.

In particular instances, (iv) said first polypeptide in step (a) and said second polypeptide in step (b) may be expressed from a CH1-CL domain set library in which the CH1 and/or CL domains comprises one or more random amino acid modification(s).

In some instances, the step (b) may comprise quantifying the binding preference between the variant CH1 domain polypeptide and the variant CLκ or CLλ domain polypeptide.

In particular instances, the binding preference may be based on the strength of CH1-CL interdomain hydrogen bond(s) and/or of CH1-CL interdomain binding energy, further optionally wherein the quantifying is performed in silico or in vitro.

In some instances, the step (c) may comprise selecting one or more sets of a variant CH1 domain polypeptide and a variant CLκ or CLλ domain polypeptide which provide preferential CH1-CL paring. In some cases, the preferential CH1-CL pairing may be equivalent or higher preferential pairing relative to a reference CH1-CL domain polypeptide set. In certain cases, the reference CH1-CL domain polypeptide set may comprise a wildtype CH1 domain, a wildtype CLκ or CLλ domain, any of the variant CH1 domain polypeptides described above, and/or any of the variant CLκ or CLλ domain polypeptides described above. In certain cases, the reference CH1-CL domain polypeptide set may be a CH1-CL domain polypeptide set shown in Table 1.

In some embodiments, method of identifying may utilize the combinations of the amino acid substitutions in CH1 and/or CLκ or CLλ that were identified herein as influencing the light-heavy pairing.

In certain embodiments, the one or more predetermined CH1 domain amino acid positions may comprise or consist of positions 145, 147, and/or 181, and/or the one or more predetermined CLκ or CLλ domain amino acid positions may comprise or consist of positions 129, 178, and/or 180.

In certain embodiments, the one or more predetermined CH1 domain amino acid positions may comprise or consist of positions 128 and/or 147, and/or the one or more predetermined CLκ or CLλ domain amino acid positions may comprise or consist of positions 124, 133, and/or 178.

In certain embodiments, the one or more predetermined CH1 domain amino acid positions may comprise or consist of positions 168, 185, and/or 187, and/or the one or more predetermined CLκ or CLλ domain amino acid positions may comprise or consist of position 135.

In certain embodiments, the one or more predetermined CH1 domain amino acid positions may comprise or consist of positions 147 and/or 185, and/or the one or more predetermined CLκ or CLλ domain amino acid positions may comprise or consist of positions 135 and/or 178.

In certain embodiments, the one or more predetermined CH1 domain amino acid positions may comprise or consist of position 148, and/or the one or more predetermined CLκ or CLλ domain amino acid positions may comprise or consist of positions 124 and/or 129.

In certain embodiments, the one or more predetermined CH1 domain amino acid positions may comprise or consist of positions 139, 141, and/or 187, and/or the one or more predetermined CLκ or CLλ domain amino acid positions may comprise or consist of positions 114, 135, and/or 138.

In certain embodiments, the one or more predetermined CH1 domain amino acid positions may comprise or consist of positions 166 and/or 187, and/or the one or more predetermined CLκ or CLλ domain amino acid positions may comprise or consist of positions 137 and/or 138.

In certain embodiments, the one or more predetermined CH1 domain amino acid positions may comprise or consist of positions 168 and/or 185, and/or the one or more predetermined CLκ or CLλ domain amino acid positions may comprise or consist of position 135.

In certain embodiments, the one or more predetermined CH1 domain amino acid positions may comprise or consist of positions 124 and/or 147, and/or the one or more predetermined CLκ or CLλ domain amino acid positions may comprise or consist of positions 127 and/or 129.

In certain embodiments, the one or more predetermined CH1 domain amino acid positions may comprise or consist of positions 147 and/or 148, and/or the one or more predetermined CLκ or CLλ domain amino acid positions may comprise or consist of positions 127 and/or 129.

In certain embodiments, the one or more predetermined CH1 domain amino acid positions may comprise or consist of position 145, and/or the one or more predetermined CLκ or CLλ domain amino acid positions may comprise or consist of position 133.

In certain embodiments, the one or more predetermined CH1 domain amino acid positions may comprise or consist of positions 145 and/or 181, and/or the one or more predetermined CLκ or CLλ domain amino acid positions may comprise or consist of position 133.

In certain embodiments, the one or more predetermined CH1 domain amino acid positions may comprise or consist of position 145, and/or the one or more predetermined CLκ or CLλ domain amino acid positions may comprise or consist of positions 124 and/or 133.

In certain embodiments, the one or more predetermined CH1 domain amino acid positions may comprise or consist of positions 145 and/or 181, and/or the one or more predetermined CLκ or CLλ domain amino acid positions may comprise or consist of positions 120, 178, and/or 180.

In certain embodiments, the one or more predetermined CH1 domain amino acid positions may comprise or consist of positions 124, 145, and/or 147, and/or the one or more predetermined CLκ or CLλ domain amino acid positions may comprise or consist of positions 127, 129, and/or 178.

In certain embodiments, the one or more predetermined CH1 domain amino acid positions may comprise or consist of positions 166 and/or 187, and/or the one or more predetermined CLκ or CLλ domain amino acid positions may comprise or consist of positions 114, 137, and/or 138.

In certain embodiments, the one or more predetermined CH1 domain amino acid positions may comprise or consist of positions 147 and/or 175, and/or the one or more predetermined CLκ or CLλ domain amino acid positions may comprise or consist of positions 129, 178, and/or 180.

In certain embodiments, the one or more predetermined CH1 domain amino acid positions may comprise or consist of positions 147, 175, and/or 181, and/or the one or more predetermined CLκ or CLλ domain amino acid positions may comprise or consist of positions 129 and/or 180.

In certain embodiments, the one or more predetermined CH1 domain amino acid positions may comprise or consist of positions 145 and/or 147, and/or the one or more predetermined CLκ or CLλ domain amino acid positions may comprise or consist of positions 133 and/or 180.

In certain embodiments, the one or more predetermined CH1 domain amino acid positions may comprise or consist of positions 147 and/or 185, and/or the one or more predetermined CLκ or CLλ domain amino acid positions may comprise or consist of positions 129 and/or 180.

In some embodiments of the methods, the first polypeptide may comprise or may be linked to a first label; and/or the second polypeptide may comprise or may be linked to a second label.

In such embodiments, the quantifying step (b) may comprise detecting the first label and/or the second label.

In some embodiments of the methods, in step (a), the first polypeptide and the second polypeptide may be provided in step (a) in silico (e.g., computationally modeled in complex); and, in such cases, in step (b), the quantifying may comprise calculating a score which for example indicates the binding energy between the CH1 and CLκ domains, such as but not limited to the total energy or the energy from a hydrogen bond(s).

In such embodiments, the score may optionally be selected from: ΔΔG; ΔΔG_{cognate total score}; ΔΔG_{cognate hbond_all}; RBPP; RBPP_{total score}; RBPP_{hbond_all}; and/or RBPP_{bond elec backrub 18k}.

In some embodiments of the methods, in step (a), the first polypeptide and the second polypeptide may be provided in silico.

In such embodiments, the quantifying in step (b) may be performed in silico using Rosetta Monte Carlo (MC) Hydrogen Bond Network (HBNet).

In some embodiments of the methods, in step (a), the first polypeptide and the second polypeptide may be provided in vitro (e.g., recombinantly co-expressed); and, in such cases, in step (b), the quantifying comprises measuring the amounts of CH1-CLκ pairs via liquid chromatography-mass spectrometry (LC-MS), ion exchange chromatography (IEX), AlphaLISA®, and/or flow cytometry.

In some embodiments, the method of identifying may further comprise a step of selecting one or more CH1-CL domain sets based on one or more characteristics of an antibody comprising a set of first and second polypeptides selected in step (c).

In some embodiments, the one or more characteristics may be selected from the following: (i) (i-1) production yield, optionally assessed in one or more cell types, optionally mammalian cells such as CHO cells and HEK cells, yeast cells, insect cells, and/or plant cells and/or (i-2) compatibility to one or more antibody purification methods, optionally comprising protein A affinity purification; (ii) degree of aggregation, optionally presence of multimers of a full-size antibody; (iii) the rate of correct pairing, optionally correct paring between CH1 domains and/or between CH1 and CL domains; (iv) melting temperature (Tm) and/or aggregation temperature (Tagg), optionally Tagg266; (v) isoelectric point (“pI”); (vi) the level of interaction with polyspecificity reagent (“PSR”); (vii) hydrophobic interaction of the antibody; (viii) self-interaction; (ix) stability to high or low pH stress; (x) solubility; (xi) production costs and/or time; (xii) other stability parameters; (xiii) shelf life; (xiv) in vivo half-life; and/or (xv) immunogenicity.

Such antibody characteristics may be measured or assessed using any appropriate methods used in the field.

In certain embodiments, degree of aggregation, optionally presence of multimers of a full-size antibody, may be quantified using chromatography, optionally size exclusion chromatography (SEC) or electrophoresis, optionally SDS-PAGE.

In certain embodiments, the rate of correct pairing, optionally correct paring between CH1 domains and/or between CH1 and CL domains, may be assessed using LC-MS.

In certain embodiments, Tm and/or Tagg, optionally Tagg266, may be measured using Differential scanning fluorimetry (DSF) and/or Differential scanning calorimetry (DSC) and/or using an instrument, optionally Uncle®.

In certain embodiments, the level of interaction with PSR” may be measured the method described in in WO2014/179363.

In certain embodiments, hydrophobic interaction of the antibody may be measured using hydrophobic interaction chromatography (“HIC”), optionally as described in Estep P, et al. MAbs. 2015 May-June; 7(3): 553-561.

In certain embodiments, self-interaction may be measured by affinity-capture self-interaction nanoparticle spectroscopy (AC-SINS), optionally as described in Liu Y et al., MAbs. March-April 2014; 6(2):483-92.

In certain embodiments, self-interaction may be measured by dynamic light scattering (DLS).

Therefore, in another aspect, provided herein are methods of screening for a combination of (i) a first set of a first variant CH1 domain polypeptide and a first variant CL domain polypeptide (“first CH1-CL domain polypeptide set”) and (ii) a second set of a second variant CH1 domain polypeptide and a second variant CL domain polypeptide (“second CH1-CL domain polypeptide set”), wherein such a combination is suited for a multi-specific antibody or antigen-binding antibody fragment of interest which has an antibody or antibody fragment structure of interest (e.g., having the any of the structures described herein including structures in FIGS. 2-7 and/or optionally an IgG, still further optionally an IgG1, IgG2, IgG3 or IgG4) and/or which has antigen specificities of interest, optionally having variable region sequences of interest.

Such a method may comprise: (a) expressing a plurality of multi-specific antibodies and/or antigen-binding antibody fragments, comprising different combinations of (i) a first CH1-CL domain polypeptide set candidate and (ii) a second CH1-CL domain polypeptide set candidate; and (b) selecting one or more combinations of (i) a first CH1-CL domain polypeptide set and (ii) a second CH1-CL domain polypeptide set based on one or more characteristics of a plurality of the multi-specific antibodies and/or antigen-binding antibody fragments expressed in step (a).

In certain embodiments, at least one of the one or more characteristics may be selected from the characteristics (i)-(xv) above.

In some embodiments, the multiple multi-specific antibodies and/or antigen-binding antibody fragments comprise: (I) a first polypeptide comprising a first variant CH1 domain polypeptide and a first antigen-binding domain polypeptide; (II) a second polypeptide comprising a second variant CH1 domain polypeptide and a second antigen-binding domain polypeptide; (III) a third polypeptide comprising a first variant CL domain polypeptide and a third antigen-binding domain polypeptide; and (IV) a fourth polypeptide comprising a second variant CL domain polypeptide and a fourth antigen-binding domain polypeptide, optionally wherein the first and third polypeptide preferentially pair with each other and the second and fourth polypeptide preferentially pair with each other.

In particular embodiments, the plurality of multi-specific antibodies and/or antigen-binding antibody fragments may comprise a structure depicted in any of FIGS. 2-7.

In some instances, (i) the first variant CH1 domain polypeptide may be any of the variant CH1 domain polypeptides described herein; (ii) the second variant CH1 domain polypeptide may be any of the variant CH1 domain polypeptides described herein; (iii) the first CLκ or CLλ domain polypeptide may be any of the variant CLκ or CLλ domain polypeptides described herein; and/or (iv) the second CLκ or CLλ domain polypeptide may be any of the variant CLκ or CLλ domain polypeptides described herein.

In certain instances, the first antigen-binding domain and the third antigen-binding domain may form a first antigen-binding site specific for a first epitope of interest, and the second antigen-binding domain and the fourth antigen domain may form a second antigen-binding site specific for a second epitope of interest, optionally wherein the first epitope and second epitopes of interest differ from each other.

In certain instances, the first antigen-binding domain and the third antigen-binding domain may form a first antigen-binding site specific for a first epitope of interest, the second antigen-binding domain may form a second antigen-binding site specific for a second epitope of interest, and the fourth antigen-binding domain may form a third antigen-binding site specific for a third epitope of interest, optionally wherein the first epitope of interest differs from the second and/or third epitope(s) of interest.

In certain instances, the first antigen-binding domain may form a first antigen-binding site specific for a first epitope of interest, the second antigen-binding domain and the fourth antigen-binding domain may form a second antigen-binding site specific for a second epitope of interest, and the third antigen-binding domain may form a third antigen-binding site specific for a third epitope of interest, optionally wherein the second epitope of interest differs from the first and/or third epitope(s) of interest.

In certain instances, the first antigen-binding domain may form a first antigen-binding site specific for a first epitope of interest, and the second antigen-binding domain may form a second antigen-binding site specific for a second epitope of interest, the third antigen-binding domain may form a third antigen-binding site specific for a third epitope of interest, and the fourth antigen-binding domain may form a fourth antigen-binding site specific for a fourth epitope of interest, optionally wherein the first and/or third epitope(s) differ(s) from the second and/or fourth epitope(s).

In some embodiments, at least one of the one or more characteristics may be selected from the characteristics (i)-(xv) described above.

Also provided herein are libraries and methods for identifying one or more sets of a first polypeptide and a second polypeptide, which may preferentially pair with each other.

In one aspect, provided herein are methods of generating a library of sets of a first candidate polypeptide-encoding polynucleotide and a second candidate polypeptide-encoding polynucleotide, wherein (i) the first candidate polypeptide is the same as or is a variant of a first parent polypeptide; and (ii) the second candidate polypeptide is the same as or is a variant of a second parent polypeptide.

In some embodiments, the method may comprise (a) providing a set of a polynucleotide encoding the first parent polypeptide and a polynucleotide encoding the second parent polypeptide; and (b) in silico or in vitro incorporating a mutation at or randomizing the nucleic acid at one or more pre-determined nucleotide positions in the polynucleotide set of step (a), wherein at least one of the one or more pre-determined nucleotide positions is within the codon(s) encoding the amino acid at one or more of pre-determined amino acid positions of the first and/or second parent polypeptides.

In some embodiments, the one or more of pre-determined amino acid positions of the first and/or second parent polypeptides may be present in or proximate to the interface of the first parent polypeptide and the second parent polypeptide, optionally wherein the amino acid position(s) present in or proximate to the interface is predicted in silico or in vitro; and/or

In some embodiments, the one or more of pre-determined amino acid positions of the first and/or second parent polypeptides may be predicted to affect interaction between the first parent polypeptide and the second parent polypeptide, optionally inter-polypeptide hydrogen bond-mediated interaction and/or inter-polypeptide binding energy, optionally wherein the prediction is performed in silico or in vitro, further optionally wherein the prediction is performed in silico using Rosetta Monte Carlo (MC) Hydrogen Bond Network (HBNet).

In some embodiments, the one or more mutations may be generated via a degenerate codon, optionally a degenerate RMW codon representing six naturally occurring amino acids (D, T, A, E, K, and N) or a degenerate NNK codon representing all 20 naturally occurring amino acid residues.

In some embodiments, the library may be for identifying a first polypeptide and a second polypeptide which preferentially pair with each other, optionally relative to a set of the first parent polypeptide and the second parent polypeptide.

Libraries of sets of a first candidate polypeptide-encoding polynucleotide and a second candidate polypeptide-encoding polynucleotide generated using a method as described herein are also provided herein.

In another aspect, provided herein are methods of generating a library of sets of a first candidate polypeptide and a second candidate polypeptide, wherein: (i) the first candidate polypeptide is the same as or is a variant of a first parent polypeptide; and (ii) the second candidate polypeptide is the same as or is a variant of a second parent polypeptide.

In some embodiments, the method may comprise in silico or in vitro obtaining multiple sets of a first candidate polypeptide and a second candidate polypeptide corresponding to the first candidate polypeptide-encoding polynucleotides and the second candidate polypeptide-encoding polynucleotides contained in the polynucleotide set library as described above; or

In some embodiments, the method may comprise in silico or in vitro incorporating a substitution at one or more pre-determined amino acid positions of the first and/or second parent polypeptide(s).

In certain embodiments, the one or more of the one or more pre-determined amino acid position(s) may be present in or proximate to the interface of the first parent polypeptide and the second parent polypeptide, optionally wherein the amino acid position(s) present in or proximate to the interface is predicted in silico or in vitro; and/or

In certain embodiments, the one or more of the one or more pre-determined amino acid position(s) may be predicted to affect interaction between the first parent polypeptide and the second parent polypeptide, optionally inter-polypeptide hydrogen bond-mediated interaction and/or inter-polypeptide binding energy, optionally wherein the prediction is performed in silico or in vitro, further optionally wherein the prediction is performed in silico using Rosetta MC HBNet.

In some embodiments, the library may be for identifying a first polypeptide and a second polypeptide which preferentially pair with each other, optionally relative to a set of the first parent polypeptide and the second parent polypeptide.

In some embodiments, the first candidate polypeptides in the library may comprise a pre-determined number(s) of substitutions relative to the first parent polypeptide, optionally wherein the pre-determined number(s) is/are 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

In some embodiments, the second candidate polypeptides in the library may comprise a pre-determined number(s) of substitutions relative to the second parent polypeptide, optionally wherein the pre-determined number(s) is/are 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

Libraries of sets of a first candidate polypeptide and a second candidate polypeptide generated using a method described above are further provided herein.

In another aspect, provided herein are methods of identifying one or more sets of a first polypeptide and a second polypeptide, wherein: (i) the first polypeptide is the same as or is a variant of a first parent polypeptide; (ii) the second polypeptide is the same as or is a variant of a second parent polypeptide; (iii) the first polypeptide is a variant of the first parent polypeptide and/or the second polypeptide is a variant of the second parent polypeptide; and (iv) the first and second polypeptides preferentially pair with each other, optionally more preferentially compared to the first and second parent polypeptides.

In some embodiments, the method may comprise: (a) providing multiple sets of a first candidate polypeptide and a second candidate polypeptide, optionally wherein the providing is performed in silico or in vitro; (b) quantifying the binding preference between the first candidate polypeptide and the second candidate polypeptide, optionally wherein the binding preference is based on the strength of inter-polypeptide hydrogen bond(s) and/or of inter-polypeptide binding energy, further optionally wherein the quantifying is performed in silico or in vitro; and (c) selecting one or more sets of a first polypeptide and a second polypeptide which provide preferential inter-polypeptide paring, optionally equivalent or higher preferential pairing relative to a reference polypeptide set, further optionally wherein the reference polypeptide set is a set of (I) a first parent polypeptide or a variant thereof and (II) a second parent polypeptide or a variant thereof.

In some embodiments, at least one set of the first candidate polypeptide and the second candidate polypeptide in step (a) may be (i-1) derived from the library of sets of a first candidate polypeptide and a second candidate polypeptide as described above or (i-2) expressed from the library of sets of a first candidate polypeptide-encoding polynucleotide and a second candidate polypeptide-encoding polynucleotide as described above; and/or (ii) may be (ii-1) derived from a library of sets of a first candidate polypeptide and a second candidate polypeptide, in which the first and/or second candidate polypeptide(s) comprises one or more random amino acid modification(s), or (ii-2) expressed from a library of sets of a first candidate polypeptide-encoding polynucleotide and a second candidate polypeptide-encoding polynucleotide in which the first candidate polypeptide-encoding polynucleotide and/or the second candidate polypeptide-encoding polynucleotide comprise one or more random mutation(s).

In some embodiments, the first polypeptides may comprise or are linked to a first label; and/or the second polypeptides comprise or are linked to a second label, and in such an embodiment, optionally, the quantifying step (b) comprises detecting the first label and/or the second label.

In some embodiments, in step (a), the providing may be performed in silico; and in step (b), the quantifying may comprise calculating a score, optionally selected from: ΔΔG; ΔΔGcognate total score; ΔΔGcognate hbond_all; RBPP; RBPPtotal score; RBPPhbond_all; and/or RBPPbond elec backrub 18k; and/or the quantifying may be performed in silico using Rosetta Monte Carlo (MC) Hydrogen Bond Network (HBNet).

In some embodiments, in step (a), the providing may be performed in vitro, optionally recombinantly; and in step (b), the quantifying comprises measuring the amounts of CH1-CL pairs via liquid chromatography-mass spectrometry (LC-MS), ion exchange chromatography (IEX), AlphaLISA®, and/or flow cytometry.

### BRIEF DESCRIPTION OF THE DRAWINGS

FIGS. 1A-1D provide schematics which overall show the benefit of preferential pairing of a CH1 domain with a CL domain in various multi-specific antibody designs. In FIGS. 1A-1D, the bispecific antibody of interest (boxed) comprises: (a) a half antibody specific to epitope A, which comprises: (a-1) a heavy chain (“heavy chain A”) comprising a VH specific to epitope A (brick) and (a-2) a light chain (“light chain A”) comprising a VL specific to epitope A (horizontal stripe); and (b) a half antibody specific to epitope B, which comprises: (b-1) a heavy chain (“heavy chain B”) comprising a VH specific to epitope B (checker) and (b-2) a light chain (“light chain B”) comprising a VL specific to epitope B (vertical stripe).

FIG. 1A shows an exemplary production of such a bispecific antibody, when the heavy chain A, light chain A, heavy chain B, and light chain A all comprise wild-type constant domains. When such four chains are co-expressed, co-provided, or mixed at approximately a 1:1:1:1 ratio, ten different antibody products can be generated with the respective percentages as shown, if there is perfect promiscuity in inter-heavy-light chain pairing and inter-heavy-heavy chain pairing. Approximately 12.5% of the products will correspond to the bispecific antibody of interest (boxed).

FIG. 1B shows an exemplary production of a bispecific antibody of FIG. 1A but comprising a heavy chain heterodimerizing technology in heavy chains A and B. Any appropriate heavy chain heterodimerizing technology, may be used, such as but not limited to the “knobs-into-holes” technology (see, e.g., U.S. Pat. No. 5,731,168), which is CH3 domain modifications that promote CH3 heterodimerization. Although FIG. 1B depicts heavy-heavy chain heterodimerization technology only in the CH3 domains (a triangle added on one CH3 and a triangle taken out from the other, pairing CH3), a heterodimerizing modification(s) may exist in the hinge, CH2, and/or CH3 domain(s). When such heavy chain A, light chain A, heavy chain B, and light chain B are co-expressed, co-provided, or mixed at approximately a 1:1:1:1 ratio, and if the heavy chain heterodimerizing technology exclusively allows heavy-heavy hetero pairing, four different antibody products can be generated with the respective percentages as shown. Approximately 25% of the products will correspond to the bispecific antibody of interest (boxed).

FIG. 1C shows an exemplary production of such a bispecific antibody, when the heavy chain A comprises a variant CH1 domain (filled) which preferentially pairs with light chain A's variant CL domain (dot) rather than with light chain B's CL domain. The variant CH1 domain (filled) may be a variant CH1 domain according to the present disclosure and/or the variant CL domain (dot) may be a variant CL domain according to the present disclosure. The variant CH1 domain and the variant CL domain may be a variant CH1-CL domain set according to the present disclosure. The CH1 domain of heavy chain B and the CL domain of light chain B may be any appropriate CH1 and CL domains, wild-type or modified (such as another variant CH1-CL domain set according to the present disclosure, e.g., a variant CH1-CLκ domain set or a variant CH1-CLλ domain set). When such heavy chain A, light chain A, heavy chain B, and light chain B are co-expressed, co-provided, or mixed at approximately a 1:1:1:1 ratio, and if the variant CH1 domain (filled) and the variant CL domain (dot) exclusively pairs with each other, and if there is perfect promiscuity in inter-heavy-heavy chain pairing, three different antibody products can be generated with the respective percentages as shown. Approximately 50% of the products will correspond to the bispecific antibody of interest (boxed). When the pairing preference between the CH1 domain (filled) and the variant CL domain (dot) is closer to exclusive preference, the variety of products and the percentages of individual products will more resemble those shown in FIG. 1C. With a most ideal CH1-CH domain set, approximately 50% of the products will be the bispecific antibody of interest, but even if it does not reach 50%, CH1-CH domain sets that provide the bispecific antibody of interest at more than 12.5% (i.e., higher than when the corresponding wildtype CH1-CL set was used without a heavy chain heterodimerizing technology) facilitate efficient manufacturing of bispecific antibodies.

FIG. 1D shows an exemplary production of a bispecific antibody of FIG. 1C but which further comprises a heavy chain heterodimerizing technology in heavy chains A and B. Any appropriate heavy chain heterodimerizing technology, may be combined with a bispecific antibody comprising the variant CH1 domain and/or variant CL domain according to the present disclosure. Various heterodimerizing technologies are available, such as but not limited to the “knobs-into-holes” technology (see, e.g., U.S. Pat. No. 5,731,168), which is CH3 domain modifications that promote CH3 heterodimerization. Although FIG. 1D depicts heavy-heavy chain heterodimerization technology only in the CH3 domains (a triangle added on one CH3 and a triangle taken out from the other, pairing CH3), a heterodimerizing modification(s) may exist in the hinge, CH2, and/or CH3 domain(s). When such heavy chain A, light chain A, heavy chain B, and light chain B are co-expressed, co-provided, or mixed at approximately a 1:1:1:1 ratio, and if the variant CH1 domain (filled) and the variant CL domain (dot) exclusively pairs with each other, and if the heavy chain heterodimerizing technology exclusively allows heavy-heavy hetero pairing, only the intended antibody product (boxed) may be generated, i.e., 100%. With a most ideal CH1-CH domain set, approximately 100% of the products will be the bispecific antibody of interest, but even if it does not reach 100%, CH1-CHκ domain sets that provide the bispecific antibody of interest at more than 50% (i.e., higher than when the corresponding wildtype CH1-CL set was used with a heavy chain heterodimerizing technology) facilitate efficient manufacturing of bispecific antibodies.

FIGS. 2-8 provide exemplary and non-limiting embodiments of various multi-specific antibody structures with which the variant CH1 and/or variant CL domain disclosed herein may be used. In FIGS. 2-8, the following applies unless otherwise indicated: (1) Each domain is presented as a rectangle with the text therein showing the domain name (e.g., CH1, CL, etc); (2) a set of domains connected with each other represents a polypeptide (e.g., a heavy chain polypeptide, a light chain polypeptide, etc); (3) the direction of domains within a polypeptide is according to the direction of the text showing domain names, from the N-terminus to the C-terminus; (4) a linker or a hinge may be used between domains as necessary and a disulfide bond(s) may exist between polypeptides and/or between domains, perhaps to allow correct formation of an antigen-binding site(s), even when FIGS do not explicitly show a linker, a hinge, and/or a disulfide bond; (5) a CH2 and/or CH3 domain(s) shown in figures may be omitted whenever possible and, when appropriate, may be replaced with a hinge or a linker; (6) rectangles with no pattern (i.e., open) are domains which individually may comprise a corresponding wild-type sequence or may comprise one or more amino acid substitutions relative to a wild-type sequence; (7) CH1, CH2, and CH3 domains may individually be of any (heavy chain) isotype; (8) when more than one CH1 domains are present in a structure, the CH1 domains may or may not be of the same isotype, when more than one CH2 domains are present in a structure, the CH2 domains may or may not be of the same isotype, and when more than one CH3 domains are present in a structure, the CH3 domains may or may not be of the same isotype; (9) the hinge, CH2, and/or CH3 domains may comprise a modification(s) such as one(s) that facilitate(s) hetero pairing between two different hinges, two different CH2 domains, and/or two different CH3 domains; (10) a “CL” domain, unless particularly specified, may be a kappa or lambda CL domain; (11) a CH1 domain with a number separated by a hyphen (e.g., CH1-1, CH1-2, CH1-3, etc) preferentially or non-preferentially pair with a CL domain with the matching number (e.g., CL-1, CL-2, CL-3, etc, respectively); (12) a filled CH1 domain with a number separated by a hyphen (e.g., “CH1-1”, “CH1-2”, “CH1κ-3”, etc) is a variant CH1 domain that preferentially pairs with the dotted CL with the matching number (e.g., “CL-1”, “CL-2”, “CL-3”, etc, respectively), which may be a variant CLκ or CLλ domain according to the present disclosure, and the pair of the filled CH1 domain and the dotted CL pair may be a CH1-CL set according to the present disclosure; (13) CH1-CL sets with same numbering (e.g., a set of CH1-1 and CL-1 and another set of CH1κ-1 and CLκ-1) represents two of the same CH1-CLκ set (i.e., the substitutions are same between sets), and CH1-CL sets with different numbering (e.g., a set of CH1-1 and CL-1 and another set of CH1-2 and CL-2) are two CH1-CL sets that may be same or different from each other (the substitutions in the two sets may or may not be identical, and the CL isotype (kappa or lambda) may be same or different); (14) the pairing between an open CH1 domain with a number (e.g., CH1-1, CH1-2, CH1-3, etc) with an open CL domain with the matching number (e.g., CL-1, CL-2, CL-3, etc) may or may not be preferential pairing; (15) when such pairing between an open CH1 domain and an open CL domain is preferential pairing, the CH1-CL set may be any CH1-CL set that preferentially pairs with each other, for example, a CH1-CLκ or CH1-CLλ set according to the present disclosure, which may or may not be same as another preferentially pairing CH1-CL set used within the same structure; (16) VH-1 and VL-1 form an antigen-binding site for a first epitope, VH-2 and VL-2 form an antigen-binding site for a second epitope, VH-3 and VL-3 form an antigen-binding site for a third epitope, VH-4 and VL-4 form an antigen-binding site for a fourth epitope, VH-5 and VL-5 form an antigen-binding site for a fifth epitope, and VH-6 and VL-6 form an antigen-binding site for a sixth epitope; (17) all of the first through sixth epitopes may be different from each other, or not all of the first through sixth epitopes may be different from each other; and (18) in a given VH-VL pair, the VL may be omitted even if VL is shown in FIGS, if the VH alone gives sufficient specificity to a cognate antigen (e.g., nanobody).

FIG. 2A provides some exemplary and non-limiting embodiments of various multi-specific antibody structures with which the variant CH1 and/or CL domains disclosed herein may be used. The antibody on the top left (boxed) is an exemplary basic full-size bispecific antibody, in which hinges or disulfide bods are not explicitly shown. The boxed antibody may, for example, comprise a hinge between CH1-1 and CH2-1 and between CH1-2 and CH2-2 and a disulfide bond(s) may be present between the hinges, between CH1-1 and CL-1 domains, and between CH1-2 and CL-2 domains (center). Alternatively, the boxed antibody may, for example, comprise a hinge between CH1-1 and CH2-1 and between CH1-2 and CH2-2 and a disulfide bond may be present between hinges, between CL-1 and the hinge, and between CL-2 and the hinge (right). Hinges and disulfide bonds, such as those shown in middle and right antibody structures may be present, even if not explicitly shown, in any structures shown in FIGS and described herein.

FIG. 2B provides exemplary variations of the antibody structures shown in FIG. 2A. In some variants of the boxed antibody, the CH3 domains may be absent (top left), the CH2 domains may be absent (top right). In some variants, both the CH2 and CH3 domains may be absent (middle and bottom). In such cases, the hinges and disulfide bonds may be present as shown in middle. Alternatively, the multi-specificity may be provided by a mixture of two different Fab fragments of different specificity (bottom left) or a mix of two different Fab′ fragments of different specificity (bottom right). Even when not explicitly shown, any constant domain may be omitted as appropriate, in any of the structures in FIGS. 2-7 or variations thereof.

FIG. 2C provides exemplary variations of the boxed antibody structures shown in FIG. 2A. In addition to a first CH1-CL set, a second CH1-CL set is used. When the first and second sets are different sets, and the CH1 of the first set (i.e., CH1-1) preferentially binds to the CL of the first set (i.e., CL-1) rather than the CL of the second set (i.e., CL-2) and the CH1 of the second set (i.e., CH1-2) preferentially binds to the CL of the second set (i.e., CL-2) rather than the CL of the first set (i.e., CL-1), the structure facilitates improved efficiency in manufacturing. Even when not explicitly shown, an equivalent modification (additional, preferentially paring CH1-CL set) depicted in FIG. 2C may be applied as appropriate or desired, to any of the other structures in FIGS. 2-7 or variations thereof.

FIG. 2D provides exemplary variations of the boxed antibody structure shown in FIG. 2A. The structures comprise a heavy-heavy chain hetero pairing modification(s) (depicted as a triangle added on one domain and a triangle taken out from the other, pairing domain), in the CH3 (left), CH2 (middle), and/or CH3 (right) domain(s). Two different modification orientations (top vs bottom) are depicted. Even when not explicitly shown, an equivalent modification (addition of a heavy-heavy chain hetero paring technology) depicted in FIG. 2D may be applied as appropriate, to any of the structures in FIGS. 2-7 or variations thereof.

FIG. 3 provides further exemplary variations of the boxed antibody structure shown in FIG. 2A. The VH and VL positions are varied relative to the boxed structure in FIG. 2A. Even when not explicitly shown, an equivalent modification (switching VH and VL positions) depicted in FIG. 3 may be applied as appropriate, to any of the structures in FIGS. 2-7 or variations thereof.

FIG. 4A-4D provides further exemplary variations of the boxed antibody structure shown in FIG. 2A and of the variations in FIG. 3. CH1 and CL positions are varied relative to the FIGS. 2-3 structures. Equivalent variations (switching CH1 and CL positions) depicted in FIGS. 4A-4D may be further applied to any structures shown in FIGS. 2-7 or variations thereof as appropriate, even if not explicitly shown.

FIG. 5A provides exemplary variations of the boxed antibody structure of FIG. 2. Specifically, a VH-VL pair specific to a third epitope and a VH-VL pair specific to a fourth epitope are added to the N-terminus of the heavy and light chains in different orientations. Although both a VH-VL pair specific to a third epitope and a VH-VL pair specific to a fourth epitope are depicted, if desired, only one pair (only a pair specific to a third epitope or a pair specific to a fourth epitope) may be added. The third or fourth additional VH-VL pair may or may not be identical in paratope sequence composition and epitope specificity to the first or second VH-VL pair, respectively. Equivalent variations (addition of one or more VH-VL pairs) depicted in FIG. 5A may be further applied to any structures shown in FIGS. 2-7 or variations thereof as appropriate, even if not explicitly shown.

FIG. 5B provides exemplary variations of the boxed antibody structure of FIG. 5A. Specifically, a constant domain is further added between the two variable domains within the same polypeptide. In other words, a Fab (or Fab-like) fragment specific to a third epitope and a Fab (or Fab-like) fragment specific to a fourth epitope is added to the N-terminal side of the boxed antibody structure of FIG. 2. Although both a Fab (or Fab-like) fragment specific to a third epitope and a Fab (or Fab-like) fragment specific to a fourth epitope are depicted, if desired, only one Fab (or Fab-like) fragment (only a Fab (or Fab-like) fragment specific to a third epitope or a Fab (or Fab-like) fragment specific to a fourth epitope) may be added (see, e.g., FIG. 2C of Klein C. et al., Methods. 2019 Feb. 1; 154:21-31.). The third or fourth additional Fab (or Fab-like) fragment may or may not be identical in paratope sequence composition and epitope specificity to the first or second Fab (or Fab-like) fragment, respectively.

FIG. 5C provide additional variations of the boxed antibody structure of FIG. 2. Similar to structures in FIG. 5A, a VH-VL pair specific to a third epitope and a VH-VL pair specific to a fourth epitope are added in different orientations, and the order of VH and VL on light chains differ from that in FIG. 5A. Equivalent variations (addition of one or more VH-VL pairs) depicted in FIG. 5C may be further applied to any structures shown in FIGS. 2-7 or variations thereof as appropriate, even if not explicitly shown.

FIG. 6A-6E provide further variations of the boxed antibody structure of FIG. 2. Specifically, in FIGS. 6A-6D, a scFv specific to a third epitope and a scFv specific to a fourth epitope are added. Although two scFvs are depicted, if desired only one scFv may be added. In FIG. 6A, the scFvs are added to the C-terminus of the heavy chains. The four structures in FIG. 6A differ by the VH-VL order within each scFv. In FIG. 6B, the scFvs are added to the C-terminus of the light chains. The four structures in FIG. 6B differ by the VH-VL order within each scFv. In FIG. 6C, the scFvs are added to the N-terminus of the heavy chains. The four structures in FIG. 6C differ by the VH-VL order within each scFv. In FIG. 6D, the scFvs are added to the N-terminus of the light chains. The four structures in FIG. 6D differ by the VH-VL order within each scFv. Although not shown in FIGS. 6A-6D, the two scFvs may be added to different positions (e.g., one at the C-end of a heavy chain and one at the N-end of a light chain). In FIG. 6E, four scFvs are added to the N-terminus of the heavy and light chains. Although not explicitly shown, the VH-VL order within any one or more of the scFvs may be switched in the same manner as in FIG. 6A-6D. Furthermore, more than one scFvs may be added to any of the appropriate locations and location combinations (e.g. light chain N-terminus, light chain C-terminus, heavy chain N-terminus, and/or heavy chain C-terminus). Equivalent variations (addition of one or more scFvs) depicted in FIG. 6 may be further applied to any structures shown in FIGS. 2-7 or variations thereof as appropriate, even if not explicitly shown.

FIGS. 7A-7D provide further variations of the boxed antibody structure of FIG. 2. Specifically, a Fab fragment specific to a third epitope and a Fab fragment specific to a fourth epitope are added to the C-terminus of the heavy chains. Although two Fab fragments are depicted, only one Fab may be added, if desired. In FIG. 7A, the structure comprises at least one CH1-CL set, which may or may not be identical to a variant CH1-CL set according the present disclosure. As shown in FIG. 7B, at least two preferentially pairing CH1-CL sets, same or different, each of which may or may not be identical to a CH1-CL set according the present disclosure, may be used. For example, in case of the bispecific antibody structure in the middle, if the two heavy chains are identical to each other and if the two light chains are identical to each other, and when the CH1 domain and the CL domain of the CH1-CL set exclusively pair with each other, the use of the CH1-CL set will allow for an excellent production efficiency, providing only the intended bispecific antibody (i.e., approximately 100% of the products), without the need for a heavy-heavy chain hetero pairing technology. Alternatively, as shown in FIG. 7C, at least three CH1-CL sets, same or different from each other, each of which may or may not be identical to a CH1-CL set according the present disclosure, may be incorporated, and as shown in FIG. 7D, at least four CH1-CL sets, same or different from each other, each of which may or may not be identical to a CH1-CL set according the present disclosure, may be incorporated. Equivalent variations (addition of one or more Fab fragments) depicted in FIG. 7A-7D may be further applied to any structures shown in FIGS. 2-7 or variations thereof as appropriate, even if not explicitly shown.

FIGS. 8A-8D show results and an overall scheme of the screening in Example 1. FIG. 8A provides a graph showing the number of amino acid substitutions in the CH1 and CLκ domains in each of the unique CH1-CLκ pairs identified in the first stage of Example 1 (N=3164). FIG. 8B provides a matrix of the number of CLκ substitutions versus CH1 substitutions in each of the unique CH1-CLκ sets identified in the first stage of Example 1 (N=3164). FIG. 8C provides a plot generated in the second stage of Example 1, showing the distribution of the Rosetta sidechain hydrogen bond score term (ΔG_{hbond_sc_total}) as a function of the total number of substitutions in each of the CH1-CLκ sets. FIG. 8D provides a schematic of the screening in Example 1. In the first stage, MC HBNet was used for sampling sequence space with sidechain rotamer flexibility and fixed protein backbone, which resulted in 3164 unique CH1+CLκ sequence sets (results shown in FIGS. 8A-8B). In the second stage a Rosetta optimization step tested if the HBNet predicted hydrogen bonds hold up under optimization with both backbone and sidechain flexibility (results shown in FIG. 8C). The CH1-CLκ sets selected in Example 1 were subjected to in silico screening based on the interface binding energy in Example 2.

FIGS. 9A-9C show an overall scheme and the results of the screening in Example 2. FIG. 9A provides a scheme of screening steps of Example 2. 20 CH1-CLκ sets selected in Example 2 were subjected to experimental characterization as single interface design (SID) format in Example 3. FIG. 9B provides a graph comparing the interface binding energy changes ΔΔG_{total score backrub 18k} before (left) and after (right) WT reversion substitution(s) in the CH1-CLκ sets (individual sets referred to as individual networks in FIG. 9B) which were determined in Step 6 of Example 6 that the WT reverted sets rather than the non-reverted set will be carried forward. For example, the CH1-CLκ set referred as “network_2529” showed better interface binding energy profile once the substitution 145Q in CH1 was reverted to WT amino acid residue and the substitution 137Q in CLκ were was reverted to WT amino acid residue, and therefore the WT reverted set which comprises the WT amino acid residue at position 145 in CH1 and the WT amino acid residue at position 129 in CLκ were selected for experimental characterization in Example 3. The graph in FIG. 9B further compares ΔΔG_{total score backrub 18k} of design CH1-CLκ sets with that of mis-paired CH1-CLκ sets (i.e., sets in which either CH1 or CLκ is designed (i.e., not WT) but the other is WT). FIG. 9C provides a graph comparing the interface binding energy changes ΔΔG_{total score backrub 18k} for CH1-CLκ sets that were carried forward without a reversion(s),

FIG. 10 provides the scheme of LC-MS used in Examples for assessing bsAb production products. The left schematic shows the workflow. Part of the produced IgGs may be used for reduced full-length LC-MS. This may be used for confirming sequences and/or quantifying relative expression of different antibody chains. Part of the produced IgGs may be subjected to digestion to produce Fab fragments. A portion of the Fab fragments may be used for reduced LC-MS and another portion of the Fab fragments may be used for non-reduced LC-MS. Non-reduced LC-MS results provide % correctly paired (correct pairing between heavy and light) and reduced LC-MS results may be used to quantify relative amounts of different antibody chains after digestion. The right is an exemplary LC-MS data showing different peaks corresponding to different heavy-light chain pairs.

FIG. 11 shows a matrix which provides RBPP_{hbond+electrostatic backrun 18k} scores calculated for Abs comprising two different CH1-CLκ sets. Negative values indicate preferential pairing between the indicated CH1 and CLκ domains, and more negative values indicate more preferential pairing. For example, when network 1443 and Network 1993 are used as the two CH1-CLκ sets in a DID Ab, the RBPP_{hbond+electrostatic backrun 18k} score is as low as −6.1.

FIG. 12 shows wildtype CH1-CLκ interface and its electron density (paired with (shown in an orientation to be compared with) a human Fab named ADI-64596 of FIG. 13). Representative electron density in the region of interest for the Fab crystal structure of the panitumumab variable fragment (Fv) and a WT CH1 domain of IgG1 paired to a WT CLκ domain is shown. Heavy chain (HC) carbon atoms are colored light grey, kappa light chain (κLC) carbon atoms are colored white, nitrogen atoms are colored dark grey, and oxygen atoms are colored black. Protein is shown in stick representation. The 2Fo-Fc electron density map is shown as a grey mesh contoured at 1.0 with a 2.0 Å carve. Data for this crystal structure extends to 2.6 Å near-atomic resolution.

FIG. 13 shows the CH1-CLκ interface of ADI-64596 and its electron density. Representative electron density in the region of interest for the crystal structure of ADI-64596 comprising the panitumumab Fv and a variant CH1 (of IgG) domain comprising L145Q, K147E, and S181E paired to a CLκ domain comprising T129R, T178R, and T180Q (i.e., the CH1-CLκ set of Network 1443). HC carbon atoms are colored light grey, κLC carbon atoms are colored white, nitrogen atoms are colored dark grey, and oxygen atoms are colored black. Protein is shown in stick representation. The 2Fo-Fc electron density map is shown as a grey mesh contoured at 1.0σ with a 2.0 Å carve. Data for this crystal structure extend to 2.35 Å near-atomic resolution.

FIG. 14 shows the wildtype CH1-CLκ interface and its electron density (paired with (shown in an orientation to be compared with) a human Fab named ADI-64597 of FIG. 15). Representative electron density in the region of interest for the Fab crystal structure of the panitumumab Fv and a WT CH1 domain of IgG1 paired to a WT CLκ domain is shown. HC carbon atoms are colored light grey, κLC carbon atoms are colored white, nitrogen atoms are colored dark grey, and oxygen atoms are colored black. Protein is shown in stick representation. The 2Fo-Fc electron density map is shown as a grey mesh contoured at 1.06 with a 2.0 Å carve. Data for this crystal structure extend to 2.6 Å near-atomic resolution.

FIG. 15 shows the ADI-64597 CH1-CLκ interface and its electron density. Representative electron density in the region of interest for the crystal structure of ADI-64597 comprising the panitumumab Fv and a variant CH1 (of IgG) domain comprising L128R and K147R paired to a CLκ domain comprising Q124E, V133Q, and T178E (i.e., the CH1-CLκ set of Network 1993). HC carbon atoms are colored light grey, κLC carbon atoms are colored white, nitrogen atoms are colored dark grey, and oxygen atoms are colored black. Protein is shown in stick representation. The 2Fo-Fc electron density map is shown as a grey mesh contoured at 1.06 with a 2.0 Å carve. Data for this crystal structure extend to 2.2 Å near-atomic resolution.

FIG. 16 shows substitutions at the CH1-CLκ interface (Network 1443) present in ADI-64596 Fab which are responsible for nearly a dozen additional polar contacts not present in the Panitumumab WT CH1-CLκ interface. View of the novel hydrogen bond network at the ADI-64596 CH1-CLκ interface. HC carbon atoms are colored light grey, LC carbon atoms are colored white, nitrogen atoms are colored dark grey, and oxygen atoms are colored black. Side chains are shown in stick representation while main chain is shown as a cartoon. Hydrogen bonds are shown as black dotted lines while salt bridges are shown as light grey dotted lines.

FIG. 17 shows substitutions at the CH1-CLκ interface (Network 1993) present in ADI-64597 Fab which are responsible for several additional polar contacts not present in the Panitumumab WT CH1-CLκ interface. View of novel polar contacts at the ADI-64597 CH1-CLκ interface. HC carbon atoms are colored light grey, LC carbon atoms are colored white, nitrogen atoms are colored dark grey, and oxygen atoms are colored black. Side chains are shown in stick representation while main chains are shown as cartoon or a stick representation. Hydrogen bonds are shown as black dotted lines while salt bridges are shown as light grey dotted lines.

FIG. 18 shows several substitutions in ADI-64597 CH1 domain (i.e. Network 1993 CH1 domain) and in the orthogonal ADI-64596 CLκ domain (i.e., Network 1443 CLκ domain) which are predicted to sterically clash with each other, reducing propensity for mispairing. (a-c) views of the pairing interface surrounding the region of interest. Alignment of constant regions of ADI-64597 and ADI-64596 reveals steric clash at the CH1-CLκ interface of several substituted and unsubstituted positions for this potential mispaired construct including (a) L128R in CH1 and V at position 133 in CLκ, (b) K147R in CH1 and T129R in CLκ, and (c) S at position 183 in CH1 and T178R in CLκ. HC carbon atoms are colored light grey, LC carbon atoms are colored white, nitrogen atoms are colored dark grey, and oxygen atoms are colored black. Side chains are shown in stick representation, side chains involved in clashes are shown with a transparent molecular surface and main chain atoms are shown in cartoon representation.

FIG. 19 shows a matrix which provides RBPP_{hbond+electrostatic backrun 18k} scores calculated for Abs comprising two different CH1-CLκ sets. Negative values indicate preferential pairing between the indicated CH1 and CLλ domains, and more negative values indicate more preferential pairing. For example, when network 367 and Network 1612 are used as the two CH1-CLλ sets in a DID Ab, the RBPP_{hbond+electrostatic backrun 18k} score is as low as −4.8.

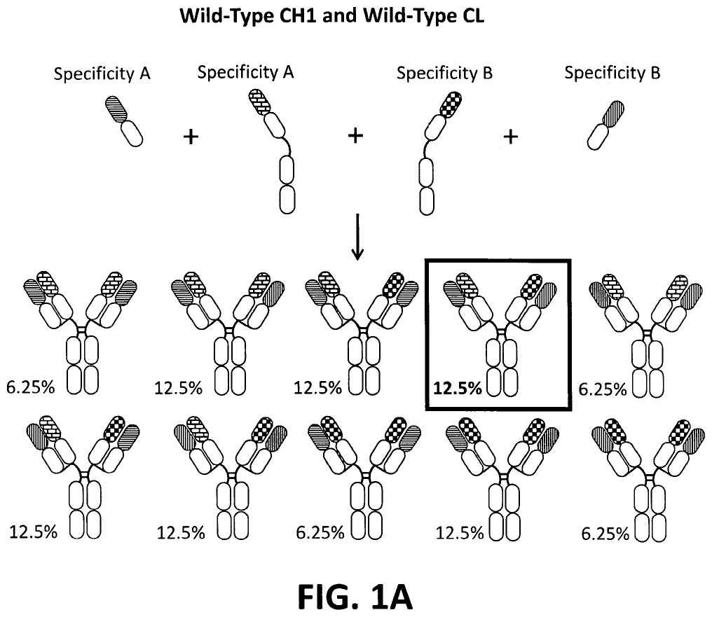

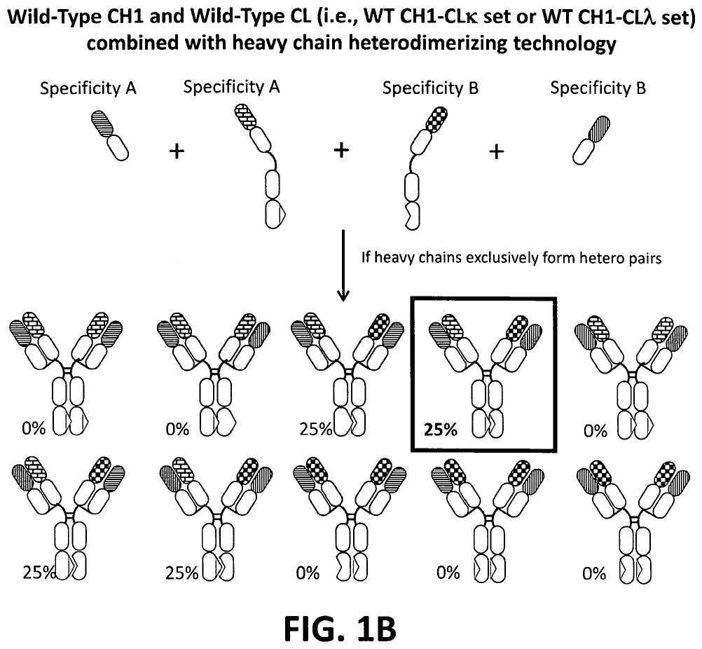

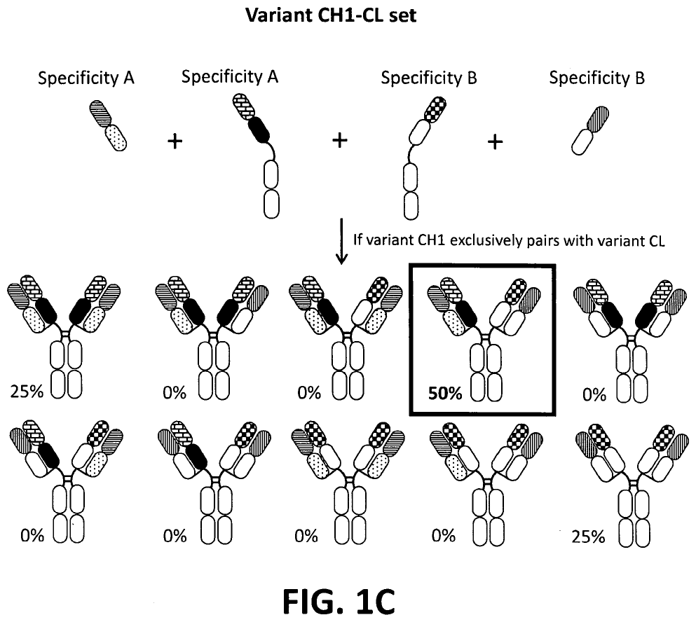

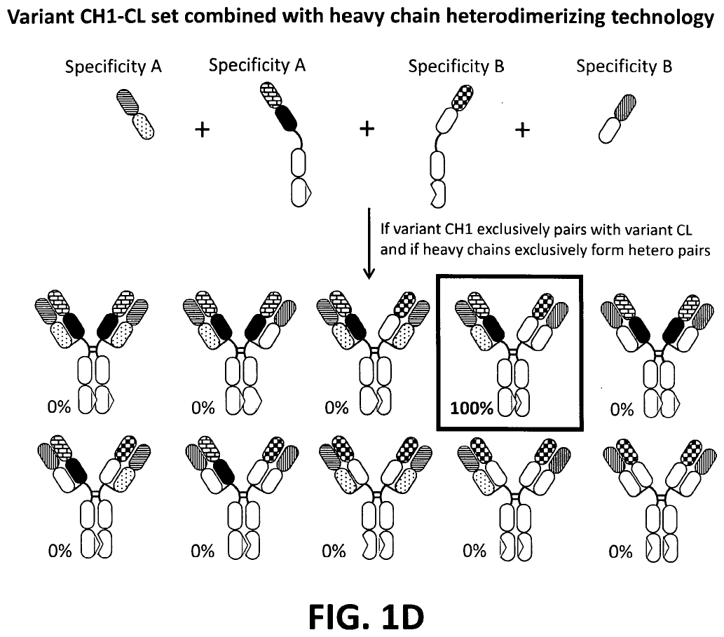

### DETAILED DESCRIPTION OF THE INVENTION

### Definitions

Unless defined otherwise, all technical and scientific terms used herein have the same meaning as commonly understood by one of ordinary skill in the art to which this disclosure belongs.

As used herein, the term “about,” when used in reference to a particular recited numerical value, means that the value may vary from the recited value by no more than 5%. For example, as used herein, the expression “about 100” includes 95 and 105 and all values in between (e.g., 96, 99, 99.5, 100.5, 104, etc.).

It is understood that aspects and embodiments of the disclosure described herein include “comprising,” “consisting,” and “consisting essentially of” aspects and embodiments.

The term “antibody” or “Ab” is used herein in the broadest sense and encompasses various antibody structures, including but not limited to monoclonal antibodies, polyclonal antibodies, multi-specific antibodies (e.g., bispecific antibodies), and/or antibody fragments (preferably those fragments that exhibit the desired antigen-binding activity, which is also referred to as “antigen-binding antibody fragments”). A “full antibody”, “full Ab”, “full-size antibody”, “full size Ab”, “full-length antibody”, “intact antibodies”, or “whole antibody”, or the like, encompasses molecules having a structure substantially similar to a native antibody. For example, an intact IgG (or IgD or IgE) antibody comprises two immunoglobulin heavy chains and two immunoglobulin light chains. An “antigen-binding fragment” or “antigen-binding antibody fragment” refers to a portion of an intact antibody or to a combination of portions derived from an intact antibody or from intact antibodies and binds the antigen(s) to which the intact antibody or antibodies bind.

In some instances, a full-size antibody, for example a full-size IgG or IgG-like antibody, comprises four polypeptide chains: two heavy chains (HCs) and two light chains (LCs) interconnected by disulfide bonds. Each HC comprises a variable region, such as a heavy chain variable region (“VH”), and a heavy chain constant region (“CH”). In case of an intact antibody, a CH comprises a CH1 domain, a hinge, a CH2 domain, and a CH3 domain. In case of an antibody fragment, when the fragment comprises a CH, the CH may comprise a CH1 domain, a hinge, a CH2 domain, and/or a CH3 domain, and in some preferred embodiments, the CH comprises at least a CH1 domain. The variant CH1 domains disclosed herein may be used in combination with wild-type CH2 and/or CH3 domains or CH2 and/or CH3 domains comprising one or more amino acid substitutions, e.g., those that alter or improve antibodies' stability and/or effector functions and/or those that promotes CH3 heterodimerization. Optionally, a hinge may also be used. Each LC comprises a variable region, such as a light chain variable region (“VL”), and a light chain constant region (“CL”). The VH and VL regions can be further subdivided into regions of hypervariability, termed complementarity determining regions (CDRs), interspersed with regions that are more conserved, termed framework regions (FRs). Each VH and VL comprises three CDRs and four FRs, arranged from amino-terminus to carboxy-terminus in the following order: FR1, CDR1, FR2, CDR2, FR3, CDR3, FR4. In certain embodiments of the disclosure, the FRs of the antibody (or antigen-binding fragment thereof) may be identical to the human germline sequences or may be naturally or artificially modified. An amino acid consensus sequence may be defined based on a side-by-side analysis of two or more CDRs. Accordingly, the three CDRs in a heavy chain are designated “CDRH1”, “CDRH2”, and “CDRH3”, respectively, and the three CDRs in a light chain are designated “CDRL1”, “CDRL2”, and “CDRL3”. In other instances, an antibody may comprise multimers thereof (e.g., IgM) or antigen-binding fragments thereof.

A light chain constant region (CL) domain of an antibody refers to the constant domain of the light chain of an antibody, located C-terminal of the variable region of the light chain. There are two major CL isotypes, kappa (“κ”) and lambda (“λ”), and such CL domains are referred to herein as kappa CL domain (“CLκ” domain) and lambda CL domain (“CLλ” domain). Unless specified, a CL domain may be CLκ or CLλ. In some instances, a CLκ domain may have the amino acid sequence encoded by any of the functional IGKC genes listed by IGMT. In some instances, a CLλ domain may have the amino acid sequence encoded by any of the functional IGLC genes listed by IGMT.

The numbering of amino acid residues in antibody variable and constant domains may be performed by the EU-index or EU numbering system, as described in Kabat et al., Sequences of Proteins of Immunological Interest, 5th Ed. Public Health Service, National Institutes of Health, Bethesda, Md. (1991). The EU numbering system is used in the present specification unless otherwise specified.

An “antigen-binding fragment of an antibody” or “antigen-binding antibody fragment” includes any naturally occurring, enzymatically obtainable, synthetic, or genetically engineered polypeptide or glycoprotein that comprises an antibody domain (e.g., a VH domain or a CH3 domain) specifically binds an antigen to form a complex. Exemplary antibody fragments include, but are not limited to: Fv; fragment antigen-binding (“Fab”) fragment; Fab′ fragment; Fab′ containing a free sulfhydryl group (‘Fab′-SH’); F(ab′)₂ fragment; diabodies; linear antibodies; single-chain antibody molecules (e.g. single-chain variable fragment (“scFv”), nanobody or VHH, or VH or VL domains only); and monospecific or multi-specific compounds formed from one or more of antibody fragments such as the foregoing. In some embodiments, the antigen-binding fragments of the bispecific antibodies described herein are scFvs or nanobodies. In some embodiments, an antigen-binding fragment comprises a variant CH1 domain, variant CLκ domain, and/or a variant CH1-CLκ set which preferentially form a CH1-CLκ pair rather than another CH1-CL pair. In some preferred embodiments, an antigen-binding fragment comprises a variant CH1 domain, variant CLλ domain, and/or a variant CH1-CLλ set which preferentially form a CH1-CLλ pair rather than forming another CH1-CL pair.

As with full antibody molecules, antigen-binding fragments may be mono-specific or multi-specific (e.g., bispecific, tri-specific, tetra-specific, etc). A multi-specific antigen-binding fragment of an antibody may comprise at least two antigen-binding sites (each containing at least one variable region such as a VH or a VL) which are capable of specifically binding to different antigens or epitopes.

A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing a naturally occurring mutation(s) and/or substitution(s) or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

A “multi-specific antibody”, which may also be referred to as “multi-specific compound” herein, refers to an antibody comprising at least two different antigen binding domains that recognize and specifically bind to at least two different antigens and/or at least two different epitopes. In some embodiments, a multi-specific antibody contains (1) a first heavy chain and a first light chain, which form a cognate pair and bind to a first antigen, and (2) a second heavy chain and a second light chain, which form a cognate pair and bind to a second antigen.

A “bispecific antibody”, which may also be referred to as “bispecific compound” herein, is a type of multi-specific antibody and refers to an antibody comprising two different antigen binding domains which recognize and specifically bind to at least two different antigens or at least two epitopes. The at least two epitopes may or may not be within the same antigen. A bispecific antibody may target, for example, two different surface receptors on the same or different (e.g., an immune cell and a cancer cell) cells, two different cytokines/chemokines, a receptor and a ligand.

In some embodiments, the at least two different antigens may be selected from the following antigens (or the at least two different epitopes may be epitopes within any of the following antigens): CD3; 0772P (CA125, MUC16; Genbank accession no. AF36148); adipophilin (perilipin-2, Adipose differentiation-related protein, ADRP, ADFP, MGC10598; NCBI Reference Sequence: NP-001113.2); AIM-2 (Absent In Melanoma 2, PYHIN4, Interferon-Inducible Protein AIM2; NCBI Reference Sequence: NP-004824.1); ALDH1 A1 (Aldehyde Dehydrogenase 1 Family, Member A1, ALDH1, PUMB1, Retinaldehyde Dehydrogenase 1, ALDC, ALDH-E1, ALHDII, RALDH 1, EC 1.2.1.36, ALDH11, HEL-9, HEL-S-53e, HEL12, RALDH1, Acetaldehyde Dehydrogenase 1, Aldehyde Dehydrogenase 1, Soluble, Aldehyde Dehydrogenase, Liver Cytosolic, ALDH Class 1, Epididymis Luminal Protein 12, Epididymis Luminal Protein 9, Epididymis Secretory Sperm Binding Protein Li 53e, Retinal Dehydrogenase 1, RaIDH1, Aldehyde Dehydrogenase Family 1 Member A1, Aldehyde Dehydrogenase, Cytosolic, EC 1.2.1; NCBI Reference Sequence: NP-000680.2); alpha-actinin-4 (ACTN4, Actinin, Alpha 4, FSGS1, Focal Segmental Glomerulosclerosis 1, Non-Muscle Alpha-Actinin 4, F-Actin Cross-Linking Protein, FSGS, ACTININ-4, Actinin Alpha4 Isoform, alpha-actinin-4; NCBI Reference Sequence: NP-004915.2); alpha-fetoprotein (AFP, HPAFP, FETA, alpha-1-fetoprotein, alpha-fetoglobulin, Alpha-1-fetoprotein, Alpha-fetoglobulin, HP; GenBank: AAB58754.1); Amphiregulin (AREG, SDGF, Schwannoma-Derived Growth Factor, Colorectum Cell-Derived Growth Factor, AR, CRDGF; GenBank: AAA51781.1); ARTC1 (ART1, ADP-Ribosyltransferase 1, Mono(ADP-Ribosyl)Transferase 1, ADP-Ribosyltransferase C2 And C3 Toxin-Like 1, ART2, CD296, RT6, ADP-Ribosyltransferase 2, GPI-Linked NAD(P)(+)-Arginine ADP-Ribosyltransferase 1, EC 2.4.2.31, CD296 Antigen; NP); ASLG659; ASPHD1 (Aspartate Beta-Hydroxylase Domain Containing 1, Aspartate Beta-Hydroxylase Domain-Containing Protein 1, EC 1.14.11., GenBank: AA144153.1); B7-H4 (VTCN1, V-Set Domain Containing T Cell Activation Inhibitor 1, B7H4, B7 Superfamily Member 1, Immune Costimulatory Protein B7-H4, B7h.5, T-Cell Costimulatory Molecule B7x, B7S1, B7X, VCTN1, H4, B7 Family Member, PRO1291, B7 Family Member, H4, T Cell Costimulatory Molecule B7x, V-Set Domain-Containing T-Cell Activation Inhibitor 1, Protein B7S1; GenBank: AAZ17406.1); BAFF-R (TNFRSF13C, Tumor Necrosis Factor Receptor Superfamily, Member 13C, BAFFR, B-Cell-Activating Factor Receptor, BAFF Receptor, BLyS Receptor 3, CVID4, BROMIX, CD268, B Cell-Activating Factor Receptor, prolixin, Tumor Necrosis Factor Receptor Superfamily Member 13C, BR3, CD268 Antigen; NCBI Reference Sequence: NP-443177.1); BAGE-1; BCLX (L); BCR-ABL fusion protein (b3a2); beta-catenin (CTNNB1, Catenin (Cadherin-Associated Protein), Beta 1, 88 kDa, CTNNB, MRD19, Catenin (Cadherin-Associated Protein), Beta 1 (88 kD), armadillo, Catenin Beta-1; GenBank: CAA61107.1); BING-4 (WDR46, WD Repeat Domain 46, C6orf11, BING4, WD Repeat-Containing Protein BING4, Chromosome 6 Open Reading Frame 11, FP221, UTP7, WD Repeat-Containing Protein 46; NP); BMPR1 B (bone morphogenetic protein receptor-type IB, Genbank accession no. NM-00120; NP); B-RAF (Brevican (BCAN, BEHAB, Genbank accession no. AF22905); Brevican (BCAN, Chondroitin Sulfate Proteoglycan 7, Brain-Enriched Hyaluronan-Binding Protein, BEHAB, CSPG7, Brevican Proteoglycan, Brevican Core Protein, Chondroitin Sulfate Proteoglycan BEHAB; GenBank: AAH27971.1); CALCA (Calcitonin-Related Polypeptide Alpha, CALC1, Calcitonin 1, calcitonin, Alpha-Type CGRP, Calcitonin Gene-Related Peptide I, CGRP-I, CGRP, CGRP1, CT, KC, Calcitonin/Calcitonin-Related Polypeptide, Alpha, katacalcin; NP); CASP-5 (CASP5, Caspase 5, Apoptosis-Related Cysteine Peptidase, Caspase 5, Apoptosis-Related Cysteine Protease, Protease ICH-3, Protease TY, ICE(rel)-111, ICE(rel)III, ICEREL-III, ICH-3, caspase-5, TY Protease, EC 3.4.22.58, ICH3, EC 3.4.22; NP); CASP-8; CD19 (CD19-B-lymphocyte antigen CD19 isoform 2 precursor, B4, CVID3 [Homo sapiens], NCBI Reference Sequence: NP-001761.3); CD20 (CD20-B-lymphocyte antigen CD20, membrane-spanning 4-domains, subfamily A, member 1, B1, Bp35, CD20, CVID5, LEU-16, MS4A2, S7; NCBI Reference Sequence: NP-690605.1); CD21 (CD21 (CR2 (Complement receptor or C3DR (C3d/Epstein Barr virus receptor) or Hs.73792 Genbank accession no. M2600); (CD22 (B-cell receptor CD22-B isoform, BL-CAM, Lyb-8, LybB, SIGLEC-2, FLJ22814, Genbank accession No. AK02646); CD22; CD33 (CD33 Molecule, CD33 Antigen (Gp67), Sialic Acid Binding Ig-Like Lectin 3, Sialic Acid-Binding Ig-Like Lectin 3, SIGLEC3, gp67, SIGLEC-3, Myeloid Cell Surface Antigen CD33, p67, Siglec-3, CD33 Antigen; GenBank: AAH28152.1); CD45; CD70 (CD70-tumor necrosis factor (ligand) superfamily, member 7; surface antigen CD70; Ki-24 antigen; CD27 ligand; CD27-L; tumor necrosis factor ligand superfamily member 7; NCBI Reference Sequence for species Homo sapiens: NP-001243.1); CD72 (CD72 (B-cell differentiation antigen CD72, Lyb-; 359 aa, μl: 8.66, MW: 40225, TM: 1 [P] Gene Chromosome: 9p13.3, Genbank accession No. NP-001773.); CD79a (CD79a (CD79A, CD79a, immunoglobulin-associated alpha, a B cell-specific protein that covalently interacts with Ig beta (CD79B) and forms a complex on the surface with Ig M molecules, transduces a signal involved in B-cell differentiation), μl: 4.84, MW: 25028 TM: 2 [P] Gene Chromosome: 19q13.2, Genbank accession No. NP-001774.1); CD79b (CD79b (CD79B, CD79b, IGb (immunoglobulin-associated beta), B29, Genbank accession no. NM-000626 or 1103867); Cdc27 (Cell Division Cycle 27, DOS1430E, D17S978E, Anaphase Promoting Complex Subunit 3, Anaphase-Promoting Complex Subunit 3, ANAPC3, APC3, CDC27Hs, H-NUC, CDC27 Homolog, Cell Division Cycle 27 Homolog (S. Cerevisiae), HNUC, NUC2, Anaphase-Promoting Complex, Protein 3, Cell Division Cycle 27 Homolog, Cell Division Cycle Protein 27 Homolog, Nuc2 Homolog; GenBank: AAH11656.1); CDK4 (Cyclin-Dependent Kinase 4, Cell Division Protein Kinase 4, PSK-J3, EC 2.7.11.22, CMM3, EC 2.7.11; NCBI Reference Sequence: NP-000066.1); CDKN2A (Cyclin-Dependent Kinase Inhibitor 2A, MLM, CDKN2, MTS1, Cyclin-Dependent Kinase Inhibitor 2A (Melanoma, P16, Inhibits CDK4), Cyclin-Dependent Kinase 4 Inhibitor A, Multiple Tumor Suppressor 1, CDK4I, MTS-1, CMM2, P16, ARF, INK4, INK4A, P14, P14ARF, P16-INK4A, P16INK4, P16INK4A, P19, P19ARF, TP16, CDK4 Inhibitor P16-INK4, Cell Cycle Negative Regulator Beta, p14ARF, p16-INK4, p16-INK4a, p16INK4A, p19ARF; NP); CEA; CLL1 (CLL-1 (CLEC12A, MICL, and DCAL, encodes a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. The protein encoded by this gene is a negative regulator of granulocyte and monocyte function. Several alternatively spliced transcript variants of this gene have been described, but the full-length nature of some of these variants has not been determined. This gene is closely linked to other CTL/CTLD superfamily members in the natural killer gene complex region on chromosome 12p13 (Drickamer, K Curr. Opin. Struct. Biol. 9:585-90 [1999]; van Rhenen, A, et al., Blood 110:2659-66 [2007]; Chen C H, et al. Blood 107:1459-67 [2006]; Marshall A S, et al. Eur. J. Immunol. 36:2159-69 [2006]; Bakker A B, et al Cancer Res. 64:8443-50 [2004]; Marshall A S, et al J. Biol. Chem. 279:14792-80, 2004. CLL-1 has been shown to be a type II transmembrane receptor comprising a single C-type lectin-like domain (which is not predicted to bind either calcium or sugar), a stalk region, a transmembrane domain and a short cytoplasmic tail containing an ITIM motif.); CLPP (Caseinolytic Mitochondrial Matrix Peptidase Proteolytic Subunit, Endopeptidase Clp, EC 3.4.21.92, PRLTS3, ATP-Dependent Protease ClpAP (E. coli), ClpP (Caseinolytic Protease, ATP-Dependent, Proteolytic Subunit, E. coli) Homolog, ClpP Caseinolytic Peptidase, ATP-Dependent, Proteolytic Subunit Homolog (E. coli), ClpP Caseinolytic Protease, ATP-Dependent, Proteolytic Subunit Homolog (E. coli), human, Proteolytic Subunit, ATP-Dependent Protease ClpAP, Proteolytic Subunit, Human, ClpP Caseinolytic Peptidase ATP-Dependent, Proteolytic Subunit, ClpP Caseinolytic Peptidase, ATP-Dependent, Proteolytic Subunit Homolog, ClpP Caseinolytic Protease, ATP-Dependent, Proteolytic Subunit Homolog, Putative ATP-Dependent Clp Protease Proteolytic Subunit, Mitochondrial; NP); COA-1; CPSF; CRIPTO (CRIPTO (CR, CR1, CRGF, CRIPTO, TDGF1, teratocarcinoma-derived growth factor, Genbank accession no. NP-003203 or NM-00321); Cw6; CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, μl: 8.54 MW: 41959 TM: 7 [P] Gene Chromosome: 11q23.3, Genbank accession No. NP-001707.); CXORF61 CXORF61-chromosome X open reading frame 61[Homo sapiens], NCBI Reference Sequence: NP-001017978.1); cyclin D1 (CCND1, BCL1, PRAD1, D11S287E, B-Cell CLL/Lymphoma 1, B-Cell Lymphoma 1 Protein, BCL-1 Oncogene, PRAD1 Oncogene, Cyclin D1 (PRAD1: Parathyroid Adenomatosis 1), G1/S-Specific Cyclin D1, Parathyroid Adenomatosis 1, U21B31, G1/S-Specific Cyclin-D1, BCL-1; NCBI Reference Sequence: NP-444284.1); Cyclin-A1 (CCNA1, CT146, Cyclin A1; GenBank: AAH36346.1); dek-can fusion protein; DKKI (Dickkopf WNT Signaling Pathway Inhibitor 1, SK, hDkk-1, Dickkopf (Xenopus laevis) Homolog 1, Dickkopf 1 Homolog (Xenopus laevis), DKK-1, Dickkopf 1 Homolog, Dickkopf Related Protein-1, Dickkopf-1 Like, Dickkopf-Like Protein 1, Dickkopf-Related Protein 1, Dickkopf-1, Dkk-1; GenBank: AAQ89364.1); DR1 (Down-Regulator Of Transcription 1, TBP-Binding (Negative Cofactor 2), Negative Cofactor 2-Beta, TATA-Binding Protein-Associated Phosphoprotein, NC2, NC2-BETA, Protein Dr1, NC2-beta, Down-Regulator Of Transcription 1; NCBI Reference Sequence: NP-001929.1); DR13 (Major Histocompatibility Complex, Class II, DR Beta 1, HLA-DR1B, DRw10, DW2.2/DR2.2, SS1, DRB1, HLA-DRB, HLA Class II Histocompatibility Antigen, DR-1 Beta Chain, Human Leucocyte Antigen DRB1, Lymphocyte Antigen DRB1, MHC Class II Antigen, MHC Class II HLA-DR Beta 1 Chain, MHC Class II HLA-DR-Beta Cell Surface Glycoprotein, MHC Class II HLA-DRw10-Beta, DR-1, DR-12, DR-13, DR-14, DR-16, DR-4, DR-5, DR-7, DR-8, DR-9, DR1, DR12, DR13, DR14, DR16, DR4, DR5, DR7, DRB, DR9, DRw11, DRw8, HLA-DRB2, Clone P2-Beta-3, MHC Class II Antigen DRB1*1, MHC Class II Antigen DRB1*10, MHC Class II Antigen DRB1*11, MHC Class II Antigen DRB1*12, MHC Class II Antigen DRB1*13, MHC Class II Antigen DRB1*14, MHC Class II Antigen DRB1*15, MHC Class II Antigen DRB1*16, MHC Class II Antigen DRB1*3, MHC Class II Antigen DRB1*4, MHC Class II Antigen DRB1*7, MHC Class II Antigen DRB1*8, MHC Class II Antigen DRB1*9; NP); E16 (E16 (LAT1, SLC7A5, Genbank accession no. NM-00348); EDAR (EDAR-tumor necrosis factor receptor superfamily member EDAR precursor, EDA-A1 receptor; downless homolog; ectodysplasin-A receptor; ectodermal dysplasia receptor; anhidrotic ectodysplasin receptor 1, DL; ECTD10A; ECTD10B; ED1R; ED3; ED5; EDA-A1R; EDA1R; EDA3; HRM1 [Homo sapiens]; NCBI Reference Sequence: NP-071731.1); EFTUD2 (Elongation Factor Tu GTP Binding Domain Containing 2, Elongation Factor Tu GTP-Binding Domain-Containing Protein 2, hSNU114, SNU114 Homolog, U5 SnRNP-Specific Protein, 116 KDa, MFDGA, KIAA0031, 116 KD, U5 SnRNP Specific Protein, 116 KDa U5 Small Nuclear Ribonucleoprotein Component, MFDM, SNRNP116, Snrp116, Snu114, U5-116 KD, SNRP116, U5-116 KDa; GenBank: AAH02360.1); EGFR (Epidermal Growth Factor Receptor, ERBB, Proto-Oncogene C-ErbB-1, Receptor Tyrosine-Protein Kinase ErbB-1, ERBB1, HER1, EC 2.7.10.1, Epidermal Growth Factor Receptor (Avian Erythroblastic Leukemia Viral (V-Erb-B) Oncogene Homolog), Erythroblastic Leukemia Viral (V-Erb-B) Oncogene Homolog (Avian), P1G61, Avian Erythroblastic Leukemia Viral (V-Erb-B) Oncogene Homolog, Cell Growth Inhibiting Protein 40, Cell Proliferation-Inducing Protein 61, mENA, EC 2.7.10; GenBank: AAH94761.1); EGFR-G719A; EGFR-G719C; EGFR-G719S; EGFR-L858R; EGFR-L861 Q; EGFR-57681; EGFR-T790M; Elongation factor 2 (EEF2, Eukaryotic Translation Elongation Factor 2, EF2, Polypeptidyl-TRNA Translocase, EF-2, SCA26, EEF-2; NCBI Reference Sequence: NP-001952.1); ENAH (hMena) (Enabled Homolog (Drosophila), MENA, Mammalian Enabled, ENA, NDPP1, Protein Enabled Homolog; GenBank: AAH95481.1)—results for just “ENAH” not “ENAH (hMena)”; EpCAM (Epithelial Cell Adhesion Molecule, M4S1, MIC18, Tumor-Associated Calcium Signal Transducer 1, TACSTD1, TROP1, Adenocarcinoma-Associated Antigen, Cell Surface Glycoprotein Trop-1, Epithelial Glycoprotein 314, Major Gastrointestinal Tumor-Associated Protein GA733-2, EGP314, KSA, DIAR5, HNPCC8, Antigen Identified By Monoclonal Antibody AUA1, EGP-2, EGP40, ESA, KS1/4, MK-1, Human Epithelial Glycoprotein-2, Membrane Component, Chromosome 4, Surface Marker (35 kD Glycoprotein), EGP, Ep-CAM, GA733-2, M1S2, CD326 Antigen, Epithelial Cell Surface Antigen, hEGP314, KS 1/4 Antigen, ACSTD1; GenBank: AAH14785.1); EphA3 (EPH Receptor A3, ETK1, ETK, TYRO4, HEK, Eph-Like Tyrosine Kinase 1, Tyrosine-Protein Kinase Receptor ETK1, EK4, EPH-Like Kinase 4, EC 2.7.10.1, EPHA3, HEK4, Ephrin Type-A Receptor 3, Human Embryo Kinase 1, TYRO4 Protein Tyrosine Kinase, hEK4, Human Embryo Kinase, Tyrosine-Protein Kinase TYRO4, EC 2.7.10; GenBank: AAH63282.1); EphB2R; Epiregulin (EREG, ER, proepiregulin; GenBank: AA136405.1); ETBR (EDNRB, Endothelin Receptor Type B, HSCR2, HSCR, Endothelin Receptor Non-Selective Type, ET-B, ET-BR, ETRB, ABCDS, WS4A, ETB, Endothelin B Receptor; NP); ETV6-AML1 fusion protein; EZH2 (Enhancer Of Zeste Homolog 2 (Drosophila), Lysine N-Methyltransferase 6, ENX-1, KMT6 EC 2.1.1.43, EZH1, WVS, Enhancer Of Zeste (Drosophila) Homolog 2, ENX1, EZH2b, KMT6A, WVS2, Histone-Lysine N-Methyltransferase EZH2, Enhancer Of Zeste Homolog 2, EC 2.1.1; GenBank: AAH10858.1); FcRH1 (FCRL1, Fc Receptor-Like 1, FCRH1, Fc Receptor Homolog 1, FcR-Like Protein 1, Immune Receptor Translocation-Associated Protein 5, IFGP1, IRTA5, hIFGP1, IFGP Family Protein 1, CD307a, Fc Receptor-Like Protein 1, Immunoglobulin Superfamily Fc Receptor, Gp42, FcRL1, CD307a Antigen; GenBank: AAH33690.1); FcRH2 (FCRL2, Fc Receptor-Like 2, SPAP1, SH2 Domain-Containing Phosphatase Anchor Protein 1, Fc Receptor Homolog 2, FcR-Like Protein 2, Immunoglobulin Receptor Translocation-Associated Protein 4, FCRH2, IFGP4, IRTA4, IFGP Family Protein 4, SPAP1A, SPAP1 B, SPAP1C, CD307b, Fe Receptor-Like Protein 2, Immune Receptor Translocation-Associated Protein 4, Immunoglobulin Superfamily Fc Receptor, Gp42, SH2 Domain Containing Phosphatase Anchor Protein 1, FcRL2, CD307b Antigen; GenBank: AAQ88497.1); FcRH5 (FCRL5, Fc Receptor-Like 5, IRTA2, Fc Receptor Homolog 5, FcR-Like Protein 5, Immune Receptor Translocation-Associated Protein 2, BXMAS1, FCRH5, CD307, CD307e, PRO820, Fc Receptor-Like Protein 5, Immunoglobulin Superfamily Receptor Translocation Associated 2 (IRTA2), FCRL5, CD307e Antigen; GenBank: AAI01070.1); FLT3-ITD; FN1 (Fibronectin 1, Cold-Insoluble Globulin, FN, Migration-Stimulating Factor, CIG, FNZ, GFND2, LETS, ED-B, FINC, GFND, MSF, fibronectin; GenBank: AA143764.1); G250 (MN, CAIX, Carbonic Anhydrase IX, Carbonic Dehydratase, RCC-Associated Protein G250, Carbonate Dehydratase IX, Membrane Antigen MN, Renal Cell Carcinoma-Associated Antigen G250, CA-IX, P54/58N, pMW1, RCC-Associated Antigen G250, Carbonic Anhydrase 9; NP);-alias results for “G250” not “G250/MN/CAIX”; GAGE-1,2,8; GAGE-3,4,5,6,7; GDNF-Ra1 (GDNF family receptor alpha 1; GFRA1; GDNFR; GDNFRA; RETL1; TRNR1; RET1 L; GDNFR-alpha1; GFR-ALPHA-; U95847; BC014962; NM-145793 NM-005264); GEDA (Genbank accession No. AY26076); GFRA1-GDNF family receptor alpha-1; GDNF receptor alpha-1; GDNFR-alpha-1; GFR-alpha-1; RET ligand 1; TGF-beta-related neurotrophic factor receptor 1 [Homo sapiens]; ProtKB/Swiss-Prot: P56159.2; glypican-3 (GPC3, Glypican 3, SDYS, Glypican Proteoglycan 3, Intestinal Protein OCI-5, GTR2-2, MXR7, SGBS1, DGSX, OCI-5. SGB, SGBS, Heparan Sulphate Proteoglycan, Secreted Glypican-3, OCIS; GenBank: AAH35972.1); GnTVf; gp100 (PMEL, Premelanosome Protein, SILV, D12S53E, PMEL17, SIL, Melanocyte Protein Pmel 17, Melanocytes Lineage-Specific Antigen GP100, Melanoma-Associated ME20 Antigen, Silver Locus Protein Homolog, ME20-M, ME20M, P1, P100, Silver (Mouse Homolog) Like, Silver Homolog (Mouse), ME20, SI, Melanocyte Protein Mel 17, Melanocyte Protein PMEL, Melanosomal Matrix Protein17, Silver, Mouse, Homolog Of, GenBank: AAC60634.1); GPC; GPNMB (Glycoprotein (Transmembrane) Nmb, Glycoprotein NMB, Glycoprotein Nmb-Like Protein, osteoactivin, Transmembrane Glycoprotein HGFIN, HGFIN, NMB, Transmembrane Glycoprotein, Transmembrane Glycoprotein NMB; GenBank: AAH32783.1); GPR172A (G protein-coupled receptor 172A; GPCR41; FLJ11856; D15Ertd747e); NP-078807.1; NM-024531.3); GPR19 (G protein-coupled receptor 19; Mm.478; NP-006134.1; NM-006143.2); GPR54 (KISS1 receptor; KISSIR; GPR54; HOT7T175; AXOR1; NP-115940.2; NM-032551.4); HAVCR1 (Hepatitis A Virus Cellular Receptor 1, T-Cell Immunoglobulin Mucin Family Member 1, Kidney Injury Molecule 1, KIM-1, KIM1, TIM, TIM-1, TIM1, TIMD-1, TIMD1, T-Cell Immunoglobulin Mucin Receptor 1, T-Cell Membrane Protein 1, HAVCR, HAVCR-1, T Cell Immunoglobin Domain And Mucin Domain Protein 1, HAVcr-1, T-Cell Immunoglobulin And Mucin Domain-Containing Protein 1; GenBank: AAH13325.1); HER2 (ERBB2, V-Erb-B2 Avian Erythroblastic Leukemia Viral Oncogene Homolog 2, NGL, NEU, Neuro/Glioblastoma Derived Oncogene Homolog, Metastatic Lymph Node Gene 19 Protein, Proto-Oncogene C-ErbB-2, Proto-Oncogene Neu, Tyrosine Kinase-Type Cell Surface Receptor HER2, MLN 19, p185erbB2, EC 2.7.10.1, V-Erb-B2 Avian Erythroblastic Leukemia Viral Oncogene Homolog 2 (Neuro/Glioblastoma Derived Oncogene Homolog), CD340, HER-2, HER-2/neu, TKR1, C-Erb B2/Neu Protein, herstatin, Neuroblastoma/Glioblastoma Derived Oncogene Homolog, Receptor Tyrosine-Protein Kinase ErbB-2, V-Erb-B2 Erythroblastic Leukemia Viral Oncogene Homolog 2, Neuro/Glioblastoma Derived Oncogene Homolog, MLN19, CD340 Antigen, EC 2.7.10; NP); HER-2/neu-alias of above; HERV-K-MEL; HLA-DOB (Beta subunit of MHC class II molecule (1a antigen) that binds peptides and presents them to CD4+T lymphocytes); 273 aa, μl: 6.56, MW: 30820.TM: 1 [P] Gene Chromosome: 6p21.3, Genbank accession No. NP-002111); hsp70-2 (HSPA2, Heat Shock 70 kDa Protein 2, Heat Shock 70 kD Protein 2, HSP70-3, Heat Shock-Related 70 KDa Protein 2, Heat Shock 70 KDa Protein 2; GenBank: AAD21815.1); IDO1 (Indoleamine 2,3-Dioxygenase 1, IDO, INDO, Indoleamine-Pyrrole 2,3-Dioxygenase, IDO-1, Indoleamine-Pyrrole 2,3 Dioxygenase, Indolamine 2,3 Dioxygenase, Indole 2,3 Dioxygenase, EC 1.13.11.52; NCBI Reference Sequence: NP-002155.1); IGF2B3; IL13Ralpha2 (IL13RA2, Interleukin 13 Receptor, Alpha 2, Cancer/Testis Antigen 19, Interleukin-13-Binding Protein, IL-13R-alpha-2, IL-13RA2, IL-13 Receptor Subunit Alpha-2, IL-13R Subunit Alpha-2, CD213A2, CT19, IL-13R, IL13BP, Interleukin 13 Binding Protein, Interleukin 13 Receptor Alpha 2 Chain, Interleukin-13 Receptor Subunit Alpha-2, IL13R, CD213a2 Antigen; NP); IL20Ru; Intestinal carboxyl esterase; IRTA2 (alias of FcRH5); Kallikrein 4 (KLK4, Kallikrein-Related Peptidase 4, PRSS17, EMSP1, Enamel Matrix Serine Proteinase 1, Kallikrein-Like Protein 1, Serine Protease 17, KLK-L1, PSTS, AI2A1, Kallikrein 4 (Prostase, Enamel Matrix, Prostate), ARM1, EMSP, Androgen-Regulated Message 1, Enamel Matrix Serine Protease 1, kallikrein, kallikrein-4, prostase, EC 3.4.21.-, Prostase, EC 3.4.21; GenBank: AAX30051.1); KIF20A (Kinesin Family Member 20A, RAB6KIFL, RAB6 Interacting, Kinesin-Like (Rabkinesin6), Mitotic a; LAGE-1; LDLR-fucosyltransferase AS fusion protein; Lengsin (LGSN, Lengsin, Lens Protein With Glutamine Synthetase Domain, GLULD1, Glutamate-Ammonia Ligase Domain-Containing Protein 1, LGS, Glutamate-Ammonia Ligase (Glutamine Synthetase) Domain Containing 1, Glutamate-Ammonia Ligase (Glutamine Synthase) Domain Containing 1, Lens Glutamine Synthase-Like; GenBank: AAF61255.1); LGR5 (leucine-rich repeat-containing G protein-coupled receptor 5; GPR49, GPR6; NP-003658.1; NM-003667.2; LY64 (Lymphocyte antigen 64 (RP10, type I membrane protein of the leucine rich repeat (LRR) family, regulates B-cell activation and apoptosis, loss of function is associated with increased disease activity in patients with systemic lupus erythematosis); 661 aa, μl: 6.20, MW: 74147 TM: 1 [P] Gene Chromosome: 5q12, Genbank accession No. NP-005573.; Ly6E (lymphocyte antigen 6 complex, locus E; Ly67, RIG-E, SCA-2, TSA-; NP-002337.1; NM-002346.2); Ly6G6D (lymphocyte antigen 6 complex, locus G6D; Ly6-D, MEGT; NP-067079.2; NM-021246.2); LY6K (lymphocyte antigen 6 complex, locus K; LY6K; HSJ001348; FLJ3522; NP-059997.3; NM-017527.3); LyPD1-LY6/PLAUR domain containing 1, PHTS [Homo sapiens], GenBank: AAH17318.1); MAGE-A1 (Melanoma Antigen Family A, 1 (Directs Expression Of Antigen MZ2-E, MAGE1, Melanoma Antigen Family A 1, MAGEA1, Melanoma Antigen MAGE-1, Melanoma-Associated Antigen 1, Melanoma-Associated Antigen MZ2-E, Antigen MZ2-E, Cancer/Testis Antigen 1.1, CT1.1, MAGE-1 Antigen, Cancer/Testis Antigen Family 1, Member 1, Cancer/Testis Antigen Family 1, Member 1, MAGE1A; NCBI Reference Sequence: NP-004979.3); MAGE-A10 (MAGEA10, Melanoma Antigen Family A, 10, MAGE10, MAGE-10 Antigen, Melanoma-Associated Antigen 10, Cancer/Testis Antigen 1.10, CT1.10, Cancer/Testis Antigen Family 1, Member 10, Cancer/Testis Antigen Family 1, Member 10; NCBI Reference Sequence: NP-001238757.1); MAGE-A12 (MAGEA12, Melanoma Antigen Family A, 12, MAGE12, Cancer/Testis Antigen 1.12, CT1.12, MAGE12F Antigen, Cancer/Testis Antigen Family 1, Member 12, Cancer/Testis Antigen Family 1, Member 12, Melanoma-Associated Antigen 12, MAGE-12 Antigen; NCBI Reference Sequence: NP-001159859.1); MAGE-A2 (MAGEA2, Melanoma Antigen Family A, 2, MAGE2, Cancer/Testis Antigen 1.2, CT1.2, MAGEA2A, MAGE-2 Antigen, Cancer/Testis Antigen Family 1, Member 2, Cancer/Testis Antigen Family 1, Member 2, Melanoma Antigen 2, Melanoma-Associated Antigen 2; NCBI Reference Sequence: NP-001269434.1); MAGE-A3 (MAGEA3, Melanoma Antigen Family A, 3, MAGE3, MAGE-3 Antigen, Antigen MZ2-D, Melanoma-Associated Antigen 3, Cancer/Testis Antigen 1.3, CT1.3, Cancer/Testis Antigen Family 1, Member 3, HIPS, HYPD, MAGEA6, Cancer/Testis Antigen Family 1, Member 3; NCBI Reference Sequence: NP-005353.1); MAGE-A4 (MAGEA4, Melanoma Antigen Family A, 4, MAGE4, Melanoma-Associated Antigen 4, Cancer/Testis Antigen 1.4, CT1.4, MAGE-4 Antigen, MAGE-41 Antigen, MAGE-X2 Antigen, MAGE4A, MAGE4B, Cancer/Testis Antigen Family 1, Member 4, MAGE-41, MAGE-X2, Cancer/Testis Antigen Family 1, Member 4; NCBI Reference Sequence: NP-001011550.1); MAGE-A6 (MAGEA6, Melanoma Antigen Family A, 6, MAGE6, MAGE-6 Antigen, Melanoma-Associated Antigen 6, Cancer/Testis Antigen 1.6, CT1.6, MAGE3B Antigen, Cancer/Testis Antigen Family 1, Melanoma Antigen Family A 6, Member 6, MAGE-3b, MAGE3B, Cancer/Testis Antigen Family 1, Member 6; NCBI Reference Sequence: NP-787064.1); MAGE-A9 (MAGEA9, Melanoma Antigen Family A, 9, MAGE9, MAGE-9 Antigen, Melanoma-Associated Antigen 9, Cancer/Testis Antigen 1.9, CT1.9, Cancer/Testis Antigen Family 1, Member 9, Cancer/Testis Antigen Family 1, Member 9, MAGEA9A; NCBI Reference Sequence: NP-005356.1); MAGE-C1 (MAGEC1, Melanoma Antigen Family C, 1, Cancer/Testis Antigen 7.1, CT7.1, MAGE-C1 Antigen, Cancer/Testis Antigen Family 7, Member 1, CT7, Cancer/Testis Antigen Family 7, Member 1, Melanoma-Associated Antigen C1; NCBI Reference Sequence: NP-005453.2); MAGE-C2 (MAGEC2, Melanoma Antigen Family C, 2, MAGEE1, Cancer/Testis Antigen 10, CT10, HCA587, Melanoma Antigen, Family E, 1, Cancer/Testis Specific, Hepatocellular Carcinoma-Associated Antigen 587, MAGE-C2 Antigen, MAGE-E1 Antigen, Hepatocellular Cancer Antigen 587, Melanoma-Associated Antigen C2; NCBI Reference Sequence: NP-057333.1); mammaglobin-A (SCGB2A2, Secretoglobin, Family 2A, Member 2, MGB1, Mammaglobin 1, UGB2, Mammaglobin A, mammaglobin-A, Mammaglobin-1, Secretoglobin Family 2A Member 2; NP); MART2 (H HAT, Hedgehog Acyltransferase, SKI1, Melanoma Antigen Recognized By T-Cells 2, Skinny Hedgehog Protein 1, Skn, Melanoma Antigen Recognized By T Cells 2, Protein-Cysteine N-Palmitoyltransferase HHAT, EC 2.3.1.-; GenBank: AAH39071.1); M-CSF (CSF1, Colony Stimulating Factor 1 (Macrophage), MCSF, CSF-1, lanimostim, Macrophage Colony-Stimulating Factor 1, Lanimostim; GenBank: AAH21117.1); MCSP (SMCP, Sperm Mitochondria-Associated Cysteine-Rich Protein, MCS, Mitochondrial Capsule Selenoprotein, HSMCSGEN1, Sperm Mitochondrial-Associated Cysteine-Rich Protein; NCBI Reference Sequence: NP-109588.2); XAGE-1b/GAGED2a; WT1 (Wilms Tumor 1, WAGR, GUD, WIT-2, WT33, Amino-Terminal Domain Of EWS, NPHS4, Last Three Zinc Fingers Of The DNA-Binding Domain Of WT1, AWT1, Wilms Tumor Protein, EWS-WT1; GenBank: AAB33443.1); VEGF; Tyrosinase (TYR; OCAIA; OCA1A; tyrosinase; SHEP; NP-000363.1; NM-000372.4; GenBank: AAB60319.1); TrpM4 (BR22450, FLJ20041, TRPM4, TRPM4B, transient receptor potential cation channel, subfamily M, member 4, Genbank accession no. NM-01763); TRP2-INT2; TRP-2; TRP-1/gp7S (Tyrosinase-Related Protein 1, 5,6-Dihydroxyindole-2-Carboxylic Acid Oxidase, CAS2, CATB, TYRP, OCAS, Catalase B, b-PROTEIN, Glycoprotein 7S, EC 1.14.18., Melanoma Antigen Gp7S, TYRP1, TRP, TYRRP, TRP1, SHEP11, DHICA Oxidase, EC 1.14.18, GP7S, EC 1.14.18.1; Triosephosphate isomerase (Triosephosphate isomerase 1, TPID, Triose-Phosphate Isomerase, HEL-S-49, TIM, Epididymis Secretory Protein Li 49, TPI, Triosephosphate Isomerase, EC 5.3.1.1; TRAG-3 (CSAG Family Member 2, Cancer/Testis Antigen Family 24, CSAG3B, Member 2, CSAG Family Member 3B, Cancer/Testis Antigen Family 24 Member 2, Cancer/Testis Antigen 24.2, Chondrosarcoma-Associated Gene 2/3 Protein, Taxol-Resistant-Associated Gene 3 Protein, Chondrosarcoma-Associated Gene 2/3 Protein-Like, CT24.2, Taxol Resistance Associated Gene 3, TRAG-3, CSAG3A, TRAG3;); TMEM46 (shisa homolog 2 (Xenopus laevis); SHISA; NP-001007539.1; NM-001007538.1; TMEM118 (ring finger protein, transmembrane2; RNFT2; FLJ1462; NP-001103373.1; NM-001109903.1; TMEFF1 (transmembrane protein with EGF-like and two follistatin-like domains 1; Tomoregulin-; H7365; C9orf2; C90RF2; U19878; X83961; NM-080655; NM-003692; TGF-betaRII (TGFBR2, Transforming Growth Factor, Beta Receptor II (70/80 kDa), TGFbeta-RII, MFS2, tbetaR-II, TGFR-2, TGF-Beta Receptor Type IIB, TGF-Beta Type II Receptor, TGF-Beta Receptor Type-2, EC 2.7.11.30, Transforming Growth Factor Beta Receptor Type IIC, AAT3, TbetaR-II, Transforming Growth Factor, Beta Receptor 11(70-80 kD), TGF-Beta Receptor Type II, FAA3, Transforming Growth Factor-Beta Receptor Type II, LDS1 B, HNPCC6, LDS2B, LDS2, RITC, EC 2.7.11, TAAD2; TENB2 (TMEFF2, tomoregulin, TPEF, HPP1, TR, putative transmembrane proteoglycan, related to the EGF/heregulin family of growth factors and follistatin); 374 aa, NCBI Accession: AAD55776, AAF91397, ΔΔG49451, NCBI RefSeq: NP-057276; NCBI Gene: 23671; OMIM: 605734; SwissProt Q9UIK5; Genbank accession No. AF179274; AY358907, CAF85723, CQ782436; TAG-2; TAG-1 (Contactin 2 (Axonal), TAG-1, AXT, Axonin-1 Cell Adhesion Molecule, TAX, Contactin 2 (transiently Expressed), TAXI, Contactin-2, Axonal Glycoprotein TAG-1, Transiently-Expressed Axonal Glycoprotein, Transient Axonal Glycoprotein, Axonin-1, TAX-1, TAGI, FAMES; PRF: 444868); SYT-SSX1 or -SSX2 fusion protein; survivin; STEAP2 (HGNC 8639, IPCA-1, PCANAP1, STAMPI, STEAP2, STMP, prostate cancer associated gene 1, prostate cancer associated protein 1, six transmembrane epithelial antigen of prostate 2, six transmembrane prostate protein, Genbank accession no. AF45513; STEAP1 (six transmembrane epithelial antigen of prostate, Genbank accession no. NM-01244; SSX-4; SSX-2 (SSX2, Synovial Sarcoma, X Breakpoint2, X Breakpoint 2, SSX, X Breakpoint 2B, Cancer/Testis Antigen 5.2, X-Chromosome-Related 2, Tumor Antigen HOM-MEL-40, CT5.2, HD21, Cancer/Testis Antigen Family 5, HOM-MEL-40, Isoform B, Cancer/Testis Antigen Family 5 member 2a, member 2a, Protein SSX2, Sarcoma, Sarcoma, Synovial, X-Chromosome-Related 2, synovial, Synovial Sarcoma, X Breakpoint 2B, Synovial Sarcomam, SSX2A; Sp17; SOX10 (SRY (Sex Determining Region Y)-Box 10, mouse, PCWH, DOM, WS4, WS2E, WS4C, Dominant Megacolon, mouse, Human Homolog Of, Dominant Megacolon, SRY-Related HMG-Box Gene 10, Human Homolog Of, transcription Factor SOX-10; GenBank: CAG30470.1); SNRPD1 (Small Nuclear Ribonucleoprotein D1, Small Nuclear Ribonucleoprotein D1, Polypeptide 16 kDa, Polypeptide (16 kD), SNRPD, HsT2456, Sm-D1, SMD1, Sm-D Autoantigen, Small Nuclear Ribonucleoprotein D1 Polypeptide 16 kDa Pseudogene, SnRNP Core Protein D1, Small Nuclear Ribonucleoprotein Sm D1; SLC35D3 (Solute Carrier Family 35, Member D3, FRCL1, Fringe Connection-Like Protein 1, bA55K22.3, Frc, Fringe-Like 1, Solute Carrier Family 35 Member D3; NCBI GenBank: NC-000006.11 NC-018917.2 NT-025741.16); SIRT2 (Sirtuin 2, NAD-Dependent Deacetylase Sirtuin-2, SIRL2, Silent Information Regulator 2, Regulatory Protein SIR2 Homolog 2, Sir2-Related Protein Type 2, SIR2-Like Protein 2, Sirtuin Type 2, Sirtuin (Silent Mating Type Information Regulation 2 Homolog) 2 (S. cerevisiae), Sirtuin-2, Sirtuin (Silent Mating Type Information Regulation 2, S. cerevisiae, Homolog) 2, EC 3.5.1., SIR2; GenBank: AAK51133.1); Sema 5b (FLJ10372, KIAA1445, Mm.42015, SEMA5B, SEMAG, Semaphorin 5b Hlog, sema domain, seven thrombospondin repeats (type 1 and type 1-like), Transmembrane Domain™ and short cytoplasmic domain, (semaphorin) 5B, Genbank accession no. AB04087; secernin 1 (SCRN1, SES1, KIAA0193, secerin-1; GenBank: EAL24458.1); SAGE (SAGE1, Sarcoma Antigen 1, Cancer/Testis Antigen 14, CT14, Putative Tumor Antigen; NCBI Reference Sequence: NP-061136.2); RU2AS (KAAG1, Kidney Associated Antigen 1, RU2AS, RU2 Antisense Gene Protein, Kidney-Associated Antigen 1; GenBank: AAF23613.1); RNF43-E3 ubiquitin-protein ligase RNF43 precursor [Homo sapiens], RNF124; URCC; NCBI Reference Sequence: NP-060233.3; RhoC (RGS5 (Regulator Of G-Protein Signaling 5, MSTP032, Regulator Of G-Protein Signalling 5, MSTP092, MST092, MSTP106, MST106, MSTP129, MST129; GenBank: AAB84001.1); RET (ret proto-oncogene; MEN2A; HSCR1; MEN2B; MTC1; PTC; CDHF12; Hs.168114; RET51; RET-ELE; NP-066124.1; NM-020975.4); RBAF600 (UBR4, Ubiquitin Protein Ligase E3 Component N-Recognin 4, Zinc Finger, UBR1 Type 1, ZUBR1, E3 Ubiquitin-Protein Ligase UBR4, RBAF600, 600 KDa Retinoblastoma Protein-Associated Factor, Zinc Finger UBR1-Type Protein 1, EC 6.3.2., N-recognin-4, KIAA0462, p600, EC 6.3.2, KIAA1307; GenBank: AAL83880.1); RAGE-1 (MOK, MOK Protein Kinase, Renal Tumor Antigen, RAGE, MAPK/MAK/MRK Overlapping Kinase, Renal Tumor Antigen 1, Renal Cell Carcinoma Antigen, RAGE-1, EC 2.7.11.22, RAGE1; UniProtKB/Swiss-Prot: Q9UQ07.1); RAB38/NY-MEL-1 (RAB38, NY-MEL-1, RAB38, Member RAS Oncogene Family, Melanoma Antigen NY-MEL-1, Rab-Related GTP-Binding Protein, Ras-Related Protein Rab-38, rrGTPbp; GenBank: AAH15808.1); PTPRK (DJ480J14.2.1 (Protein Tyrosine Phosphatase, Receptor Type, K R-PTP-KAPPA, Protein Tyrosine Phosphatase Kappa, Protein Tyrosine Phosphatase Kappa), Protein Tyrosine Phosphatase, Receptor Type, K, Protein-Tyrosine Phosphatase Kappa, Protein-Tyrosine Phosphatase, Receptor Type, Kappa, R-PTP-kappa, Receptor-Type Tyrosine-Protein Phosphatase Kappa, EC 3.1.3.48, PTPK; GenBank: AAI44514.1); PSMA; PSCA hIg(2700050C12Rik, C530008016Rik, RIKEN cDNA 2700050C12, RIKEN cDNA 2700050C12 gene, Genbank accession no. AY358628); PSCA (Prostate stem cell antigen precursor, Genbank accession no. AJ29743; PRDX5 (Peroxiredoxin 5, EC 1.11.1.15, TPx Type VI, B166, Antioxidant Enzyme B166, HEL-S-55, Liver Tissue 2D-Page Spot 71 B, PMP20, Peroxisomal Antioxidant Enzyme, PRDX6, Thioredoxin Peroxidase PMP20, PRXV, AOEB166, Epididymis Secretory Protein Li 55, Alu Co-Repressor 1, Peroxiredoxin-5, Mitochondrial, Peroxiredoxin V, prx-V, Thioredoxin Reductase, Prx-V, ACR1, Alu Corepressor, PLP; GenBank: CAG33484.1); PRAME (Preferentially Expressed Antigen In Melanoma, Preferentially Expressed Antigen Of Melanoma, MAPE, 01P-4, OIPA, CT130, Cancer/Testis Antigen 130, Melanoma Antigen Preferentially Expressed In Tumors, Opa-Interacting Protein 4, Opa-Interacting Protein 01P4; GenBank: CAG30435.1); pml-RARalpha fusion protein; PMEL17 (silver homolog; SILV; D12S53E; PMEL17; SI; SIL); ME20; gp10 BC001414; BT007202; M32295; M77348; NM-006928; PBF (ZNF395, Zinc Finger Protein 395, PRF-1, Huntington disease regulatory, HD Gene Regulatory Region-Binding Protein, Region-Binding Protein 2, Protein 2, Papillomavirus Regulatory Factor 1, HD-Regulating Factor 2, Papillomavirus-Regulatory Factor, PRF1, HDBP-2, Si-1-8-14, HDBP2, Huntington'S Disease Gene Regulatory Region-Binding Protein 2, HDRF-2, Papillomavirus Regulatory Factor PRF-1, PBF; GenBank: AAH01237.1); PAX5 (Paired Box 5, Paired Box Homeotic Gene 5, BSAP, Paired Box Protein Pax-5, B-Cell Lineage Specific Activator, Paired Domain Gene 5, Paired Box Gene 5 (B-Cell Lineage Specific Activator Protein), B-Cell-Specific Transcription Factor, Paired Box Gene 5 (B-Cell Lineage Specific Activator); PAP (REG3A, Regenerating Islet-Derived 3 Alpha, INGAP, PAP-H, Hepatointestinal Pancreatic Protein, PBBCGF, Human Proislet Peptide, REG-III, Pancreatitis-Associated Protein 1, Regi, Reg III-Alpha, hepatocarcinoma-intestine-pancreas, Regenerating Islet-Derived Protein III-Alpha, Pancreatic Beta Cell Growth Factor, HIP, PAP Homologous Protein, HIP/PAP, Proliferation-Inducing Protein 34, PAP1, Proliferation-Inducing Protein 42, REG-3-alpha, Regenerating Islet-Derived Protein 3-Alpha, Pancreatitis-Associated Protein; GenBank: AAH36776.1); p53 (TP53, Tumor Protein P53, TPR53, P53, Cellular Tumor Antigen P53, Antigen NY-CO-13, Mutant Tumor Protein 53, Phosphoprotein P53, P53 Tumor Suppressor, BCC7, Transformation-Related Protein 53, LFS1, tumor Protein 53, Li-Fraumeni Syndrome, Tumor Suppressor P53; P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), μl: 7.63, MW: 47206 TM: 1 [P] Gene Chromosome: 17p13.3, Genbank accession No. NP-002552.; OGT (0-Linked N-Acetylglucosamine (GlcNAc) Transferase, O-GlcNAc Transferase P110 Subunit, 0-Linked N-Acetylglucosamine (GlcNAc) Transferase (UDP-N-Acetylglucosamine:Polypeptide-N-Acetylglucosaminyl Transferase, UDP-N-Acetylglucosamine-Peptide N-Acetylglucosaminyltransferase 110 KDa Subunit, UDP-N-Acetylglucosamine:Polypeptide-N-Acetylglucosaminyl Transferase, Uridinediphospho-N-Acetylglucosamine:Polypeptide Beta-N-Acetylglucosaminyl Transferase, O-GlcNAc Transferase Subunit P110, EC 2.4.1.255, 0-Linked N-Acetylglucosamine Transferase 110 KDa Subunit, EC 2.4.1, HRNT1, EC 2.4.1.186, 0-GLCNAC; GenBank: AAH38180.1); 0A1 (Osteoarthritis QTL 1, OASD; GenBank: CAA88742.1); NY-ESO-1/LAGE-2 (Cancer/Testis Antigen 1 B, CTAG1 B, NY-ESO-1, LAGE-2, ESO1, CTAG1, CTAG, LAGE2B, Cancer/Testis Antigen 1, Autoimmunogenic Cancer/Testis Antigen NY-ESO-1, Ancer Antigen 3, Cancer/Testis Antigen 6.1, New York Esophageal Squamous Cell Carcinoma 1, L Antigen Family Member 2, LAGE2, CT6.1, LAGE2A; GenBank: AA130365.1); NY-BR-1 (ANKRD30A, Ankyrin Repeat Domain 30A, Breast Cancer Antigen NY-BR-1, Serologically Defined Breast Cancer Antigen NY-BR-1, Ankyrin Repeat Domain-Containing Protein 30A; NCBI Reference Sequence: NP-443723.2); N-ras (NRAS, Neuroblastoma RAS Viral (V-Ras) Oncogene Homolog, NRAS1, Transforming Protein N-Ras, GTPase NRas, ALPS4, N-Ras Protein Part 4, NS6, Oncogene Homolog, HRAS1; GenBank: AAH05219.1); NFYC (Nuclear Transcription Factor Y, Gamma, HAPS, HSM, Nuclear Transcription Factor Y Subunit C, Transactivator HSM-1/2, CCAAT Binding Factor Subunit C, NF-YC, CCAAT Transcription Binding Factor Subunit Gamma, CAAT Box DNA-Binding Protein Subunit C, Histone H1 Transcription Factor Large Subunit 2A, CBFC, Nuclear Transcription Factor Y Subunit Gamma, CBF-C, Transactivator HSM-1, H1TF2A, Transcription Factor NF-Y, C Subunit; neo-PAP (PAPOLG, Poly(A) Polymerase Gamma, Neo-Poly(A) Polymerase, Nuclear Poly(A) Polymerase Gamma, Polynucleotide Adenylyltransferase Gamma, SRP RNA 3′ Adenylating Enzyme/Pap2, PAP-gamma, Neo-PAP, SRP RNA 3′-Adenylating Enzyme, PAP2, EC 2.7.7.19, PAPG; NCBI Reference Sequence: NP-075045.2); NCA (CEACAM6, Genbank accession no. M1872); Napi3b (NAPI-3B, NPTIIb, SLC34A2, solute carrier family 34 (sodium phosphate), member 2, type II sodium-dependent phosphate transporter 3b, Genbank accession no. NM-00642); Myosin class I; MUM-3; MUM-2 (TRAPPC1, Trafficking Protein Particle Complex 1, BETS, BETS Homolog, MUM2, Melanoma Ubiquitous Mutated 2, Multiple Myeloma Protein 2, Trafficking Protein Particle Complex Subunit 1; MUM-If, Mucin (MUC1, Mucin 1, Cell Surface Associated, PEMT, PUM, CA 15-3, MCKD1, ADMCKD, Medullary Cystic Kidney Disease 1 (Autosomal Dominant), ADMCKD1, Mucin 1, Transmembrane, CD227, Breast Carcinoma-Associated Antigen DF3, MAM6, Cancer Antigen 15-3, MCD, Carcinoma-Associated Mucin, MCKD, Krebs Von Den Lungen-6, MUC-1/SEC, Peanut-Reactive Urinary Mucin, MUC1/ZD, Tumor-Associated Epithelial Membrane Antigen, DF3 Antigen, Tumor-Associated Mucin, episialin, EMA, H23 Antigen, H23AG, Mucin-1, KL-6, Tumor Associated Epithelial Mucin, MUC-1, Episialin, PEM, CD227 Antigen; UniProtKB/Swiss-Prot: P15941.3); MUCSAC (Mucin SAC, Oligomeric Mucus/Gel-Forming, Tracheobronchial Mucin’ MUC5, TBM, Mucin 5, Subtypes A And C, Tracheobronchial/Gastric, leB, Gastric Mucin, Mucin SAC, Oligomeric Mucus/Gel-Forming Pseudogene, Lewis B Blood Group Antigen, LeB, Major Airway Glycoprotein, MUC-SAC, Mucin-5 Subtype AC, Tracheobronchial; MUC1 (Mucin 1, Cell Surface Associated, PEMT, PUM, CA 15-3, MCKD1, ADMCKD, Medullary Cystic Kidney Disease 1 (Autosomal Dominant), ADMCKD1, Mucin 1, Transmembrane, CD227, Breast Carcinoma-Associated Antigen DF3, MAM6, Cancer Antigen 15-3, MCD, Carcinoma-Associated Mucin, MCKD, Krebs Von Den Lungen-6, MUC-1/SEC, Peanut-Reactive Urinary Mucin, MUC-1/X, Polymorphic Epithelial Mucin, MUC1/ZD, Tumor-Associated Epithelial Membrane Antigen, DF3 Antigen, Tumor-Associated Mucin, episialin, EMA, h23 Antigen, H23AG, mucin-1, KL-6, Tumor Associated Epithelial Mucin, MUC-1, Episialin, PEM, CD227 Antigen; MSG783 (RNF124, hypothetical protein FLJ20315, Genbank accession no. NM-01776; MRP4-multidrug resistance-associated protein 4 isoform 3, MOAT-B; MOATB [Homo sapiens]; NCBI Reference Sequence: NP-001288758.1; MPF (MPF, MSLN, SMR, megakaryocyte potentiating factor, mesothelin, Genbank accession no. NM-00582; MMP-7 (MMP7, matrilysin, MPSL1, matrin, Matrix Metalloproteinase 7 (Matrilysin, Uterine), Uterine Matrilysin, Matrix Metalloproteinase-7, EC 3.4.24.23, Pump-1 Protease, Matrin, Uterine Metalloproteinase, PUMP1, MMP-7, EC 3.4.24, PUMP-1; GenBank: AAC37543.1); MMP-2 (MMP2, Matrix Metallopeptidase 2 (Gelatinase A, 72 kDa Gelatinase, 72 kDa Type IV Collagenase), MONA, CLG4A, Matrix Metalloproteinase 2 (Gelatinase A, 72 kD Gelatinase, 72 kD Type IV Collagenase), CLG4, 72 kDa Gelatinase, 72 kDa Type IV Collagenase), Matrix Metalloproteinase-2, MMP-II, 72 KDa Gelatinase, Collagenase Type IV-A, MMP-2, Matrix Metalloproteinase-II, TBE-1, Neutrophil Gelatinase, EC 3.4.24.24, EC 3.4.24; GenBank: AAH02576.1); and Meloe;

In some embodiments, the at least two different antigens may be selected from the following antigens (or the at least two different epitopes may be the epitopes with in any of the following antigens): 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL1 7, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxin1, EpCAM, Ephrin B2/EphB4, EPO, ERCC, E-selectin, ET-1, Factor I1a, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC18, Muellerian-inhibitin substance, Mug, MuSK, NAIP, NAP, NCAD, N-Cadherin, NCA 90, NCAM, NCAM, Neprilysin, Neurotrophin-3,-4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSFIOB (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TRI), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p7S NTR), TNFRSF17 (BCMA), TNFRSF18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL RI TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-RI, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (fit-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), receptors for hormones, and growth factors.

In certain embodiments, the multispecific (e.g., bispecific) antibody according to the present disclosure may have a first antigen binding domain having specificity for CD3 and a second binding domain having specificity for a second antigen selected from the group consisting of 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL1 7, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxin1, EpCAM, Ephrin B2/EphB4, EPO, ERCC, E-selectin, ET-1, Factor I1a, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC18, Muellerian-inhibitin substance, Mug, MuSK, NAIP, NAP, NCAD, N-Cadherin, NCA 90, NCAM, NCAM, Neprilysin, Neurotrophin-3,-4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta R11b, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSFIOB (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-RI, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (fit-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), receptors for hormones, and growth factors.

In particular embodiments, combinations of antigens that may be targeted by a bispecific antibody may be any antigen combinations, as the present invention is universally applicable to a variety of bsAbs having different cognate antigen combinations. Non-limiting examples include: CD3 and Her2; CD3 and Her3; CD3 and EGFR; CD3 and CD19; CD3 and CD20; CD3 and EpCAM; CD3 and CD33; CD3 and PSMA; CD3 and CEA; CD3 and gp100; CD3 and gpA33; CD3 and B7-H3; CD64 and EGFR; CEA and HSG; TRAIL-R2 and LTbetaR; EGFR and IGFR; VEGFR2 and VEGFR3; VEGFR2 and PDGFR alpha; PDGFRalpha and PDGFR beta; EGFR and TGF-beta; EGFR and IFN-alpha; EGFR and IL-12p40; EGFR and MET; EGFR and EDV-miR16; EGFR and CD64; EGFR and Her2; EGFR and Her3; Her2 domain ECD2 and Her2 domain ECD4; Her2 and Her3; IGF-1R and HER3; CD19 and CD22; CD20 and CD22; CD20 and IFN-alpha; CD20 and TFG-beta; CD30 and CD16A; FceRI and CD32B; CD32B and CD79B; BCMA and HEL; MP65 and SAP-2; IL-17A and IL-23; IL-Ialpha and IL-Ibeta; IL-12 and IL-18; VEGF and osteopontin; VEGF and Ang-2; VEGF and PDGFRbeta; VEGF and Her2; VEGF and DLL4; FAP and DR5; FcgRII and IgE; PD-1 and PD-L1; CEA and DTPA; CEA and IMP288; and LukS-PV and LukF-PV.

“Different antigens” may refer to different and/or distinct proteins, polypeptides, or molecules; as well as different and/or distinct epitopes, which epitopes may be contained within one protein, polypeptide, or another type of molecule. Consequently, a bispecific antibody may bind to two epitopes on the same polypeptide.

The term “epitope” is used herein in the broadest sense and encompasses both a region or regions of an antigen interacting with a corresponding paratope. Protein or peptide epitopes may include amino acid residues interacting directly with a paratope (e.g., through hydrogen bonding or hydrophobic interactions) and amino acid residues that do not (e.g., those residues contributing generally to epitope conformation). Epitopes may be defined as structural and/or functional. Functional epitopes are generally epitopes with residues directly contributing to some function of the antigen (e.g., affinity for another protein or enzymatic activity). Structural epitopes are epitopes with residues contributing to antigen structure that may not significantly contribute to antigen function. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody.

According to IMGT (the international ImMunoGeneTics information system for immunoglobulins or antibodies, T cell receptors, MH, immunoglobulin superfamily IgSF and MhSF), the CH1 domain is the amino acid positions (or simply referred to as “positions” herein) 118-215 (EU numbering) and the hinge region is the amino acid positions 216-230 (EU numbering). The term “CH1 domain” is used in a broad sense herein to refer to a heavy chain region comprising at least seven consecutive amino acid positions of the heavy chain positions 118-215 (EU numbering)) and in some instances also comprising a portion of the hinge region (a portion of heavy chain positions 216-230 (EU numbering)) is included (e.g., up to position 218). A CH1 domain reference sequence, corresponding to the amino acid positions 118-220 according to EU numbering, is provided herein as SEQ ID NO: 1, which corresponds to the CH1 domain sequence of human IgG1 Allotype “IGHG1*01 (J00228)”, “IGHG1*04 (JN582178)”, or “IGHG1*07” and is an exemplary amino acid sequence of a wild-type (WT) CH1 domain.

### CH1 Domain Reference Sequence:

(SEQ ID NO: 1)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSC
(positions 118-220 according to EU numbering).

Alternative CH1 domain reference sequences of human IgG1 may include but are not limited to SEQ ID NO: 3, which corresponds to the CH1 domain sequence of human IgG1 Allotype “IGHG1*03 (Y14737)” or “IGHG1*08”.

Alternative CH1 domain reference sequence (214R relative to SEQ ID NO: 6):

(SEQ ID NO: 3)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSC
(positions 118-220 according to EU numbering).

These CH1 domain reference sequences are intended to be exemplary as Applicant intends for “CH1 domain” reference sequences to include any naturally occurring CH1 domain allotype or allelic variant.

Accordingly, an amino acid modification(s) in variant CH1 domain polypeptides according to the present disclosure may be relative to and/or incorporated to any parent CH1 domain polypeptides, for example but not limited to a wild-type sequence, such as SEQ ID NO: 1 or any allelic variants thereof such as but not limited to SEQ ID NO: 3.

According to IMGT, the CH2 domain is the amino acid positions (or simply referred to as “positions” herein) 231-340 (EU numbering). The term “CH2 domain” is used in a broad sense herein to refer to a heavy chain region comprising at least seven consecutive amino acid positions of the heavy chain positions 231-340 (EU numbering)). A CH2 domain reference sequence, corresponding to the amino acid positions 231-340 according to EU numbering, is provided herein as SEQ ID NO: 7, which is an exemplary amino acid sequence of a wild-type (WT) CH2 domain.

CH2 Domain Reference Sequence:

(SEQ ID NO: 7)
APELLGGPSVFLFPPKPKDTLMISRTPEVTCVVVDVSHEDPEVKF
NWYVDGVEVHNAKTKPREEQYNSTYRVVSVLTVLHQDWLNGKEYK
CKVSNKALPAPIEKTISKAK.

This CH2 domain reference sequence is intended to be exemplary as Applicant intends for “CH2 domain” reference sequences to include any naturally occurring CH2 domain allotype or allelic variant.

According to IMGT, the CH3 domain is the amino acid positions (or simply referred to as “positions” herein) 341-446 (EU numbering). The term “CH3 domain” is used in a broad sense herein to refer to a heavy chain region comprising at least seven consecutive amino acid positions of the heavy chain positions 341-446 (EU numbering)). A CH3 domain reference sequence, corresponding to the amino acid positions 341-446 according to EU numbering, is provided herein as SEQ ID NO: 8, which corresponds to the CH3 domain sequence of human IgG1 Allotype “IGHG1*01 (J00228)” or “IGHG1*08” and is an exemplary amino acid sequence of a wild-type (WT) CH3 domain.

CH3 Domain Reference Sequence:

(SEQ ID NO: 8)
GQPREPQVYTLPPSRDELTKNQVSLTCLVKGFYPSDIAVEWESNG
QPENNYKTTPPVLDSDGSFFLYSKLTVDKSRWQQGNVFSCSVMHE
ALHNHYTQKSLSLSPGK.

Alternative CH3 domain reference sequences of human IgG1 may include but are not limited to SEQ ID NO: 4, which corresponds to the CH3 domain sequence of human IgG1 Allotype “IGHG1*03 (Y14737)”, SEQ ID NO: 5, which corresponds to the CH3 domain sequence of human IgG1 Allotype “IGHG1*04 (JN582178)”, and SEQ ID NO: 6, which corresponds to the CH3 domain sequence of human IgG1 Allotype “IGHG1*07”.

Alternative CH3 domain reference sequence (356E and 358M relative to SEQ ID NO: 1):

(SEQ ID NO: 4)
GQPREPQVYTLPPSREEMTKNQVSLTCLVKGFYPSDIAVEWESNG
QPENNYKTTPPVLDSDGSFFLYSKLTVDKSRWQQGNVFSCSVMHE
ALHNHYTQKSLSLSPG.

Alternative CH3 domain reference sequence (422I relative to SEQ ID NO: 1):

(SEQ ID NO: 5)
GQPREPQVYTLPPSRDELTKNQVSLTCLVKGFYPSDIAVEWESNG
QPENNYKTTPPVLDSDGSFFLYSKLTVDKSRWQQGNIFSCSVMHE
ALHNHYTQKSLSLSPG.

Alternative CH3 domain reference sequence (431G relative to SEQ ID NO: 1):

(SEQ ID NO: 6)
GQPREPQVYTLPPSRDELTKNQVSLTCLVKGFYPSDIAVEWESNG
QPENNYKTTPPVLDSDGSFFLYSKLTVDKSRWQQGNVFSCSVMHE
GLHNHYTQKSLSLSPG.

Again it is expressly noted that these CH3 domain reference sequences are intended to be exemplary as Applicant intends for “CH3 domain” reference sequences to include any naturally occurring CH3 domain allotype or allelic variant.

There are two major CL isotypes, κ and λ, and such CL domains are referred to herein as CLκ domain and CLλ domain.

According to IMGT, the CLκ domain is the amino acid positions 108-214 (EU numbering). The term “CLκ domain” is used in a broad sense herein to refer to a light chain region comprising at least seven consecutive amino acid positions of the kappa light chain positions 108-214 (EU numbering). A CLκ domain reference sequence, corresponding to the amino acid positions 108-214 (EU numbering), is provided herein as SEQ ID NO: 2, which is an exemplary amino acid sequence of a wild-type (WT) CLκ domain.

### CLκ Domain Reference Sequence:

(SEQ ID NO: 2)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC
(positions 108 to 214 according to EU numbering).

According to IMGT, the CLλ domain is the amino acid positions 107-215 (EU numbering). The term “CLλ domain” is used in a broad sense herein to refer to a light chain region comprising at least seven consecutive amino acid positions of the lambda light chain positions 107-215 (EU numbering). A CLλ domain reference sequence, corresponding to the amino acid positions 107-215 (EU numbering), is provided herein as SEQ ID NO: 9, which is an exemplary amino acid sequence of a wild-type (WT) CLλ domain.

### CLλ Domain Reference Sequence:

(SEQ ID NO: 9)
GQPKAAPSVTLFPPSSEELQANKATLVCLISDFYPGAVTVAWKAD
SSPVKAGVETTTPSKQSNNKYAASSYLSLTPEQWKSHRSYSCQVT
HEGSTVEKTVAPTECS
(positions 107 to 215 according to EU numbering).

Various naturally occurring sequences (corresponding to different allotypes) of the constant domains of human IgG1, IgG2, IgG3, and IgG4 are known in the field and may be found for example in Vidarsson et al., Front Immunol. 2014 Oct. 20; 5:520 and U.S. Pat. No. 9,150,663, the disclosures of which are hereby incorporated by reference herein in their entirety herein. These constant domain reference sequences are intended to be exemplary as Applicant intends for constant domain sequences to include any naturally occurring constant domain allotype or allelic variant.

The term “cognate”, “cognate pair”, or “cognate pairing” as used herein, when referring to the relationship between CH1 and CL domains, means that at least one of the CH1 and CL domains comprises an amino acid substitution(s) so that the CH1 and CL domains preferentially pair with each other.

The term “cognate pair” or “cognate pairing” used herein, when referring to antigen binding or epitope binding, refers to a pair or pairing of two antibody chains (e.g., a heavy chain and a light chain), each containing a variable region (e.g., a VH and a VL, respectively), in which the combination of the variable regions provides intended binding specificity to an epitope or to an antigen. The term “non-cognate pair” or “non-cognate pairing” used herein refers to a pair or pairing of two antibody chains (e.g., a heavy chain and a light chain) each containing a variable region (e.g., a VH and a VL, respectively), in which the combination of the variable regions does not provide intended binding specificity to an epitope or to an antigen.

Provided herein are engineered variant CH1 domains and variant CL domains containing at least one amino acid substitution that promotes pairing between CH1 and CL domains. Such pairing may be more preferentially formed compared to another CH1-CL set, e.g., compared to a WT CH1-CL set or another variant CH1-CL set.

As used herein, the term “variant CH1 domain” (also referred to as CH1 domain variant) refers to a CH1 domain (a CH1 domain may also comprise a portion of the hinge region as described above, such as in SEQ ID NO: 1) having an amino acid sequence in which one or more amino acid substitutions are made to a CH1 domain sequence. The CH1 sequence to which such an amino acid substitution(s) is made includes but is not limited to the CH1 domain reference sequence SEQ ID NO: 1. In the libraries screened to identify the described variant CH1 domains, the nucleic acid sequence encoding SEQ ID NO: 1 was variegated. When one or more amino acid substitutions in a variant CH1 domain promotes pairing with a particular CL domain, e.g., a variant CLκ domain, such variant CH1 domain may be also referred to as a CH1 design, a design CH1 domain, or the like, and the term “design” thus used indicates that the CH1 domain is designed (i.e., modified) to pair with a particular CL domain.

There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The constant domains according to the present disclosure may be of any antibody isotype, e.g., IgG1, IgG2, IgG3, IgG4, IgA1, IgA2, IgD, IgM, and IgE. The CH1 domain, as used herein, may be derived from the CH1 of any antibody isotypes, e.g., IgG1, IgG2, IgG3, IgG4, IgA1, IgA2, IgD, IgM, and IgE. The CH1 substitution(s) according to the present disclosure may be made to any CH1 domain sequences, such as but not limited to the CH1 reference sequence SEQ ID NO: 1. While SEQ ID NO: 1 is a human IgG1 CH1 domain sequence, for example, given the sequence similarity between human IgG1 and human IgG2 or human IgG4, the CH1 substitution(s) according to the present disclosure may also be incorporated to human IgG2 or IgG4 CH1 sequences and still similar preferential CH1-CL pairing is expected. When CH2 and/or CH3 domain(s) are used with the variant CH1 domain of the present disclosure, the CH2 and/CH3 domain(s) may be derived from any antibody isotypes and the CH2 and/or CH3 domain isotype(s) does not necessarily need to be the same as the CH1 domain isotype. Additionally, the CH2 and/or CH3 domains used with the variant CH1 domains may be wild-type, e.g., germline, or variants thereof.

As used herein, the term “variant CLκ domain” (also referred to as CLκ domain variant) refers to a CLκ domain having an amino acid sequence in which one or more amino acid substitutions are made to a CLκ domain sequence. The CLκ sequence to which such an amino acid substitution(s) is made includes but is not limited to the CLκ domain reference sequence SEQ ID NO: 2. In the libraries screened to identify the described variant CLκ domains, the nucleic acid sequence encoding SEQ ID NO: 2 was variegated. When one or more amino acid substitutions in a variant CLκ domain promotes pairing with a particular CH1 domain, e.g., a variant CH1 domain as disclosed herein, such variant CLκ domain may be also referred to as a CLκ design, a design CLκ domain, or the like, and the term “design” thus used indicates that the CLκ domain is designed (i.e., modified) to pair with a particular CH1 domain.

As used herein, the term “variant CLλ domain” (also referred to as CLλ domain variant) refers to a CLλ domain having an amino acid sequence in which one or more amino acid substitutions are made to a CLλ domain sequence. The CLλ sequence to which such an amino acid substitution(s) is made includes but is not limited to the CLλ domain reference sequence SEQ ID NO: 9. In the libraries screened to identify the described variant CLλ domains, the nucleic acid sequence encoding SEQ ID NO: 9 was variegated. When one or more amino acid substitutions in a variant CLλ domain promotes pairing with a particular CH1 domain, e.g., a variant CH1 domain as disclosed herein, such variant CLλ domain may be also referred to as a CLλ design, a design CLλ domain, or the like, and the term “design” in this case thus used indicates that the CLλ domain is designed (i.e., modified) to pair with a particular CH1 domain.

The term “variant CL domain” (also referred to as CL domain variant) is used herein to encompass variant CLκ domains and variant CLλ domains.

The term “CH1-CL domain set”, “CH1-CL set”, or “CH1-CL pair” refers to a combination of a CH1 domain and a CL domain (kappa or lambda). The term “CH1-CL domain polypeptide set” may be used to highlight that the CH1 and CL domains are polypeptides. A “CH1-CL domain set” may be a “CH1-CLκ domain set” (also referred to as “CH1-CLκ set” or “CH1-CLκ pair”), which refers to a combination of a CH1 domain and a CLκ domain, or a “CH1-CLλ domain set” (also referred to as “CH1-CLλ set” or “CH1-CLλ pair”), which refers to a combination of a CH1 domain and a CLλ domain. The term “CH1-CL domain-encoding polynucleotide set” refers to a combination of a CH1 domain-encoding polynucleotide and a CL domain-encoding polynucleotide (the CL domain may be kappa or lambda).

A set name may be given to each CH1-CL set. A “CH1-CLκ set name” may be given to each “CH1-CLκ set” based on the specific amino acid substitution(s) at a specific position(s) of the CH1 and CLκ domains of the set (substitutions are relative to the WT CH1 and CLκ sequences), and a “CH1-CLλ set name” may be given to each “CH1-CLλ set” based on the amino acid substitution(s) at a specific position(s) of the CH1 and CLλ domains of the set (substitutions are relative to the WT CH1 and CLλ sequences), as explained more in detail herein below (e.g., the explanation related to Table 2 and Table 28). When a CH1-CL set comprises a non-wildtype CH1 domain and/or a non-wildtype CL domain, such a set may also be referred to as a variant CH1-CL domain set or variant CH1-CL set (the terms “variant CH1-CLκ domain set”, “variant CH1-CLκ set”, “variant CH1-CLλ domain set”, or “variant CH1-CLλ set” may be also used to specify the CL isotype). When the CH1 domain in a CH1-CL set comprises one or more amino acid substitutions to promote particular pairing with a given CL domain, such a CH1 domain may also be referred to as CH1 design domain or a design CH1 domain. When the CL domain in a CH1-CL set comprises one or more amino acid substitutions to promote particular pairing with a given CH1 domain, such a CL domain may also be referred to as CL design domain or a design CL domain (the term “CLκ design domain”, “design CLκ domain”, “CLλ design domain”, or “design CLλ domain” may be also used to specify the CL isotype).

When the amino acid substitutions in the CH1 and/or CL domains in a CH1-CL set promotes particular pairing with each other (as compared to pairing with other like domains), such CH1-CL set may be also referred to as a CH1-CL design, a CH1-CL design set, a design CH1-CL set, a design CH1-CL, or the like (the term “CH1-CLκ design”, “CH1-CLκ design set”, “design CH1-CLκ set”, “design CH1-CLκ”, “CH1-CLλ design”, “CH1-CLλ design set”, “design CH1-CLλ set”, “design CH1-CLλ” may be also used to specify the CL isotype). The term “design” thus used indicates that the CH1 and/or CL domains are designed (i.e., modified) to pair with each other.

The term “CH1-CL design set” encompasses CH1-CL design sets referred to herein by “Network” names. Networks were originally identified by Applicant by screening CH1-CLκ sets as described in Examples 1-2, but the same “Network” names are also used for referring to the corresponding CH1-CLλ sets. A “Network” defines that the design CLκ and design CLλ domains belonging to the Network comprise the same, specified amino acid residue(s) at a specified position(s). However, because the WT CLκ and WT CLλ sequences are not the same, even if the design CLκ domain may comprise the specified amino acid residue at the specified position because of a substitution to a WT CLκ domain sequence, the design CLλ domain may comprise the same, specified amino acid residue because the specified amino acid residue is the WT residue and not necessarily because of a substitution to a WT CLλ domain sequence.

For example, “Network 1993” defines that, regardless of the light chain isotype, the CH1 domain of the CH1-CL set belonging to “Network 1039” comprises 128R and 147R (R at position 128 and R at position 147) and the CL domain of the CH1-CL set belonging to “Network 1039” comprises 124E, 133Q, and 178E (E at position 124, Q at position 133, and E at position 178). When Network 1993 is referring to a CH1-CLκ set, the CH1-CLκ design set has the CH1-CLκ set name “H_128R_147R-L_124E_133Q_178E” and comprises a variant CH1 domain comprising 128R and 147R, which may be as a result of two substitutions L128R and K147R (substitutions relative to SEQ ID NO: 1) and a variant CLκ domain comprising 124E, 133Q, and 178E, which may be as a result of three substitutions Q124E, V133Q, and T178E (substitutions relative to SEQ ID NO: 2). An exemplary variant CH1 domain sequence for Network 1993 is provided by SEQ ID NO: 21, and an exemplary variant CLκ domain sequence for Network 1993 is provided by SEQ ID NO: 22. When Network 1993 is referring to a CH1-CLλ design set, the CH1 domain again comprises R at position 128 and R at position 147, and the CLλ domain again comprises E at position 124, Q at position 133, and E at position 178. The R at position 128 and R at position 147 in the CH1 may be again as a result of the two substitutions L128R and K147R (substitutions relative to SEQ ID NO: 1), but as for the CLλ domain, since the WT amino acid residue at position 124 is E in case of the λ isotype (unlike the κ isotype), the E at position 124 may not be because of a substitution, while Q at position 133 and E at position 178 may be again as a result of the substitutions V133Q and T178E. Therefore the CH1-CLλ set name for Network 1993 is “H_128R_147R-L_133Q_178E”. An exemplary variant CH1 domain sequence for Network 1993 is provided by SEQ ID NO: 21, and an exemplary variant CLλ domain sequence for Network 1993 is provided by SEQ ID NO: 29.

By a CH1 domain or variant CH1 domain “preferentially” pairing with a CL domain or variant CL domain, a variant CH1 domain providing “preferential” pairing with a CL domain or variant CL domain, or “preferential” CH1-CL pairing, it is meant that the CH1 domain or variant CH1 domain pairs with a given CL domain or variant CL domain rather than with another CL domain, such as a wildtype CL (CLκ or CLλ) domain, another variant CL (CLκ or CLλ), a CL domain or a variant CL domain of a different light chain isotype. By a CL domain or variant CL domain “preferentially” pairing with a CH1 domain or variant CH1 domain, a CL domain or variant CL domain providing “preferential” pairing with a CH1 domain or variant CH1 domain, or “preferential” CH1-CL pairing, it is meant that the CL domain or variant CL domain pairs with a given CH1 domain or variant CH1 domain rather than with another CH1 domain or another variant CH1 domain, such as a wildtype CH1 domain or another variant CH1 domain.

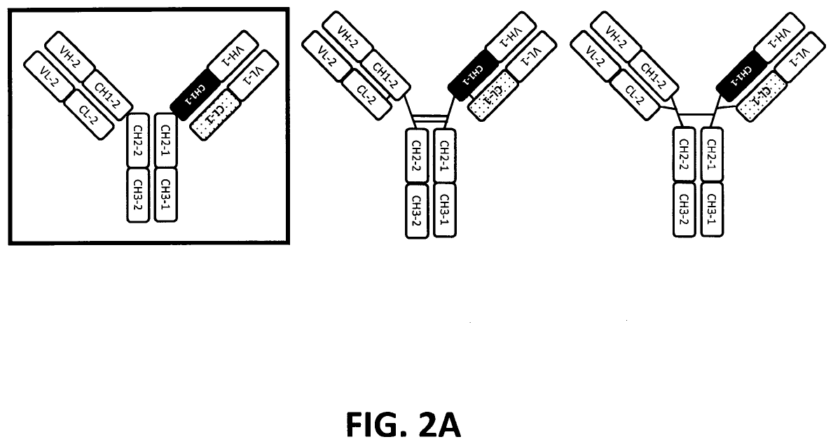

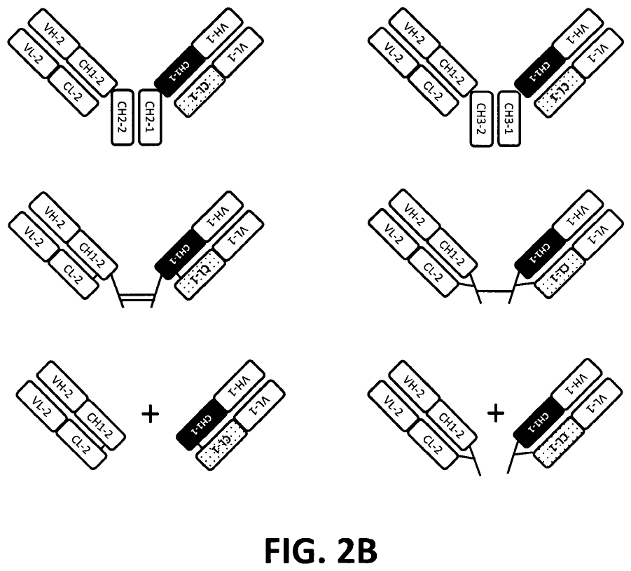

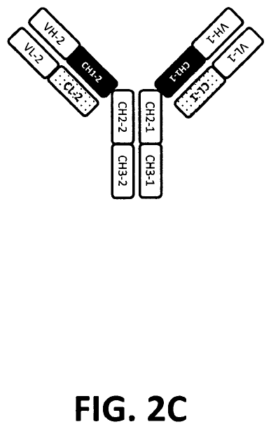

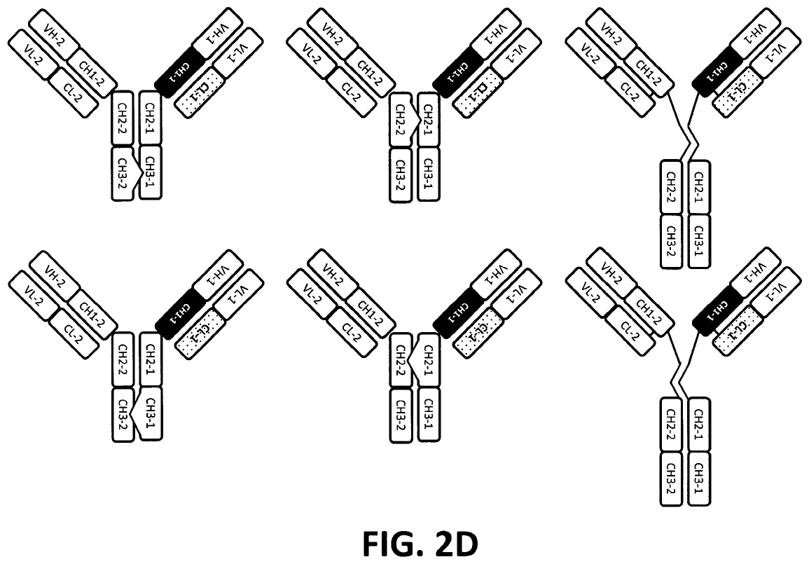

Such preferential CH1-CL pairing may be shown, for example, by formation of more of the pair of a given CH1 domain or variant CH1 domain and a given CL domain or variant CL domain than other CH1-CL pairs when the given CH1 domain or variant CH1 domain is computationally or recombinantly mixed, co-expressed, or co-provided with an approximate 1:1 mix of the given CL domain or variant CL domain and another CL domain (wildtype or another variant) and/or when the given CL domain or variant CL domain is computationally or recombinantly mixed, co-expressed, or co-provided with an approximate 1:1 mix of the given CH1 domain or variant CH1 domain and another CH1 domain (wildtype or variant). Such preferential pairing or the degree of preferential pairing between a given CH1 domain or variant CH1 domain and a CL domain or variant CL domain may be numerically shown, for example, by a computationally calculated score (such as ΔΔG; ΔΔG_{cognate total score}; ΔΔG_{cognate hbond_all}; RBPP; RBPP_{total score}; RBPP_{hbond_all}; and/or RBPP_{bond elec backrub 18k}), or by the percentage of the intended CH1-CL pairs (also referred to as, e.g., “% CH1-CL pairs” or “% CH1-CL pair”, or “% CH1-CL” (such as “% CH1-CLκ pair” or “% CH1-CLλ pair”)) among all CH1-CL pairs formed or by direct comparison of the amounts of the intended CH1-CL pairs and other CH1-CL pairs. In some cases, “preferential” CH1-CL pairing may be quantified by expressing a full-size bispecific antibody having a structure such as one shown in FIG. 2A (boxed), and in certain cases, the full-size bispecific antibody may comprise a heavy chain heterodimerizing technology, e.g., as shown in FIG. 2D (such as the “knob-in-hole” technology) and evaluating the relative amount of the intended bispecific antibodies among all full-size antibodies produced.

The degree of preferential CH1-CL pairing may be quantified by any available computational methods such as the Rosetta scoring and/or any available laboratory assays, such as but not limited to, liquid chromatography-mass spectrometry (LC-MS), ion exchange chromatography (IEX), AlphaLISA®, or flow cytometry. For example, a full-size bispecific antibody designed to comprise a heavy chain heterodimerizing technology (e.g., having a structure shown in FIG. 2D) by co-expressing first and second heavy chains at an approximately 1:1 ratio and first and second light chains at an approximately 1:1 ratio (first and second heavy chains and first and second light chains as described in the detailed description for FIG. 2D), and % of the intended bispecific antibodies (i.e., pairs that are correctly paired (also referred to as “PC” herein) among all full-size antibodies may be quantified. In such cases, the % PC, when a variant CH1 domain disclosed herein and/or a variant CL domain disclosed herein are used, may be about 55%, about 60%, about 65%, about 70%, about 75%, about 80%, about 85%, about 90%, about 95%, about 96%, about 97%, about 98%, about 99%, or about 100%. In some preferred embodiments, the % PC may be about 70% or higher. In some more preferred embodiments, the % CH1-CL pair may be about 75% or higher. In some more preferred embodiments, the % CH1-CL pair may be about 80% or higher. In some more preferred embodiments, the % CH1-CL pair may be about 85% or higher. In some more preferred embodiments, the % CH1-CL pair may be about 90% or higher. In some more preferred embodiments, the % CH1-CL pair may be about 95% or higher. In some more preferred embodiments, the % CH1-CL pair may be about 100%. A similar full-size bispecific antibody but designed to comprise two different CH1-CL sets (e.g., having a structure shown in FIG. 2C) and to further comprise a heavy chain heterodimerizing technology may be produced in a same manner and the % PC may be measured and evaluated in a same manner.

The variant CH1 domains, the variant CL domains, and/or variant CH1-CL domain sets or antibodies and antibody fragments comprising such a variant CH1 domain(s) and/or such a variant CH1-CL domain set(s)) may be further evaluated based on an additional property or properties, such as but not limited to: the degree of aggregation (e.g., presence of multimers of a full antibody) (also referred to as purity herein), which may be quantified by, e.g., chromatography such as size exclusion chromatography (SEC) or electrophoresis such as SDS-PAGE; melting temperature (Tm), which may be measured by, e.g., Differential scanning fluorimetry (DSF); production yields in a n appropriate cell type (e.g., HEK293 cells or yeast cells); “pI”, isoelectric point (“pI”); the level of interaction with polyspecificity reagent (“PSR”), which may be measured as in WO2014/179363; hydrophobic interaction of the antibody which may be measured by hydrophobic interaction chromatography (“HIC”) as measured as in e.g., Estep P, et al. MAbs. 2015 May-June; 7(3): 553-561.; solubility; production costs and/or time; stability; shelf life; in vivo half-life; and/or immunogenicity. Any of these or other properties may be used as an assessment criterion in addition to % CH1-CL values when assessing a given variant CH1 domain or CH1-CL set. Therefore, a variant CH1 domain or variant CH1-CL set of interest which gives a relatively lower % CH1-CL pair paired correctly (“PC”) value may just as ideal as another variant CH1 domain or CH1-CL set with a relatively higher % PC value, if the variant CH1 domain or variant CH1-CL set of interest provides a good profile on one or more of the above mentioned properties. For example, a variant CH1 domain or variant CH1-CL set which gives 80% PC with 3% aggregation (3% of the expression products are multimers of a full antibody) may be just as ideal as another variant CH1 domain or variant CH1-CL set which gives 90% PC with 10% aggregation.

A “library” is used herein to encompass any collections of biological materials such as nucleic acids, peptides, proteins, and sequence information thereof. For example, a “CH1 domain-encoding polynucleotide library” refers to a collection of polynucleotides encoding different CH1 domain polypeptides or of the polynucleotide sequences thereof, and a “CH1 domain polypeptide library” refers to a collection of different CH1 domain polypeptides or of the amino acid sequences thereof. Similarly, a “CL domain-encoding polynucleotide library” refers to a collection of polynucleotides encoding different CL domain polypeptides or of the polynucleotide sequences thereof, and a “CL domain polypeptide library” refers to a collection of different CL domain polypeptides or of the amino acid sequences thereof. The CL domain may be CLκ and/or CLU. Further, “a CH1-CL domain-encoding polynucleotide set library” refers to a collection of different sets of (i) a polynucleotide encoding a CH1 domain polypeptide (WT or variant) and (ii) a polynucleotide encoding a CL (CLκ and/or CLλ) domain polypeptide (WT or variant) or of the polynucleotide sequences thereof, and a “CH1-CL domain polypeptide set library” refers to a collection of different sets of (i) a CH1 domain polypeptide (WT or variant) and (ii) a CL (CLκ and/or CLλ) domain polypeptide (WT or variant) or of the amino acid sequences thereof.

A “pharmaceutical carrier”, as used herein, includes any and all solvents, dispersion media, coatings, antibacterial and antifungal agents, isotonic, and absorption delaying agents that are physiologically compatible. In one embodiment, the carrier is suitable for parenteral, intravenous, intraperitoneal, intramuscular, or sublingual administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions. In some embodiments, the carrier may be a liquid, in which an active therapeutic agent is formulated. The excipient generally does not provide any pharmacological activity to the formulation, though it may provide chemical and/or biological stability, and release characteristics. Exemplary formulations can be found, for example, in Remington's Pharmaceutical Sciences, Gennaro, A. editor, 19th edition, Philadelphia, PA. Williams and Wilkins (1995), which is incorporated by reference.

“Conservative amino acid substitutions” are known in the art and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a β-branched side-chain substituted for another amino acid with a β-branched side-chain (e.g., Ile, Thr, and Val), an amino acid with an aromatic side-chain substituted for another amino acid with an aromatic side chain (e.g., His, Phe, Trp, and Tyr), etc.

### Variant CH1 Domains, Heavy Chains Comprising, Variant CLκ Domains, Light Chain Comprising, and Variant CH1-CLκ Domain Sets

As described herein, certain positions within the CH1 domain and certain amino acid substitution(s) within the CH1 domain were found to influence the pairing of a CH1 domain with a CL domain. The CL domain may be a CLκ domain or a CLλ domain.

In some embodiments, the variant CH1 domains described herein may contain an amino acid substitution(s) at one or more of the following amino acid positions: 124, 128, 139, 141, 145, 147, 148, 166, 168, 175, 181, 185, and/or 187, according to EU numbering. In some embodiments, the variant CH1 domains described herein may contain any of the following position combinations: 168, 185, and 187; 128 and 147; 145, 147, and 181; 147 and 185; 148; 139, 141, and 187; 166 and 187; 168 and 185; 124 and 147; 147 and 148; 145; 145 and 181; 124, 145, and 147; 166 and 187; 147 and 175; 147R, 175, and 181; 145 and 147; or 147 and 185.

In some embodiments, the variant CH1 domains described herein may contain one or more of the following amino acid substitution(s): 124R, 128R, 139R, 141Q, 145Q, 145S, 147E, 147H, 147N, 147Q, 147R, 147T, 148E, 148R, 166K, 168R, 168S, 175D, 175E, 181E, 181Q, 185E, 185Q, 185S, 185Y, 187D, 187K, and/or 187Q.

In some embodiments, the variant CH1 domains described herein may contain any of the CH1 substitution combinations listed in Table 2.

In some embodiments, the variant CH1 domains described herein may contain any of the following amino acid substitution combinations: 168S, 185S, and 187D; 128R and 147R; 145Q, 147E, and 181E; 147T and 185Q; 148R; 139R, 141Q, and 187Q; 166K and 187K; 168R and 185E; 124R and 147R; 147H and 148E; 145S; 145S and 181Q; 145S; 145Q and 181E; 124R, 145S, and 147Q; 166K and 187K; 147R and 175D; 147R, 175E, and 181Q; 145S and 147N; or 147N and 185Y.

The parent CH1 domain sequence to which such an amino acid substitution(s) may be incorporated may comprise a wild-type or naturally occurring CH1 domain sequence or a variant or engineered version thereof. An exemplary sequence of such a parent polypeptide includes but is not limited to the reference CH1 sequence SEQ ID NO: 1.

In certain embodiments, the amino acid sequence of the variant CH1 domains described herein may comprises or consists of the amino acid sequence of SEQ ID NO: 11, 21, 31, 41, 51, 61, 71, 81, 91, 101, 111, 121, 131, 141, 151, 161, 171, 181, 191, or 201.

In particular embodiments, the amino acid sequence of the variant CH1 domains described herein may comprises or consists of the amino acid sequence of SEQ ID NO: 11, 21, 31, or 41.

As described herein, certain positions within the CL domain and certain amino acid substitution(s) within the CL domain were found to influence the pairing of a CL domain with a CH1 domain.

In some embodiments, the variant CL domains (variant CLκ or CLλ domains) described herein may contain an amino acid substitution(s) at one or more of the following amino acid positions: 114, 120, 124, 127, 129, 133, 135, 137, 138, 178, and 180, according to EU numbering. In some embodiments, the variant CLκ domains described herein may contain any of the following position combinations: 135; 124, 133, and 178; 129, 178, and 180; 135 and 178; 124 and 129; 114, 135, and 138; 137 and 138; 127 and 129; 133; 124 and 133; 120, 178, and 180; 127, 129, and 178; 114, 137, and 138; 129, 178, and 180; 133 and 180; or 129 and 180. In some embodiments, the variant CLλ domains described herein may contain any of the following position combinations: 135; 133 and 178; 129, 178, and 180; 135 and 178; 124 and 129; 114, 135, and 138; 138; 127 and 129; 133; 120, 178, and 180; 127, 129, and 178; 114, 137, and 138; 129, 178, and 180; 133 and 180; or 129.

In some embodiments, the variant CLκ domains described herein may contain one or more of the following amino acid substitution(s): 114D, 114Q, 120S, 124E, 124S, 127D, 127R, 127T, 129D, 129E, 129R, 133Q, 133Y, 135R, 135S, 137S, 137T, 138E, 138R, 178E, 178H, 178R, and 180H, 180Q, 180R, and/or 180S. In some embodiments, the variant CLκ domains described herein may contain any of the CLκ substitution combinations listed in Table 2 or Appendix Table B. In some embodiments, the variant CLλ domains described herein may contain one or more of the following amino acid substitution(s): 114D, 114Q, 120S, 124S, 127D, 127R, 127T, 129D, 129E, 129R, 133Q, 133Y, 135R, 135S, 137T, 138E, 138R, 178E, 178H, 178R, and 180H, 180Q, and/or 180R. In some embodiments, the variant CLκ domains described herein may contain any of the CLλ substitution combinations listed in Table 28 or Appendix Table C.

In some embodiments, the variant CLκ domains described herein may contain any of the following amino acid substitution combinations: 135R; 124E, 133Q, and 178E; 129R, 178R, and 180Q; 135S and 178R; 124S and 129E; 114D, 135S, and 138R; 137S and 138E; 135S; 127D and 129E; 127R and 129R; 133Y; 133Y; 124E and 133Y; 120S, 178H, and 180Q; 127T, 129D, and 178R; 114Q, 137T, and 138E; 129D, 178R, and 180H; 129D and 180Q; 133Y and 180R; or 129R and 180S. In some embodiments, the variant CLλ domains described herein may contain any of the following amino acid substitution combinations: 135R; 133Q and 178E; 129R, 178R, and 180Q; 135S and 178R; 124S and 129E; 114D, 135S, and 138R; 138E; 135S; 127D and 129E; 127R and 129R; 133Y; 133Y; 120S, 178H, and 180Q; 127T, 129D, and 178R; 114Q, 137T, and 138E; 129D, 178R, and 180H; 129D and 180Q; 133Y and 180R; or 129R.

The parent CL domain sequence to which such an amino acid substitution(s) may be incorporated may comprise a wild-type or naturally occurring CL domain sequence or a variant or engineered version thereof. An exemplary sequence of such a parent CLκ polypeptide includes but is not limited to the reference CLκ sequence SEQ ID NO: 2. An exemplary sequence of such a parent CLλ polypeptide includes but is not limited to the reference CLλ sequence SEQ ID NO: 9.

In certain embodiments, the amino acid sequence of the variant CLκ domains described herein may comprises or consists of the amino acid sequence of SEQ ID NO: 12, 22, 32, 42, 52, 62, 72, 82, 92, 102, 112, 122, 132, 142, 152, 162, 172, 182, 192, or 202. In certain embodiments, the amino acid sequence of the variant CLλ domains described herein may comprises or consists of the amino acid sequence of SEQ ID NO: 19, 29, 39, 49, 59, 69, 79, 89, 99, 109, 119, 129, 139, 149, 159, 169, 179, 189, 199, or 209.

In particular embodiments, the amino acid sequence of the variant CLκ domains described herein may comprises or consists of the amino acid sequence of SEQ ID NO: 12, 22, 32, or 42. In particular embodiments, the amino acid sequence of the variant CLκ domains described herein may comprises or consists of the amino acid sequence of SEQ ID NO: 59, 99, 39, 199, 49, or 29.

As described herein, certain amino acid position combinations in a CH1 domain and a CL domain were found to influence pairing between the CH1 and CL domain. In some embodiments, the CH1-CLκ sets described herein may comprise an amino acid substitution(s) at one or more of the following amino acid positions in the CH1 and CLκ domains: CH1 positions 168, 185, and 187, along with CLκ position 135 (e.g., Network 1039); CH1 positions 128 and 147, along with CLκ positions 124, 133, and 178 (e.g., Network 1993); CH1 positions 145, 147, and 181, along with CLκ positions 129, 178, and 180 (e.g., Network 1443); CH1 positions 147 and 185, along with CLκ positions 135 and 178 (e.g., Network 2529); CH1 position 148, along with CLκ 124 and 129 (e.g., Network 367); CH1 positions 139, 141, and 187, along with CLκ positions 114, 135, and 138 (e.g., Network 1888); CH1 positions 166 and 187, along with CLκ positions 137 and 138 (e.g., Network 1328); CH1 positions 168 and 185, along with CLκ position 135 (e.g., Network 2366); CH1 positions 124 and 147, along with CLκ positions 127 and 129 (e.g., Network 964); CH1 positions 147 and 148, along with CLκ positions 127 and 129 (e.g., Network 767); CH1 position 145, along with CLκ position 133 (e.g., Network 1148); CH1 positions 145 and 181, along with CLκ position 133 (e.g., Network 384); CH1 position 145, along with CLκ positions 124 and 133 (e.g., Network 454); CH1 positions 145 and 181, along with CLκ positions 120, 178, and 180 (e.g., Network 1048); CH1 positions 124, 145, and 147, along with CLκ positions 127, 129, and 178 (e.g., Network 534); CH1 positions 166 and 187, along with CLκ positions 114, 137, and 138 (e.g., Network 838); CH1 positions 147 and 175, along with CLκ positions 129, 178, and 180 (e.g., Network 919); CH1 positions 147, 175, and 181, along with CLκ positions 129 and 180 (e.g., Network 394); CH1 positions 145 and 147, along with CLκ positions 133 and 180 (e.g., Network 1621); CH1 positions 147 and 185, along with CLκ positions 129 and 180 (e.g., Network 742).

In some embodiments, the CH1-CLλ sets described herein may comprise an amino acid substitution(s) at one or more of the following amino acid positions in the CH1 and CLλ domains: CH1 positions 168, 185, and 187, along with CLλ position 135 (e.g., Network 1039); CH1 positions 128 and 147, along with CLλ positions 133 and 178 (e.g., Network 1993); CH1 positions 145, 147, and 181, along with CLλ positions 129, 178, and 180 (e.g., Network 1443); CH1 positions 147 and 185, along with CLλ positions 135 and 178 (e.g., Network 2529); CH1 position 148, along with CLλ 124 and 129 (e.g., Network 367); CH1 positions 139, 141, and 187, along with CLλ positions 114, 135, and 138 (e.g., Network 1888); CH1 positions 166 and 187, along with CLλ position 138 (e.g., Network 1328); CH1 positions 168 and 185, along with CLλ position 135 (e.g., Network 2366); CH1 positions 124 and 147, along with CLλ positions 127 and 129 (e.g., Network 964); CH1 positions 147 and 148, along with CLλ positions 127 and 129 (e.g., Network 767); CH1 position 145, along with CLλ position 133 (e.g., Network 1148); CH1 positions 145 and 181, along with CLλ position 133 (e.g., Network 384); CH1 position 145, along with CLλ position 133 (e.g., Network 454); CH1 positions 145 and 181, along with CLλ positions 120, 178, and 180 (e.g., Network 1048); CH1 positions 124, 145, and 147, along with CLλ positions 127, 129, and 178 (e.g., Network 534); CH1 positions 166 and 187, along with CLλ positions 114, 137, and 138 (e.g., Network 838); CH1 positions 147 and 175, along with CLκ positions 129, 178, and 180 (e.g., Network 919); CH1 positions 147, 175, and 181, along with CLκ positions 129 and 180 (e.g., Network 394); CH1 positions 145 and 147, along with CLκ positions 133 and 180 (e.g., Network 1621); CH1 positions 147 and 185, along with CLκ position 129 (e.g., Network 742). In some embodiments, the CH1-CLκ sets according to the present invention may contain any of the CH1 and CLκ substitution combinations in the CH1-CLκ sets listed in Table 2.

In some embodiments, the CH1-CLκ, sets according to the present invention may contain any of the CH1 and CLκ substitution combinations in the CH1-CLκ, sets listed in Table 28.

In some embodiments, such a CH1-CLκ set according to the present invention may be any of the following CH1-CLκ sets: H_168S_185S_187D-L_135R (e.g., Network 1039); H_128R_147R-L_124E_133Q_178E (e.g., Network 1993); H_145Q_147E_181E-L_129R_178R_180Q (e.g., Network 1443); H_147T_185Q-L_135S_178R (e.g., Network 2529); H_148R-L_124S_129E (e.g., Network 367); H_139R_141Q_187Q-L_114D_135S_138R (e.g., Network 1888); H_166K_187K-L_137S_138E (e.g., Network 1328); H_168R_185E-L_135S (e.g., Network 2366); H_124R_147R-L_127D_129E (e.g., Network 964); H_147H_148E-L_127R_129R (e.g., Network 767); H_145S-L_133Y (e.g., Network 1148); H_145S_181Q-L_133Y (e.g., Network 384); H_145S-L_124E_133Y (e.g., Network 454); H_145Q_181E-L_120S_178H_180Q (e.g., Network 1048); H_124R_145S_147Q-L_127T_129D_178R (e.g., Network 534); H_166K_187K-L_114Q_137T_138E (e.g., Network 838); H_147R_175D-L_129D_178R_180H (e.g., Network 919); H_147R_175E_181Q-L_129D_180Q (e.g., Network 394); H_145S_147N-L_133Y_180R (e.g., Network 1621); or H_147N_185Y-L_129R_180S (e.g., Network 742). In some embodiments, such a CH1-CLλ set according to the present invention may be any of the following CH1-CLλ sets: H_168S_185S_187D-L_135R (e.g., Network 1039); H_128R_147R-L_133Q_178E (e.g., Network 1993); H_145Q_147E_181E-L_129R_178R_180Q (e.g., Network 1443); H_147T_185Q-L_135S_178R (e.g., Network 2529); H_148R-L_124S_129E (e.g., Network 367); H_139R_141Q_187Q-L_114D_135S_138R (e.g., Network 1888); H_166K_187K-L_138E (e.g., Network 1328); H_168R_185E-L_135S (e.g., Network 2366); H_124R_147R-L_127D_129E (e.g., Network 964); H_147H_148E-L_127R_129R (e.g., Network 767); H_145S-L_133Y (e.g., Network 1148); H_145S_181Q-L_133Y (e.g., Network 384); H_145S-L_133Y (e.g., Network 454); H_145Q_181E-L_120S_178H_180Q (e.g., Network 1048); H_124R_145S_147Q-L_127T_129D_178R (e.g., Network 534); H_166K_187K-L_114Q_137T_138E (e.g., Network 838); H_147R_175D-L_129D_178R_180H (e.g., Network 919); H_147R_175E_181Q-L_129D_180Q (e.g., Network 394); H_145S_147N-L_133Y_180R (e.g., Network 1621); or H_147N_185Y-L_129R (e.g., Network 742).

In certain embodiments, the amino acid sequence of the variant CH1 domain and the variant CLκ domain of such CH1-CLκ sets may comprise the amino acid sequence of: SEQ ID NOs: 11 and 12, respectively; SEQ ID NOs: 21 and 22, respectively; SEQ ID NOs: 31 and 32, respectively; SEQ ID NOs: 41 and 42, respectively; SEQ ID NOs: 51 and 52, respectively; SEQ ID NOs: 61 and 62, respectively; SEQ ID NOs: 71 and 72, respectively; SEQ ID NOs: 81 and 82, respectively; SEQ ID NOs: 91 and 92, respectively; SEQ ID NOs: 101 and 102, respectively; SEQ ID NOs: 111 and 112, respectively; SEQ ID NOs: 121 and 122, respectively; SEQ ID NOs: 131 and 132, respectively; SEQ ID NOs: 141 and 142, respectively; SEQ ID NOs: 151 and 152, respectively; SEQ ID NOs: 161 and 162, respectively; SEQ ID NOs: 171 and 172, respectively; SEQ ID NOs: 181 and 182, respectively; SEQ ID NOs: 191 and 192, respectively; or SEQ ID NOs: 201 and 202, respectively.

In certain embodiments, the amino acid sequence of the variant CH1 domain and the variant CLλ domain of such CH1-CLλ sets may comprise the amino acid sequence of: SEQ ID NOs: 11 and 19, respectively; SEQ ID NOs: 21 and 29, respectively; SEQ ID NOs: 31 and 39, respectively; SEQ ID NOs: 41 and 49, respectively; SEQ ID NOs: 51 and 59, respectively; SEQ ID NOs: 61 and 69, respectively; SEQ ID NOs: 71 and 79, respectively; SEQ ID NOs: 81 and 89, respectively; SEQ ID NOs: 91 and 99, respectively; SEQ ID NOs: 101 and 109, respectively; SEQ ID NOs: 111 and 119, respectively; SEQ ID NOs: 121 and 129, respectively; SEQ ID NOs: 131 and 139, respectively; SEQ ID NOs: 141 and 149, respectively; SEQ ID NOs: 151 and 159, respectively; SEQ ID NOs: 161 and 169, respectively; SEQ ID NOs: 171 and 179, respectively; SEQ ID NOs: 181 and 189, respectively; SEQ ID NOs: 191 and 199, respectively; or SEQ ID NOs: 201 and 209, respectively.

In some preferred embodiments, the CH1-CLκ set according to the present invention may be H_168S_185S_187D-L_135R (e.g., Network 1039); H_128R_147R-L_124E_133Q_178E (e.g., Network 1993); H_145Q_147E_181E-L_129R_178R_180Q (e.g., Network 1443); or H_147T_185Q-L_135S_178R (e.g., Network 2529).

In some preferred embodiments, the CH1-CLλ set according to the present invention may be H_148R-L_124S_129E (Network 367); H_124R_147R-L_127D_129E (Network 964); H_145Q_147E_181E-L_129R_178R_180Q (Network 1443); H_145S_147N-L_133Y_180R (Network 1621); H_168R_185E-L_135S (Network 2366); H_147T_185Q-L_135S_178R (Network 2529); H_128R_147R-L_133Q_178E (Network 1993).

In particular embodiments, the amino acid sequence of the variant CH1 domain and the variant CLκ domain of such CH1-CLκ sets may comprise the amino acid sequence of: SEQ ID NOs: 11 and 12, respectively; SEQ ID NOs: 21 and 22, respectively; SEQ ID NOs: 31 and 32, respectively; or SEQ ID NOs: 41 and 42, respectively.

In particular embodiments, the amino acid sequence of the variant CH1 domain and the variant CLλ domain of such CH1-CLλ sets may comprise the amino acid sequence of SEQ ID NOs: 19 and 59, respectively; SEQ ID NOs: 91 and 99, respectively; SEQ ID NOs: 31 and 39, respectively; SEQ ID NOs: 191 and 199, respectively; SEQ ID NOs: 81 and 89, respectively; SEQ ID NOs: 41 and 49, respectively; or SEQ ID NOs: 21 and 29, respectively.

Exemplary amino acid sequences of the CH1 and CLκ domains in some of the CH1-CLκ sets according to the present invention are shown in Appendix Tables A-B.

Exemplary amino acid sequences of the CH1 and CLλ domains in some of the CH1-CLλ sets according to the present invention are shown in Appendix Tables A and C.

The resultant variant CH1 and CL domains preferentially pair with each other, rather than the variant CH1 domain pairing with another CL domain (e.g., a wildtype CLκ domain, another variant CLκ domain, a wildtype CLλ domain, or a variant CLλ domain) or the variant CL domain pairing with another CH1 domain (e.g., a wildtype CH1 domain or another variant CH1 domain).

Such variant CH1 domains, variant CL domains, and/or CH1-CL sets disclosed herein may be useful in producing heterodimeric (or multimeric) polypeptides and molecules such as multi-specific antibodies and antibody fragments, by improving the fidelity of heavy-light chain pairing while maintaining the native IgG structure of a bispecific antibody, which is favorable due to its well-established properties as a therapeutic molecule, including a long in vivo half-life and the ability to elicit effector functions. Such variant CH1 domains, variant CL domains, and/or CH1-CL sets disclosed herein may also facilitate the creation of a bispecific antibody based on two existing and desirable mAbs. These variant CH1 domains, variant CL domains, and/or CH1-CL sets may be used to solve, in whole or in part, heavy-light chain mispairing when generating multi-specific, e.g., bispecific, antibodies by promoting proper heavy-light chain pairing. More specifically, multi-specific antibodies comprising a variant CH1 domain, a variant CL domain, and/or a CH1-CL set as disclosed herein will form fewer unwanted product-related contaminants, i.e., molecules containing mis-paired domains or chains, whose elimination during manufacturing can be challenging.

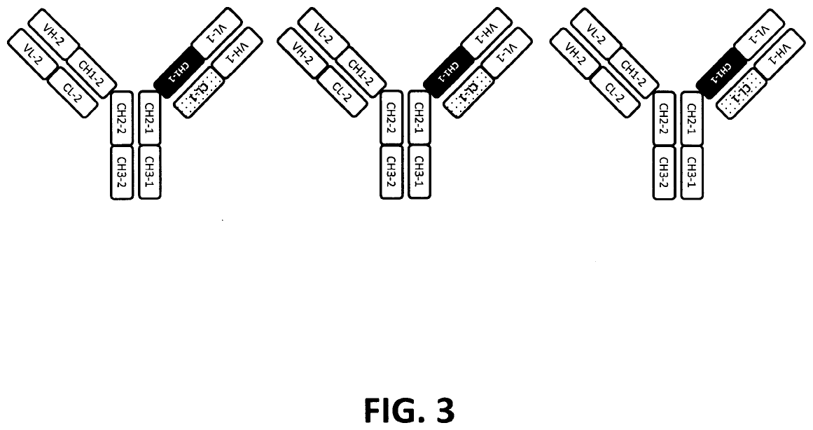

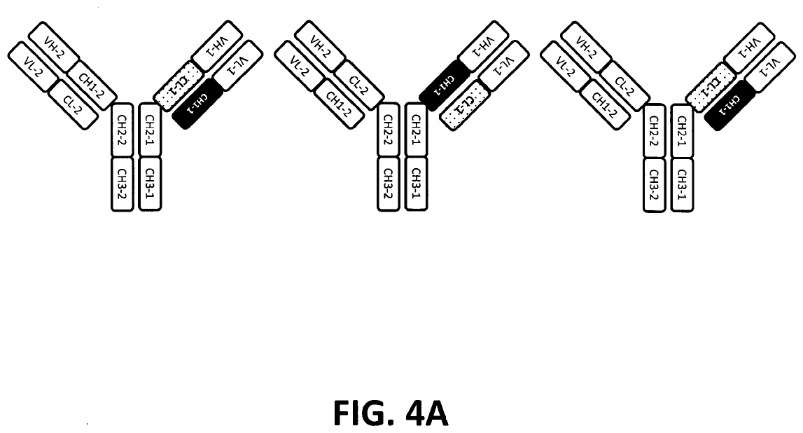

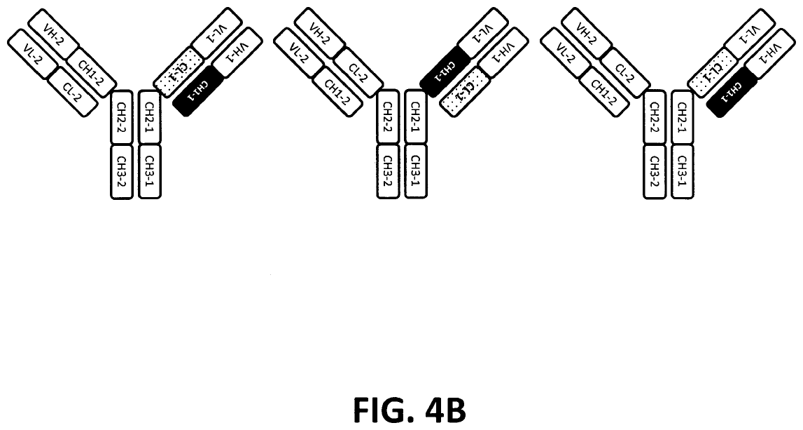

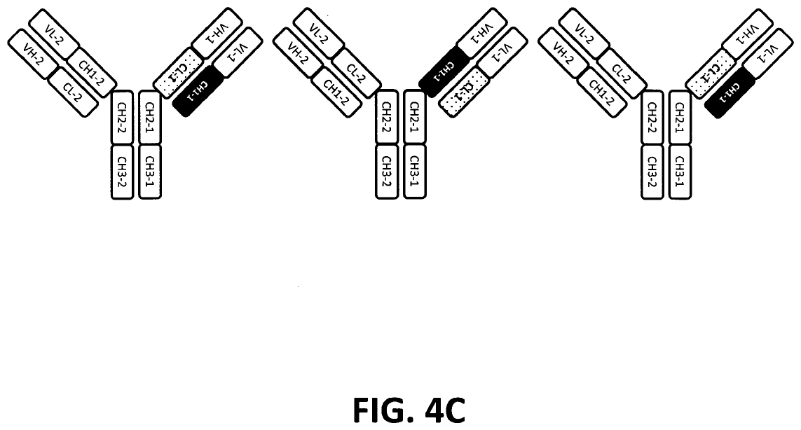

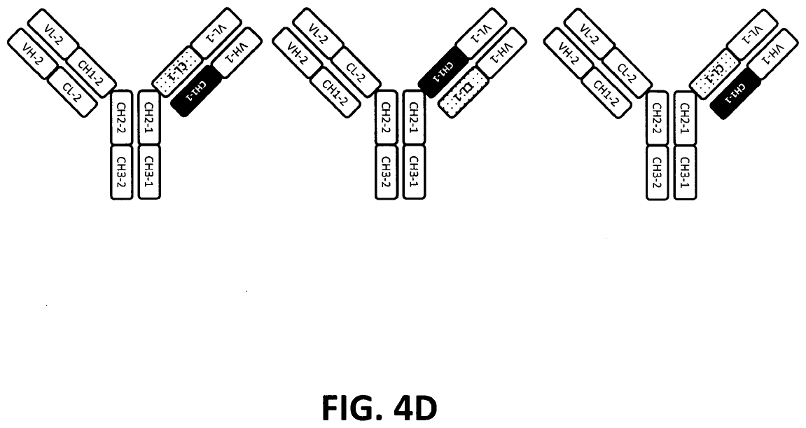

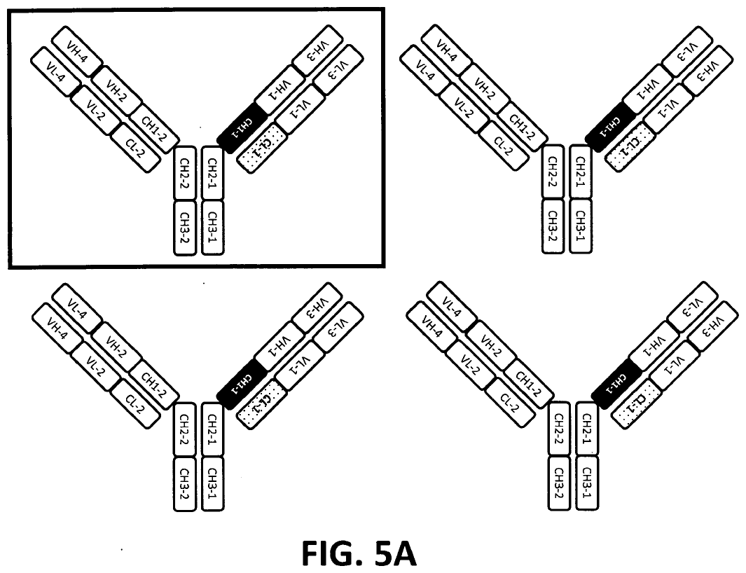

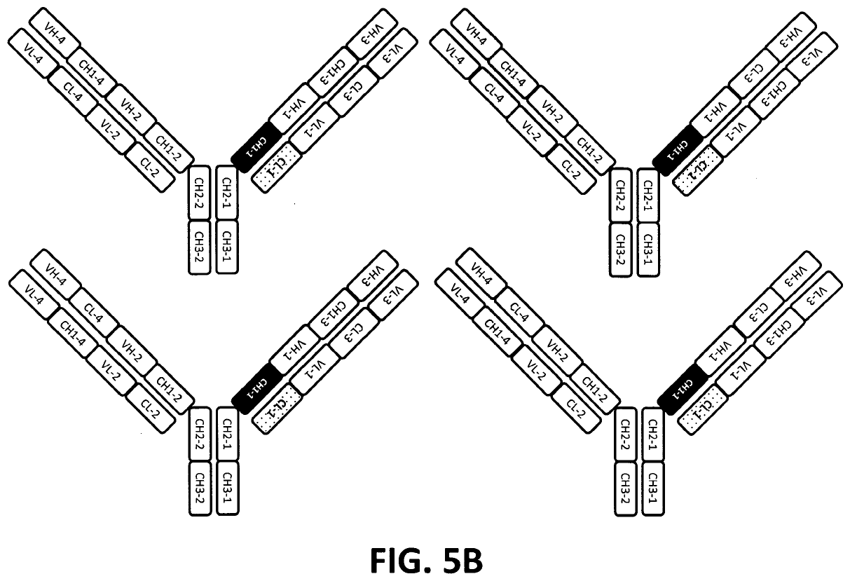

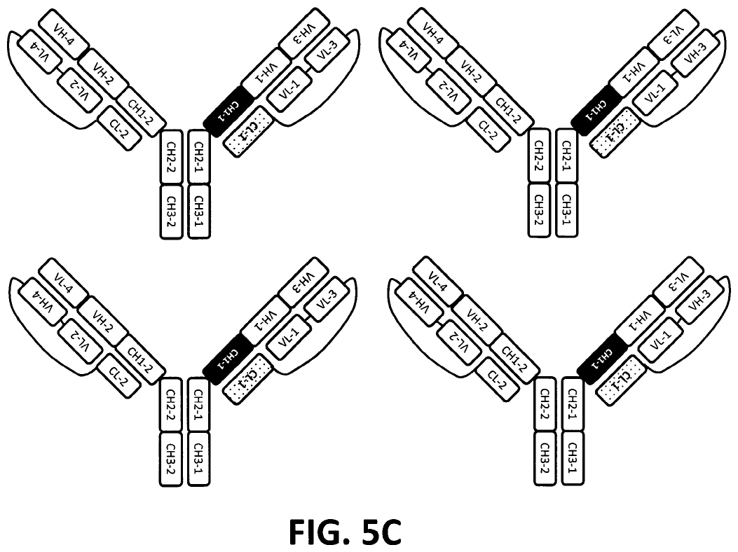

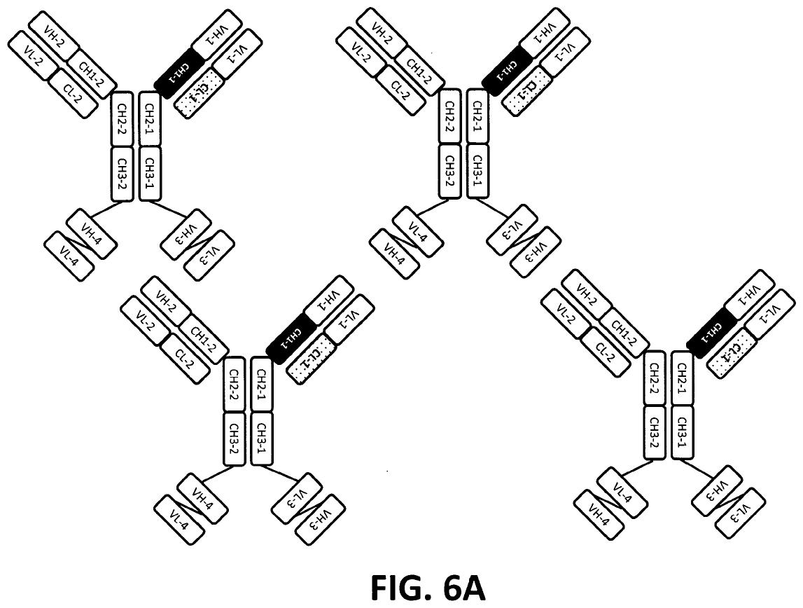

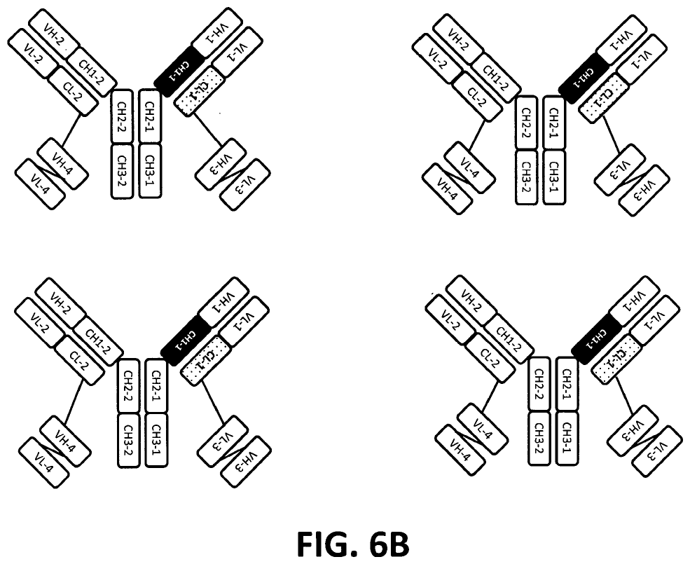

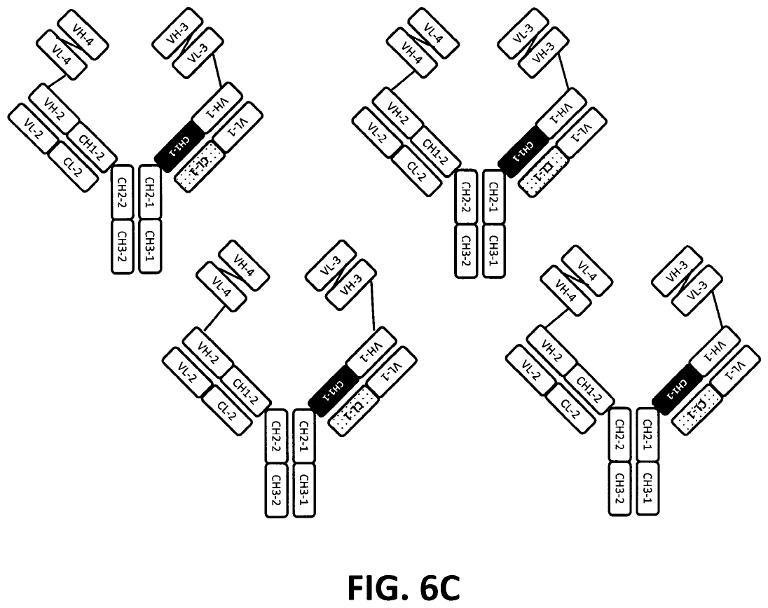

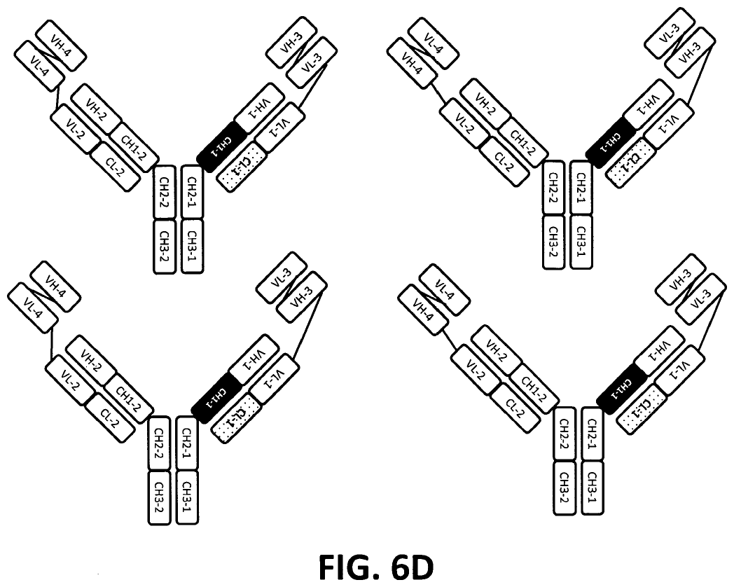

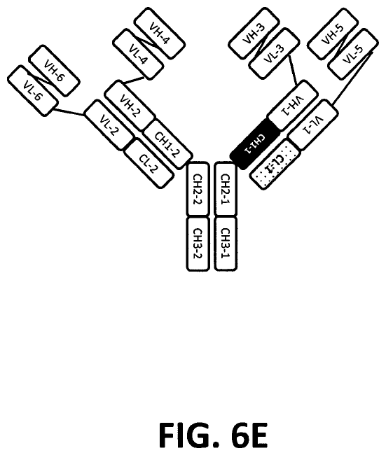

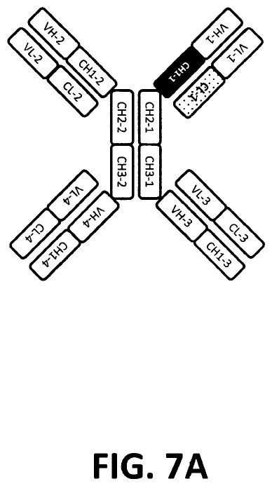

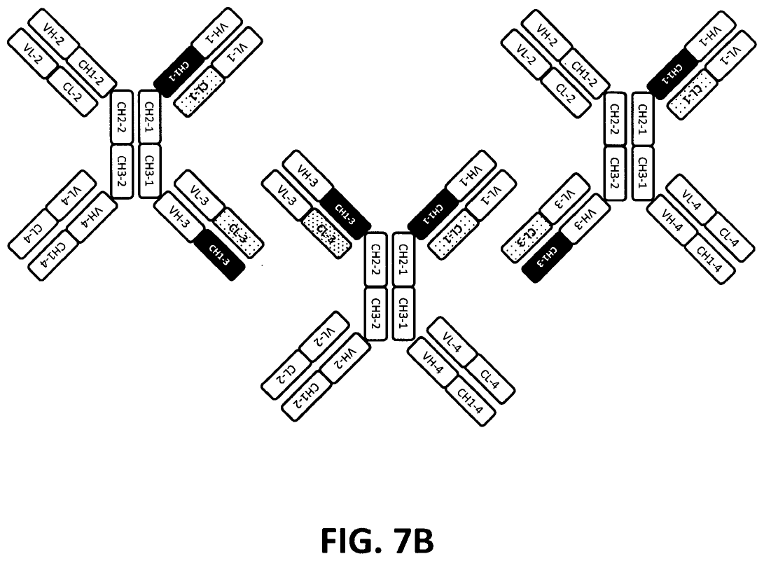

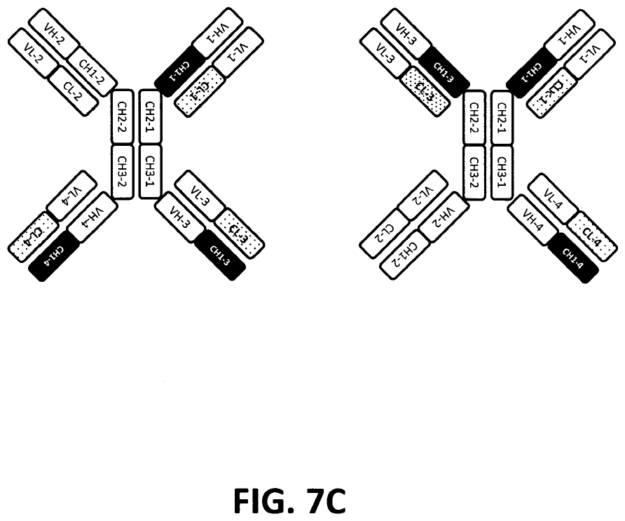

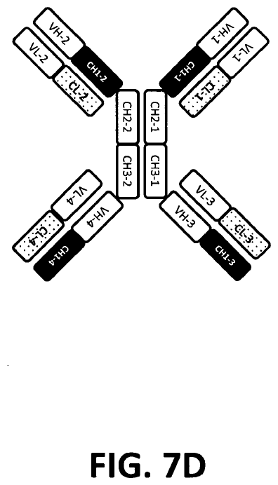

The variant CH1-CLκ sets according to the present disclosure that preferentially form a CH1-CLκ pair are not identical to those identified as pre-existing CH1-CLκ sets, such as the pre-existing CH1-CLκ sets listed in Table 1. The variant CH1-CLλ sets according to the present disclosure that preferentially form a CH1-CLλ pair are not identical to those identified as pre-existing CH1-CLλ sets, such as the pre-existing CH1-CLλ set “CTL31” shown in Table 1. However, any of the inventive variant CH1 domains, variant CL domains, and/or variant CH1-CL sets described herein may be combined with one or more of the pre-existing CH1-CL sets such as those in Table 1. For example, one or more of the substitutions in Table 1 may be added to the variant CH1 and/or variant CL domain and/or the CH1-CL sets of the present invention. In some cases, a molecule, such as a multi-specific antibody having a structure shown in FIGS. 2-7, which comprises one or more CH1-CL sets according to the present invention may comprise one or more CH1-CLκ sets of Table 1.

TABLE 1
Exemplary pre-existing preferential CH1-CL pairing technologies.

| | Company | | | |
|---|---|---|---|---|
| Technology | or | CH1 | CL | Additional |
| Name | Institution | substitutions | substitutions | modification(s) |
| v23 | Amgen | S183D | S176K | Need a substitution to K |
| | | | | in the other heavy chain; |
| | | | | a substitution to D in the |
| | | | | other light chain |
| CR3 | Biomunex | T187E | N137K; S114A | |
| MUT4 | Biomunex | L145Q; | V133T; S176V | |
| | | S183V | | |
| 14.1.2 (also | Dualogics | K147A; | S131R; E123K | |
| referred to as | | K213E | | |
| CTL1a) | | | | |
| 14.3.1 A | Dualogics | S183A; | S176I; S131R | |
| | | K147A | | |
| 14.3.1 B | Dualogics | K213E | E123K | |
| 14.3.2 A | Dualogics | S183G; | S176I; S131R | |
| | | K147A | | |
| 14.3.2 B | Dualogics | K213E | E123K | |
| 15.1 | Dualogics | K147A; | S131R; E123K; | |
| | | K213E; | S176I | |
| | | S183A | | |
| 15.2 | Dualogics | K147A; | S131R; E123K; | |
| | | K213E; | S176I | |
| | | S183G | | |
| v5 (also | Genentech | S183K | V133E | Need a substitution to E |
| referred to as | | | | in the other heavy chain; |
| CTL2b) | | | | a substitution to K in the |
| | | | | other light chain |
| v6/yt65H | Genentech | A141I; | F116A; L135V; | Need a substitution to E |
| (also referred | | F170S; | S174A; S176F; | in the other heavy chain; |
| to as CTL2a) | | S181M; | T178V | a substitution to K in the |
| | | S183A; | | other light chain |
| | | V185A | | |
| KGaA 1 | Merck | A141L | F118 to S, A, or | |
| | | | V | |
| KGaA 2 | Merck | K147D | T129R | |
| NIH Dimitrov | NIH | S181E; | S176; T178 | |
| | | S183V | | |
| Zymeworks | Zymeworks | F170V; | S176L | |
| C526 | | P171S; | | |
| | | S183G | | |
| Zymeworks | Zymeworks | S183L; | V133S | |
| C525 | | V185Y | | |
| CTL3l | Zymeworks; | S183K; | S176E; Y178E; | |
| | Eli Lilly | K214R | T212A | |
| CTL3k | Zymeworks; | L128E; | S131R; V133G; | |
| | Eli Lilly | K147T; | S176R; T178A | |
| | | Q175E; | | |
| | | S183W; | | |
| | | K214R | | |

* Positions are according to EU numbering.
** In some instances, the listed technologies may further require a modification(s) in the variable region(s). When a CH1-CL pairing technology of Table 1 is used as a control in Examples described herein, such a modification(s) in the variable region(s) was not incorporated to allow for proper comparison between different CH1-CL sets.
*** CTL3l is a CH1-CLλ set and all other sets are CH1-CLκ sets.

In further embodiments, any of the CH1-CL design sets according to the present invention may be combined with one or more other CH1-CL design sets, i.e., multiple different CH1-CL design sets may be incorporated in, e.g., one polypeptide or one molecule such as a multi-specific antibody or antibody fragment, as described more in detail below.

In some embodiments the one or more other CH1-CL design sets may comprise a CH1-CL design set according to the present invention, i.e., at least two different CH1-CL sets according to the present invention may be incorporated in one polypeptide or one molecule such as a multi-specific antibody or antibody fragment.

An antibody or antibody fragment comprising a CH1-CL set in one Fab arm and a WT CH1-CL set (i.e., both CH1 and CL domains are WT) in the other Fab arm may be referred to as having single interface design (SID) or a SIG format. A monospecific SID antibody (a “monospecific SID”) is a SID antibody in which one Fab and the other Fab arm have the same specificity. A bispecific SID antibody (a “bispecific SID”) is a SID antibody in which one Fab and the other Fab have different specificities. An antibody or antibody fragment comprising two different CH1-CL sets may be referred to as having double interface design (DID) or a DID format. A monospecific DID antibody (a “monospecific DID”) is a DID antibody in which one Fab and the other Fab arm have the same specificity. A bispecific DID antibody (a “bispecific DID”) is a DID antibody in which one Fab and the other Fab have different specificities. Furthermore, for each of the specific amino acid substitution(s) in the CH1 and/or CL domains disclosed herein as providing preferential pairing with each other, the amino acid included as a result of substitution may be further substituted via a conservative amino acid substitution to obtain another variant CH1 and/or variant CL domain(s) that provide equivalent (or even higher) pairing preference. Alternatively, for each of the variant CH1 and/or variant CL domains of an inventive CH1-CL set, one or more amino acid positions that were not affected (i.e., having the same amino acid relative to the wild-type sequence of CH1 or CL) may be altered via a conservative substitution to obtain another variant CH1 and/or variant CL domain that provide(s) equivalent or even higher CH1-CL pairing preference.

Provided below are a brief summary of some CH1-CL sets among many identified as shown in Examples, which provide at least one superior property such as higher correct heavy-light chain pairing compared to a WT CH1-CL set. For example, all sets in (1)-(7) show improved binding energy between the variant CH1 domain and the variant CLκ relative to the binding energy between WT CH1 and WT CLκ domains, based on the Rosetta score-based comparison, as shown in Examples 1 and 2. Some of the additional superior properties (non-exhaustive) for each of (1)-(7) are also provided below.

### (1) Network 1039

CH1-CL sets of Network 1039 comprise a CH1 domain comprising amino acid S at position 168, S at position 185, and D at position 187 (168S, 185S, and 187D) and a CL domain comprising amino acid R at position 135 (135R). CH1-CLκ sets of Network 1039 comprise a CH1 domain comprising amino acid substitutions (relative to the WT CH1 sequence) at positions 168, 185, and 187 to provide 168S, 185S, and 187D and a CLκ domain comprising an amino acid substitution (relative to the WT CLκ sequence) at position 135 to provide 135R and has the set name “H_168S_185S_187D-L_135R”. CH1-CLλ sets of Network 1039 comprise a CH1 domain comprising amino acid substitutions (relative to the WT CH1 sequence) at positions 168, 185, and 187 to provide 168S, 185S, and 187D and a CLλ domain comprising an amino acid substitution (relative to the WT CLλ sequence) at position 135 to provide R at 135R and has the set name “H_168S_185S_187D-L_135R”.

For example, the “H_168S_185S_187D-L_135R” (Network 1039) set shows a higher % correct CH1-CLκ pairing value when used in a SID in an exemplary BsAb, i.e., the variant CH1-CLκ set is used in one Fab arm of a full-size IgG-like bispecific antibody, as measured by LC-MS compared to a WT CH1-CLκ set (see Table 6 and Table 10). The “H_168S_185S_187D-L_135R” set (Network 1039) further improves the % correct CH1-CLκ pairing value when used in addition to another CH1-CLκ set such as the “H_145Q_147E_181E-L_129R_178R_180Q” set (Network 1443) (to achieve 95% correct pairing) in an exemplary DID, i.e., Network 1039 is used in one Fab arm while Network 1443 is used in the other Fab arm of a full-size IgG-like bispecific antibody) as measured by LC-MS (see Table 10). Additionally, combination of Network 1039 with the “H_128R_147R-L_124E_133Q_178E” set (Network 1993) in an exemplary DID achieved 97% correct CH1-CLκ pairing in an exemplary BsAb (see Table 10).

In this example, when Network 1039 substitutions are made to the reference CH1 and CLκ domain sequences of SEQ ID NOS: 1 and 2, the variant CH1 and CLκ domains comprise the amino acid sequences of SEQ ID NO: 11 and 12, respectively. When Network 1039 substitutions are made to the reference CH1 and CLλ domain sequences of SEQ ID NOS: 1 and 9, the variant CH1 and CLλ domains comprise the amino acid sequences of SEQ ID NO: 11 and 19, respectively. Network 1039 substitutions can be engineered into any reference CH1 and CL domain sequences to provide preferential pairing between the heavy and light chains containing the engineered variant domains.

### (2) Network 1993

CH1-CL sets of Network 1993 comprise a CH1 domain comprising amino acid R at position 128 and R at position 147 (128R and 147R) and a CL domain comprising amino acid E at position 124, Q at position 133, and E at position 178 (124E, 133Q, and 178E). CH1-CLκ sets of Network 1993 comprise a CH1 domain comprising amino acid substitutions (relative to the WT CH1 sequence) at positions 128 and 147 to provide 128R and 147R and a CLκ domain comprising an amino acid substitution (relative to the WT CLκ sequence) at positions 124, 133, and 178 to provide 124E, 133Q, and 178E and has the set name “H_128R_147R-L_124E_133Q_178E” . . . CH1-CLλ sets of Network 1993 comprise a CH1 domain comprising amino acid substitutions (relative to the WT CH1 sequence) at positions 128 and 147 to provide 128R and 147R and a CLλ domain comprising an amino acid substitution (relative to the WT CLλ sequence) at positions 133 and 178 to provide 133Q and 178E (it is noted that position 124 is E in WT CLλ) and has the set name “H_128R_147R-L_133Q_178E”.

For example, the “H_128R_147R-L_124E_133Q_178E” set (Network 1993) shows a higher % correct CH1-CLκ pairing value when used in an exemplary SID as measured by LC-MS compared to a WT CH1-CLκ set (see Table 6). Furthermore, the “H_128R_147R-L_124E_133Q_178E” set (Network 1993) dramatically improves the % correct CH1-CLκ pairing value when used in addition to another CH1-CLκ set such as the “H_145Q_147E_181E-L_129R_178R_180Q” set (Network 1443) (to achieve 100% correct pairing) or the “H_168S_185S_187D-L_135R” set (Network 1039) (to achieve 95% correct pairing) in an exemplary DID as measured by LC-MS (see Table 10). The very high % correct CH1-CLκ paring when Network 1993 and Network 1443 are used together in an exemplary DID with various specificity combinations are further confirmed in, e.g., Table 16.

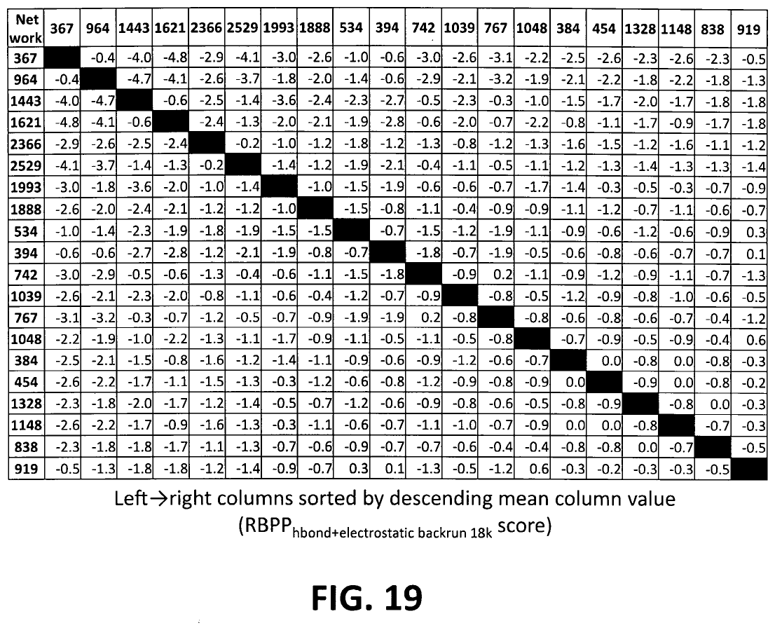

Furthermore, when the CH1-CLλ set of H_128R_147R-L_133Q_178E (Network 1993) was used in combination with the CH1-CLλ set of H_145Q_147E_181E-L_129R_178R_180Q (Network 1443) in a bsAb, it was predicted to provide particularly preferential pairing between the CH1 and CLλ domains in both CH1-CLλ sets, e.g., as shown in FIG. 19.

In this example, when Network 1993 substitutions are made to the reference CH1 and CLκ domain sequences of SEQ ID NOS: 1 and 2, the variant CH1 and CLκ domains comprise the amino acid sequences of SEQ ID NO: 21 and 22, respectively. When Network 1993 substitutions are made to the reference CH1 and CLλ domain sequences of SEQ ID NOS: 1 and 9, the variant CH1 and CLλ domains comprise the amino acid sequences of SEQ ID NO: 21 and 29, respectively. Network 1993 substitutions can be engineered into any reference CH1 and CL domain sequences to provide preferential pairing between the heavy and light chains containing the engineered variant domains.

### (3) Network 1443

CH1-CL sets of Network 1443 comprise a CH1 domain comprising amino acid Q at position 145, E at position 147, and E at position 181 (145Q, 147E, and 181E) and a CL domain comprising amino acid R at position 129, R at position 178, and Q at position 180 (129R, 178R, and 180Q). CH1-CLκ sets of Network 1443 comprise a CH1 domain comprising amino acid substitutions (relative to the WT CH1 sequence) at positions 145, 147, and 181 to provide 145Q, 147E, and 181E and a CLκ domain comprising an amino acid substitution (relative to the WT CLκ sequence) at positions 129, 178, and 180 to provide 129R, 178R, and 180Q and has the set name “H_145Q_147E_181E-L_129R_178R_180Q”-. CH1-CLλ sets of Network 1443 also comprise a CH1 domain comprising amino acid substitutions (relative to the WT CH1 sequence) at positions 145, 147, and 181 to provide 145Q, 147E, and 181E and a CLλ domain comprising an amino acid substitution (relative to the WT CLλ sequence) at positions 129, 178, and 180 to provide 129R, 178R, and 180Q and has the set name “H_145Q_147E_181E-L_129R_178R_180Q”.

For example, the “H_145Q_147E_181E-L_129R_178R_180Q” set (Network 1443) shows a higher % correct CH1-CLκ pairing value when used in an exemplary SID as measured by LC-MS compared to a WT CH1-CLκ set (see Table 6 and Table 10). Furthermore, the “H_145Q_147E_181E-L_129R_178R_180Q” set (Network 1443) dramatically improves the % correct CH1-CLκ pairing value when used in addition to another CH1-CLκ set such as the “H_168S_185S_187D-L_135R” set (Network 1039) (to achieve 97% correct pairing) in an exemplary DID as measured by LC-MS (see Table 10). Additionally, combination with the “H_128R_147R-L_124E_133Q_178E” set (Network 1993) in an exemplary DID achieved 100% correct CH1-CLκ pairing, combination with the “H_124R_147R-L_127D_129E” set (Network 964) in an exemplary DID achieved 95% correct CH1-CLκ pairing, combination with the “H_148R-L_124S_129E” set (Network 367) in an exemplary DID achieved 94% correct CH1-CLκ pairing, and combination with the “H_168R_185E-L_135S” set (Network 2366) in an exemplary DID achieved 91% correct CH1-CLκ pairing, all of which achieved much higher % correct pairing compared to when two WT CH1-CLκ sets are used (see Table 10). The very high % correct CH1-CLκ paring when Network 1993 and Network 1443 are used together in an exemplary DID with various specificity combinations are further confirmed in, e.g., Table 16.

Furthermore, when the CH1-CLλ set of H_145Q_147E_181E-L_129R_178R_180Q (Network 1443) was used in combination with the CH1-CLλ set of H_128R_147R-L_133Q_178E (Network 1993), H_124R_147R-L_127D_129E (Network 964), or H_148R-L_124S_129E (Network 367) in a bsAb, it was predicted to provide particularly preferential pairing between the CH1 and CLλ domains in both CH1-CLλ sets, e.g., as shown in FIG. 19.

In this example, when Network 1443 substitutions are made to the reference CH1 and CLκ domain sequences of SEQ ID NOS: 1 and 2, the variant CH1 and CLκ domains comprise the amino acid sequences of SEQ ID NO: 31 and 32, respectively. When Network 1443 substitutions are made to the reference CH1 and CLλ domain sequences of SEQ ID NOS: 1 and 9, the variant CH1 and CLλ domains comprise the amino acid sequences of SEQ ID NO: 31 and 39, respectively. Network 1443 substitutions can be engineered into any reference CH1 and CL domain sequences to provide preferential pairing between the heavy and light chains containing the engineered variant domains.

### (4) Network 2529

CH1-CL sets of Network 2529 comprise a CH1 domain comprising amino acid T at position 147 and Q at position 185 (147T and 185Q) and a CL domain comprising amino acid S at position 135 and R at position 178 (135S and 178R). CH1-CLκ sets of Network 2529 comprise a CH1 domain comprising amino acid substitutions (relative to the WT CH1 sequence) at positions 147 and 185 to provide 147T and 185Q and a CLκ domain comprising an amino acid substitution (relative to the WT CLκ sequence) at positions 135 and 178 to provide 135S and 178R and has the set name “H_147T_185Q-L_135S_178R” set. CH1-CLλ sets of Network 2529 also comprise a CH1 domain comprising amino acid substitutions (relative to the WT CH1 sequence) at positions 147 and 185 to provide 147T and 185Q and a CLκ domain comprising an amino acid substitution (relative to the WT CLκ sequence) at positions 135 and 178 to provide 135S and 178R and has the set name “H_147T_185Q-L_135S_178R” set.

For example, the “H_147T_185Q-L_135S_178R” set (Network 2529) shows a higher % correct CH1-CLκ pairing value when used in an exemplary SID as measured by LC-MS compared to a WT CH1-CLκ set (see Table 6).

Furthermore, when the CH1-CLλ set of H_147T_185Q-L_135S_178R (Network 2529) was used in combination with the CH1-CLλ set of H_148R-L_124S_129E (Network 367) or H_124R_147R-L_127D_129E (Network 964) in a bsAb, it was predicted to provide particularly preferential pairing between the CH1 and CLλ domains in both CH1-CLλ sets, e.g., as shown in FIG. 19.

In this example, when Network 2529 substitutions are made to the reference CH1 and CLκ domain sequences of SEQ ID NOS: 1 and 2, the variant CH1 and CLκ domains comprise the amino acid sequences of SEQ ID NO: 41 and 42, respectively. When Network 2529 substitutions are made to the reference CH1 and CLλ domain sequences of SEQ ID NOS: 1 and 9, the variant CH1 and CLλ domains comprise the amino acid sequences of SEQ ID NO: 41 and 49, respectively.

Network 2529 substitutions can be engineered into any reference CH1 and CL domain sequences to provide preferential pairing between the heavy and light chains containing the engineered variant domains.

### (5) Network 367

CH1-CL sets of Network 367 comprise a CH1 domain comprising amino acid R at position 148 (148R) and a CL domain comprising amino acid S at position 124 and E at position 129 (124S and 129E). CH1-CLκ sets of Network 367 comprise a CH1 domain comprising amino acid substitutions (relative to the WT CH1 sequence) at position 148 to provide 148R and a CLκ domain comprising an amino acid substitution (relative to the WT CLκ sequence) at positions 124 and 129 to provide 124S and 129E and has the set name “H_148R-L_124S_129E”. CH1-CLλ sets of Network 367 also comprise a CH1 domain comprising amino acid substitutions (relative to the WT CH1 sequence) at position 148 to provide 148R and a CLλ domain comprising an amino acid substitution (relative to the WT CLλ sequence) at positions 124 and 129 to provide 124S and 129E and has the set name “H_148R-L_124S_129E”.

For example, “H_148R-L_124S_129E” set (Network 367) improves the % correct CH1-CLκ pairing value when used in addition to another CH1-CLκ set such as the “H_145Q_147E_181E-L_129R_178R_180Q” set (Network 1443) or the “H_168S_185S_187D-L_135R” set (Network 1039) in an exemplary DID as measured by LC-MS (see Table 10).

Furthermore, when the CH1-CLλ set of H_148R-L_124S_129E (Network 367) was used in combination with the CH1-CLλ set of H_145S_147N-L_133Y_180R (Network 1621), H_147T_185Q-L_135S_178R (Network 2529), or H_145Q_147E_181E-L_129R_178R_180Q (Network 1443) in a bsAb, it was predicted to provide particularly preferential pairing between the CH1 and CLλ domains in both CH1-CLλ sets, e.g., as shown in FIG. 19.

In this example, when Network 367 substitutions are made to the reference CH1 and CLκ domain sequences of SEQ ID NOS: 1 and 2, the variant CH1 and CLκ domains comprise the amino acid sequences of SEQ ID NO: 51 and 52, respectively. When Network 367 substitutions are made to the reference CH1 and CLλ domain sequences of SEQ ID NOS: 1 and 9, the variant CH1 and CLλ domains comprise the amino acid sequences of SEQ ID NO: 51 and 59, respectively.

Network 367 substitutions can be engineered into any reference CH1 and CL domain sequences to provide preferential pairing between the heavy and light chains containing the engineered variant domains.

### (6) Network 964

CH1-CL sets of Network 964 comprise a CH1 domain comprising amino acid R at position 124 and R at position 147 (124R and 147R) and a CL domain comprising amino acid D at position 127 and E at position 129 (127D and 129E). CH1-CLκ sets of Network 964 comprise a CH1 domain comprising amino acid substitutions (relative to the WT CH1 sequence) at positions 124 and 147 to provide 124R and 147R and a CLκ domain comprising an amino acid substitution (relative to the WT CLκ sequence) at positions 127 and 129 to provide 127D and 129E and has the set name “H_124R_147R-L_127D_129E”. CH1-CLλ sets of Network 964 also comprise a CH1 domain comprising amino acid substitutions (relative to the WT CH1 sequence) at positions 124 and 147 to provide 124R and 147R and a CLλ domain comprising an amino acid substitution (relative to the WT CLλ sequence) at positions 127 and 129 to provide 127D and 129E and has the set name “H_124R_147R-L_127D_129E”.

For example, the “H_124R_147R-L_127D_129E” set (Network 964) improves the % correct CH1-CLκ pairing value when used in addition to another CH1-CLκ set such as the “H_145Q_147E_181E-L_129R_178R_180Q” set (Network 1443) (to achieve 95% correct CH1-CLκ pairing) in an exemplary DID as measured by LC-MS (see Table 10).

Furthermore, when the CH1-CLλ set H_124R_147R-L_127D_129E (Network 964) was used in combination with the CH1-CLλ set of H_145Q_147E_181E-L_129R_178R_180Q (Network 1443), H_145S_147N-L_133Y_180R (Network 1621), or H_147T_185Q-L_135S_178R (Network 2529) in a bsAb, it was predicted to provide particularly preferential pairing between the CH1 and CLλ domains in both CH1-CLλ sets, e.g., as shown in FIG. 19.

In this example, when Network 964 substitutions are made to the reference CH1 and CLκ domain sequences of SEQ ID NOS: 1 and 2, the variant CH1 and CLκ domains comprise the amino acid sequences of SEQ ID NO: 91 and 92, respectively. When Network 964 substitutions are made to the reference CH1 and CLλ domain sequences of SEQ ID NOS: 1 and 9, the variant CH1 and CLλ domains comprise the amino acid sequences of SEQ ID NO: 91 and 99, respectively.

Network 964 substitutions can be engineered into any reference CH1 and CL domain sequences to provide preferential pairing between the heavy and light chains containing the engineered variant domains.

### (7) Network 742

CH1-CL sets of Network 742 comprise a CH1 domain comprising amino acid N at position 147 and Y at position 185 (147N and 185Y) and a CL domain comprising amino acid R at position 129 and S at position 180 (129R and 180S). CH1-CLκ sets of Network 742 comprise a CH1 domain comprising amino acid substitutions (relative to the WT CH1 sequence) at positions 147 and 185 to provide 147N and 185Y and a CLκ domain comprising an amino acid substitution (relative to the WT CLκ sequence) at positions 129 and 180 to provide 129R and 180S and has the set name “H_147N_185Y-L_129R_180S”. CH1-CLλ sets of Network 742 comprise a CH1 domain comprising amino acid substitutions (relative to the WT CH1 sequence) at positions 147 and 185 to provide 147N and 185Y and a CLλ domain comprising an amino acid substitution (relative to the WT CLλ sequence) at position 129 to provide 129R (it is noted that position 180 is S in WT CLλ) and has the set name “H_147N_185Y-L_129R”.

For example, the “H_147N_185Y-L_129R_180S” set (Network 742) shows a higher % correct CH1-CLκ pairing value when used in an exemplary SID as measured by LC-MS compared to a WT CH1-CLκ set (see Table 6).

In this example, when Network 742 substitutions are made to the reference CH1 and CLκ domain sequences of SEQ ID NOS: 1 and 2, the variant CH1 and CLκ domains comprise the amino acid sequences of SEQ ID NO: 201 and 202, respectively. When Network 742 substitutions are made to the reference CH1 and CLλ domain sequences of SEQ ID NOS: 1 and 9, the variant CH1 and CLλ domains comprise the amino acid sequences of SEQ ID NO: 201 and 209, respectively.

Network 742 substitutions can be engineered into any reference CH1 and CL domain sequences to provide preferential pairing between the heavy and light chains containing the engineered variant domains.

It is noted that heavy chain polypeptides comprising any of the variant CH1 domain polypeptide described above and light chain polypeptides comprising any of the variant CLκ or CLλ domain polypeptide described above are also encompassed by the present invention.

### Polypeptides, Molecules, and Multi-Specific Antibodies

A variant CH1 domain, variant CL domain, and/or a variant CH1-CL domain set according to the present disclosure may exist in a polypeptide, a molecule, and/or a multi-specific antibody.

The “immunoglobulin polypeptide” as used herein refers to a polypeptide comprising at least one domain (or a variant thereof) of an immunoglobulin (e.g., a CH1 domain, a CL domain, etc). In certain instances, a CH1 domain may exist in a first polypeptide. The CH1 domain may be a variant CH1 domain according to the present disclosure. In certain instances, a CL domain may exist in a second polypeptide. The CL domain may be a variant CL domain (e.g., a variant CLκ domain or a variant CLλ domain) according to the present disclosure. When the CH1 domain in the first polypeptide preferentially forms a pair with the CL domain in the second polypeptide (e.g., the CH1 and the CL are a variant CH1-CL set according to the present invention), a heterodimer molecule may be formed between the first polypeptide and the second polypeptide. Such a molecule may be a multi-specific antibody having a structure such as but not limited to the structure disclosed in FIGS. 2-7.

In some embodiments, such a CH1-CL set may be any of the following CH1-CLκ sets: H_168S_185S_187D-L_135R (Network 1039); H_128R_147R-L_124E_133Q_178E (Network 1993); H_145Q_147E_181E-L_129R_178R_180Q (Network 1443); H_147T_185Q-L_135S_178R (Network 2529); H_148R-L_124S_129E (Network 367); H_139R_141Q_187Q-L_114D_135S_138R (Network 1888); H_166K_187K-L_137S_138E (Network 1328); H_168R_185E-L_135S (Network 2366); H_124R_147R-L_127D_129E (Network 964); H_147H_148E-L_127R_129R (Network 767); H_145S-L_133Y (Network 1148); H_145S_181Q-L_133Y (Network 384); H_145S-L_124E_133Y (Network 454); H_145Q_181E-L_120S_178H_180Q (Network 1048); H_124R_145S_147Q-L_127T_129D_178R (Network 534); H_166K_187K-L_114Q_137T_138E (Network 838); H_147R_175D-L_129D_178R_180H (Network 919); H_147R_175E_181Q-L_129D_180Q (Network 394); H_145S_147N-L_133Y_180R (Network 1621); or H_147N_185Y-L_129R_180S (Network 742). Exemplary CH1 and CLκ sequences in these CH1-CLκ sets are provided in Appendix Tables A-B and sequence listing.

In certain embodiments, such a CH1-CLκ set may be any of the following CH1-CLκ sets: H_168S_185S_187D-L_135R (Network 1039); H_128R_147R-L_124E_133Q_178E (Network 1993); H_145Q_147E_181E-L_129R_178R_180Q (Network 1443); or H_147T_185Q-L_135S_178R (Network 2529).

In some embodiments, such a CH1-CL set may be any of the following CH1-CLλ sets: H_168S_185S_187D-L_135R (Network 1039); H_128R_147R-L_133Q_178E (Network 1993); H_145Q_147E_181E-L_129R_178R_180Q (Network 1443); H_147T_185Q-L_135S_178R (Network 2529); H_148R-L_124S_129E (Network 367); H_139R_141Q_187Q-L_114D_135S_138R (Network 1888); H_166K_187K-L_138E (Network 1328); H_168R_185E-L_135S (Network 2366); H_124R_147R-L_127D_129E (Network 964); H_147H_148E-L_127R_129R (Network 767); H_145S-L_133Y (Network 1148); H_145S_181Q-L_133Y (Network 384); H_145S-L_133Y (Network 454); H_145Q_181E-L_120S_178H_180Q (Network 1048); H_124R_145S_147Q-L_127T_129D_178R (Network 534); H_166K_187K-L_114Q_137T_138E (Network 838); H_147R_175D-L_129D_178R_180H (Network 919); H_147R_175E_181Q-L_129D_180Q (Network 394); H_145S_147N-L_133Y_180R (Network 1621); or H_147N_185Y-L_129R (Network 742). Exemplary CH1 and CLλ sequences in these CH1-CLλ sets are provided in Appendix Tables A and C and sequence listing.

In certain embodiments, such a CH1-CLλ set may be any of the following CH1-CLλ sets: H_148R-L_124S_129E (Network 367); H_124R_147R-L_127D_129E (Network 964); H_145Q_147E_181E-L_129R_178R_180Q (Network 1443); H_145_147N-L_133Y_180R (Network 1621); H_168R_185E-L_135S (Network 2366); H_147T_185Q-L_135S_178R (Network 2529); or H_128R_147R-L_133Q_178E (Network 1993).

Such an immunoglobulin polypeptide may further comprise one or more antigen-binding domains (such as VH, VL, scFv, or nanobody), CH1, CH2, CH3, and/or CL domain(s). Such a polypeptide may be part of a multi-specific antibody molecule.

In some embodiments, a polypeptide may comprise an antigen-binding domain (such as a VH, VL, scFv, or nanobody) and a variant CH1 domain and optionally a CH2, CH3, and/or CL domain(s). In some embodiments, a polypeptide may comprise an antigen-binding domain (such as a VH, VL, scFv, or nanobody) and a variant CL domain and optionally a CH1, CH2, and/or CH3 domain(s). In some embodiments, such two polypeptides may pair with each other. In such a case, if the antigen-binding domain of the two polypeptides are a cognate VH and VL pair or a cognate VL and VH pair, the VH and VL may form an antigen-binding site for the cognate epitope.

Alternatively, the immunoglobulin polypeptide may not comprise a VH, VL, CH1, or CH2 domains. For example, a first polypeptide may comprise a first domain in addition to a variant CH1 domain. If a second polypeptide further comprises a second domain in addition to a variant CL domain which preferentially pairs with the variant CH1 domain, and if it is desired to form a heterodimer between the first and second domains, the preferential pairing between the variant CH1 domain and the variant CL domain will facilitate heterodimerization of the first and second domains.

In one embodiment, such a polypeptide may be comprised in a molecule such as a multi-specific antibody or a fragment thereof. When the molecule is a multi-specific antibody or a fragment thereof, various structures are possible, including but not limited to those shown in FIGS. 2-7.

In some embodiments, such a molecule may comprise a first polypeptide comprising a variant CH1 domain and a second polypeptide comprising a variant CL domain, in which the variant CH1 domain and the variant CL domain preferentially form a pair. For example, the variant CH1 domain and the variant CL domain may be a first CH1-CL set, which may be, for example, any of the CH1-CL sets according to the present invention. The CL isotype may be κ or λ.

In some embodiments, such a molecule may further comprise a third polypeptide comprising a variant CH1 domain and a fourth polypeptide comprising a variant CL domain, in which the variant CH1 domain and the variant CL domain preferentially form a pair. For example, the variant CH1 domain and the variant CL domain may be a second CH1-CL set, which may be, for example, any of the CH1-CL sets according to the present invention and may be different from the first CH1-CL set. The CL isotype in the second CH1-CL set may be κ or λ and may be same as or different from the CL isotype in the first CH1-CL set.

In some instances, the CH1 in the first set does not preferentially pair with the CL in the second set, the CL in the first set does not preferentially pair with the CH1 in the second set, the CH1 in the second set does not preferentially pair with the CL in the first set, the CL in the second set does not preferentially pair with the CH1 in the first set.

In certain instances, when such a molecule comprises two or more CH1-CL sets that are different from each other (all sets may be CH1-CLκ sets, all sets may be CH1-CLλ sets, or the two or more CH1-CL sets may be a mixture of a CH1-CLκ set(s) and a CH1-CLλ set(s)) but are both according to the present invention. Each of the two or more CH1-CL sets may be a CH1-CL set of Network selected from: Network 1039); Network 1993; Network 1443; Network 2529; Network 367; HNetwork 1888; Network 1328; Network 2366; Network 964; Network 767; Network 1148; Network 384; Network 454; Network 1048; Network 534; Network 838; Network 919; Network 394; Network 1621; or Network 742.

In particular instances, when such a molecule comprises two CH1-CLκ sets that are different from each other but are both according to the present invention, the CH1-CLκ set combination may be, for example, (i) H_145Q_147E_181E-L_129R_178R_180Q (Network 1443) and H_128R_147R-L_124E_133Q_178E (Network 1993); (ii) H_168S_185S_187D-L_135R (Network 1039) and H_128R_147R-L_124E_133Q_178E (Network 1993); (iii) H_145Q_147E_181E-L_129R_178R_180Q (Network 1443) and H_124R_147R-L_127D_129E (Network 964); (iv) H_145Q_147E_181E-L_129R_178R_180Q (Network 1443) and H_168S_185S_187D-L_135R (Network 1039); (v) H_145Q_147E_181E-L_129R_178R_180Q (Network 1443) and H_148R-L_124S_129E (Network 367); (vi) H_145Q_147E_181E-L_129R_178R_180Q (Network 1443) and H_168R_185E-L_135S (Network 2366); (vii) H_168S_185S_187D-L_135R (Network 1039) and H_148R-L_124S_129E (Network 367); (viii) H_168S_185S_187D-L_135R (Network 1039) and H_147T_185Q-L_135S_178R (Network 2529); (ix) H_168S_185S_187D-L_135R (Network 1039) and H_147N_185Y-L_129R_180S (Network 742); or (x) H_168S_185S_187D-L_135R (Network 1039) and H_168R_185E-L_135S (Network 2366).

In further instances, when such a molecule comprises two CH1-CLκ sets that are different from each other but are both according to the present invention, the CH1-CLκ set combination may be, (i) H_145Q_147E_181E-L_129R_178R_180Q (Network 1443) and H_128R_147R-L_124E_133Q_178E (Network 1993); (ii) H_168S_185S_187D-L_135R (Network 1039) and H_128R_147R-L_124E_133Q_178E (Network 1993); (iii) H_145Q_147E_181E-L_129R_178R_180Q (Network 1443) and H_124R_147R-L_127D_129E (Network 964); or (iv) H_145Q_147E_181E-L_129R_178R_180Q (Network 1443) and H_168S_185S_187D-L_135R (Network 1039) and may preferably be (i) H_145Q_147E_181E-L_129R_178R_180Q (Network 1443) and H_128R_147R-L_124E_133Q_178E (Network 1993). In some instances, the network combinations provide at least 95% correct pairing. In particular instances, when such a molecule comprises two CH1-CLλ sets that are different from each other but are both according to the present invention, the CH1-CLλ set combination may be, for example, (i) H_148R-L_124S_129E (Network 367) and H_145S_147N-L_133Y_180R (Network 1621); (ii) H_124R_147R-L_127D_129E (Network 964) and H_145Q_147E_181E-L_129R_178R_180Q (Network 1443); (iii) H_148R-L_124S_129E (Network 367) and H_147T_185Q-L_135S_178R (Network 2529); (iv) H_124R_147R-L_127D_129E (Network 964) and H_145S_147N-L_133Y_180R (Network 1621); (v) H_148R-L_124S_129E (Network 367) and H_145Q_147E_181E-L_129R_178R_180Q (Network 1443); (vi) H_124R_147R-L_127D_129E (Network 964) and H_147T_185Q-L_135S_178R (Network 2529); (vii) H_145Q_147E_181E-L_129R_178R_180Q (Network 1443) and H_128R_147R-L_133Q_178E (Network 1993).

In further instances, when such a molecule comprises two CH1-CLλ sets that are different from each other but are both according to the present invention, the CH1-CLλ set combination may be (i) H_148R-L_124S_129E (Network 367) and H_145S_147N-L_133Y_180R (Network 1621); or (ii) H_124R_147R-L_127D_129E (Network 964) and H_145Q_147E_181E-L_129R_178R_180Q (Network 1443). In some instances, the network combinations provide at least 95% correct pairing.

In some embodiments, such a molecule may further comprise, in addition to a first polypeptide and a second polypeptide, a third polypeptide comprising a CH1 domain and a fourth polypeptide comprising a CL domain of an isotype different from the CL isotype of the second polypeptide, in which the CH1 domain of the third polypeptide and the CL domain of the fourth polypeptide may preferentially form a pair. Such variant CH1 domain and variant CL domain may be called a second CH1-CL set.

In some instances, the CH1 in the first set does not preferentially pair with the CL in the second set, the CL in the first set does not preferentially pair with the CH1 in the second set, the CH1 in the second set does not preferentially pair with the CL in the first set, and/or the CL in the second set does not preferentially pair with the CH1 in the first set.

In some embodiments, such a molecule may optionally utilize, in addition to the first variant CH1 and CL domains, other variants outside of the CH1 and CL domains, such as variants in the antigen-binding domain and/or the hinge, to further promote preferential hetero pairing between two polypeptides.

In some cases, the first and second polypeptides may be further linked, e.g., via one or more disulfide bond(s), linker(s), etc. In some cases, the third and fourth polypeptides may be further linked, e.g., via one or more disulfide bond(s), linker(s), etc.

Such a molecule may be a multi-specific antibody having a structure such as but not limited to the structure disclosed in FIGS. 2-7. A multi-specific antibody according to the present disclosure may be bispecific, tri-specific, tetra-specific, or specific to five, six, or more epitopes. A multi-specific antibody according to the present disclosure may be divalent, trivalent, or tetravalent or have valency of five, six, or higher.

In some embodiments, a multi-specific antibody or antibody fragment according to the present disclosure may comprise multiple CH1-CL design sets. In certain embodiments, all of the multiple CH1-CL design sets may be CH1-CLκ sets. In certain embodiments, all of the multiple CH1-CL design sets may be CH1-CLλ sets. In certain embodiments, the multiple CH1-CL design sets may be a mixture of one or more CH1-CLκ sets and one or more CLλ sets.

In such a multi-specific antibody or antibody fragment, each CH1-CL set may be directly or indirectly linked to an antigen-binding site (e.g., formed by VH and VL or formed by VH in case of nanobody). Since such a multi-specific antibody or antibody fragment comprises multiple CH1-CL design sets (e.g., Set A, Set B, Set C, . . . etc) and multiple antigen-binding sites (e.g., Site A, Site B, Site C, . . . etc), multiple combinations of CH1-CL design sets with antigen-binding sites may be possible. For example, in one case, Set A may be linked to Site A, Set B may be linked to Site B, Set C may be linked to Site C, . . . , while in another case Set A may be linked to Site B, Set B may be linked to Site C, . . . etc.

In some cases, specific combinations (of CH1-CL design sets with antigen-binding sites) may yield multi-specific antibodies or fragments thereof with improved developability characteristics. Such characteristics may include but are not limited to: (i) production yield, which may be assessed in one or more cell types (e.g., mammalian cells such as CHO cells and HEK cells, yeast cells, insect cells, plant cells etc) using any appropriate methods or as described herein and/or compatibility to certain antibody purification methods (e.g., protein A affinity purification); (ii) degree of aggregation (e.g., presence of multimers of a full antibody) (also referred to as purity herein), which may be quantified using any appropriate methods or as described herein, e.g., by chromatography such as size exclusion chromatography (SEC) or electrophoresis such as SDS-PAGE; (iii) rates of correct pairing (e.g., between heavy chains and/or between heavy and light chains), which may be assessed using any appropriate methods or as described herein e.g., by LC-MS; (iv) melting temperature (Tm) and/or aggregation temperature (Tagg) (e.g., Tagg266), which may be measured using any appropriate methods or as described herein e.g., by Differential scanning fluorimetry (DSF) or Differential scanning calorimetry (DSC) or using an instrument such as Uncle®; (v) “pI”, isoelectric point (“pI”), which may be measure using any appropriate methods; (vi) the level of interaction with polyspecificity reagent (“PSR”), which may be measured using any appropriate methods or as described herein e.g., as in WO2014/179363; (vii) hydrophobic interaction of the antibody which may be measured using any appropriate methods or as described herein, e.g., by hydrophobic interaction chromatography (“HIC”) as in e.g., Estep p, et al. MAbs. 2015 May-June; 7(3): 553-561.; (viii) self-interaction; (ix) stability to high or low pH stress; (x) solubility; (xi) production costs and/or time; (xii) other stability parameters; (xiii) shelf life; (xiv) in vivo half-life; and/or (xv) immunogenicity, which may be assessed using any appropriate methods.

Reductions in self-interaction may be predicted in silico or measured by in vitro assay. Such in vitro assays may include, but are not limited to, affinity-capture self-interaction nanoparticle spectroscopy (AC-SINS) and dynamic light scattering (DLS) analysis. In some embodiments, various combinations of CH1-CL design sets with antigen-binding sites, each with equivalent multi-specific antigen binding functionality, may be screened for selection of combinations with improved developability characteristics (e.g., reduced self-interaction).

In certain cases, self-interaction may be measured in vitro by AC-SINS using a previously described protocol (Liu y et al., MAbs. March-April 2014; 6(2):483-92). For example, polyclonal goat anti-human IgG Fc antibodies (capture; Jackson ImmunoResearch Laboratories) and polyclonal goat non-specific antibodies (non-capture; Jackson ImmunoResearch Laboratories) may be buffer exchanged into 20 mM sodium acetate (pH 4.3) and concentrated to 0.4 mg/ml. A 4:1 volume ratio of capture:non-capture may be prepared and further incubated at a 1:9 volume ratio with 20 nm gold nanoparticles (AuNP; Ted Pella Inc.) for 1 hour at room temperature. Thiolated PEG (Sigma-Aldrich) may then be used to block empty sites on the AuNP and filtered via a 0.22 μm PVDF membrane (Millipore). Coated particles may be subsequently added to the test antibody solution and incubated for 2 hours at room temperature before measuring absorbance from 510 to 570 nm on a plate reader. Data points may be fit with a second-order polynomial in Excel to obtain wavelengths at maximum absorbance. Values may be reported as the difference between plasmon wavelengths of the sample and background (Δλₘₐₓ). Self-interaction levels may be determined based on Δλₘₐₓ. Self-interaction may be considered: low when Δλₘₐₓ<5 nm; medium when Δλₘₐₓ≥5 nm and <20 nM; and high when Δλₘₐₓ≥20 nm.

In certain cases, self-interaction may be measured in vitro by DLS. Diffusion Interaction Parameter (kD) of monoclonal antibodies, usually measured at concentrations lower than 12 mg/mL, has strong correlation with their solution behavior in very high concentrations (>100 mg/mL). Positive kD values indicate repulsive interaction among the molecules and has positive correlation with low viscosity at high concentration, in the same formulation buffer. kD values may be obtained by measuring mutual diffusion coefficient (D) for a series of different concentrations (C), by DLS. For example, DLS kD measurements at multiple concentrations between 0.5-12 mg/mL, in 10 mM Histidine buffer, pH 6.0 may be taken. Method may be easily modified for different formats of antibodies including bsAbs and in different formulation buffers.

Stability to high or low pH stress may be measured by placing antibodies or fragments thereof in a high or low pH environment for a certain period of time followed by one or more biochemical analyses. For example, for testing stability to high pH, 100 μL of 2 mg/mL IgG samples may be buffer-exchanged into 20 mM Tris, 10 mM EDTA (pH 8.5) and incubated at 40° C. After 7 days, stressed samples may be collected and subjected to tryptic peptide mapping and CZE analysis; and for testing stability to low pH, 100 μL of 2 mg/mL IgG samples may be buffer-exchanged into 50 mM sodium acetate buffer (pH 5.5) and incubated at 40° C. After 14 days, stressed samples may be collected and subjected to tryptic peptide mapping and reduced intact mass analysis.

### Polynucleotides, Vectors, Cells, and Compositions

Polypeptides, molecule, and/or multi-specific antibodies comprising variant CH1 and/or CL domains described herein may be encoded by a polynucleotide or polynucleotides. Such polynucleotide or polynucleotides may be a DNA or RNA or a combination thereof.

Any of the polypeptide(s) described herein may be present in a vector.

Any of the variant CH1 domain(s), variant CL domain(s), CH1-CL set(s), polypeptide(s), molecule(s), multi-specific antibody(ies), polynucleotide(s), and/or vector(s) may be present in a cell, e.g., a eukaryotic cell. In some embodiments, such polypeptides may be expressed in mammalian cells, such as HEK923 cells or Chinese hamster ovary (CHO) cells. In some embodiments, variant CH1 and/or CL domain(s) are expressed in yeast.

Any of the variant CH1 domain(s), variant CL domain(s), CH1-CL set(s), polypeptide(s), molecule(s), multi-specific antibody(ies), polynucleotide(s), vector(s), and/or cells may be present in a composition. If the composition is a therapeutic composition, the composition may further comprise a pharmaceutically acceptable carrier.

### CH1 Domain Libraries, CL Domain Libraries, and CH1-CL Set Screening/Selection

Also contemplated by the present disclosure are methods of generating a CH1 domain library. The library may be particularly used to screen for CH1 sequences and that preferentially pair with a CL domain or a variant CL domain (which may be κ or λ isotype).

In some embodiments, at least one nucleic acid position within the codon encoding any of the amino acid positions of CH1 present in or proximate to the CH1-CL interface may be variegated. In certain embodiments, proximate may mean 1, 2, 3, 4, or 5 amino acids upstream or downstream of an amino acid present in the CH1-CL interface.

In some embodiments, at least one nucleic acid position within the codon encoding any of the amino acid positions of CH1 at which an amino acid substitution is present in any of the inventive variant CH1 domains may be variegated. For example, such pre-determined amino acid position(s) may be position(s) 124, 128, 139, 141, 145, 147, 148, 166, 168, 175, 181, 185, and/or 187, according to EU numbering.

In some embodiments, any of the amino acid position combinations selected from: 168, 185, and 187; 128 and 147; 145, 147, and 181; 147 and 185; 148; 139, 141, and 187; 166 and 187; 168 and 185; 124 and 147; 147 and 148; 145; 145 and 181; 124, 145, and 147; 166 and 187; 147 and 175; 147R, 175, and 181; 145 and 147; or 147 and 185 may be variegated.

In some embodiments, a degenerate codon, optionally a degenerate RMW codon representing six naturally occurring amino acids (D, T, A, E, K, and N) or a degenerate NNK codon representing all 20 naturally occurring amino acid residues may be used, to induce variegation at a pre-determined position.

Also contemplated by the present disclosure are CH1 domain libraries. In some embodiments, the CH1 domain library may be the library generated by any methods of generating a CH1 domain library described herein.

Also contemplated by the present disclosure are methods of generating a CL domain library. The library may be particularly used to screen for CL sequences and that preferentially pair with a variant CH1 domain. The library may be a CLκ domain library, a CLλ domain library, or a library containing both CLκ and CLλ domains.

In some embodiments, at least one nucleic acid position within the codon encoding any of the amino acid positions of CL present in or proximate to the CH1-CL interface may be variegated. In certain embodiments, proximate may mean 1, 2, 3, 4, or 5 amino acids upstream or downstream of an amino acid present in the CH1-CL interface.

In some embodiments, at least one nucleic acid position within the codon encoding any of the amino acid positions of CL at which an amino acid substitution is present in any of the inventive variant CL domains may be variegated. For example, such pre-determined amino acid position(s) may be position(s) 114, 120, 124, 127, 129, 133, 135, 137, 138, 178, and 180, according to EU numbering.

In some embodiments, any of the amino acid position combinations selected from: 135; 124, 133, and 178; 129, 178, and 180; 135 and 178; 124 and 129; 114, 135, and 138; 137 and 138; 127 and 129; 133; 124 and 133; 120, 178, and 180; 127, 129, and 178; 114, 137, and 138; 129, 178, and 180; 133 and 180; or 129 and 180 may be variegated in CLκ.

In some embodiments, any of the amino acid position combinations selected from: 135; 133 and 178; 129, 178, and 180; 135 and 178; 124 and 129; 114, 135, and 138; 138; 127 and 129; 133; 120, 178, and 180; 127, 129, and 178; 114, 137, and 138; 129, 178, and 180; 133 and 180; or 129 may be variegated in CLU.

In some embodiments, a degenerate codon, optionally a degenerate RMW codon representing six naturally occurring amino acids (D, T, A, E, K, and N) or a degenerate NNK codon representing all 20 naturally occurring amino acid residues may be used, to induce variegation at a pre-determined position.

Also contemplated by the present disclosure are CL domain libraries. In some embodiments, the CL domain library may be the library generated by any methods of generating a CL domain library described herein. The CL library may be a CLκ domain library, a CLλ domain library, or a library containing both CLκ and CLλ domains.

Also contemplated by the present disclosure are methods of generating a CH1-CL domain set library. The library may be particularly used to screen for CH1-CL domain sets in which the CH1 and CL domains in a set preferentially pair with each other. The CL domains included in such a library may be all CLκ domains, all CLλ domains, or a mixture of both CLκ and CLλ domains.

In some embodiments, the method may comprise a step of selecting combinations of CH1 domain position(s) and CL domain position(s) which are predicted to affect the CH1-CL interdomain interaction, such as an interaction mediated by a hydrogen bond. In certain embodiments, the prediction may be made in silico. In certain embodiments, the prediction may be made in vitro. In certain embodiments, the in silico or in vitro prediction may be made based on a model antibody or antibody fragment, which may be for example a full-size Ig molecule such as an IgG (IgG1, IgG2, IgG3, or IgG4), a Fab fragment, an scFv, a bispecific antibody or antibody fragment such as one having the structure in any of FIGS. 2-7. In particular embodiments, published CH1+CLκ domain coordinates may be used for prediction, such as CH1+CLκ domain coordinates from PDB (Protein Data Bank), e.g., ID 1fvd, using any appropriate method (e.g., Maguire J. B., et al., J Chem Theory Comput. 2018 May 8; 14(5):2751-2760.)

In some embodiments, the method may comprise a step of pre-selecting combinations of CH1 domain substitution(s) and CL domain substitution(s) which are predicted to increase the CH1-CL interdomain interaction, such as an interaction mediated by a hydrogen bond. In certain embodiments, the prediction may be made in silico. For example, Rosetta Monte Carlo (MC) Hydrogen Bond Network (HBNet) (see, e.g., Maguire J. B., et al., J Chem Theory Comput. 2018 May 8; 14(5):2751-2760.), a computational protocol for in silico modeling of amino acid substitutions at protein-protein interfaces to design self-contained hydrogen bond networks may be used. In certain embodiments, the prediction may be made in vitro. In certain embodiments, the in silico or in vitro prediction may be made based on a model antibody or antibody fragment, which may be for example a full-size Ig molecule such as an IgG (IgG1, IgG2, IgG3, or IgG4), a Fab fragment, an scFv, a bispecific antibody or antibody fragment such as one having the structure in any of FIGS. 2-7. In particular embodiments, published CH1+CLκ domain coordinates may be used for prediction, such as CH1+CLκ domain coordinates from PDB (Protein Data Bank), e.g., ID 1fvd, using any appropriate method (e.g., Maguire J. B., et al., J Chem Theory Comput. 2018 May 8; 14(5):2751-2760.)

In some embodiments, the number of CH1 substitution positions contained in the CH1-CL domain set library may be pre-determined. For example, the number may be predetermined to be: 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

In some embodiments, the number of CL substitution positions contained in the CH1-CL domain set library may be pre-determined. For example, the number may be predetermined to be: 1 or more, 2 or more, 3 or more, 4 or more, 5 or more, or 6 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, 5, 6, 7, 8, 9, or 10.

In some embodiments, a certain known CH1-CL design set or all known CH1-CL design sets may be removed from the CH1-CL domain set library.

In some embodiments, the method may comprise variegating any combinations of (i) the CH1 substitution positions contained in any of the CH1 domain libraries described herein and (ii) the CL substitution positions contained in any of the CL domain libraries described herein.

In certain embodiments, a degenerate codon, optionally a degenerate RMW codon representing six naturally occurring amino acids (D, T, A, E, K, and N) or a degenerate NNK codon representing all 20 naturally occurring amino acid residues may be used, to induce variegation at a pre-determined position.

In some embodiments, the method may comprise variegating any combinations of (i) the CH1 substitutions contained in any of the CH1 domain libraries described herein and (ii) the CL substitutions contained in any of the CL domain libraries described herein.

In certain embodiments, the method may comprise introducing any combinations of (i) the CH1 substitutions contained in any of the CH1 domain libraries described herein and (ii) the CLκ substitutions contained in any of the CLκ domain libraries described herein may be incorporated. In certain embodiments, in a CH1-CLλ domain set library, any combinations of (i) the CH1 substitutions contained in any of the CH1 domain libraries described herein and (ii) the CLλ substitutions contained in any of the CLλ domain libraries described herein.

Also contemplated by the present disclosure are CH1-CL domain set libraries. In some embodiments, the CH1-CL domain set library may be the library generated by any methods of generating a CH1-CL domain set library described herein. The CH1-CL domain set library may be a CH1-CLκ domain set library, a CH1-CLλ domain set library, or a library containing both CH1-CLκ domain sets and CH1-CLλ domain sets. Also provided herein are methods of identifying one or more variant CH1 domains that preferentially pair with a CL domain or a variant CL domain, identifying one or more CL domain and/or variant CL domains that preferentially pair with a variant CH1 domain, and/or identifying one or more sets of a CH1 domain and a CL domain that preferentially pair with each other.

In some embodiments, the method is a method of identifying one or more sets of a CH1 domain and a CL domain that preferentially pair with each other. Such a method may comprise at least three steps.

The first step may comprise computationally or recombinantly co-expressing or combining (a-1) a first polypeptide or a first set of polypeptides each comprising a wild-type CH1 domain or a variant CH1 domain and (a-2) a second polypeptide or a second set of polypeptides each comprising a wild-type CL domain or a variant CL domain. In certain embodiments, the variant CH1 domain(s) may be expressed from the variant CH1 domain library as described above. In certain embodiments, the variant CL domain(s) may be expressed from a variant CL domain library as described above. In certain embodiments, the variant CH1 domain(s) and the variant CL domain(s) may be expressed from the CH1-CL domain set library as described above. In certain embodiments, the variant CH1 domain(s) and the variant CL domain(s) may be expressed from a CH1-CL domain set library comprising random mutation(s) which cause random amino acid alteration(s) in CH1 and/or CL domains.

The second step may comprise quantifying the binding or binding preference between the CH1 domain or variant CH1 domain and the CL domain or variant CL domain. In some embodiments, the CH1-CL interdomain interaction, such as an interaction mediated by a hydrogen bond may be quantified. In certain embodiments, the CH1-CL interdomain interaction may be quantified in silico. In certain embodiments, the CH1-CL interdomain interaction may be quantified in vitro. In certain embodiments, the in silico or in vitro quantification may be performed using Rosetta Monte Carlo (MC) Hydrogen Bond Network (HBNet) (see, e.g., Maguire J. B., et al., J Chem Theory Comput. 2018 May 8; 14(5):2751-2760.), a computational protocol for in silico modeling of amino acid substitutions at protein-protein interfaces to design self-contained hydrogen bond networks may be used. In certain embodiments, the in silico or in vitro quantification may be performed based on a model antibody or antibody fragment, which may be for example a full-size Ig molecule such as an IgG (IgG1, IgG2, IgG3, or IgG4), a Fab fragment, an scFv, a bispecific antibody or antibody fragment such as one having the structure in any of FIGS. 2-7. In particular embodiments, published CH1+CLκ domain coordinates may be used for the CH1-CL interdomain interaction quantification, such as CH1+CLκ domain coordinates from PDB (Protein Data Bank), e.g., ID 1fvd, using any appropriate method (e.g., Maguire J. B., et al., J Chem Theory Comput. 2018 May 8; 14(5):2751-2760.).

The third step may comprise selecting one or more sets of a CH1 domain or variant CH1 domain and a CL domain or variant CL domain which provide preferential CH1-CL paring. Such preferential CH1-CL pairing may optionally be equivalent or higher relative to the paring provided by a reference CH1-CL set. In certain instances, the reference CH1-CL set may optionally comprise a wildtype CH1 domain, a wildtype CL domain, a variant CH1 domain according to the present invention, and/or a variant CL domain according to the present invention. In certain instances, the reference CH1-CL set may optionally be a wild type CH1-CL domain set and/or a CH1-CL domain set according to the present invention.

The variegation may be made to any available CH1 and/or CL sequences, i.e., wild-type or modified sequences. In some embodiments, the CH1 variegation may be made to the reference CH1 sequence of SEQ ID NO: 1. In some embodiments, the CL variegation may be made to the reference CLκ sequence of SEQ ID NO: 2 and/or the reference CLλ sequence of SEQ ID NO: 9.

In some embodiments, the first polypeptide may contain or expressed with a first tag and the second polypeptide may contain or expressed with a second tag that is different from the first tag. This would allow specifically identifying the presence of a cognate CH1-CL pair (i.e., proper paring between polypeptides as intended) by techniques such as AlphaLISA® (signal is generated when the first and second tags are in the proximity, i.e., the first and second polypeptides are paired) and/or flow cytometry.

In certain embodiments, in the first step, a full-size bispecific antibody in which a test CH1-CL set and a reference CH1-CL set (e.g., a WT CH1-CL set) are comprised may be expressed. In such cases, the preferential pairing may be assed based on the % correctly paired antibodies, e.g., among all the full-size antibodies produced. In such cases, if the % correctly paired is higher when using a test CH1-CL set with a WT CH1-CL set rather that when two reference CH1-CL sets (e.g., two WT CH1-CL sets) are used, the test CH1-CL set may be considered to provide preferential pairing.

A method of identifying one or more sets of a CH1 domain and a CL domain that preferentially pair with each other according to the present disclosure may comprise one or more additional steps.

In some embodiments, the method may further comprise a step of selecting CH1-CL domain sets based on the number of CH1 substitutions and/or the number of CL substitutions.

In some embodiments, CH1-CL domain sets meeting a certain criterion of the number of CH1 substitution positions. For example, CH1-CL domain sets comprising 1 or more, 2 or more, 3 or more, 4 or more, 5 or more CH1 substitutions; 10 CH1 substitutions or below, 9 CH1 substitutions or below, 8 CH1 substitutions or below, 7 CH1 substitutions or below, 6 CH1 substitutions or below, 5 CH1 substitutions or below, 4 CH1 substitutions or below, 3 CH1 substitutions or below, or 2 CH1 substitutions or below; between 1-10 CH1 substitutions, between 1-9 CH1 substitutions, between 1-8 CH1 substitutions, between 1-7 CH1 substitutions, between 1-6 CH1 substitutions, between 1-5 CH1 substitutions, between 1-4 CH1 substitutions; between 1-3 CH1 substitutions; between 1-2 CH1 substitutions; and/or 1, 2, 3, 4, or 5 CH1 substitutions may be selected.

In some embodiments, CH1-CL domain sets meeting a certain criterion of the number of CL substitution positions. For example, CH1-CL domain sets comprising 1 or more, 2 or more, 3 or more, 4 or more, 5 or more CL substitutions; 10 CL substitutions or below, 9 CL substitutions or below, 8 CL substitutions or below, 7 CL substitutions or below, 6 CL substitutions or below, 5 CL substitutions or below, 4 CL substitutions or below, 3 CL substitutions or below, or 2 CL substitutions or below; between 1-10 CL substitutions, between 1-9 CL substitutions, between 1-8 CL substitutions, between 1-7 CL substitutions, between 1-6 CL substitutions, between 1-5 CL substitutions, between 1-4 CL substitutions; between 1-3 CL substitutions; between 1-2 CL substitutions; and/or 1, 2, 3, 4, or 5 CL substitutions may be selected. In some embodiments, the method may further comprise a step of selecting CH1-CL domain sets based on the CH1-CL interface binding energy and/or changes in the CH1-CL interface binding energy protein complex stability relative to a reference CH1-CL set such as a WT CH1-CL set (e.g., as predicted by Rosetta). For example, prediction of the CH1-CL interface binding energy and/or changes in the CH1-CL interface binding energy protein complex stability may be performed as described in the “no backrub-generated backbone flexibility” protocol from Barlow K. A. et al (J Phys Chem B. 2018 May 31; 122(21):5389-5399.) For example, selection may be performed as described herein in Step 3 of Example 2.

In some embodiments, one or more (or all) known CH1-CL design sets may be removed from the CH1-CL domain set library.

In some embodiments, the method may further comprise a step of introducing one or more amino acid modifications to one or more of pre-selected CH1-CL domain sets. In certain embodiments, such modifications may comprise reversion of certain amino acid substitution(s) back to WT residue. In certain embodiments, such modifications may comprise introducing conservative amino acid changes. In certain embodiments, such modifications may introduce another CH1 and/or CL domain substitution(s) from another CH1-CL set. In some cases, the another CH1-CL sets may be a pre-existing CH1-CL set, a CH1-CL design set according to the present disclosure, or a CH-CL design set pre-selected during the method of identifying one or more sets of a CH1 domain and a CL domain that preferentially pair with each other.

In some embodiments, the method may further comprise a step of selecting CH1-CL domain sets based on antibody characteristics. Such characteristics may include but are not limited to: (i) production yield, which may be assessed in one or more cell types (e.g., mammalian cells such as CHO cells and HEK cells, yeast cells, insect cells, plant cells etc) using any appropriate methods or as described herein and/or compatibility to certain antibody purification methods (e.g., protein A affinity purification); (ii) degree of aggregation (e.g., presence of multimers of a full antibody) (also referred to as purity herein), which may be quantified using any appropriate methods or as described herein, e.g., by chromatography such as size exclusion chromatography (SEC) or electrophoresis such as SDS-PAGE; (iii) rates of correct pairing (e.g., between heavy chains and/or between heavy and light chains), which may be assessed using any appropriate methods or as described herein e.g., by LC-MS; (iv) melting temperature (Tm) and/or aggregation temperature (Tagg) (e.g., Tagg266), which may be measured using any appropriate methods or as described herein e.g., by Differential scanning fluorimetry (DSF) or Differential scanning calorimetry (DSC) or using an instrument such as Uncle®; (v) “pI”, isoelectric point (“pI”), which may be measure using any appropriate methods; (vi) the level of interaction with polyspecificity reagent (“PSR”), which may be measured using any appropriate methods or as described herein e.g., as in WO2014/179363; (vii) hydrophobic interaction of the antibody which may be measured using any appropriate methods or as described herein, e.g., by hydrophobic interaction chromatography (“HIC”) as in e.g., Estep P, et al. MAbs. 2015 May-June; 7(3): 553-561.; (viii) self-interaction, which may be measured, e.g., by AC-SINS or DLS as described above; (ix) stability to high or low pH stress, which may be measured as described herein; (x) solubility; (xi) production costs and/or time; (xii) other stability parameters; (xiii) shelf life; (xiv) in vivo half-life; and/or (xv) immunogenicity, which may be assessed using any appropriate methods.

In particular embodiments, a heavy chain heterodimerizing technology may be further used (e.g., “whole-in-knob” modifications and/or “S=S” modifications in the CH3 domain) to ensure correct heavy-heavy heterodimerization. In such cases, desired % pairs paired correctly (“PC”) may be about >50%, about >55%, about >60%, about >65%, about >70%, about >75%, about >80%, about >85%, about >90%, about >95%, about >96%, about >97%, about >98%, about >99%, or about 100%.

In certain embodiments, the desired % PC may be relative to a reference CH1-CL set, e.g., a pre-existing set of CH1 and CL that preferentially pair with each other (e.g., in Table 1).

In further embodiments, a full-size, IgG-like bispecific antibody utilizing two different variant CH1-CL sets may be expressed and assessed.

In some methods, the method may further comprise expressing the selected variant CH1 domain(s), variant CL domain(s), and/or CH1-CL set(s) as a bispecific antibody or antibody fragment and assessing the manufacturing feasibility. For example, this may evaluate the degree of aggregation or purity (e.g., presence of multimers of a full antibody) and/or the amount of half antibody (i.e., one heavy chain and one light chain in a molecule), both of which may be quantified by, e.g., chromatography such as size exclusion chromatography (SEC) or electrophoresis such as SDS-PAGE; melting temperature (Tm), which may be measured by, e.g., Differential scanning fluorimetry (DSF); production yields in a n appropriate cell type (e.g., HEK293 cells or yeast cells); “pI”, isoelectric point (“pI”); the level of interaction with polyspecificity reagent (“PSR”), which may be measured as in WO2014/179363; hydrophobic interaction of the antibody which may be measured by hydrophobic interaction chromatography (“HIC”) as measured as in e.g., Estep P, et al. (2015) or MAbs 7(3):553-561); solubility; production costs; and/or production time. In addition, or alternatively, the method may further comprise expressing the selected variant CH1 domain(s), variant CL domain(s), and/or CH1-CL set(s) as a bispecific antibody or antibody fragment and assessing other parameters such as: stability; shelf life; in vivo half-life; and/or immunogenicity.

In some embodiments, any of such characteristics may depend on (a) the particular structure of the molecule or multi-specific antibody or antigen-binding antibody fragment structure which incorporates a variant CH1-CL domain set and/or (b) the variable domains providing particular binding specificities. Therefore, in some cases, when one contemplates to design a multi-specific antibody or antigen-binding antibody fragment having specified/given antigen specificities, such as specified variable region sequences, multiple CH1-CL domain sets and/or multiple combinations of CH1-CL domain sets may be tested in the particular antibody or antibody fragment structure and/or antigen specificity settings.

Also provided herein are methods of screening for a combination of (i) a first CH1-CL set and (ii) a second CH1-CL set suited for a multi-specific antibody or antigen-binding antibody fragment (e.g., having a particular antibody structure or format) having antigen specificities of interest, such as having variable region sequences of interest. The first CH1-CL set in this case is a set of a first variant CH1 domain polypeptide and a first variant CL domain polypeptide. The second CH1-CL set in this case is a set of a second variant CH1 domain polypeptide and a second variant CL domain polypeptide. I.e., the methods are for determining combinations of CH1-CL sets particularly useful in the context of a multi-specific antibody or antigen-binding antibody fragment having a given structure and/or specificities.

Such a method may comprise: (a) expressing a plurality of multi-specific antibodies and/or antigen-binding antibody fragments, comprising different combinations of (i) a first CH1-CL set candidate and (ii) a second CH1-CL set candidate; and (b) selecting one or more combinations of (i) a first CH1-CL set and (ii) a second CH1-CL set based on one or more characteristics of the multi-specific antibodies and/or antigen-binding antibody fragments expressed in step (a). In some embodiments, at least one of the one or more characteristics may be selected from the characteristics (i)-(xv) described above.

In some embodiments, the multiple multi-specific antibodies and/or antigen-binding antibody fragments expressed in step (b) may comprise: (I) a first polypeptide comprising a first variant CH1 domain polypeptide and a first antigen-binding domain polypeptide; (II) a second polypeptide comprising a second variant CH1 domain polypeptide and a second antigen-binding domain polypeptide; (III) a third polypeptide comprising a first variant CL domain polypeptide and a third antigen-binding domain polypeptide; and (IV) a fourth polypeptide comprising a second variant CL domain polypeptide and a fourth antigen-binding domain polypeptide, wherein the first and third polypeptide preferentially pair with each other and the second and fourth polypeptide preferentially pair with each other. The two sets of preferential pairing may render the resulting antibody or antibody fragment multi-specific.

In some instances, the first variant CH1 domain polypeptide, the second variant CH1 domain polypeptide, the first CLκ or CLλ domain polypeptide, and/or the second CLκ or CLλ domain polypeptide may be any of the variant domain polypeptides described herein. In certain embodiments, the first CH1-CL set candidate and/or the second CH1-CL set candidate may be any of the CH1-CL sets described herein.

Examples are provided below to illustrate the present invention. These examples are not meant to constrain the present invention to any particular application or theory of operation.

Libraries and Methods for Identifying Two Polypeptides which Preferentially Pair with Each Other

In the Examples, various CH1 domain polypeptides (WT or variant) and various CL (CLκ or CLλ) domain polypeptides (WT or variant) were provided together in silico and the binding preference was calculated in silico. This strategy successfully resulted in identification of CH1-CL domain polypeptide sets in which the CH1 and CL domains preferentially pair with each other. Based on the results, the strategy is readily applicable to identifying two polypeptides which preferentially pair with each other (which may be referred to as first polypeptide and second polypeptide and may not be limited to polypeptides comprising a CH1 domain or a CL domain).

Therefore, in some aspects, methods of generating libraries (which may be a library of sets of a first candidate polypeptide-encoding polynucleotide and a second candidate polypeptide-encoding polynucleotide or a library of sets of a first candidate polypeptide and a second candidate polypeptide), libraries generated using such a method, and methods of identifying one or more sets of a first polypeptide and a second polypeptide are also provided.

In such an aspect, (i) the first candidate polypeptide is the same as or is a variant of a first parent polypeptide; and (ii) the second candidate polypeptide is the same as or is a variant of a second parent polypeptide.

Essentially, in some embodiments, a library of sets of a first candidate polypeptide-encoding polynucleotide and a second candidate polypeptide-encoding polynucleotide may be generated using a method analogous to a method of generating a CH1-CL domain-encoding polynucleotide set library.

Essentially, in some embodiments, a library of sets of a first candidate polypeptide and a second candidate polypeptide may be generated using a method analogous to a method of generating a CH1-CL domain polypeptide set library.

Essentially, in some embodiments, one or more sets of a first polypeptide and a second polypeptide which preferentially pair may be identified using a method analogous to a method of identifying one or more CH1-CL domain polypeptide sets.

Such libraries and methods may be useful in a variety of situations. For example, when a given first parent polypeptide and a given second parent polypeptide do not preferentially pair with each other but one hopes to prepare a dimer between the first parent polypeptide (or a variant thereof) and the second parent polypeptide (or a variant thereof), libraries and methods described herein would allow one to efficiently modify the first and/or second parent polypeptide to obtain first and second polypeptides which preferentially pair with each other.

### EXAMPLES

In Examples described herein, the CH1 domain reference sequence (SEQ ID NO: 1) was used as a wild-type CH1 domain sequence of IgG1, the CLκ domain reference sequence (SEQ ID NO: 2) was used as a wild-type CLκ domain sequence of IgG1, and the CLλ domain reference sequence (SEQ ID NO: 9) was used as a wild-type CLλ domain sequence of IgG1. Various amino acid substitutions were incorporated to the CH1 and CL (CLκ or CLλ) reference sequences for testing the preferential CH1-CL pairing potential. Some of the CH1 and CL sequences used in Examples are provided in Appendix Tables A-C and sequence listing. Although SEQ ID NO: 1 was used as the CH1 domain reference sequence in Examples, the present invention relating to a CH1 domain sequence modification(s) may also be applied to other naturally occurring CH1 domain reference sequences, such as but not limited to SEQ ID NO: 3 (for IgG1) or another naturally occurring CH1 sequence, i.e., another IgG1, IgG2, IgG3, or IgG4 CH1 sequence.

Unless otherwise noted, the CH2 and CH3 reference sequences (SEQ ID NOS: 7 and 8, respectively) were used in the Examples, when applicable. It is noted that antibodies expressed in CHO cells (but not HEK cells) did not contain the C-terminal lysine at amino acid position 447 (i.e., the C-terminal “K” of the sequence of SEQ ID NO: 8 was omitted).

### Example 1: Rosetta Design Generation (HBNet Algorithm)

To modulate heavy chain (HC):light chain (LC) interfaces and the HC-LC interaction, Rosetta Monte Carlo (MC) Hydrogen Bond Network (HBNet) (see, e.g., Maguire J. B., et al., J Chem Theory Comput. 2018 May 8; 14(5):2751-2760.), a computational protocol for in silico modeling of amino acid substitutions at protein-protein interfaces to design self-contained hydrogen bond networks, was used. Since hydrogen bonds at and across protein-protein interfaces contribute to binding specificity (see, e.g., Kortemme T. et al., J Mol Biol. 2003 Feb. 28; 326(4):1239-59.), the MC HBNet computational approach was chosen to design HC:LC interfaces with a specific pairing preference for use in multi-specific antibody (such as bispecific antibody (bsAb)) constructs. Protein design algorithms such as MC HBNet which are motivated explicitly by polar hydrogen bond interactions may sample a portion of the so-called “sequence space” that is orthogonal to the “traditional” sampling biased towards van der Waals-type interactions (Stranges P. B. and Kuhlman B., Protein Sci. 2013 January; 22(1):74-82.), thus potentially leading to novel bsAb pairing solutions.

In the first stage of the screening in Example 1, the correctly paired “cognate” designed interface was considered and optimized as described below (“cognate” as used herein, when referring to the relationship between a CH1 and a CLκ, means that each of a variant CH1 domain and a variant CLκ domain comprises an amino acid substitution(s) so that the variant CH1 domain and variant CLκ domain preferentially pair with each other):

First, we ran the HBNet protocol on the exemplary CH1+CLκ domain coordinates from PDB (Protein Data Bank) ID 1fvd, using the published (see, e.g., Maguire J. B., et al., J Chem Theory Comput. 2018 May 8; 14(5):2751-2760.) Rosetta script and parameters for protein interface design, modified slightly to permit two-sided interface design i.e., allow amino acid substitutions in both CH1 and CLκ. The HC in the input structure contained 103 residues (EU numbering Ala118 to Cys220) and the LC contained 107 residues (EU numbering Arg108 to Cys214). The HBNet output contained a total of 3571 CH1-CLκ sequence pairs; there were 1959 unique (i.e. not repeated) CLκ sequences, 1657 CH1 unique sequences, and a total of 3164 unique CH1-CLκ pairs (3164/3571=89% were unique).

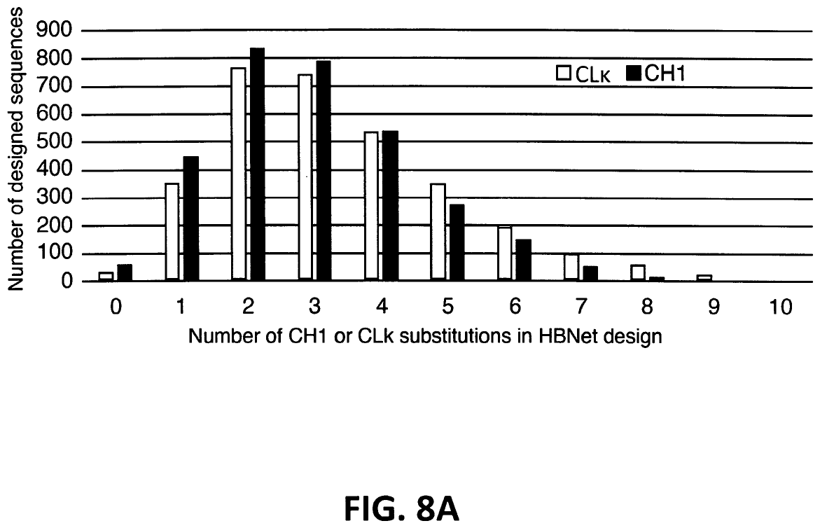

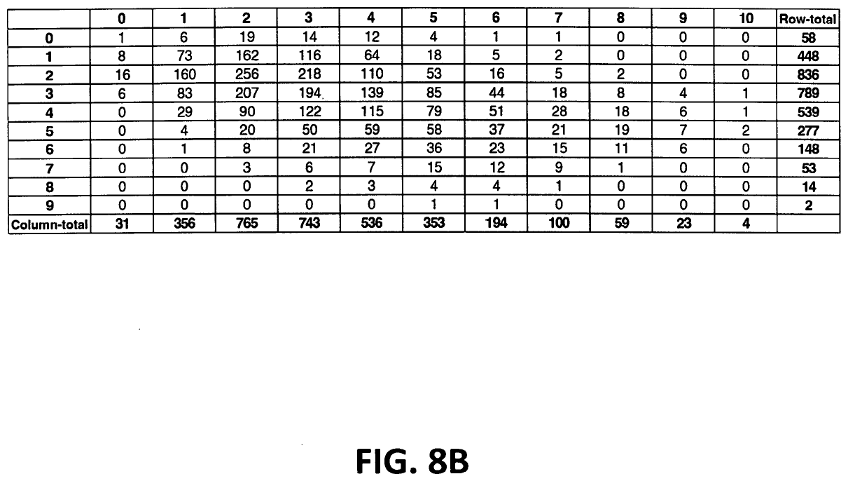

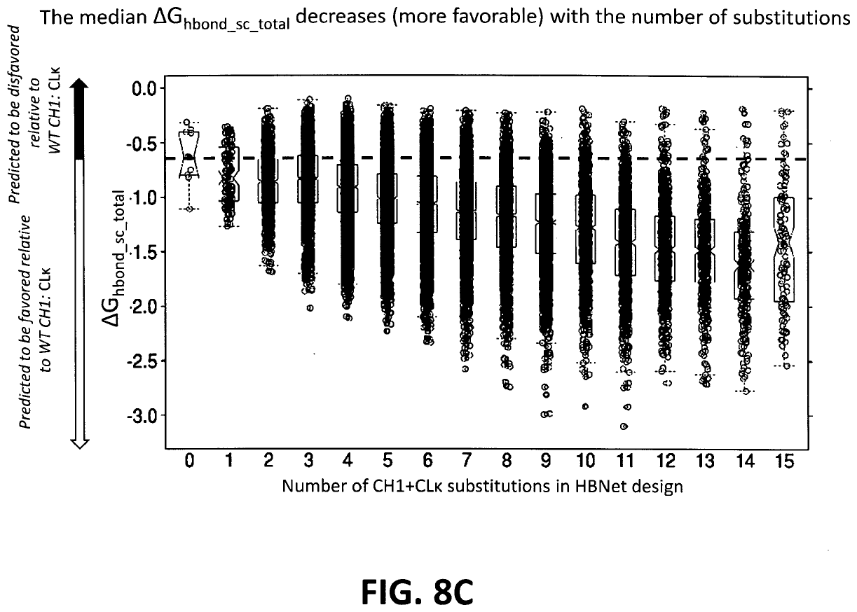

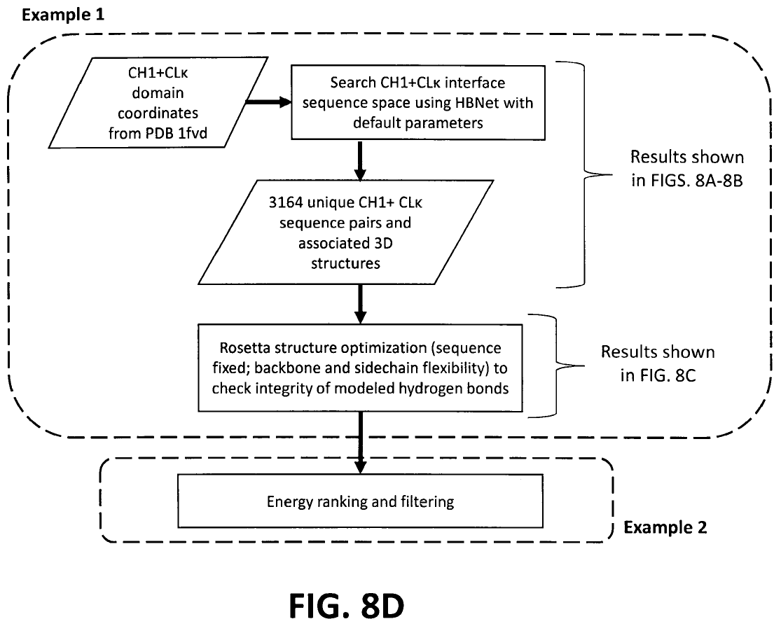

FIG. 8A shows a histogram of the number of amino acid (AA) substitutions found in the unique pairs of CH1 and CLκ (N=3164). The CLκ domains had on average 3.0 substitutions (range 0-9), while CH1 domains had on average 3.4 substitutions (range 0-10).

FIG. 8B shows a matrix of the number of CLκ substitutions versus CH1 substitutions in the unique CH1-CLκ sequence sets (N=3164 sets). The most frequently observed substitution number combination was two substitutions each in CH1 and CLκ (representing 256/3164 or 8% of the unique CH1-CLκ sets). Some sets contained 6 or more substitutions in each of CH1 and CLκ (83/3164 or 2.6% of the unique CH1-CLκ sets).

The second stage of Example 1 analyzed whether the substitutions in the CH1-CLκ sequence sets sampled by HBnet lead to CH1-CLκ hydrogen-bond interactions.

Briefly, the PDB 1fvd template with the HBNET-generated substitutions was optimized using a RosettaScripts protocol (see, e.g., Fleishman S. J., et al, PLoS One. 2011 Jun. 24; 6(6):e20161) that makes use of rigid-body docking, backbone and side-chain minimization and packing. Subsequently, the distribution of the Rosetta interface sidechain-mediated hydrogen bond score term ΔG_{hbond_sc_total} was computed. AG here refers to the value of the CH1-CLκ interface binding energy, or a component thereof, such as hydrogen bonding. ΔG_{hbond_sc_total} was computed using the Talaris2014 energy function as the sum of (1) the backbone-sidechain hydrogen bond term, ΔG_{hbond_bb_sc} and (2) the sidechain-sidechain hydrogen bond term, ΔG_{hbond_sc}) (see, e.g., O'Meara M. J., et al, J Chem Theory Comput. 2015 Feb. 10; 11(2):609-22.; and Alford R. F. et al., J. Chem. Theory Comput. 2017, 13, 6, 3031-3048) and plotted as a function of the number of CH1-CLκ substitutions (FIG. 8C). In the plot, more negative ΔG_{hbond_sc_total} values are suggestive of stronger and/or more numerous interface hydrogen bonds. The ΔG_{hbond_sc_total} value for the WT CH1-WT CLκ pair (i.e., x-axis value 0) is ˜-0.6 units (dash line). With increasing number of substitutions, a clear trend for stronger and/or more hydrogen bond interactions relative to this WT reference was observed, exemplified by the continuously decreasing median ΔG_{hbond_sc_total} values in the boxplot. On the other hand, there was also a proportion of the HBNet designs (HBNet designs encompass variant CH1-CLκ sets, variant CH1 domains, and/or variant CLκ domains identified by the HBNet-based screening in the first stage of Example 1) with overall weaker hydrogen bond interactions than the WT reference (circles above the dashed line).

The overall scheme for the screening in the first stage of Example 1 (i.e., MC HBNet for sampling sequence space with sidechain rotamer flexibility and fixed protein backbone) and the second stage of Example 1 (i.e., a “standard” Rosetta optimization step to check if the HBNet predicted hydrogen bonds hold up under optimization with both backbone and sidechain flexibility) is visualized in FIG. 8D.

The sequences from Example 1 were then subjected to energetic comparisons in the context of mis-paired interfaces in the following stage (Example 2).

### Example 2: In Silico Screening—Rosetta Scoring/Evaluation of SIDS (Single Interface Designs)

In this example, changes in the binding energy between the variant CH1 and CLκ domains relative to the binding energy between WT CH1 and WT CLκ domains were analyzed using Rosetta scoring of sequences for some of the CH1-CLκ sets identified in Example 1.

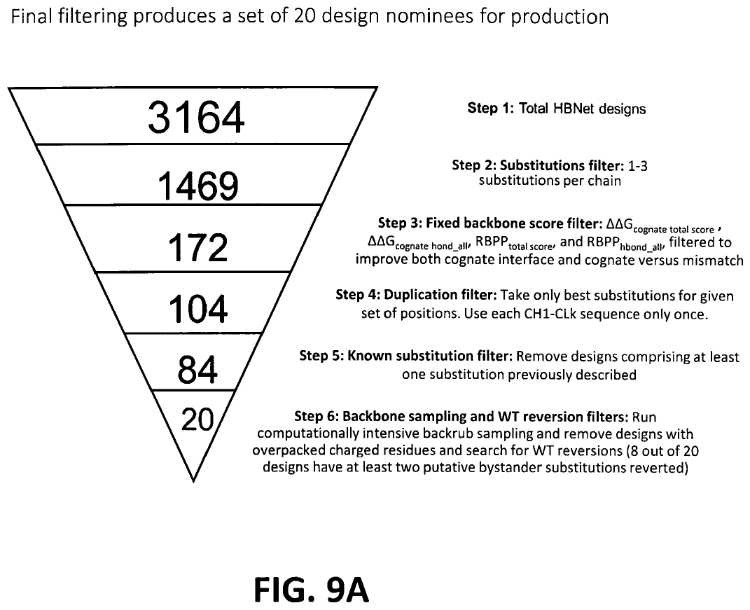

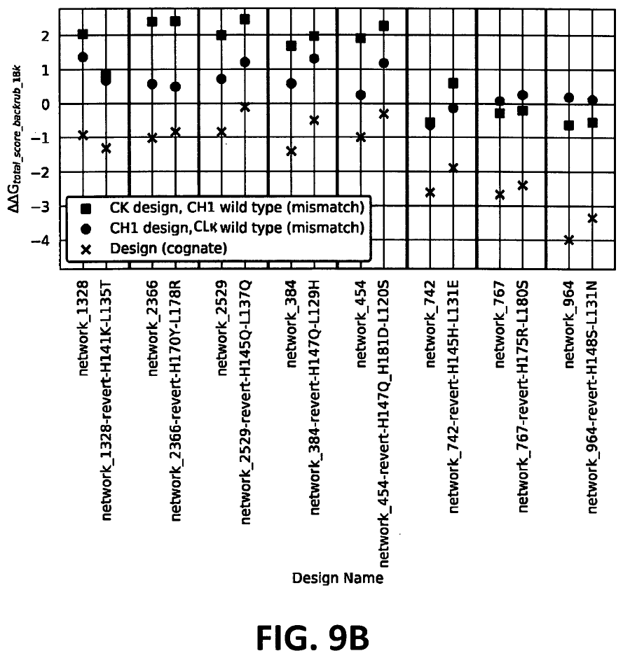

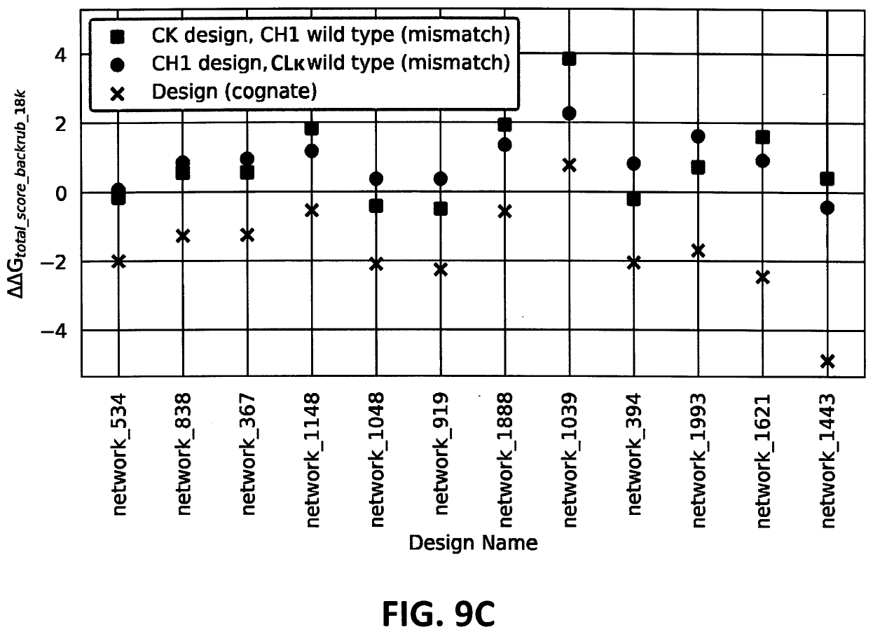

In earlier work, Rosetta scoring of sequences helped validate the use of the Rosetta “flex ΔΔG” protocol (ΔΔG is defined as change in interface binding energy (ΔG) after substitution, compared to WT interface binding energy) (Barlow K. A. et al., J Phys Chem B. 2018 May 31; 122(21):5389-5399.) to predict ΔΔG. This protocol was extended to screen for preferentially pairing variant CH1-CLκ domains and also helped determine parameters of the flex ΔΔG protocol for the following in silico screening and characterization. Accordingly, further screening steps based on the interchain binding energy were performed as follows and as visualized in FIG. 9A.

### From Step 1 (HBNet Designs) to Step 2 (Substitution Filter)

First, from the 3164 CH1-CLκ sets identified in Example 1 (Step 1 in FIG. 9A), only CH1-CLκ sets in which each of CH1 and CLκ contains at least one substitution but no more than three substitutions were selected, to exclude sets for which Rosetta modeling may not be optimal. This resulted in 1469 CH1-CLκ sets (Step 2 in FIG. 9A).

### Step 3 (Fixed Backbone Score Filter)

Subsequently, interface binding energy and changes in bound protein complex stability (as predicted by Rosetta) were calculated as described in the “no backrub-generated backbone flexibility” protocol from Barlow K. A. et al (J Phys Chem B. 2018 May 31; 122(21):5389-5399.) Briefly, four input WT crystal structures were first selected from the set of available antibody structures in the RCSB Protein Data Bank (PDB) based on the completeness of backbone coordinates in the structure and high resolution, among other considerations: 1aj7, 117i, 4olv, 6b14.

The “no backrub-generated backbone flexibility” protocol was then used to estimate the energetic effect of the substitutions of each HBNet CH1/CLκ sequence pair in each input WT PDB context, and the resulting energies averaged across calculations with all four input PDBs. In addition to modeling the energetic effects of the “cognate” designed interface (where both CH1 and CLκ contain their corresponding HBNet-generated substitutions), CH1_{design}-CLκ_{WT} pairs (i.e., CH1 is a variant CH1 domain identified in Example 1 but CLκ is wildtype (WT)) and CH1_{WR}-CLκ_{design} pairs (CH1 is wildtype (WT) but CLκ is a variant CLκ identified in Example 1) (pairs of a WT domain and a design domain are also referred to as mis-paired, mis-paired interface, or mis-paired sets herein) were also modeled for the “Rosetta Bispecific Pairing Propensity (RBPP)” score metric (RBPP metric). Briefly, a RBPP metric was then used to rank the relative predicted propensity of each design to correctly pair in the intended cognate/heterodimer state. RBPP (Rosetta Bispecific Pairing Propensity) for a cognate CH1-CLκ pair was defined as RBPP=ΔΔG_{cognate designed CH1/CLκ interface}−(ΔΔG_{CH1 WT-CLκ Design}+ΔΔG_{CH1 Design-CLκ WT})/2. ΔΔG_{cognate designed CH1/CLκ interface} is the ΔΔG value for a cognate CH1-CLκ1 set identified in Example 1. ΔΔG_{CH1 WT-CLκ Design} and ΔΔG_{CH1 Design-CLκ WT} are the ΔΔG values for the relevant mis-paired CH1-CLκ1 sets (i.e., a pair of a WT CH1 and a design CLκ from a cognate set identified in Example 1 and a pair of a design CH1 from a cognate set identified in Example 1 and a WT CLκ, respectively).

Screening based on the interface binding energy applied the following four filters to the 1439 CH1-CLκ sets:

(1) ΔΔG_{cognate total score}≤0 REU (Rosetta energy units). ΔΔG_{cognate total score}, which is the same as ΔΔG_{cognate designed CH1/CLκ interface}, represents the predicted change in interface binding energy for the “cognate” (correctly paired, i.e., pairs as identified in Example 1) designed CH1-CLκ interface, compared to the WT CH1/CLκ interface, with the full Rosetta “total score” (sum of all score terms). As lower Rosetta scores correspond to more stable (lower energy) models, setting this filter below 0 ensured that no design was predicted to have weaker interface interactions compared to the WT interface. 265 of 1469 designs passed this filter.

(2) ΔΔG_{cognate hbond_all}≤0 REU. ΔΔG_{cognate hbond_all} represents the predicted change in interface binding energy for the cognate interface (i.e., the interface between the variant CH1 domain and variant CLκ of a CH1-CLκ set identified in Example 1), compared to WT interface (i.e., the interface between WT CH1 and WT CLκ), for only the summation of the score terms of the Talaris score function in Rosetta relating to the energetics of hydrogen bonds (see, e.g., Leaver-Fay A. et al., Methods Enzymol. 2013; 523:109-43.). As the goal of HBNet design was to create novel hydrogen bond interactions across the interface, including this filter made sure that favorable predicted hydrogen bond interactions were predicted by this screening protocol. 991 of 1469 designs passed this filter.

(3) RBPP_{total score} 0−1 REU. RBPP_{total score} is the same as RBPP, defined above as ΔΔG_{cognate designed CH1/CLκ interface}−(ΔΔG_{CH1 WT-CLκ Design}30 ΔΔG_{CH1 Design-CLκ WT)/}2. With this metric, the total Rosetta score of the cognate designed interface was filtered to be more energetically favorable than in the mis-paired interfaces. 283 out of 1469 designs passed this filter.

(4) RBPP_{hbond_all}≤0 REU. RBPP_{hbond_all} is defined as ΔΔG_{cognate hbond_all}−(ΔΔG_{CH1 WT-CLκ Design_hbond_all}+ΔΔG_{CH1 Design-CLκ WT_hbond_all})/2. With this metric, the hydrogen bonding score terms of the cognate designed interface were also filtered to be more energetically favorable than in the mis-paired interfaces. 1092 out of 1469 designs passed this filter.

When these four score filters were applied simultaneously to the set of 1469 designs, 172 designs passed all filters (Step 3 in FIG. 9A).

### Step 4 (Duplication Filter)

Of the 172 designs that passed all score filters in the above tests, there were 147 designs with unique sets of substituted positions (i.e., the combination of positions, not just the combination of amino acid residues, are unique). Among the 147 designs, for any design with a duplicate set of substituted positions as another design, only the design with the best ΔΔG_{cognate total score} was kept (more specifically, only the best scoring (by ΔΔG_{cognate total score}) instance of each unique CH1 or CLκ substituted sequence was kept). This means that, for example, if a particular designed CH1 sequence appeared paired with multiple designed CLκ sequences, only the CH1/CLκ pair with the best score was kept. 104 designs remained after this filter. (Step 4 in FIG. 9A).

### Step 5 (Known Substitution Filter)

20 of the 104 designs from Step 4 contained some substitutions previously reported. These 20 designs were filtered out leaving 84 novel designs (Step 5 in FIG. 9A).

### Step 6 (Backbone Sampling and WT Reversion Filters)

The 84 designs from Step 5 were then run through the “computationally intensive” flex ΔΔG protocol with “backrub-generated backbone flexibility”. 20 best scoring designs of the 84 were selected using RBPP_{total score backrub 18k} (where backrub 18k represents the 18,000 backrub simulation steps used to introduce protein backbone flexibility) score as the primary ranking metric. The selection also included manual visual inspection of Rosetta-generated models in the Pymol protein visualization software, which discarded three designs that contained designed potentially resulting in tightly packed charged residue interactions that might result in charge-charge repulsion.

Subsequently, reversion(s) to the WT amino acid residue at some of the substituted positions were tested on the 20 designs. This was done in order to minimize the number of designed amino acid substitutions relative to WT CH1 and CLκ domains. Briefly, for each of the 20 designs, an exhaustive scoring of all possible single and multiple substitution reversions (leaving at least one substitution on each chain) was performed using the “no backrub-generated backbone flexibility” flex ΔΔG protocol. The best scoring set of substitutions to revert (lowest scoring set of WT reversions by RBPP_{total score}) was then evaluated using the “backrub-generated backbone flexibility” flex ΔΔG protocol. If the RBPP_{total score backrub 18k} increased by ≤1 REU and the RBPP_{hbond all backrub 18k} score increased by ≤0.5 REU, then the reversion set of substitutions was chosen to be carried forward. Energies for reversions carried forward, along with the energies of their corresponding original designs, are shown in FIG. 9B. Energies for designs that were carried forward without reversions are shown in in FIG. 9C.

20 designs thus selected were subjected to experimental characterization in the “single interface design (SID)” format (SID refers to an antibody or antibody fragment in which the variant CH1-CLκ domain set is used in one pair of HC and LC on one Fab arm of the IgG) in Example 3.

### Example 3: SID: Experimental Production in HEK Cells and Validation

Table 2 summarizes the 20 CH1-CLκ sets selected in Example 2 for experimental production and characterization in Example 3. Table 2 also provides SEQ ID NOs assigned to exemplary variant CH1 and CLκ domain sequences in which the indicated CH1-CLκ substitutions are incorporated to the reference CH1 and CLκ domain sequences (SEQ ID NO: 1 and 2, respectively). However, it is noted that the variant CH1 and CLκ domains according to the present invention are not limited to those specific CH1 and CLκ sequences but rather any variant CH1 and/or CLκ domain(s) comprising such CH1 and/or CLκ substitutions are encompassed (i.e., CH1 and/or CLκ substitutions may be incorporated to a CH1 and/or CLκ sequences different from the reference sequences, and/or additional substitution(s) may be further added, and/or one or more substitution(s) may be reverted back to the WT amino acid residue).

The CH1-CLκ sets of Table 2 were used in production of a bispecific antibody (bsAb) of a single interface design (SID) format (full-size, IgG-like bispecific antibody having the bottom left structure in FIG. 2D). Specifically, intended bsAb was designed to have: (1) a first heavy chain comprising a VH domain, a CH1 domain, a CH2 domain, and a CH3 domain (referred to as VH-1, CH1-1, CH2-1, and CH3-1, respectively); (2) a first light chain comprising a VL domain and a CLκ domain (referred to as VL-1 and CLκ-1, respectively); (3) a second heavy chain comprising a VH domain, a CH1 domain, a CH2 domain, and a CH3 domain (referred to as VH-2, CH1-2, CH2-2, and CH3-2, respectively); and (4) a second light chain comprising a VL domain and a CLκ domain (referred to as VL-2 and CL-2, respectively). The VH and VL sequences of Panitumumab (anti-EGFR; the VL is kappa isotype) were used as the VH-1 and VL-1. The VH and VL sequences of Ustekinumab (anti-IL-12 p40; the VL is kappa isotype) were used as the VH-2 and VL-2. The design variant CH1 domain of a test CH1-CLκ set was used as the CH1-1 and the design variant CLκ domain of the test CH1-CLκ set was used as the CLκ-1. WT CH1 domain was used as the CH1-2 and WT CLκ domain was used as the CL-2. The “knob-in-hole” substitutions in the CH3 domains and additional CH3 domain substitutions that allow a disulfide bond between CH3 to facilitate CH3 heterodimerization were also incorporated. Specifically, S354C and T366W were incorporated to the CH43-1, and Y349C, T366S, L368A, and Y407V were incorporated in the CH43-2 (EU numbering). T366W in CH43-1 (Knob substitution) and T366S, L368A, and Y407V in CH43-2 (Hole substitutions) facilitate CH3 heterodimerization and S354C in CH43-1 and Y349C in CH43-2 form a disulfide bond to support such CH3-CH3 dimerization. Panitumumab and Ustekinumab were selected as control antibodies to demonstrate the functionality of the identified variant CH1 domains, variant CLκ domains, and CH1-CLκ sets due to, e.g., high yield of the bsAb, and the decent Tm (melting temperature) values of the bsAb.

TABLE 2
CH1-CLκ sets used for experimental production in Example 3.

| | Network | | SEQ ID | CLκ | SEQ ID |
|---|---|---|---|---|---|
| CH1-CLκ Set Name | Name | CH1 substitution(s) | NO: | substitution(s) | NO: |
| H_168S_185S_187D- | 1039 | 168S; 185S; 187D | 11 | 135R | 12 |
| L_135R | | | | | |
| H_128R_147R- | 1993 | 128R; 147R | 21 | 124E; 133Q; | 22 |
| L_124E_133Q_178E | | | | 178E | |
| H_145Q_147E_181E- | 1443 | 145Q; 147E; 181E | 31 | 129R; 178R; | 32 |
| L_129R_178R_180Q | | | | 180Q | |
| H_147T_185Q- | 2529 | 147T; 185Q | 41 | 135S; 178R | 42 |
| L_135S_178R | | | | | |
| H_148R-L_124S_129E | 367 | 148R | 51 | 124S; 129E | 52 |
| H_139R_141Q_187Q- | 1888 | 139R; 141Q; 187Q | 61 | 114D; 135S; | 62 |
| L_114D_135S_138R | | | | 138R | |
| H_166K_187K- | 1328 | 166K; 187K | 71 | 137S; 138E | 72 |
| L_137S_138E | | | | | |
| H_168R_185E-L_135S | 2366 | 168R; 185E | 81 | 135S | 82 |
| H_124R_147R- | 964 | 124R; 147R | 91 | 127D; 129E | 92 |
| L_127D_129E | | | | | |
| H_147H_148E- | 767 | 147H; 148E | 101 | 127R; 129R | 102 |
| L_127R_129R | | | | | |
| H_145S-L_133Y | 1148 | 145S | 111 | 133Y | 112 |
| H_145S_181Q-L_133Y | 384 | 145S; 181Q | 121 | 133Y | 122 |
| H_145S-L_124E_133Y | 454 | 145S | 131 | 124E; 133Y | 132 |
| H_145Q_181E- | 1048 | 145Q; 181E | 141 | 120S; 178H; | 142 |
| L_120S_178H_180Q | | | | 180Q | |
| H_124R_145S_147Q- | 534 | 124R; 145S; 147Q | 151 | 127T; 129D; | 152 |
| L_127T_129D_178R | | | | 178R | |
| H_166K_187K- | 838 | 166K; 187K | 161 | 114Q; 137T; | 162 |
| L_114Q_137T_138E | | | | 138E | |
| H_147R_175D- | 919 | 147R; 175D | 171 | 129D; 178R; | 172 |
| L_129D_178R_180H | | | | 180H | |
| H_147R_175E_181Q- | 394 | 147R; 175E; 181Q | 181 | 129D; 180Q | 182 |
| L_129D_180Q | | | | | |
| H_145S_147N- | 1621 | 145S; 147N | 191 | 133Y; 180R | 192 |
| L_133Y_180R | | | | | |
| H_147N_185Y- | 742 | 147N; 185Y | 201 | 129R; 180S | 202 |
| L_129R_180S | | | | | |

* The CH1-CLκ set names (also identifiable by the Network Name together with the light chain isotype) as used herein are named by the amino acid positions (according to EU numbering) substituted in the CH1 domain (substitutions specified after “H_” (H followed by underscore), separated by “_” (underscore)) and the CLκ domain (substitutions specified after “L_” (L followed by underscore), separated by “_” (underscore)), with a dash to separate domains. For example, the H_168S_185S_187D-L_135R set has S, S, and D in the variant CH1 domain at positions 168, 185, and 187, along with R in the variant CLκ domain at position 135.
** The sequences assigned with the SEQ ID NOs shown are exemplary CH1 and CLκ sequences. Any CH1 and CLκ sequences comprising the indicated amino acid substitution(s) are encompassed by each applicable Network of the present invention.

The bsAbs comprising different CH1-CLκ sets of Table 2 were produced using the exemplary CH1 and CLκ sequences assigned with the SEQ ID NOs shown in Table 2 and compared based on the production yield, purity, and proper pairing between CH1-1 and CLκ-1.

### 1: Production Yield

BsAbs were produced in HEK293 cells and purified via protein A-based purification.

The yields were determined by measuring the protein concentration using A280 NanoDrop™. The process yields are summarized along with the total number or substitutions in the CH1 and CLκ domain combined in Table 3. Yields from two separate productions (#1 and #2) are shown.

TABLE 3
Process yields of SID bsAbs

| | Total | Process yield | Process yield |
|---|---|---|---|
| | number of | (mg/L), | (mg/L), |
| CH1-CLκ Set Name | substitution(s) | production #1 | production #2 |
| H_168S_185S_187D-L_135R | 4 | 171.83 | 194.90 |
| H_128R_147R-L_124E_133Q_178E | 5 | 223.59 | 261.44 |
| H_145Q_147E_181E- | 6 | 70.98 | 199.87 |
| L_129R_178R_180Q | | | |
| H_147T_185Q-L_135S_178R | 4 | 186.67 | 173.07 |
| H_148R-L_124S_129E | 3 | 91.11 | ND |
| H_139R_141Q_187Q- | 6 | 136.60 | ND |
| L_114D_135S_138R | | | |
| H_166K_187K-L_137S_138E | 4 | 191.90 | ND |
| H_168R_185E-L_135S | 3 | 180.20 | ND |
| H_124R_147R-L_127D_129E | 4 | 273.73 | ND |
| H_147H_148E-L_127R_129R | 4 | 145.62 | ND |
| H_145S-L_133Y | 2 | 200.45 | ND |
| H_145S_181Q-L_133Y | 3 | 175.26 | ND |
| H_145S-L_124E_133Y | 3 | 243.83 | ND |
| H_145Q_181E-L_120S_178H_180Q | 5 | 51.57 | ND |
| H_124R_145S_147Q- | 6 | 219.28 | ND |
| L_127T_129D_178R | | | |
| H_166K_187K-L_114Q_137T_138E | 5 | 68.50 | 298.50 |
| H_147R_175D-L_129D_178R_180H | 5 | 67.45 | ND |
| H_147R_175E_181Q-L_129D_180Q | 5 | 38.63 | 233.53 |
| H_145S_147N-L_133Y_180R | 4 | 22.01 | 289.35 |
| H_147N_185Y-L_129R_180S | 4 | 191.30 | 273.90 |
| WT CH1 - WT CLκ | 0 | ND | 368.76 |

*ND: no data

### 2: Purity

The HEK293 production and protein A purification products were further analyzed for purity (as determined by the percentage of monomer full-size antibodies among all antibody products) by size exclusion chromatography (SEC). Briefly, an Agilent 1260 HPLC was employed to monitor the column chromatography (TSKgel Super SW mAb HTP column). The column was equilibrated with wash buffer (200 mM Sodium Phosphate, 250 mM Sodium Chloride pH 6.8) at a flow rate adjusted to 0.400 mL/min prior to use. Approximately 2-5 μg of protein sample was injected onto column. Protein migration was monitored at wavelength 280 nm. Total assay time was approximately 6 minutes. Data was analyzed using ChemStation software. Purity values from two separate productions (#1 and #2) are shown.

The purity values are summarized in Table 4.

TABLE 4
Purity of SID bsAb production products (by SEC)

| | Purity by SEC (% | Purity by SEC (% |
|---|---|---|
| | monomer full size | monomer full size |
| CH1-CLκ Set Name | bsAb), production #1 | bsAb), production #2 |
| H_168S_185S_187D-L_135R | 95.7 | 95.80 |
| H_128R_147R-L_124E_133Q_178E | 93.2 | 94.10 |
| H_145Q_147E_181E-L_129R_178R_180Q | 95.1 | 96.50 |
| H_147T_185Q-L_135S_178R | 91.4 | 97.20 |
| H_148R-L_124S_129E | 93.3 | ND |
| H_139R_141Q_187Q-L_114D_135S_138R | 93.3 | ND |
| H_166K_187K-L_137S_138E | 89.8 | ND |
| H_168R_185E-L_135S | 95.3 | ND |
| H_124R_147R-L_127D_129E | 94.9 | ND |
| H_147H_148E-L_127R_129R | 95.4 | ND |
| H_145S-L_133Y | 93.6 | ND |
| H_145S_181Q-L_133Y | 92.1 | ND |
| H_145S-L_124E_133Y | 95 | ND |
| H_145Q_181E-L_120S_178H_180Q | 97.9 | ND |
| H_124R_145S_147Q-L_127T_129D_178R | 96.7 | ND |
| H_166K_187K-L_114Q_137T_138E | 98.2 | 89.20 |
| H_147R_175D-L_129D_178R_180H | 97.3 | ND |
| H_147R_175E_181Q-L_129D_180Q | 97.5 | 93.00 |
| H_145S_147N-L_133Y_180R | 98.4 | 96.30 |
| H_147N_185Y-L_129R_180S | 97.1 | 94.90 |
| WT CH1 - WT CLκ | ND | 95.30 |

*ND: no data

### 3: Proper Pairing

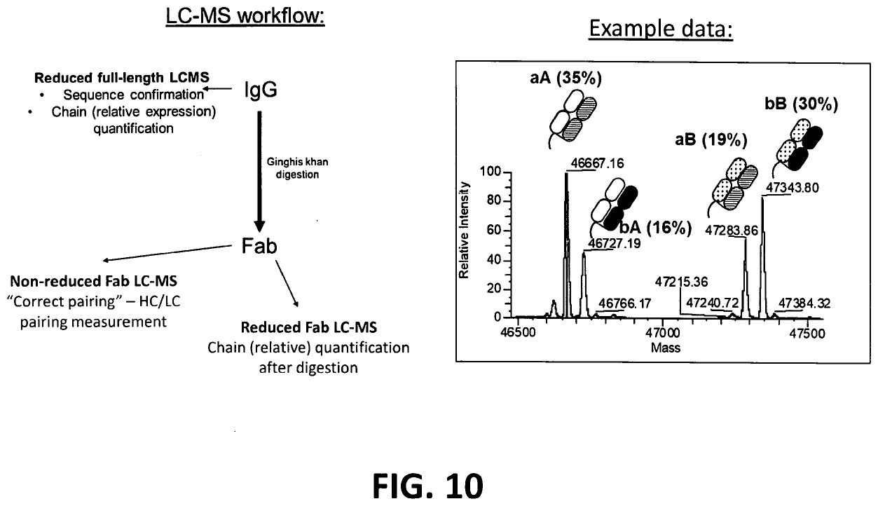

The HEK293 production products were further analyzed for proper pairing between CH1-1 and CLκ-1 using liquid chromatography-mass spectrometry (LC-MS). The workflow of the LC-MS-based analysis is provided in FIG. 10.

Briefly, before the pairing analysis, all full-length IgG samples were tested by LC-MS after DTT reduction to confirm sequence identity Samples were then subjected to Ginghis Khan digestion to obtain Fab fragments. Part of the Ginghis Khan digest was used for non-reduced LC-MS (for analyzing correct HC-LC pairing) the remaining part of the Ginghis Khan digest was used for reduced LC-MS (for analyzing relative chain quantification after digestion), Non-Reduced Fab LC-MS data are useful due to increased resolution without complications caused from the heterogeneity of the Fc glycosylation. Reduced full-length IgG LC-MS data and reduced Fab LC-MS data aid in ensuring relatively even chain expression and relative chain ionization efficiency post-Gingis Khan digestion, respectively. Samples were subsequently injected onto an Acquity Ultra Performance liquid chromatography (UPLC) system (Waters), equipped with a with a Thermo Scientific MabPac RP® 4 μm Column, (2.1×100 mm) maintained at 80° C. After injection, samples were eluted from the column using a 13-minute gradient from 20-55% acetonitrile at a flow rate of 0.3 mL/min (mobile phase A: 0.1% formic acid in H2O; mobile phase B: 0.1% formic acid in acetonitrile). Species eluted from the column were detected by a Q Exactive mass spectrometer (Thermo) in positive electrospray ionization mode. The instrument parameters were set as spray voltage of 3.5 kV, capillary temperature of 350° C., sheath gas flow rate at 35 and aux gas flow rate at 10 and S-lens RF level at 90. MS spectra were acquired at the scan range of 750-4000 m/z. Acquired MS data were analyzed using Biopharma Finder software (Thermo Scientific) followed by manual inspection to ensure correct assignment and relative quantification accuracy. Relative quantitation for each of the pairs and pair species were calculated based on the intensities of the peaks with respect to the sum of all the pairs and pair peak intensities.

The CH1-CL pairing analysis results from two separate bsAb productions (#1 and #2) are summarized in Table 5 and Table 6, respectively. 00 Correct pairing is the sum of % pairs of Panitumumab VH and Panitumumab VL (% value shown under “Pani/Pani”) and % pairs of Ustekinumab VH and Ustekinumab VL (% value shown under “Uste/Uste”).

TABLE 5
Pairing between CH1 and CL domains
in SID (by LC-MS), production #1

| | Correct | Pani/ | Pani/ | Uste/ | Uste/ |
|---|---|---|---|---|---|
| CH1-CLκ Set Name | pairing | Pani | Uste | Pani | Uste |
| H_168S_185S_187D- | 86% | 33% | 14% | 0% | 53% |
| L_135R | | | | | |
| H_128R_147R- | 82% | 39% | 6% | 12% | 43% |
| L_124E_133Q_178E | | | | | |
| H_145Q_147E_181E- | 82% | 39% | 4% | 14% | 43% |
| L_129R_178R_180Q | | | | | |
| H_147T_185Q- | 82% | 25% | 15% | 3% | 57% |
| L_135S_178R | | | | | |
| H_148R-L_124S_129E | 80% | 36% | 8% | 12% | 44% |
| H_139R_141Q_187Q- | 79% | 33% | 9% | 12% | 46% |
| L_114D_135S_138R | | | | | |
| H_166K_187K- | 79% | 33% | 6% | 15% | 46% |
| L_137S_138E | | | | | |
| H_168R_185E-L_135S | 78% | 33% | 11% | 11% | 45% |
| H_124R_147R- | 76% | 36% | 12% | 12% | 40% |
| L_127D_129E | | | | | |
| H_147H_148E- | 76% | 35% | 10% | 14% | 41% |
| L_127R_129R | | | | | |
| H_145S-L_133Y | 72% | 34% | 10% | 18% | 38% |
| H_145S_181Q-L_133Y | 72% | 32% | 9% | 19% | 40% |
| H_145S-L_124E_133Y | 71% | 36% | 11% | 18% | 35% |
| H_145Q_181E- | 66% | 21% | 24% | 10% | 45% |
| L_120S_178H_180Q | | | | | |
| H_124R_145S_147Q- | ND | ND | ND | ND | ND |
| L_127T_129D_178R | | | | | |
| H_166K_187K- | ND | ND | ND | ND | ND |
| L_114Q_137T_138E | | | | | |
| H_147R_175D- | ND | ND | ND | ND | ND |
| L_129D_178R_180H | | | | | |
| H_147R_175E_181Q- | ND | ND | ND | ND | ND |
| L_129D_180Q | | | | | |
| H_145S_147N- | ND | ND | ND | ND | ND |
| L_133Y_180R | | | | | |
| H_147N_185Y- | ND | ND | ND | ND | ND |
| L_129R_180S | | | | | |
| WT CH1 - WT CLκ | ND | ND | ND | ND | ND |

*ND: no data

TABLE 6
Pairing between CH1 and CL domains
in SID (by LC-MS), production #2

| | Correct | Pani/ | Pani/ | Uste/ | Uste/ |
|---|---|---|---|---|---|
| CH1-CLκ Set Name | pairing | Pani | Uste | Pani | Uste |
| H_168S_185S_187D- | 88% | 32% | 12% | 0% | 56% |
| L_135R | | | | | |
| H_128R_147R- | 80% | 34% | 6% | 14% | 46% |
| L_124E_133Q_178E | | | | | |
| H_145Q_147E_181E- | 86% | 35% | 7% | 7% | 50% |
| L_129R_178R_180Q | | | | | |
| H_147T_185Q- | 81% | 28% | 18% | 2% | 53% |
| L_135S_178R | | | | | |
| H_148R-L_124S_129E | ND | ND | ND | ND | ND |
| H_139R_141Q_187Q- | ND | ND | ND | ND | ND |
| L_114D_135S_138R | | | | | |
| H_166K_187K- | ND | ND | ND | ND | ND |
| L_137S_138E | | | | | |
| H_168R_185E-L_135S | ND | ND | ND | ND | ND |
| H_124R_147R- | ND | ND | ND | ND | ND |
| L_127D_129E | | | | | |
| H_147H_148E- | ND | ND | ND | ND | ND |
| L_127R_129R | | | | | |
| H_145S-L_133Y | ND | ND | ND | ND | ND |
| H_145S_181Q-L_133Y | ND | ND | ND | ND | ND |
| H_145S-L_124E_133Y | ND | ND | ND | ND | ND |
| H_145Q_181E- | ND | ND | ND | ND | ND |
| L_120S_178H_180Q | | | | | |
| H_124R_145S_147Q- | ND | ND | ND | ND | ND |
| L_127T_129D_178R | | | | | |
| H_166K_187K- | 73% | 23% | 6% | 21% | 50% |
| L_114Q_137T_138E | | | | | |
| H_147R_175D- | ND | ND | ND | ND | ND |
| L_129D_178R_180H | | | | | |
| H_147R_175E_181Q- | 72% | 31% | 6% | 21% | 41% |
| L_129D_180Q | | | | | |
| H_145S_147N- | 78% | 37% | 7% | 15% | 41% |
| L_133Y_180R | | | | | |
| H_147N_185Y- | 81% | 33% | 9% | 10% | 48% |
| L_129R_180S | | | | | |
| WT CH1 - WT CLκ | 79% | 37% | 9% | 12% | 42% |

*ND: no data

### Example 4: In Silico Screening—Rosetta Scoring/Evaluation of DIDs

Next, various double interface designs (DIDs) using the identified CH1-CLκ sets, referring herein to an antibody molecule or a fragment thereof which has one Fab incorporating a CH1-CLκ design and another Fab incorporating a different CH1-CLκ design, were evaluated.

15 out of the 20 CH1-CLκ sets were selected for designing DIDs, based on the production titer, correct HC/LC pairing (as measured by LCMS), and/or purity (as measured by SEC) in Example 3. RBPP_{hbond elec backrub 18k} scores for all possible combinations among the 15 CH1-CLκ sets were calculated. Mispair states in the RBPP calculation in Example 4 is now a result of pairing with the complementary chain from the opposing SID, rather than the WT CH1 or WT CLκ domain. The “hbond_elec” (all Rosetta Talaris energy function hydrogen bond score terms+the coulomb-based electrostatic score term) was used to continue to select for designs that used hydrogen bonds along with electrostatic repulsion/attraction as the mechanism of driving correct pairing.

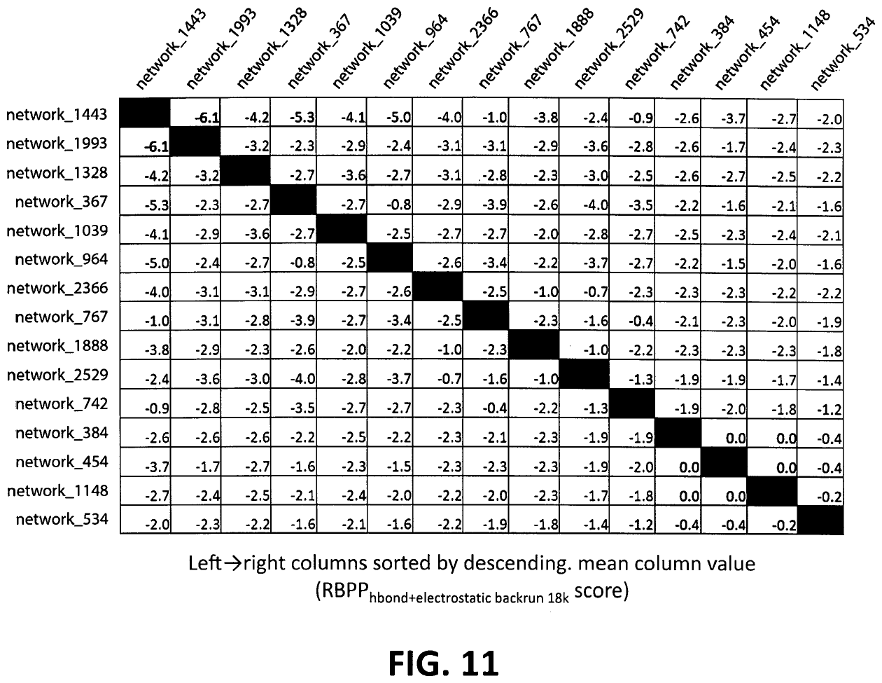

The RBPP_{hbond elec backrub 18k} scores are provided as a matrix table in FIG. 11. The Network names in FIG. 11 are the networks names shown in Table 2. As shown in FIG. 11, most CH1-CLκ set combinations were predicted to have negative RBPP_{hbond+electrostatic backrun 18k} scores, indicating that preferential pairing between the CH1 and CLκ domains in both CH1-CLκ sets would be expected to occur.

### Example 5: DID: Experimental Production in HEK Cells and Validation, Part 1

Next, various DIDs using the identified CH1-CLκ sets were experimentally produced and characterized. BsAbs having a full-size antibody structure in which (i) one Fab has the specificity of Ustekinumab (i.e., comprising VH and VL of Ustekinumab and a CH1-CLκ set identified above), (ii) the other Fab has the specificity of Panitumumab (i.e., comprising VH and VL of Panitumumab and another, different CH1-CLκ set identified above), and (iii) the CH3 domains comprise the Knob-in-Hole substitutions as described in Example 3 were produced (i.e., the structure depicted in FIG. 2C (left) further comprising the Knob-in-Hole substitutions in the CH3 domains).

Specifically, the intended bsAbs were designed to have: (1) a first heavy chain comprising a VH domain, a CH1 domain, a CH2 domain, and a CH3 domain (referred to as VH-1, CH1-1, CH2-1, and CH3-1, respectively); (2) a first light chain comprising a VL domain and a CLκ domain (referred to as VL-1 and CLκ-1, respectively); (3) a second heavy chain comprising a VH domain, a CH1 domain, a CH2 domain, and a CH3 domain (referred to as VH-2, CH1-2, CH2-2, and CH3-2, respectively); and (4) a second light chain comprising a VL domain and a CLκ domain (referred to as VL-2 and CLκ-2, respectively). The VH and VL sequences of Ustekinumab (the VL is lambda isotype) were used as the VH-1 and VL-1. The VH and VL sequences of Panitumumab (the VL is kappa isotype) were used as the VH-2 and VL-2. A first test CH1-CLκ set (having a 1st Network) was used for CH1-1 and CLκ-1. A second test CH1-CLκ set (having a 2nd Network) was used for CH1-2 and CLκ-2. The “knob-in-hole” substitutions in the CH3 domains and additional CH3 domain substitutions that allow a disulfide bond between CH3 to facilitate CH3 heterodimerization were also incorporated. Specifically, S354C and T366W (referred to as “Knob S=S” in Table 7) were incorporated to the CH3-2, and Y349C, T366S, L368A, and Y407V (referred to as “Hole S=S” in Table 7) were incorporated in the CH3-1 (EU numbering) (except for one of the two control Abs comprising two of WT-CH1-WT CLκ sets (bottom row in Table 7), which comprises “Knob S=S” in CH3-1 and “Hole S=S” in CH3-2). T366W in CH3-2 (Knob substitution) and T366S, L368A, and Y407V in CH3-1 (Hole substitutions) facilitate CH3 heterodimerization and S354C in CH3-2 and Y349C in CH3-1 form a disulfide bond to support such CH3-CH3 dimerization.

As an example, the CH1-CLκ set of Network_1443 (i.e., H_145Q_147E_181E-L_129R_178R_180Q) or Network_1039 (i.e., H_168S_185S_187D-L_135R) or the WT CH1-WT CLκ set was used in the Ustekinumab arm (i.e., first test CH1-CLκ set), and the CH1-CLκ set of Network_1993 (i.e., H_128R_147R-L_124E_133Q_178E), Network_964 (i.e., H_124R_147R-L_127D_129E), Network_1039 (i.e., H_168S_185S_187D-L_135R), Network_367 (i.e., H_148R-L_124S_129E), Network_2366 (i.e., H_168R_185E-L_135S), Network_2529 (i.e., H_147T_185Q-L_135S_178R), Network_742 (i.e., H_147N_185Y-L_129R_180S), or Network_1443 (i.e., H_145Q_147E_181E-L_129R_178R_180Q) or the WT CH1-WT CLκ set was used in the Panitumumab arm (i.e., second test CH1-CLκ set).

DID bsAbs produced in Example 5 are summarized in Table 7 with RBPP_{bond elec backrub 18k} scores calculated.

TABLE 7
CH1-CLκ sets used for experimental production in Example 5.

| | 1^{st} | 2^{nd} | Rosetta Score | | |
|---|---|---|---|---|---|
| | Network | Network | (RBPP hbond_elec | CH3-1 | CH3-2 |
| Type | (Uste arm) | (Pani arm) | 18k) | substitutions | substitutions |
| DID | 1443 | 1993 | −6.1 | Hole S═S | Knob S═S |
| DID | 1039 | 1993 | −2.9 | Hole S═S | Knob S═S |
| DID | 1443 | 964 | −5.0 | Hole S═S | Knob S═S |
| DID | 1443 | 1039 | −4.1 | Hole S═S | Knob S═S |
| DID | 1443 | 367 | −5.3 | Hole S═S | Knob S═S |
| DID | 1443 | 2366 | −4.0 | Hole S═S | Knob S═S |
| DID | 1039 | 367 | −2.7 | Hole S═S | Knob S═S |
| DID | 1039 | 2529 | −2.8 | Hole S═S | Knob S═S |
| DID | 1039 | 742 | −2.7 | Hole S═S | Knob S═S |
| DID | 1039 | 2366 | −2.7 | Hole S═S | Knob S═S |
| SID | 1443 | WT | | Hole S═S | Knob S═S |
| SID | 1039 | WT | | Hole S═S | Knob S═S |
| SID | WT | 1443 | | Hole S═S | Knob S═S |
| SID | WT | 1039 | | Hole S═S | Knob S═S |
| WT | WT | WT | | Hole S═S | Knob S═S |
| (Hole/Knob) | | | | | |
| WT | WT | WT | | Knob S═S | Hole S═S |
| (Knob/Hole) | | | | | |

As shown in Table 7, the best combination predicted based on the Rosetta Score was the combination of Network 1443 and 1993.

The DID bsAbs comprising these different combinations of CH1-CLκ sets were compared based on the production yield, purity, and proper pairing between CH1-1 and CLκ-1. The DID bsAbs were further evaluated based on the developability parameters PSR and HIC. In addition, dual binding to two different antigens were also confirmed.

1: Production Yield

Abs of Table 7 were produced in HEK293 cells and purified via protein A-based purification. The yields were determined as described in Example 3. The CH1-CLκ sets used in each DID bsAb (1^{st} Network refers to the CH1-CLκ set used in the Ustekinumab arm and 2^{nd} Network refers to the CH1-CLκ set used in the Panitumumab arm) and the process yields are summarized in Table 8.

TABLE 8
Process yields of DID bsAbs

| | 1^{st} Network | 2^{nd} Network | Yield |
|---|---|---|---|
| Type | (Uste) | (Pani) | (mg/L) |
| DID | 1443 | 1993 | 159 |
| DID | 1039 | 1993 | 131 |
| DID | 1443 | 964 | 140 |
| DID | 1443 | 1039 | 113 |
| DID | 1443 | 367 | 136 |
| DID | 1443 | 2366 | 147 |
| DID | 1039 | 367 | 107 |
| DID | 1039 | 2529 | 85 |
| DID | 1039 | 742 | 84 |
| DID | 1039 | 2366 | 109 |
| SID | 1443 | WT | 148 |
| SID | 1039 | WT | 123 |
| SID | WT | 1443 | 172 |
| SID | WT | 1039 | 165 |
| WT | WT | WT | 231 |
| (Hole/Knob) | | | |
| WT | WT | WT | 191 |
| (Knob/Hole) | | | |

### 2: Purity

The HEK293 production and protein A purification products of Table 8 were further analyzed for purity (as determined by the percentage of monomer full-size antibodies among all antibody products) by size exclusion chromatography (SEC), as described in Example 3. The purity values are summarized in Table 9.

TABLE 9
Purity of DID bsAb production products (by SEC)

| | | | Purity by SEC |
|---|---|---|---|
| | 1^{st} Network | 2^{nd} Network | (% monomer |
| Type | (Uste) | (Pani) | full size Ab) |
| DID | 1443 | 1993 | 97.6 |
| DID | 1039 | 1993 | 86.3 |
| DID | 1443 | 964 | 98.1 |
| DID | 1443 | 1039 | 96.2 |
| DID | 1443 | 367 | 97.6 |
| DID | 1443 | 2366 | 97.2 |
| DID | 1039 | 367 | 91.1 |
| DID | 1039 | 2529 | 91.3 |
| DID | 1039 | 742 | 94.7 |
| DID | 1039 | 2366 | 90.0 |
| SID | 1443 | WT | 98.0 |
| SID | 1039 | WT | 92.9 |
| SID | WT | 1443 | 95.9 |
| SID | WT | 1039 | 96.0 |
| WT | WT | WT | 94.6 |
| (Hole/Knob) | | | |
| WT | WT | WT | 96.2 |
| (Knob/Hole) | | | |

### 3: Proper Pairing (by LC-MS)

The HEK293 production products of Table 8 were further analyzed for proper pairing between cognate CH1-1 and CLκ-1 using liquid chromatography-mass spectrometry (LC-MS), as described in Example 3. The CH1-CL pairing analysis results are summarized in Table 10. Percent correctly paired in a DID bsAb design (“DID PC” in Table 10) is the sum of % pairs of Panitumumab VH and Panitumumab VL (00 value shown under “Pani/Pani”) and % pairs of Ustekinumab VH and Ustekinumab VL (% value shown under “Uste/Uste”). Table 10 also shows correct pairing results obtained when the indicated 1^{st} Network and 2^{nd} Network were used in a SID bsAb in Example 3 for comparison. “SID 1 PC” is the PC value when 1^{st} Network was used in a SID bsAb and “SID 2 PC” is the PC value when 2^{nd} Network was used in a SID bsAb.

TABLE 10
Pairing between CH1 and CL domains in DID (by LC-MS)

| | 1^{st} | 2^{nd} | | | | | | SID | SID |
|---|---|---|---|---|---|---|---|---|---|
| | Network | Network | Pani/ | Pani/ | Uste/ | Uste/ | DID | 1 | 2 |
| Type | (Uste) | (Pani) | Pani | Uste | Pani | Uste | PC | PC | PC |
| DID | 1443 | 1993 | 53% | 0% | 0% | 47% | 100% | 92% | 81% |
| DID | 1039 | 1993 | 59% | 0% | 3% | 38% | 97% | 79% | 81% |
| DID | 1443 | 964 | 59% | 2% | 3% | 36% | 95% | 92% | 76% |
| DID | 1443 | 1039 | 50% | 5% | 0% | 45% | 95% | 92% | 87% |
| DID | 1443 | 367 | 52% | 6% | 0% | 42% | 94% | 92% | 80% |
| DID | 1443 | 2366 | 49% | 4% | 2% | 42% | 91% | 92% | 78% |
| DID | 1039 | 367 | 59% | 0% | 13% | 28% | 87% | 79% | 80% |
| DID | 1039 | 2529 | 41% | 21% | 0% | 38% | 79% | 79% | 81% |
| DID | 1039 | 742 | 49% | 0% | 15% | 27% | 76% | 79% | 81% |
| DID | 1039 | 2366 | 37% | 22% | 5% | 36% | 73% | 79% | 78% |
| SID | 1443 | WT | 54% | 4% | 4% | 38% | 92% | | |
| SID | 1039 | WT | 55% | 3% | 18% | 24% | 79% | | |
| SID | WT | 1443 | 39% | 8% | 8% | 45% | 84% | | |
| SID | WT | 1039 | 40% | 10% | 0% | 50% | 90% | | |
| WT | WT | WT | 41% | 8% | 21% | 30% | 71% | | |
| (Hole/Knob) | | | | | | | | | |
| WT | WT | WT | 43% | 12% | 16% | 29% | 72% | | |
| (Knob/Hole) | | | | | | | | | |

As shown in Table 10, an impressive 100% correct paring was observed with the combination of Network 1443 and Network 1993 in DID. This is in accordance with the Rosetta Score-based prediction shown in Table 7.

### 4: Confirmation of Correct Pairing Trends (by IEX)

The correct pairing trends of the HEK293 production products of Table 8 were further evaluated by cation ion exchange chromatography (IEX). Briefly, IEX chromatographic separations were performed on a computer controlled AKTA Avant 150 preparative chromatography system equipped with an integrated pH electrode, enabling in-line pH monitoring, and a Mono S 5/50 GL column. The cation exchange buffer was composed of 15.6 mM CAPS, 9.4 mM CHES, 4.6 mM TAPS, 9.9 mM HEPPSO, 8.7 mM MOPSO, 11.0 mM MES, 13.0 mM Acetate, 9.9 mM Formate, 10 mM NaCl, and the pH was adjusted up to 4.0 (buffer A) or 11.0 (buffer B) using NaOH. 500 ug of protein was buffer exchanged into 25% buffer B and filtered through a 0.2 mm filter. Before each separation, the column was equilibrated with 10 column volumes of 25% buffer B. The protein was then loaded onto the column via a capillary loop, followed by a 10 column volume wash with 25% buffer B, a 20 column volume linear pH gradient from 25% to 100% buffer B, and a 10 column volume hold at 100% B.

As full IgG is used in IEX, this analysis using IEX can potentially identify mis-paired species that are preferentially lost/degraded by GinghisKhan Fab digestion. The IEX main peak % values are shown in Table 11.

TABLE 11
IEX profile of DID bsAbs (by IEX)

| | 1^{st} Network | 2^{nd} Network | IEX |
|---|---|---|---|
| Type | (Uste) | (Pani) | Main Peak |
| DID | 1443 | 1993 | 88% |
| DID | 1039 | 1993 | 78% |
| DID | 1443 | 964 | 85% |
| DID | 1443 | 1039 | 80% |
| DID | 1443 | 367 | 80% |
| DID | 1443 | 2366 | 79% |
| DID | 1039 | 367 | 62% |
| DID | 1039 | 2529 | 54% |
| DID | 1039 | 742 | 54% |
| DID | 1039 | 2366 | 53% |
| SID | 1443 | WT | 83% |
| SID | 1039 | WT | 57% |
| SID | WT | 1443 | 84% |
| SID | WT | 1039 | 90% |
| WT | WT | WT | 48% |
| (Hole/Knob) | | | |
| WT | WT | WT | 50% |
| (Knob/Hole) | | | |

The general correct pairing trends evaluated by IEX generally matched with the correct pairing results from LC-MS.

### 5: Developability (PSR)

Polyspecificity (also referred to as polyreactivity) is a highly undesirable property that has been linked to poor antibody pharmacokinetics (Wu et al., J Mol Biol 368:652-665, 2007; Hotzel et al., 2012, MAbs 4(6):753-760) and, thus, potentially to poor developability. Antibodies can be detected as possessing decreased or increased developability by virtue of their level of interaction with polyspecificity reagent (PSR). See WO2014/179363. Antibodies displaying increased interaction with PSR are referred to as “polyspecific” polypeptides, with poor(er) developability. DID bsAbs were thus tested for polyspecificity.

Polyspecificity of each bsAb of Table 8 was measured as described previously (L. Shehata et al., Affinity Maturation Enhances Antibody Specificity but Compromises Conformational Stability. Cell reports 28, 3300-3308 e3304 (2019)). Briefly, soluble membrane protein (SMP) and soluble cytosolic protein (SCP) fractions obtained from Chinese hamster ovary (CHO) cells were biotinylated using NHS-LC-Biotin (Thermo Fisher Scientific Cat #21336). IgGs presented on the surface of yeast were incubated with 1:10 diluted biotinylated CHO cell preparations on ice for 20 minutes. Cells were then washed twice with ice-cold PBS containing 0.1% BSA (PBSF) and incubated in 50 μL of a secondary labelling mix containing ExtrAvidin-R-PE (Sigma-Aldrich), anti-human LC-FITC (Southern Biotech) and propidium iodide) for 15 minutes. The cells were washed twice with PBSF and resuspended in PBSF to be run on a FACSCanto II (BD Biosciences). The mean fluorescence intensity of binding was normalized using control antibodies that display low, medium or high polyspecificity to assess the non-specific binding. Antibodies were rated as clean (PSR score below 0.11), low (PSR score below 0.33), medium (PSR score below 0.66), and high polyspecificity (PSR score above 0.66).

The results are summarized in Table 12.

TABLE 12
PSR assessment in DID

| | 1^{st} Network 1 | 2^{nd} Network | PSR | PSR |
|---|---|---|---|---|
| Type | (Uste arm) | (Pani arm) | (Normalized MFI) | Score |
| DID | 1443 | 1993 | 92.6 | 0.045 |
| DID | 1039 | 1993 | 82.0 | 0.016 |
| DID | 1443 | 964 | 111.9 | 0.096 |
| DID | 1443 | 1039 | ND | ND |
| DID | 1443 | 367 | 93.4 | 0.047 |
| DID | 1443 | 2366 | 86.0 | 0.027 |
| DID | 1039 | 367 | 80.3 | 0.012 |
| DID | 1039 | 2529 | 71.5 | 0 |
| DID | 1039 | 742 | 67.6 | 0 |
| DID | 1039 | 2366 | 85.8 | 0.027 |
| SID | 1443 | WT | 82.5 | 0.018 |
| SID | 1039 | WT | 71.1 | 0 |
| SID | WT | 1443 | 77.7 | 0.005 |
| SID | WT | 1039 | 81.5 | 0.015 |
| WT | WT | WT | 77.3 | 0.004 |
| (Hole/Knob) | | | | |
| WT | WT | WT | 73.0 | 0 |
| (Knob/Hole) | | | | |

* MFI stands for mean fluorescence intensity.

As shown in Table 12, all tested bsAbs had a PSR score below 0.11 and thus were determined to be “clean”.

### 6: Developability (HIC)

Hydrophobicity is another undesirable property linked to poor developability of an antibody. DID bsAbs were thus tested for hydrophobicity.

Briefly, hydrophobic interaction chromatography (HIC) was performed to assess hydrophobic interaction of the lead antibodies. The methodology for this assay was described previously (see Estep P, et al. (2015) An alternative assay to hydrophobic interaction chromatography for high-throughput characterization of monoclonal antibodies. MAbs 7(3):553-561). In brief, 5 μg IgG samples (1 mg/mL) were spiked in with a mobile phase A solution (1.8 M ammonium sulfate and 0.1 M sodium phosphate at pH 6.5) to achieve a final ammonium sulfate concentration of about 1 M before analysis. A Sepax Proteomix HIC butyl-NP5 column was used with a liner gradient of mobile phase A and mobile phase B solution (0.1 M sodium phosphate, pH 6.5) over 20 min at a flow rate of 1 mL/min with UV absorbance monitoring at 280 nm. Hydrophobicity levels were determined based on the retention time of the chromatographic analysis. Hydrophobicity is: clean to low when the retention time is <10.5 min; medium when the retention time is ≥10.5 and <11.5 min; and high when the retention time is ≥11.5 min.

All tested bsAbs were determined to have low hydrophobicity.

### 7: Dual Binding

Finally, all bsABs listed in Table 7 were tested for the ability to bind both cognate antigens (IL-12 for Ustekinumab and EGFR for Panitumumab).

All experiments were performed at 25° C. on a ForteBio Octet HTX instrument (Sartorius, Göttingen, Germany). All reagents were formulated into phosphate buffered saline with 0.1% (w/w) BSA (PBSF). Monomeric human EGFR-moFc (100 nM) was first loaded to anti-mouse Fc IgG capture sensor tips (Sartorius, Göttingen, Germany) and then allowed to stand in PBSF for a minimum of 15 minutes. These loaded sensor tips were initially exposed (60 s) to wells containing PBSF to establish a stable baseline for the assay before exposure (180 s) to the bispecific IgG (100 nM) and then finally (600 s) to human IL-12 (100 nM). Bispecific IgGs with sufficient binding responses in the final two steps of the assay were classified as dual binders.

All tested bsAbs were confirmed to bind both antigens.

### Example 6: DID: Experimental Production in HEK Cells and Validation, Part 2

Next, whether the CH1-CLκ sets are broadly applicable to bsAbs containing an arbitrary combination of Fv fragments, different from the combination of panitumumab and ustekinumab Fvs was tested. The DID combination of Network 1443 and Network 1993, which achieved 100% correct pairing in Example 5, was further used to produce and compare full size DID bsAbs (i.e., human IgG-like bsAbs having the structure depicted in FIG. 2C (left) further comprising the Knob-in-Hole substitutions in the CH3 domains) with different binding specificity combinations as summarized in Table 13. As shown in Table 13, bsAbs comprising WT CH1-CLκ sets and monospecific antibody controls were also produced. In Table 13, Hole S=S means the substitution combination of Y349C, T366S, L368A, and Y407V and Knob S=S means the substitution combination of S354C and T366W.

TABLE 13
Specificity and CH3 modification combinations in DID
Abs produced with Network 1443 and Network 1993.

| | Specificity | | CH1-CLκ set | VH/VL | CH3 |
|---|---|---|---|---|---|
| Ab Name | Type | Arm | (Network #) | Specificity | Modifications |
| Ofat1443: HoleS═S_{—} | Bispecific | 1 | 1443 | ofatumumab | Hole S═S |
| Sifa1993: KnobS═S | | 2 | 1993 | sifalimumab | Knob S═S |
| Sifa1443: HoleS═S_{—} | Bispecific | 1 | 1443 | sifalimumab | Hole S═S |
| Ofat1993: KnobS═S | | 2 | 1993 | ofatumumab | Knob S═S |
| OfatWT: HoleS═S_{—} | Bispecific | 1 | WT | ofatumumab | Hole S═S |
| SifaWT: KnobS═S | | 2 | WT | sifalimumab | Knob S═S |
| Fres1443HoleS═S_{—} | Bispecific | 1 | 1443 | fresolimumab | Hole S═S |
| Neci1993: KnobS═S | | 2 | 1993 | necitumumab | Knob S═S |
| Neci1443: HoleS═S_{—} | Bispecific | 1 | 1443 | necitumumab | Hole S═S |
| Fres1993: KnobS═S | | 2 | 1993 | fresolimumab | Knob S═S |
| FresWT: HoleS═S_{—} | Bispecific | 1 | WT | fresolimumab | Hole S═S |
| NeciWT: KnobS═S | | 2 | WT | necitumumab | Knob S═S |
| Neci1443: HoleS═S_{—} | Bispecific | 1 | 1443 | necitumumab | Hole S═S |
| Sifa1993: KnobS═S | | 2 | 1993 | sifalimumab | Knob S═S |
| Sifa1443: HoleS═S_{—} | Bispecific | 1 | 1443 | sifalimumab | Hole S═S |
| Neci1993: KnobS═S | | 2 | 1993 | necitumumab | Knob S═S |
| NeciWT: HoleS═S_{—} | Bispecific | 1 | WT | necitumumab | Hole S═S |
| SifaWT: KnobS═S | | 2 | WT | sifalimumab | Knob S═S |
| Fres1443: HoleS═S_{—} | Bispecific | 1 | 1443 | fresolimumab | Hole S═S |
| Ofat1993: KnobS═S | | 2 | 1993 | ofatumumab | Knob S═S |
| Ofat1443: HoleS═S_{—} | Bispecific | 1 | 1443 | ofatumumab | Hole S═S |
| Fres1993: KnobS═S | | 2 | 1993 | fresolimumab | Knob S═S |
| FresWT: HoleS═S_{—} | Bispecific | 1 | WT | fresolimumab | Hole S═S |
| OfatWT: KnobS═S | | 2 | WT | ofatumumab | Knob S═S |
| Pani1443: HoleS═S_{—} | Bispecific | 1 | 1443 | panitumumab | Hole S═S |
| Uste1993: KnobS═S | | 2 | 1993 | ustekinumab | Knob S═S |
| Uste1443: HoleS═S_{—} | Bispecific | 1 | 1443 | ustekinumab | Hole S═S |
| Pani1993: KnobS═S | | 2 | 1993 | panitumumab | Knob S═S |
| PaniWT: HoleS═S_{—} | Bispecific | 1 | WT | panitumumab | Hole S═S |
| UsteWT: KnobS═S | | 2 | WT | ustekinumab | Knob S═S |
| Pani1443: WT_{—} | Monospecific | 1 | 1443 | panitumumab | WT |
| Pani1443: WT | | 2 | 1443 | panitumumab | WT |
| Pani1993: WT_{—} | Monospecific | 1 | 1993 | panitumumab | WT |
| Pani1993: WT | | 2 | 1993 | panitumumab | WT |
| Uste1443: WT_{—} | Monospecific | 1 | 1443 | ustekinumab | WT |
| Uste1443: WT | | 2 | 1443 | ustekinumab | WT |
| Uste1993: WT_{—} | Monospecific | 1 | 1993 | ustekinumab | WT |
| Uste1993: WT | | 2 | 1993 | ustekinumab | WT |

Specifically, the intended bsAbs were designed to have: (1) a first heavy chain comprising a VH domain, a CH1 domain, a CH2 domain, and a CH3 domain (referred to as VH-1, CH1-1, CH2-1, and CH3-1, respectively); (2) a first light chain comprising a VL domain and a CLκ domain (referred to as VL-1 and CLκ-1, respectively); (3) a second heavy chain comprising a VH domain, a CH1 domain, a CH2 domain, and a CH3 domain (referred to as VH-2, CH1-2, CH2-2, and CH3-2, respectively); and (4) a second light chain comprising a VL domain and a CLκ domain (referred to as VL-2 and CLκ-2, respectively). The first heavy chain and the first light chain provide Arm 1 in Table 13 and the second heavy chain and the second light chain provide Arm 2 in Table 13.

The VH and VL sequences of the indicated antibodies (the antibody indicated in the “VH/VL specificity” column of Table 13; i.e., panitumumab (anti-EGFR), ustekinumab (anti-IL-12), ofatumumab (anti-CD20), sifalimumab (anti-IFN-alpha), fresolimumab (anti-TGF-beta), or necitumumab (anti-EGFR)) were used as Arm 1's VH and VL (i.e., VH-1 and VL-1) and as Arm 2's VH and VL (i.e., VH-2 and VL-2). The antibodies from which the specificity of DID bsAbs were derived were selected so as to allow testing of diverse variable region sequences and of VH/VL pairs that provide diverse correct pairing % when WT CH1-CLκ is used, including VH/VL pairs with low intrinsic pairing with WT CH1-CLκ. VH/VL pairs were also selected based on molecular weight delta filters, ensuring that the molecular weight difference between any two species of interest would be resolvable by LC-MS (>20 dalton difference for Fab species; the higher the better and >270 daltons when possible for Fd regions (i.e., from VH to hinge): and >40 daltons for light chains). A number of heavy chain germlines are represented among the variable regions that were chosen.

The indicated CH1-CLκ sets (the set indicated in the “CH1-CLκ set (Network #)” column of Table 13) were used for Arm 1's CH1-CLκ (i.e., CH1-1 and CLκ-1) and for Arm 2's CH1-CLκ (i.e., CH1-2 and CLκ-2). The “knob-in-hole” substitutions in the CH3 domains and additional CH3 domain substitutions that allow a disulfide bond between CH3 to facilitate CH3 heterodimerization were also incorporated as shown in Table 13. T366W (Knob substitution) in CH3-2 and T366S, L368A, and Y407V (Hole substitutions) in CH3-1 facilitate CH3 heterodimerization and S354C in CH3-2 and Y349C in CH3-1 form a disulfide bond to support such CH3-CH3 dimerization.

The DID bsAbs listed in Table 13 were compared based on the production yield, purity, and proper pairing between CH1-1 and CLκ-1. The DID bs Abs were further evaluated based on the melting temperature (Tm).

### 1: Production Yield

BsAbs were produced in HEK293 cells and purified via protein A-based purification. The yields were determined as described in Example 3. The process yields are summarized in Table 14.

TABLE 14
Process yields of HEK-produced DID Abs
with different specificity combinations

| Ab Name | Yield (mg/L) |
|---|---|
| Ofat1443: HoleS═S_Sifa1993: KnobS═S | 138.2 |
| Sifa1443: HoleS═S_Ofat1993: KnobS═S | 109.3 |
| OfatWT: HoleS═S_SifaWT: KnobS═S | 199.7 |
| Fres1443: HoleS═S_Neci1993: KnobS═S | 123.7 |
| Neci1443: HoleS═S_Fres1993: KnobS═S | 134.8 |
| FresWT: HoleS═S_NeciWT: KnobS═S | 150.4 |
| Neci1443: HoleS═S_Sifa1993: KnobS═S | 86.4 |
| Sifa1443: HoleS═S_Neci1993: KnobS═S | 92.4 |
| NeciWT: HoleS═S_SifaWT: KnobS═S | 257.2 |
| Fres1443: HoleS═S_Ofat1993: KnobS═S | 164.8 |
| Ofat1443: HoleS═S_Fres1993: KnobS═S | 212.0 |
| FresWT: HoleS═S_OfatWT: KnobS═S | 191.9 |
| Pani1443: HoleS═S_Uste1993: KnobS═S | 153.7 |
| Uste1443: HoleS═S_Pani1993: KnobS═S | 113.0 |
| PaniWT: HoleS═S_UsteWT: KnobS═S | 268.3 |
| Pani1443: WT_Pani1443: WT | 256.0 |
| Pani1993: WT_Pani1993: WT | 217.6 |
| Uste1443: WT_Uste1443: WT | 143.1 |
| Uste1993: WT_Uste1993: WT | 156.8 |

### 2: Purity

The HEK293 production and protein A purification products of Table 14 were further analyzed for purity (as determined by the percentage of monomer full-size antibodies among all antibody products) by size exclusion chromatography (SEC), as described in Example 3. The purity values are summarized in Table 15.

TABLE 15
Purity of DID Ab with different specificity combinations (by SEC)

| | Purity by SEC |
|---|---|
| | (% monomer full |
| Ab Name | size bsAb) |
| Ofat1443: HoleS═S_Sifa1993: KnobS═S | 95.8 |
| Sifa1443: HoleS═S_Ofat1993: KnobS═S | 96.9 |
| OfatWT: HoleS═S_SifaWT: KnobS═S | 95.1 |
| Fres1443: HoleS═S_Neci1993: KnobS═S | 97.1 |
| Neci1443: HoleS═S_Fres1993: KnobS═S | 97.5 |
| FresWT: HoleS═S_NeciWT: KnobS═S | 95.1 |
| Neci1443: HoleS═S_Sifa1993: KnobS═S | 94.7 |
| Sifa1443: HoleS═S_Neci1993: KnobS═S | 96.9 |
| NeciWT: HoleS═S_SifaWT: KnobS═S | 95.7 |
| Fres1443: HoleS═S_Ofat1993: KnobS═S | 94.9 |
| Ofat1443: HoleS═S_Fres1993: KnobS═S | 95.9 |
| FresWT: HoleS═S_OfatWT: KnobS═S | 94.6 |
| Pani1443: HoleS═S_Uste1993: KnobS═S | 96.5 |
| Uste1443: HoleS═S_Pani1993: KnobS═S | 96.6 |
| PaniWT: HoleS═S_UsteWT: KnobS═S | 94.8 |
| Pani1443: WT_Pani1443: WT | 98.3 |
| Pani1993: WT_Pani1993: WT | 97.5 |
| Uste1443: WT_Uste1443: WT | 99.3 |
| Uste1993: WT_Uste1993: WT | 98.5 |

### 3: Proper Pairing (by LC-MS)

The HEK293 production products of Table 14 were further analyzed for proper pairing between cognate CH1-1 and CLκ-1 using liquid chromatography-mass spectrometry (LC-MS), as described in Example 3. The CH1-CL pairing analysis results are summarized in Table 16. In Table 16. “aA” corresponds to the pairing between CH1-1 and CLκ-1 (i. e., correct heavy-light pairing to form Arm 1 of the intended bsAb), “bA” corresponds to the pairing between CH1-1 and CLκ-2 (i.e., incorrect heavy-light pairing), “aB” corresponds to the pairing between CH1-2 and CLκ-1 (i.e., incorrect pairing), and “WB” corresponds to the pairing between CH1-2 and CLκ-2 (i.e., correct heavy-light pairing to form Arm 2 of the intended bsAb). Percent correctly paired in a DID bsAb design (“PC” in Table 16) is the sum of % pairs of “aA” and “bB”.

TABLE 16
Pairing between CHI and CL domains in antibodies with
different specificity combinations (by LC-MS)

| Ab Name | aA | bA | aB | bB | PC |
|---|---|---|---|---|---|
| Ofat1443:HoleS = S_Sifa1993:KnobS = S | 61% | 0% | 0% | 39% | 100% |
| Sifa1443:HoleS = S_Ofat1993:KnobS = S | 35% | 3% | 0% | 62% | 97% |
| OfatWT:HoleS = S_SifaWT:KnobS = S | 53% | 0% | 24% | 23% | 76% |
| Fres1443: HoleS-S_Neci1993: KnobS = S | 51% | 0% | 0% | 47% | 98% |
| Neci1443:HoleS = S_Fres1993: KnobS = S | 44% | 2% | 0% | 51% | 95% |
| Fres WT:HoleS = S_NeciWT: KnobS = S | 30% | 21% | 13% | 34% | 64% |
| Neci1443:HoleS = S_Sifa1993: KnobS = S | 58% | 0% | 0% | 42% | 100% |
| Sifa1443:HoleS = S_Neci1993:KnobS = S | 37% | 0% | 0% | 63% | 100% |
| NeciWT:HoleS = S_SifaWT:KnobS = S | 42% | 9% | 37% | 12% | 54% |
| Fres1443: HoleS = S_Ofat1993: KnobS = S | 50% | 3% | 0% | 45% | 95% |
| Ofat1443:HoleS = S_Fres1993: KnobS = S | 48% | 0% | 0% | 50% | 98% |
| Fres WT:HoleS = S_OfatWT:KnobS = S | 38% | 13% | 7% | 42% | 80% |
| Pani 1443: HoleS = S_Uste1993: KnobS = S | 54% | 0% | 0% | 43% | 97% |
| Uste1443: HoleS = S_Pani1993: KnobS = S | 48% | 0% | 2% | 50% | 98% |
| PaniWT:HoleS = S_UsteWT:KnobS = S | 43% | 13% | 14% | 30% | 73% |

As shown in Table 16, the combination of Network 1443 and Network 1993 provided dramatically higher correct pairing compared to when two of WT CH1-CLκ (i.e. both CH1 and CLκ are wildtype) are used. Of note, for every tested variable region specificity combination, at least one DID bsAb did not show any incorrect pairing (0% bA and 0% aB). Two DIDs (Ofat:Sifa and Neci:Sifa), reached 100 correct pairing, while the other three DIDs (Fres:Neci, Ofat:Fres, Uste:Pani) reach 98% correct pairing.

### 4: Confirmation of Correct Pairing Trends (by IEX)

The correct pairing trends were further evaluated by cation ion exchange chromatography (IEX) as described in Example 5. The percent correctly paired (PC) values obtained by IEX and IEX main peak 00 values are shown in Table 17.

TABLE 17
IEX profile of bsAbs with different
specificity combinations (by IEX)

| Ab Name | IEX main peak % |
|---|---|
| Ofat1443:HoleS = S_Sifa1993:KnobS = S | 83% |
| Sifa1443:HoleS = S_Ofat1993:KnobS = S | 80% |
| OfatWT:HoleS = S_SifaWT:KnobS = S | 48% |
| Fres1443:HoleS-S_Neci1993:KnobS = S | ND |
| Neci1443:HoleS = S_Fres1993:KnobS = S | ND |
| FreSWT:HoleS = S_NeciWT:KnobS = S | 47% |
| Neci1443:HoleS = S_Sifa1993:KnobS = S | 85% |
| Sifa1443:HoleS = S_Neci1993:KnobS = S | 75% |
| NeciWT:HoleS = S_SifaWT:KnobS = S | 55% |
| Fres1443:HoleS = S_Ofat1993:KnobS = S | ND |
| Ofat1443:HoleS-S_FreS_1993:KnobS = S | ND |
| FreSWT:HoleS = S_OfatWT:KnobS = S | 53% |
| Pani1443:HoleS = S_Uste1993:KnobS = S | 96% |
| Uste1443:HoleS = S_Pani1993:KnobS = S | 88% |
| PaniWT:HoleS = S_UsteWT:KnobS = S | 49% |
| Pani1443:WT_Pani1443:WT | 94% |
| Pani1993:WT_Pani1993:WT | 95% |
| Uste1443:WT_Uste1443:WT | 88% |
| Uste1993:WT_Uste1993:WT | 97% |

ND: No data

As shown in Table 17, while the main peak was around 50% when two of WT CH1-CLκ were used, the combination of Network 1443 and Network 1993 dramatically increased the main peak % values in all tested DID bsAbs.

### 5: Melting Temperature

Using the monospecific antibodies listed in Table 13, along with panitumumab and ustekinumab comprising the WT CH1-CLκ set, the effect of the use of Network 1443 and Network 1993 on melting temperatures were evaluated.

Melting temperature (Tm) was measured by differential scanning fluorometry (DSF). Twenty microliters of sample, at 0.1-1 mg/ml, was mixed with 10 μl of 20× Sypro orange (Sigma-Aldrich) before being subjected to a controlled temperature increase from 40 to 95° C., at 0.5° C. intervals in a C1000 thermocycler (BioRad) to collect Fret signal. Melting temperature was obtained by taking the negative of first derivative of the raw signal. The results are shown in Table 18.

TABLE 18
Tm of panitumumab and ustekinumab with or without
Network 1443 or Network 1993 CH1-CLK set

| | | CH1-CLK | | | |
|---|---|---|---|---|---|
| | | set | VH/VL | CH3 | Tm |
| Ab Name | Arm | (Network #) | Specificity | Modifications | (° C.) |
| Pani1443:WT_Pani1443:WT | 1 | 1443 | panitumumab | WT | 77.0 |
| | 2 | 1443 | panitumumab | WT | |
| Pani1993:WT_Pani1993:WT | 1 | 1993 | panitumumab | WT | 77.5 |
| | 2 | 1993 | panitumumab | WT | |
| Uste1443:WT_Uste1443:WT | 1 | 1443 | ustekinumab | WT | 68.5 |
| | 2 | 1443 | ustekinumab | WT | |
| Uste1993:WT_Uste1993:WT | 1 | 1993 | ustekinumab | WT | 68.0 |
| | 2 | 1993 | ustekinumab | WT | |
| PaniWT:WT_PaniWT:WT | 1 | WT | panitumumab | WT | 78.0 |
| | 2 | WT | panitumumab | WT | |
| UsteWT:WT_UsteWT:WT | 1 | WT | ustekinumab | WT | 68.5 |
| | 2 | WT | ustekinumab | WT | |

As shown in Table 18, neither Network 1443 nor Network 1993 significantly affects the Tm values.

Taken together, the CH1-CLκ sets according to the present invention appear universally applicable to a variety of bsAbs having different specificity combinations. This is a marked advantage relative to many of the prior art CH1-CLκ sets.

Furthermore, while human IgG1 sequences were used in the Examples, the variant CH1 domains, variant CLκ domains, and/or CH1-CLκ sets according the present invention are expected to work in other isoforms such as IgG2 and IgG4, given the sequence similarities with IgG1.

### Example 7: Experimental Production in CHO Cells and Characterization

Next, various monospecific or bispecific, full-size Abs having a DID format of Networks 1443 and 1993 or having two WT, two Network 1443, or two Network 1993 CH1-CLκ sets with various binding specificity combinations with or without the Knob-in-Hole plus S=S CH3 modifications as listed in Table 19 were produced in CHO cells (in multiple production batches) and the products were characterized. Specifically, the intended Abs were designed to have: (1) a first heavy chain comprising a VH domain, a CH1 domain, a CH2 domain, and a CH3 domain which does not include the C-terminal lysine at position 447 (referred to as VH-1, CH1-1, C2-1, and CH3-1, respectively); (2) a first light chain comprising a VL domain and a CLκ domain (referred to as VL-1 and CLκ-1, respectively); (3) a second heavy chain comprising a VH domain, a CH1 domain, a CH2 domain, and a CH3 domain (referred to as VH-2, CH1-2, CH42-2, and CH43-2, respectively); and (4) a second light chain comprising a VL domain and a CLκ domain (referred to as VL-2 and CLκ-2, respectively). The first heavy chain and the first light chain provide Arm 1 in Table 19 and the second heavy chain and the second light chain provide Arm 2 in Table 19. In Table 19, Hole S=S means the substitution combination of Y349C, T366S, L368A, and Y407V and Knob S=S means the substitution combination of S354C and T366W.

TABLE 19
Abs produced in CHO cells and yields.

| | | CH1-CLK | | | |
|---|---|---|---|---|---|
| | | set | VH/VL | CH3 | Yield |
| Ab Name | Arm | (Network #) | Specificity | Modifications | (mg/L) |
| OfatWT:WT_OfatWT:WT | 1 | WT | ofatumumab | WT | |
| | 2 | WT | ofatumumab | WT | |
| Ofat1443:WT_Ofat1443:WT | 1 | 1443 | ofatumumab | WT | 144 |
| | 2 | 1443 | ofatumumab | WT | |
| Ofat1993:WT_Ofat1993:WT | 1 | 1993 | ofatumumab | WT | 198 |
| | 2 | 1993 | ofatumumab | WT | |
| Ofat1443:HoleS = | 1 | 1443 | ofatumumab | Hole S = S | 129 |
| S_Ofat1993:KnobS = S | 2 | 1993 | ofatumumab | Knob S = S | |
| NeciWT:WT_NeciWT:WT | 1 | WT | necitumumab | WT | |
| | 2 | WT | necitumumab | WT | |
| Neci1443:WT_Neci1443:WT | 1 | 1443 | necitumumab | WT | 238 |
| | 2 | 1443 | necitumumab | WT | |
| Neci1993:WT_Neci1993:WT | 1 | 1993 | necitumumab | WT | 279 |
| | 2 | 1993 | necitumumab | WT | |
| Neci1443:HoleS = | 1 | 1443 | necitumumab | Hole S = S | 142 |
| S_Neci1993:KnobS = S | 2 | 1993 | necitumumab | Knob S = S | |
| FresWT:WT_FresWT:WT | 1 | WT | fresolimumab | WT | |
| | 2 | WT | fresolimumab | WT | |
| Fres1443:WT_Fres1443:WT | 1 | 1443 | fresolimumab | WT | 252 |
| | 2 | 1443 | fresolimumab | WT | |
| Fres1993:WT_Fres1993:WT | 1 | 1993 | fresolimumab | WT | 264 |
| | 2 | 1993 | fresolimumab | WT | |
| Fres1443:HoleS = | 1 | 1443 | fresolimumab | Hole S = S | 128 |
| S_Fres1993:KnobS = S | 2 | 1993 | fresolimumab | Knob S = S | |
| PaniWT:WT_PaniWT:WT | 1 | WT | panitumumab | WT | |
| | 2 | WT | panitumumab | WT | |
| Pani1443:WT_Pani1443:WT | 1 | 1443 | panitumumab | WT | 271 |
| | 2 | 1443 | panitumumab | WT | |
| Pani1993:WT_Pani1993:WT | 1 | 1993 | panitumumab | WT | 325 |
| | 2 | 1993 | panitumumab | WT | |
| Pani1443:HoleS = | 1 | 1443 | panitumumab | Hole S = S | 138 |
| S_Pani1993:KnobS = S | 2 | 1993 | panitumumab | Knob S = S | |
| SifaWT:WT_SifaWT:WT | 1 | WT | sifalimumab | WT | |
| | 2 | WT | sifalimumab | WT | |
| Sifa1443:WT_Sifa1443:WT | 1 | 1443 | sifalimumab | WT | 125 |
| | 2 | 1443 | sifalimumab | WT | |
| Sifa1993:WT_Sifa1993:WT | 1 | 1993 | sifalimumab | WT | 262 |
| | 2 | 1993 | sifalimumab | WT | |
| Sifa1443:HoleS = | 1 | 1443 | sifalimumab | Hole S = S | 143 |
| S_Sifa1993:KnobS = S | 2 | 1993 | sifalimumab | Knob S = S | |
| UsteWT:WT_UsteWT:WT | 1 | WT | ustekinumab | WT | |
| | 2 | WT | ustekinumab | WT | |
| Uste1443:WT_Uste1443:WT | 1 | 1443 | ustekinumab | WT | 137 |
| | 2 | 1443 | ustekinumab | WT | |
| Uste1993:WT_Uste1993:WT | 1 | 1993 | ustekinumab | WT | 232 |
| | 2 | 1993 | ustekinumab | WT | |
| Uste1443:HoleS = | 1 | 1443 | ustekinumab | Hole S = S | 124 |
| S_Uste1993:KnobS = S | 2 | 1993 | ustekinumab | Knob S = S | |
| Pani1443:HoleS = | 1 | 1443 | panitumumab | Hole S = S | 226 |
| S_Uste1993:KnobS = S | 2 | 1993 | ustekinumab | Knob S = S | |
| Uste1443:HoleS = | 1 | 1443 | ustekinumab | Hole S = S | 68 |
| S_Pani1993:KnobS = S | 2 | 1993 | panitumumab | Knob S = S | |
| PaniWT:HoleS = | 1 | WT | panitumumab | Hole S = S | 234 |
| S_UsteWT:KnobS = S | 2 | WT | ustekinumab | Knob S = S | |
| Ofat1443:HoleS = | 1 | 1443 | ofatumumab | Hole S = S | 175 |
| S_Sifa1993:KnobS = S | 2 | 1993 | sifalimumab | Knob S = S | |
| Sifa1443:HoleS = | 1 | 1443 | sifalimumab | Hole S = S | 186 |
| S_Ofat1993:KnobS = S | 2 | 1993 | ofatumumab | Knob S = S | |
| OfatWT:HoleS = | 1 | WT | ofatumumab | Hole S = S | 58 |
| S_SifaWT:KnobS = S | 2 | WT | sifalimumab | Knob S = S | |
| Fres1443:HoleS = | 1 | 1443 | fresolimumab | Hole S = S | 202 |
| S_Neci1993:KnobS = S | 2 | 1993 | necitumumab | Knob S = S | |
| Neci1443:HoleS = | 1 | 1443 | necitumumab | Hole S = S | 190 |
| S_Fres1993:KnobS = S | 2 | 1993 | fresolimumab | Knob S-S | |
| FresWT:HoleS = | 1 | WT | fresolimumab | Hole S = S | 279 |
| S_NeciWT:KnobS = S | 2 | WT | necitumumab | Knob S = S | |
| Neci1443:HoleS = | 1 | 1443 | necitumumab | Hole S-S | 139 |
| S_Sifa1993:KnobS = S | 2 | 1993 | sifalimumab | Knob S = S | |
| Sifa1443:HoleS = | 1 | 1443 | sifalimumab | Hole S = S | 148 |
| S_Neci1993:KnobS = S | 2 | 1993 | necitumumab | Knob S = S | |
| NeciWT:HoleS = | 1 | WT | necitumumab | Hole S = S | 293 |
| S_SifaWT:KnobS = S | 2 | WT | sifalimumab | Knob S = S | |
| Fres1443:HoleS = | 1 | 1443 | fresolimumab | Hole S = S | 226 |
| S_Ofat1993:KnobS = S | 2 | 1993 | ofatumumab | Knob S = S | |
| Ofat1443:HoleS = | 1 | 1443 | ofatumumab | Hole S = S | 288 |
| S_Fres1993:KnobS = S | 2 | 1993 | fresolimumab | Knob S = S | |
| FresWT:HoleS = | 1 | WT | fresolimumab | Hole S-S | 195 |
| S_OfatWT:KnobS = S | 2 | WT | ofatumumab | Knob S = S | |

### 1: Proper Pairing (LC-MS)

Some of the antibodies of Table 19 produced in CHO cells were analyzed for proper pairing between cognate CH and CL using LC-MS), as described in Example 3. The results are summarized in Table 20. Percent correctly paired in an Ab (“PC” in Table 20) is the sum of % pair of CH1-1 and CLκ-1 (i.e., correct VH/LC pairing to form Arm 1 as intended) and % pair of CH1-2 and CLκ-2 (i.e., correct heavy-light pairing to form Arm 2 as intended).

TABLE 20
Pairing between CH1 and CLκ domains
in antibodies produced in CHO cells (by LC-MS)

| Ab digested | PC (%) |
|---|---|
| Pani1443:HoleS = S_Uste1993:KnobS = S | 96% |
| Uste1443:HoleS = S_Pani1993:KnobS = S | 98% |
| PaniWT:HoleS = S_UsteWT:KnobS = S | 70% |
| Ofat1443:HoleS = S_Sifa1993:KnobS = S | 98% |
| Sifa1443:HoleS = S_Ofat1993:KnobS = S | 100% |
| OfatWT:HoleS = S_SifaWT:KnobS = S | 67% |
| Fres1443:HoleS = S_Neci1993:KnobS = S | 97% |
| Neci1443:HoleS = S_Fres1993:KnobS = S | 96% |
| FresWT:HoleS = S_NeciWT:KnobS = S | 66% |
| Neci1443:HoleS = S_Sifa1993:KnobS = S | 100% |
| Sifa1443:HoleS = S_Neci1993:KnobS = S | 100% |
| NeciWT:HoleS = S_SifaWT:KnobS = S | 56% |
| Fres1443:HoleS = S_Ofat1993:KnobS = S | 100% |
| Ofat1443:HoleS = S_Fres1993:KnobS = S | 96% |
| FresWT:HoleS = S_OfatWT:KnobS = S | 79% |

Previously, it was observed that when the antibody production cell line was switched to a CHO cell line, the correct pairing between heavy and light chains decreased (see Bönisch et al, Protein Eng Des Sel. 2017 Sep. 1; 30(9):685-696.). In contrast, as shown in Table 20, the combination of Network 1443 and Network 1993 CH1-CLκ sets demonstrated very high PC (%) values, while achieving high titer as shown in Table 19.

### 2: Developability (HIC)

Some of the antibodies of Table 19 produced in CHO cells were analyzed for hydrophobicity. HIC was performed essentially as described above. The retention time (min) observed for each antibody is shown in Table 21.

TABLE 21
Retention time of antibodies produced in CHO cells.

| Ab digested | Retention Time (min) |
|---|---|
| FresWT:WT_FresWT:WT | 10.5 |
| NeciWT:WT_NeciWT:WT | 10.3 |
| OfatWT:WT_OfatWT:WT | 9.4 |
| PaniWT:WT_PaniWT:WT | 9.1 |
| SifaWT:WT_SifaWT:WT | 9.1 |
| UsteWT:WT_UsteWT:WT | 8.5 |
| Fres1443:HoleS = S_Neci1993:KnobS = S | 10.4 |
| Neci1443:HoleS = S_Fres1993:KnobS = S | 10.5 |
| Fres1443:HoleS = S_Ofat1993:KnobS = S | 10.0 |
| Ofat1443:HoleS = S_Fres1993:KnobS = S | 10.0 |
| Neci1443:HoleS = S_Sifa1993:KnobS = S | 9.7 |
| Sifa1443:HoleS = S_Neci1993:KnobS = S | 9.7 |
| Ofat1443:HoleS = S_Sifa1993:KnobS = S | 9.3 |
| Sifa1443:HoleS = S_Ofat1993:KnobS = S | 9.2 |
| Pani1443:HoleS = S_Uste1993:KnobS = S | 8.8 |
| Uste1443:HoleS = S_Pani1993:KnobS = S | 8.8 |

As shown in Table 21, use of Network 1443 and Network 1993 CH1-CLκ sets along with the Knob-in-Hole plus S=S modifications in CH3 did not significantly alter hydrophobicity.

### 3: Fab Melting Temperature (Tm)

Some of the antibodies of Table 19 produced in CHO cells were digested to obtain Fab fragments. Tm values of the Fabs treated with the amidase PNGase F were measured by DSF essentially as described above. The results are shown in Table 22. When the antibodies to be digested contained two different Fab components, the Tm data in Table 22 are data obtained for the mixture of the two Fabs.

TABLE 22
Tm of Fabs from antibodies with or without Network 1443
and Network 1993 CH1-CLκ sets produced in CHO cells.

| | | Tm |
|---|---|---|
| Ab digested | Fab Name | (° C.) |
| FresWT:WT_FresWT:WT | FresWT_FresWT | 73.5 |
| NeciWT:WT_NeciWT:WT | NeciWT_NeciWT | 79.0 |
| OfatWT:WT_OfatWT:WT | OfatWT_OfatWT | 71.0 |
| PaniWT:WT_PaniWT:WT | PaniWT_PaniWT | 78.5 |
| SifaWT:WT_SifaWT:WT | SifaWT_SifaWT | 65.5 |
| UsteWT:WT_UsteWT:WT | UsteWT_UsteWT | 69.0 |
| Fres1443:HoleS = S_Neci1993:KnobS = S | Fres1443_Neci1993 | 78.5 |
| Neci1443:HoleS = S_Fres1993:KnobS = S | Neci1443_Fres1993 | 77.0 |
| Fres1443:HoleS = S_Ofat1993:KnobS = S | Fres1443_Ofat1993 | 71.0 |
| Ofat1443:HoleS = S_Fres1993:KnobS = S | Ofat1443_Fres1993 | 71.0 |
| Neci1443:HoleS = S_Sifa1993:KnobS = S | Neci1443_Sifa1993 | 76.0 |
| Sifa1443:HoleS = S_Neci1993:KnobS = S | Sifa1443_Neci1993 | 80.0 |
| Ofat1443:HoleS = S_Sifa1993:KnobS = S | Ofat1443_Sifa1993 | 64.0 |
| Sifa1443:HoleS = S_Ofat1993:KnobS = S | Sifa1443_Ofat199 | 64.5 |
| Pani1443:HoleS = S_Uste1993:KnobS = S | Pani1443_Uste199 | 78.0 |
| Uste1443:HoleS = S_Pani1993:KnobS = S | Uste1443_Pani1993 | 77.0 |

As shown in Table 22, use of Network 1443 and Network 1993 CH-CLκ sets did not significantly reduce the Tm values of Fabs.

### 4: Aggregation Temperature (Tagg)

Tagg for some of the antibodies of Table 19 produced in CHO cells was measured briefly as follows. 8.8 μL of sample was loaded in duplicate to 16×9 μL micro cuvettes (Unchained Labs, Norton, MA, Product Code 201); three of the 16×9 μL micro cuvettes were loaded at a time into UNcle (Unchained Labs, Norton, MA); Tagg was selected as the application with a temperature range of 15° C. to 95° C.; intrinsic fluorescence measurements and static light scattering (SLS) measurements at 266 nm and 473 nm were taken for each sample replicate at V° C. intervals; the data was subjected to analysis using Uncle Analysis V5.03 software (Unchained Labs, Norton, MA) to determine Tagg 266. The Tagg 266 results are shown in Table 23.

TABLE 23
Aggregation temperatures of antibodies with or without Network
1443 and Network 1993 CH1-CLκ sets produced in CHO cells.

| Ab Name | Tagg 266 (° C.) |
|---|---|
| Pani1443:HoleS = S_Uste1993:KnobS = S | |
| Uste1443:HoleS = S_Pani1993:KnobS = S | 68.8 |
| PaniWT:HoleS = S_UsteWT:KnobS = S | 75.6 |
| Ofat1443:HoleS = S_Sifa1993:KnobS = S | |
| Sifa1443:HoleS = S_Ofat1993:KnobS = S | 65.7 |
| OfatWT:HoleS = S_SifaWT:KnobS = S | 65 |
| Fres1443:HoleS = S_Neci1993:KnobS = S | |
| Neci1443:HoleS = S_Fres1993:KnobS = S | 71.8 |
| FresWT:HoleS = S_NeciWT:KnobS = S | 73.8 |
| Neci1443:HoleS = S_Sifa1993:KnobS = S | |
| Sifa1443:HoleS = S_Neci1993:KnobS = S | 75.2 |
| NeciWT:HoleS = S_SifaWT:KnobS = S | 72.9 |
| Fres1443:HoleS = S_Ofat1993:KnobS = S | |
| Ofat1443:HoleS = S_Fres1993:KnobS = S | 63.9 |
| FresWT:HoleS = S_OfatWT:KnobS = S | 67.1 |

As shown in Table 23, use of Network 1443 and Network 1993 CH1-CLκ sets along with Knob-in-Hole plus S=S modifications did not significantly reduce the Tagg values of Fabs.

### 5: Binding Kinetics

Binding kinetics in relation to cognate antigens for some of the antibodies of Table 19 produced in CHO cells was measured using a ForteBio Octet HTX instrument (Sartorius, Göttingen, Germany) as described above. The affinity (KD) values obtained are summarized in Table 24.

TABLE 24
Cognate antigen binding by antibodies with or without Network
1443 and Network 1993 CH1-CLκ sets produced in CHO cells.

| Ab Name | Antigen | KD (nM) |
|---|---|---|
| Pani1443:HoleS = S_Uste1993:KnobS = S | EGFR | 0.78 |
| | IL-12B | 2.1 |
| Uste1443:HoleS = S_Pani1993:KnobS = S | IL-12B | 2.7 |
| | EGFR | 0.83 |
| Ofat1443:HoleS = S_Sifa1993:KnobS = S | IFNA1 | 1.0 |
| | CD20 | 2.2 |
| Sifal443:HoleS = S_Ofat1993:KnobS = S | IFNA1 | 0.44 |
| | CD20 | 2.2 |
| Neci1443:HoleS = S_Sifa1993:KnobS = S | EGFR | 1.3 |
| | IFNA1 | 0.44 |
| Sifa1443:HoleS = S_Neci1993:KnobS = S | EGFR | 2.7 |
| | IFNA1 | 0.45 |

As shown in Table 24, use of Network 1443 and Network 1993 CH1-CLκ sets did not significantly reduce binding to cognate antigens.

### Example 8: Comparison of DID Antibodies Comprising Network 1443 and Network 1993 CH1-CL Sets to DID Antibodies Comprising Pre-Existing CH1-CL Sets

Next, various monospecific or bispecific, full-size Abs having a DID format of Network 1443 and Network 1993 CH1-CLκ sets or having WT and/or pre-existing CH1-CLκ sets with various binding specificity combinations with the Knob-in-Hole plus S=S CH3 modifications as listed in Table 25 were produced in CHO cells and the products were characterized. Specifically, the intended Abs were designed to have: (1) a first heavy chain comprising a VH domain, a CH1 domain, a CH2 domain, and a CH3 domain (referred to as VH-1, CH1-1, CH2-1, and CH3-1, respectively); (2) a first light chain comprising a VL domain and a CLκ domain (referred to as VL-1 and CLκ-1, respectively); (3) a second heavy chain comprising a VH domain, a CH1 domain, a CH2 domain, and a CH3 domain (referred to as VH-2, CH1-2, CH2-2, and CH3-2, respectively); and (4) a second light chain comprising a VL domain and a CLκ domain (referred to as VL-2 and CLκ-2, respectively). The first heavy chain and the first light chain provide Arm 1 in Table 25 and the second heavy chain and the second light chain provide Arm 2 in Table 19. In Table 25, Hole S=S means the substitution combination of Y349C, T366S, L368A, and Y407V and Knob S=S means the substitution combination of S354C and T366W.

TABLE 25
Abs produced in CHO cells.

| | | CH1-CL set | VH/VL | CH3 |
|---|---|---|---|---|
| Ab Name | Arm | (Network #) | Specificity | Modifications |
| Pani1443:HoleS = | 1 | 1443 | panitumumab | HoleS = S |
| S_Pani1993:KnobS = S | 2 | 1993 | panitumumab | KnobS = S |
| PaniWT:HoleS = | 1 | WT | panitumumab | HoleS = S |
| S_PaniCTL1a:KnobS = S | 2 | CTL1a | panitumumab | KnobS = S |
| PaniCTL2a:HoleS = | 1 | CTL2a | panitumumab | HoleS = S |
| S_PaniCTL2b:KnobS = S | 2 | CTL2b | panitumumab | KnobS = S |
| PaniCTL2b:HoleS = | 1 | CTL2b | panitumumab | HoleS = S |
| S_PaniCTL2a:KnobS = S | 2 | CTL2a | panitumumab | KnobS = S |
| PaniCTL3k: HoleS = | 1 | CTL3k | panitumumab | HoleS = S |
| S_PaniCTL31:KnobS = S | 2 | CTL31 | panitumumab | KnobS = S |
| PaniCTL31:HoleS = | 1 | CTL31 | panitumumab | HoleS = S |
| S_PaniCTL3k:KnobS = S | 2 | CTL3k | panitumumab | KnobS = S |
| PaniCTLla:HoleS = | 1 | CTLla | panitumumab | HoleS = S |
| S_PaniWT:KnobS = S | 2 | WT | panitumumab | KnobS = S |
| Ofat1443:HoleS = | 1 | 1443 | ofatumumab | HoleS = S |
| S_Ofat1993:KnobS = S | 2 | 1993 | ofatumumab | KnobS = S |
| OfatCTL2a:HoleS = | 1 | CTL2a | ofatumumab | HoleS = S |
| S_OfatCTL2b:KnobS = S | 2 | CTL2b | ofatumumab | KnobS = S |
| OfatCTL3k:HoleS = | 1 | CTL3k | ofatumumab | HoleS = S |
| S_OfatCTL31:KnobS = S | 2 | CTL31 | ofatumumab | KnobS = S |
| Pani1443:HoleS = | 1 | 1443 | panitumumab | HoleS = S |
| S_Uste1993:KnobS = S | 2 | 1993 | ustekinumab | KnobS = S |
| Uste1443:HoleS = | 1 | 1443 | ustekinumab | HoleS = S |
| S_Pani1993:KnobS = S | 2 | 1993 | panitumumab | KnobS = S |
| PaniWT:HoleS = | 1 | WT | panitumumab | HoleS = S |
| S_UsteWT:KnobS = S | 2 | WT | ustekinumab | KnobS = S |

* The CH1-CL sets in Table 25 other than those with a Network name are the pre-existing CH1-CL sets as described in Table 1. The CTL31 substitutions were incorporated into the WT CLλ sequence of SEQ ID NO: 9. All other CL substitutions were incorporated to the WT CLκ sequence of SEQ ID NO: 2. All variable domains were κ isotype.

### 1: Proper Pairing (LC-MS)

Some of the monospecific antibodies of Table 25 produced in CHO cells were analyzed for proper pairing between cognate CH1 and CL using LC-MS, as described in Example 3. The results are summarized in Table 26. Percent correctly paired in an Ab (“PC” in Table 26) is the sum of % pairs of CH1-1 and CLκ-1 (i.e., correct VH/LC pairing to form Arm 1 as intended) and % pairs of CH1-2 and CLκ-2 (i.e., correct heavy-light pairing to form Arm 2 as intended).

TABLE 26
Pairing between CH1 and CL domains in antibodies
produced in CHO cells (by LC-MS)

| Ab | PC (%) |
|---|---|
| Pani1443:HoleS = S_Pani1993:KnobS = S | 98 |
| PaniWT:HoleS = S_PaniCTL1a:KnobS = S | 56 |
| PaniCTL2b:HoleS = S_PaniCTL2a:KnobS = S | 72 |
| PaniCTL2a:HoleS = S_PaniCTL2b:KnobS = S | 74 |
| PaniCTL1a:HoleS = S_PaniWT:KnobS = S | 54 |
| Ofat1443:HoleS = S_Ofat1993:KnobS = S | 100 |
| OfatCTL2a:HoleS = S_OfatCTL2b:KnobS = S | 88 |
| OfatCTL3k:HoleS = S_OfatCTL31:KnobS = S | 98 |

As shown in Table 26, Abs having a DID format of Network 1443 and Network 1993 CH1-CLκ sets provide much higher correct paring rates compared to Abs of a DID format having WT and/or pre-existing CH1-CL sets. In fact, 100% correct pairing was achieved with Ofat1443:Hole_Ofat1993:Knob.

### 2: pH3.5 Stress (by SEC)

Some of the antibodies of Table 25 produced in CHO cells were analyzed for tolerance to low pH by SEC. Briefly, Samples at 20 mg/mL were buffer exchanged into PBS (200 mM phosphate buffered with 250 mM sodium chloride, pH 7.0) and pH 3.5 buffer (50 mM sodium chloride, 200 mM acetic acid, pH 3.5). After 1 hour at room temperature (25° C.), buffer exchanged samples were diluted to 1 mg/mL in PBS (200 mM phosphate buffered with 250 mM sodium chloride, pH 7.0), and 2 μg of sample was injected into an Agilent 1260 Infinity analytical HPLC (Agilent, Santa Clara, CA) fitted with a TSKgel SuperSW mAb HTP column (TOSOH Bioscience, King of Prussia, PA, Product Code 22855). SEC data was collected and subjected to analysis using Agilent ChemStation software (Agilent, Santa Clara, CA). The SEC data (in terms of purity %) are provided in Table 27.

TABLE 27
Purity of DID antibodies at pH 3.5 or 7.0 produced in CHO cells (by SEC)

| | Purity (%) after | Purity (%) after |
|---|---|---|
| Ab | incubation at pH 3.5 | incubation at pH 7.0 |
| Pani1443:HoleS = S_Uste1993:KnobS = S | 97.1 | 96.5 |
| Uste1443:HoleS = S_Pani1993:KnobS = S | 97.3 | 96.9 |
| PaniWT:HoleS = S_UsteWT:KnobS = S | 97.2 | 97 |
| PaniCTL1a:HoleS = S_PaniWT:KnobS = S | 97.2 | 97.3 |
| PaniWT:HoleS = S_PaniCTL1a:KnobS = S | 97.2 | 97.0 |
| PaniCTL2a:HoleS = S_PaniCTL2b:KnobS = S | 96.6 | 96.8 |
| PaniCTL2b:HoleS = S_PaniCTL2a:KnobS = S | 97.3 | 97.4 |
| PaniCTL3k:HoleS = S_PaniCTL31:KnobS = S | 31.6 | 85.7 |
| PaniCTL31:HoleS = S_PaniCTL3k:KnobS = S | 31.7 | 92.7 |

As shown in Table 28, Abs having a DID format of Network 1443 and Network 1993 CH1-CLκ sets provide very high purity even after experiencing low pH stress, while some antibodies of a DID format having WT and/or pre-existing CH1-CL sets show lower purity.

### Example 9: Structural Analysis of Fabs with Network 1443 CH1-CLκ Set, Network 1993 CH1-CLκ Set, or a Mismatch Set of Network 1443 CH1 and Network 1993 CLκ

To analyze the effect of substitutions in CH1 and CL domains on the interaction between the CH1 and CL domains, a human Fab comprising a Network 1993 CH1-CLκ design set, named ADI-64597, and a human Fab comprising a Network 1443 CH1-CLκ design set, named ADI-64596, were obtained from Abs produced in HEK cells and the crystal structures were analyzed.

### Methods

### Crystallization and Structure Determination of ADI-64597 Fab:

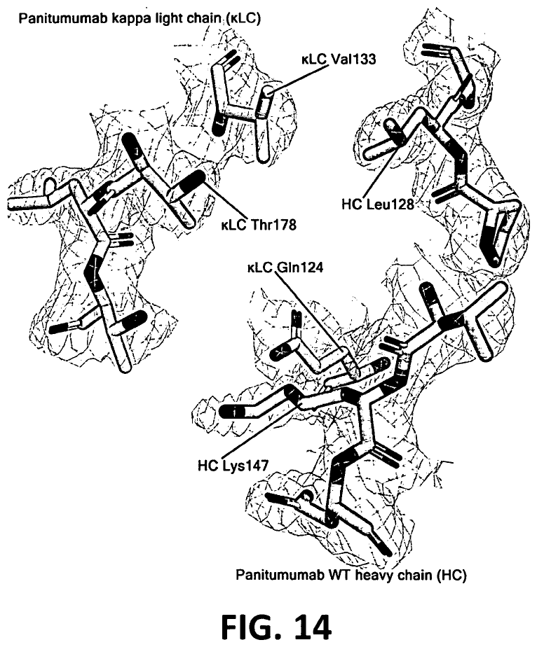

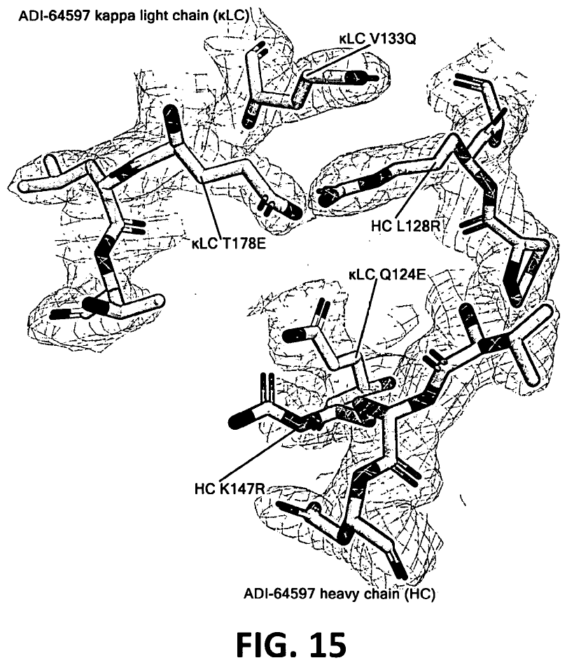

ADI-64597 (human Fab, comprising a CH1 (of IgG1) domain comprising L128R and K147R substitutions and a CLκ domain comprising Q124E, V133Q, and T178E substitutions (i.e., Network 1993 CH1-CLκ set)) concentrated to 16.5 mg/mL into a buffer containing 2 mM Tris-HCl pH 8.0 and 150 mM NaCl. PACT, BCS and JCSG+ screens (all from Molecular Dimensions Ltd.) was set up using a mosquito crystallization robot (STP Labtech). Sitting drops of 150 nL protein and 150 nL reservoir solution were left to equilibrate against a 40 μL reservoir at 20° C. After a few days, needle-like crystals were obtained in several conditions. The crystal used for data collection was obtained in the BCS screen, condition B10 (0.1 M HEPES pH 7.5, 22% w/v PEG Smear Broad). The crystal was flash-frozen in liquid nitrogen after soaking in reservoir solution supplemented with 20% glycerol as cryo-protectant. Data were collected at synchrotron beamline BioMAX, MAX IV Laboratory, Lund, Sweden, at 100 K and λ=0.9763 Å. 3600 images were collected with an oscillation range of 0.10 per image. The beamline is equipped with an Eiger 16M hybrid-pixel detector. Data extending to 2.2 Å were processed using EDNA_proc (Monaco, S., et al. (2013) “Automatic processing of macromolecular crystallography X-ray diffraction data at the ESRF”. Journal of Applied Crystallography. 46, (3), 804-810), which includes the software XDS (Kabsch W. (2010) “XDS” Acta. Crystallogr. D Biol. Crystallogr. 66, 125-132) and Aimless (Evans P. R. and Murshudov, G. N. (2013) “How good are my data and what is the resolution” Acta Crystallogr D Biol. Crystallogr. 69, 1204-1214). Crystals consisted of a single molecule in the asymmetric unit (ASU) in P3₁ space group. A molecular replacement solution for the ADI-64597 Fab was obtained by PHASER (McCoy, A. J., Grosse-Kunstleve, R. W., Adams, P. D., Winn, M. D., Storoni, L. C., & Read, R. J. (2007). Phaser crystallographic software. Journal of applied crystallography, 40(4), 658-674) using the previously disclosed Panitumumab WT CH1-CLκ Fab (WO/2021/067404). The structures were built manually in COOT (Emsley P., Lohkamp, B., Scott, W. G. and Cowtan K. (2010) “Features and development of Coot” Acta Crystallogr. D Biol. Crystallogr. 66, 486-501) and refined using PHENIX (Adams P D, et al. (2010) “PHENIX: a comprehensive Python-based system for macromolecular structure solution”. Acta Crystallogr. D Biol. Crystallogr.; 66:213-221) to a final to a final R and R_{free} of 18.0% and 23.0%, respectively (FIG. 15). FIG. 15 may be compared to the corresponding electron density diagram for the WT CH1-CLκ set shown in FIG. 14.

### Crystallization and Structure Determination of ADI-64596 Fab:

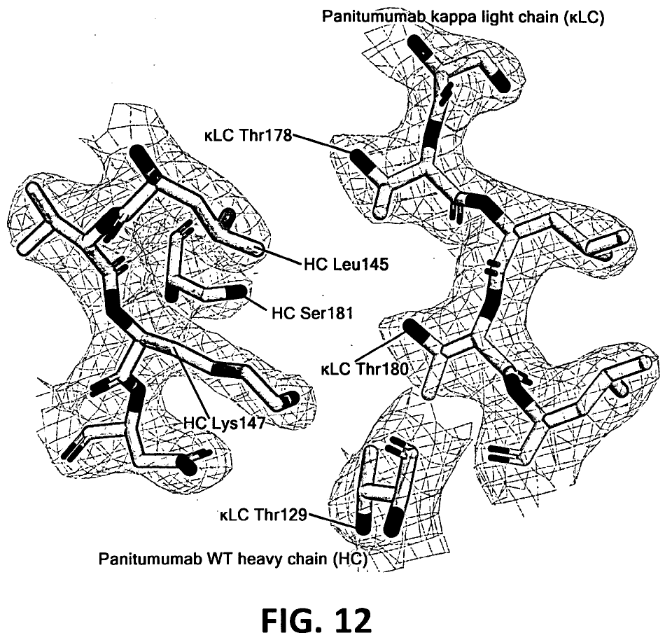

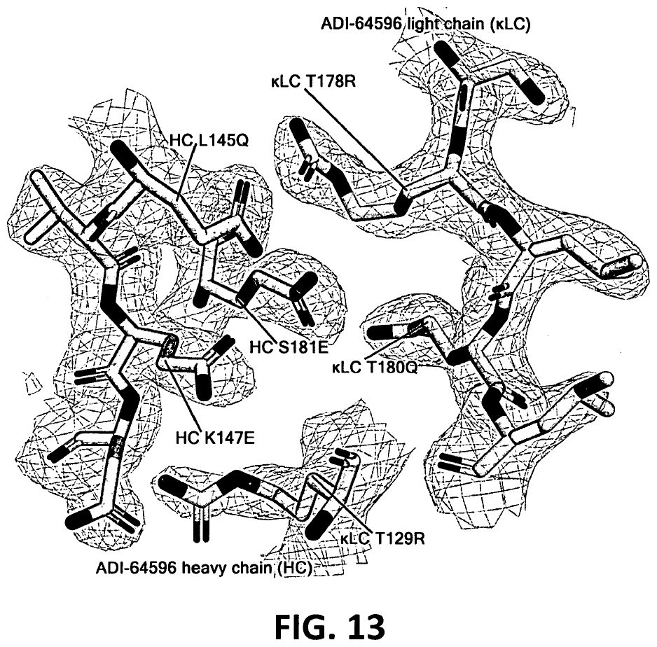

ADI-64596 (human Fab, comprising a CH1 (of IgG1) domain comprising L145Q, K147E, and S181E substitutions and a CLκ domain comprising T129R, T178R, and T180Q substitutions (i.e., Network 1443 CH1-CLκ set)) was concentrated to 11.35 mg/mL into a buffer containing 2 mM Tris-HCl pH 8.0 and 150 mM NaCl. The PACT, BCS and JCSG+ screens (all from Molecular Dimensions Ltd.) were initially set up using a mosquito crystallization robot (STP Labtech). Since crystals obtained from these initial screens only gave rise to low-resolution X-ray diffraction, crystal seed solutions were prepared and applied in the setup of the BCS, PACT, and Additive Screens (Hampton Research). Sitting drops of 160 nL protein and 160 nL precipitant solution were left to equilibrate against a 40 μL reservoir at 20° C. After a few days, plate and needle-like crystals appeared in several conditions. The precipitant solution giving rise to the best-diffracting crystal contained 75 mM Tris pH 8.5, 25 mM Bis-Tris-propane pH 8.5, 22.5% (v/v) PEG Smear Low, 5% (w/v) PEG3350, 50 mM NaBr. The crystal was flash-cooled in liquid nitrogen after soaking in precipitant solution supplemented with 10% (v/v) PEG400 as cryo-protectant. Data were collected at synchrotron beamline 104, Diamond Light Source, UK, at 100 K and λ=0.9795 Å. 3600 images were collected with an oscillation range of 0.10 per image. The beamline is equipped with a Dectris Eiger2 XE 16M detector. Data extending to 2.35 Å were processed using XDS², Aimless (Evans P. R. and Murshudov, G. N. (2013) “How good are my data and what is the resolution” Acta Crystallogr D Biol. Crystallogr. 69, 1204-1214) and reindexed to correspond with the ADI-64597 data set using the Sftools software of the CCP4i suite (Winn M. D. et al. (2011) “Overview of the CCP4 suite and current developments” Acta Crystallog. D Biol. Crystallogr. 67, 235-242. 235-242). Crystals consisted of a single molecule in the asymmetric unit (ASU) in P3₁ space group. A molecular replacement solution for the ADI-64596 Fab was obtained by PHASER (McCoy, A. J., Grosse-Kunstleve, R. W., Adams, P. D., Winn, M. D., Storoni, L. C., & Read, R. J. (2007). Phaser crystallographic software. Journal of applied crystallography, 40(4), 658-674) using the Panitumumab wildtype CH1-Ckappa Fab. The structures were built manually in COOT (Emsley P., Lohkamp, B., Scott, W. G. and Cowtan K. (2010) “Features and development of Coot” Acta Crystallogr. D Biol. Crystallogr. 66, 486-501) and refined using PHENIX (Adams P D, et al. (2010) “PHENIX: a comprehensive Python-based system for macromolecular structure solution”. Acta Crystallogr. D Biol. Crystallogr.; 66:213-221) to a final to a final R and R_{free} of 20.8% and 22.1%, respectively (FIG. 13). FIG. 13 may be compared to the corresponding electron density diagram for the WT CH1-CLκ set shown in FIG. 12.

### Results

### CH1-Cκ Pairing Mediated by Substitutions Present in ADI-64596 Fab:

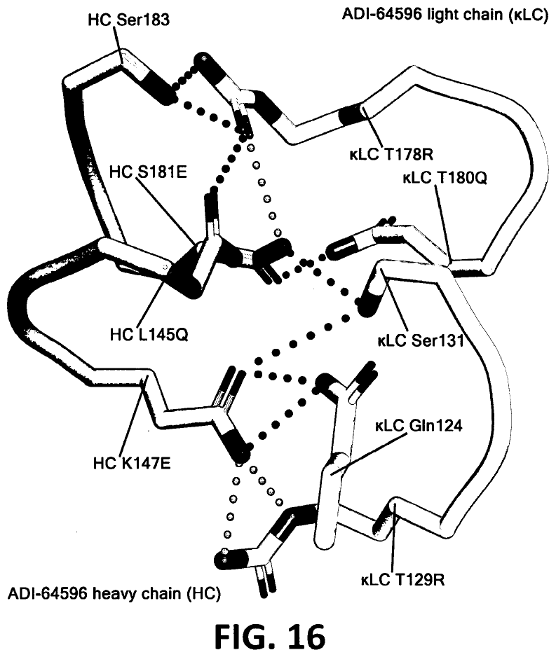

Enhanced pairing between the CH1 and CLκ domains of Network 1443 is mediated by several novel polar contacts found in the sextuple-substituted molecule (FIG. 16). These contacts include new salt-bridges formed between K147E of CH1 and T129R of CLκ and between S181E of CH1 and T178R of CLκ. Contacts also include new hydrogen bonds (i) between T178R of CLκ and two residues of CH1, L145Q and S at position 183, (ii) between K147E of CH1 and two residues of CLκ, Q at position 124 and S at position 131, and (iii) between S181E of CH1 and two residues of CLκ, S at position 131 and T180Q. I.e., every substitution was found to contribute to a salt-bridge or new hydrogen bond.

### CH1-Cκ Pairing Mediated by Substitutions Present in ADI-64597 Fab:

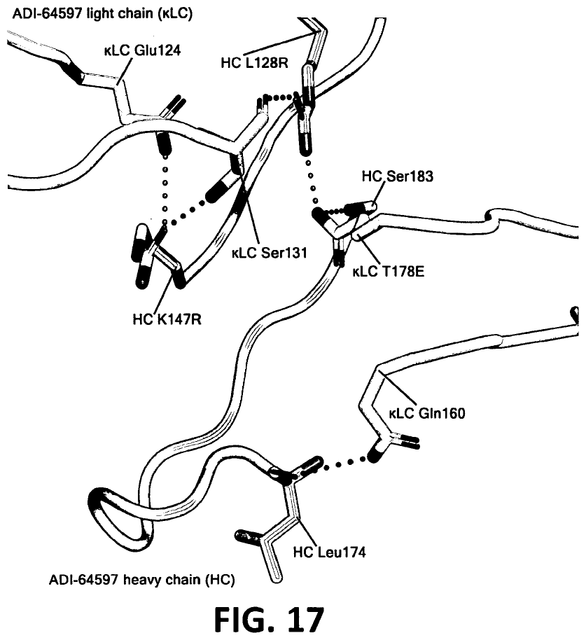

Enhanced pairing between the CH1 and CLκ domains of Network 1993 is mediated by several novel polar contacts found in this quintuple-substituted molecule (FIG. 17). These contacts include new salt-bridges formed between K147R of CH1 and E at position 124 of CLκ and between L128R of CH1 and T178E of CLκ. New contacts also include hydrogen bonds (i) between K147R in CH1 and S at position 131 of CLκ, (ii) between L128R of CH1 and S at position 131 of CLκ, and (iii) between T178E of CLκ and S at position 183 of CH1, as well as (iv) between two unsubstituted residues, L at position 174 of CH1 and Q at position 160 of CLκ.

### Steric Clashes Exist at the Interface of ADI-64597 CH1 and ADI-64596 CLκ:

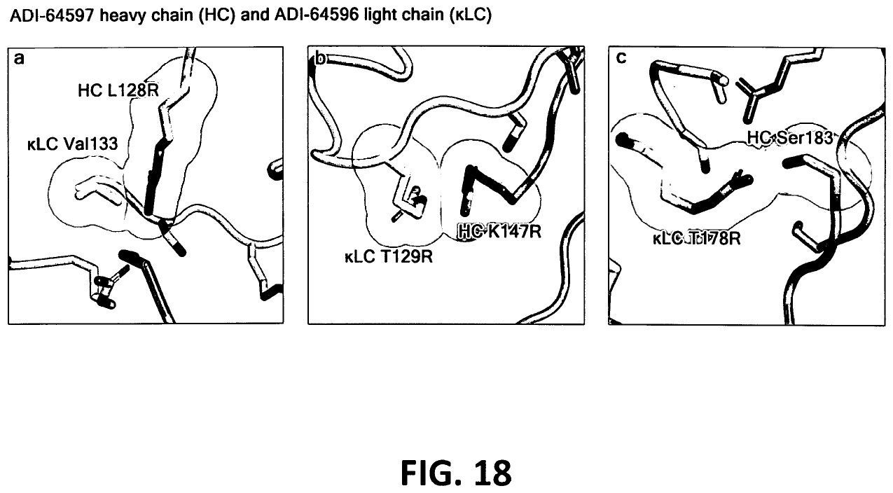

The CH1 and CLκ domains of ADI-64597 and ADI-64596 were structurally aligned and the potential mispairs (ADI-64596 HC/ADI-64597 LC and ADI-64597 HC/ADI-64596 LC) were probed for clashes in PyMol. For example, in the ADI-64597 HC/ADI-64596 LC mispair, three substantial clashes were observed (FIG. 18) including (a) L128R of CH1 and V at position 133 of CLκ, (b) HC-K147R of CH1 and T129R of CLκ and (c) S at position 183 of CH1 and T178R of CLκ. These clashes are predicted to reduce the propensity of the formation of the ADI-64597 HC/ADI-64596 LC mispaired construct, and thus when the CH1-CLκ pair of Network 1443 and the CH1-CLκ pair of Network 1993 are used to generate bispecific antibodies, the clashes are expected to enhance correct pairing.

### Example 10: Application to CH1-CLλ Sets

This Example tested whether the substitutions of CH1-CLκ design sets in Table 2 may be incorporated into the WT CLκ, domain sequence, and the corresponding CH1-CLλ, design sets obtained therefrom (Table 28) would provide preferential pairing between the design CH1 domain and the design CLλ, domain.

TABLE 28
CH1-CLλ design sets.

| | Network | CH1 | SEQ | CLλ | SEQ |
|---|---|---|---|---|---|
| CH1-CLλ Set Name | Name | substitution(s) | ID NO: | substitution(s) | ID NO: |
| H_168S_185S_187D- | 1039 | 168S; 185S; 187D | 11 | 135R | 19 |
| L_135R | | | | | |
| H_128R_147R- | 1993 | 128R; 147R | 21 | 133Q; 178E | 29 |
| L_133Q_178E | | | | | |
| H_145Q_147E_181E- | 1443 | 145Q; 147E; 181E | 31 | 129R; 178R; | 39 |
| L_129R_178R_180Q | | | | 180Q | |
| H_147T_185Q- | 2529 | 147T; 185Q | 41 | 135S; 178R | 49 |
| L_135S_178R | | | | | |
| H_148R-L_124S_129E | 367 | 148R | 51 | 124S; 129E | 59 |
| H_139R_141Q_187Q- | 1888 | 139R; 141Q; 187Q | 61 | 114D; 135S; | 69 |
| L_114D_135S_138R | | | | 138R | |
| H_166K_187K-L_138E | 1328 | 166K; 187K | 71 | 138E | 79 |
| H_168R_185E-L_135S | 2366 | 168R; 185E | 81 | 135S | 89 |
| H_124R_147R- | 964 | 124R; 147R | 91 | 127D; 129E | 99 |
| L_127D_129E | | | | | |
| H_147H_148E- | 767 | 147H; 148E | 101 | 127R; 129R | 109 |
| L_127R_129R | | | | | |
| H_145S-L_133Y | 1148 | 145S | 111 | 133Y | 119 |
| H_145S_181Q-L_133Y | 384 | 145S; 181Q | 121 | 133Y | 129 |
| H_145S-L_133Y | 454 | 145S | 131 | 133Y | 139 |
| H_145Q_181E- | 1048 | 145Q; 181E | 141 | 120S; 178H; | 149 |
| L_120S_178H_180Q | | | | 180Q | |
| H_124R_145S_147Q- | 534 | 124R; 145S; 147Q | 151 | 127T; 129D; | 159 |
| L_127T_129D_178R | | | | 178R | |
| H_166K_187K- | 838 | 166K; 187K | 161 | 114Q; 137T; | 169 |
| L_114Q_137T_138E | | | | 138E | |
| H_147R_175D- | 919 | 147R; 175D | 171 | 129D; 178R; | 179 |
| L_129D_178R_180H | | | | 180H | |
| H_147R_175E_181Q- | 394 | 147R; 175E; 181Q | 181 | 129D; 180Q | 189 |
| L_129D_180Q | | | | | |
| H_145S_147N- | 1621 | 145S; 147N | 191 | 133Y; 180R | 199 |
| L_133Y_180R | | | | | |
| H_147N_185Y-L_129R | 742 | 147N; 185Y | 201 | 129R | 209 |

* The CH1-CLλ set names (also identifiable by the Network Name together with the light chain isotype) as used herein are named by the amino acid positions (according to EU numbering) substituted in the CH1 domain (substitutions specified after “H_” (H followed by underscore), separated by “_” (underscore)) and the CLλ domain (substitutions specified after “L_” (L followed by underscore), separated by “_” (underscore)), with a dash to separate domains. For example, the H_168S_185S_187D-L_135R set has S, S, and D in the variant CH1 domain at positions 168, 185, and 187, along with R in the variant CLλ domain at position 135.
** The sequences assigned with the SEQ ID NOs shown are exemplary CH1 and CLλ sequences.
Any CH1 and CLλ sequences comprising the indicated amino acid substitution(s) are encompassed by each applicable Network according to the present invention.

RBPP_{hbond+electrostatic backrun 18k} scores were calculated for Abs comprising two different CH1-CLλ sets of Table 28 using essentially the same method as in Example 4 to produce the data for CH1-CLκ sets in FIG. 11. A matrix which provides RBPP_{hbond+electrostatic backrun 18k} scores calculated for each CH1-CLλ set combination is provided in FIG. 19. As shown in FIG. 19, most CH1-CLλ set combinations were predicted to have negative RBPP_{hbond+electrostatic backrun 18k} scores, indicating that preferential pairing between the CH1 and CLλ domains in both CH1-CLλ sets would occur.

### EXEMPLARY EMBODIMENTS

Described herein below are some exemplary embodiments according to the present disclosure.

Embodiment 1. An immunoglobulin heavy chain constant region 1 (“CH1”) domain variant polypeptide comprising an amino acid substitution(s), wherein the amino acid substitution(s) comprise(s) or consist(s) of an amino acid substitution(s) at one or more of the following amino acid positions: 124, 128, 139, 141, 145, 147, 148, 166, 168, 175, 181, 185, and/or 187, according to EU numbering, optionally such that the CH1 domain variant polypeptide preferentially pairs with an immunoglobulin kappa light chain constant region (CLκ) domain variant polypeptide comprising an amino acid substitution(s), wherein the amino acid substitution(s) in the CLκ domain variant polypeptide comprise(s) or consist(s) of an amino acid substitution(s) at one or more of the following positions: 114, 120, 124, 127, 129, 133, 135, 137, 138, 178, and/or 180, according to EU numbering, optionally wherein the CH1 domain variant polypeptide is a variant of a CH1 domain of a human IgG, optionally of a human IgG1, human IgG2, or human IgG4, further optionally wherein:
(i) when the amino acid substitution(s) in the CH1 domain variant polypeptide consists of T187E and the CH1 domain variant polypeptide preferentially pairs with a CLκ domain variant polypeptide, the amino acid substitution(s) in the CLκ domain variant polypeptide do(es) not consist of N137K and S114A;
(ii) when the amino acid substitution(s) in the CH1 domain variant polypeptide consists of L145Q and S183V and the CH1 domain variant polypeptide preferentially pairs with a CLκ domain variant polypeptide, the amino acid substitution(s) in the CLκ domain variant polypeptide do(es) not consist of V133T and S176V;
(iii) when the amino acid substitution(s) in the CH1 domain variant polypeptide consists of K147A and K213E and the CH1 domain variant polypeptide preferentially pairs with a CLκ domain variant polypeptide, the amino acid substitution(s) in the CLκ domain variant polypeptide do(es) not consist of S131R and E123K;
(iv) when the amino acid substitution(s) in the CH1 domain variant polypeptide consists of S183A and K147A and the CH1 domain variant polypeptide preferentially pairs with a CLκ domain variant polypeptide, the amino acid substitution(s) in the CLκ domain variant polypeptide do(es) not consist of S176I and S131R;
(v) when the amino acid substitution(s) in the CH1 domain variant polypeptide consists of S183G and K147A and the CH1 domain variant polypeptide preferentially pairs with a CLκ domain variant polypeptide, the amino acid substitution(s) in the CLκ domain variant polypeptide do(es) not consist of S176I and S131R;
(vi) when the amino acid substitution(s) in the CH1 domain variant polypeptide consists of K147A, K213E, and S183A and the CH1 domain variant polypeptide preferentially pairs with a CLκ domain variant polypeptide, the amino acid substitution(s) in the CLκ domain variant polypeptide do(es) not consist of S131R, E123K, and S176I;
(vii) when the amino acid substitution(s) in the CH1 domain variant polypeptide consists of K147A, K213E, and S183G and the CH1 domain variant polypeptide preferentially pairs with a CLκ domain variant polypeptide, the amino acid substitution(s) in the CLκ domain variant polypeptide do(es) not consist of S131R, E123K, and S176I;
(viii) when the amino acid substitution(s) in the CH1 domain variant polypeptide consists of A141I, F170S, S181M, S183A, and V185A and the CH1 domain variant polypeptide preferentially pairs with a CLκ domain variant polypeptide, the amino acid substitution(s) in the CLκ domain variant polypeptide do(es) not consist of F116A, L135V, S174A, S176F, and T178V;
(ix) when the amino acid substitution(s) in the CH1 domain variant polypeptide consists of A141L and the CH1 domain variant polypeptide preferentially pairs with a CLκ domain variant polypeptide, the amino acid substitution(s) in the CLκ domain variant polypeptide do(es) not consist of F118S, F118A, or F118V;
(x) when the amino acid substitution(s) in the CH1 domain variant polypeptide consists of K147D and the CH1 domain variant polypeptide preferentially pairs with a CLκ domain variant polypeptide, the amino acid substitution(s) in the CLκ domain variant polypeptide do(es) not consist of T129R;
(xi) when the amino acid substitution(s) in the CH1 domain variant polypeptide consists of S181E and S183V and the CH1 domain variant polypeptide preferentially pairs with a CLκ domain variant polypeptide, the amino acid substitution(s) in the CLκ domain variant polypeptide do(es) not consist of S176 and T178; or
(xii) when the amino acid substitution(s) in the CH1 domain variant polypeptide consists of S183L and V185Y and the CH1 domain variant polypeptide preferentially pairs with a CLκ domain variant polypeptide, the amino acid substitution(s) in the CLκ domain variant polypeptide do(es) not consist of V133S.

Embodiment 2. The CH1 domain variant polypeptide of Embodiment 1, wherein the amino acid substitution(s) of the CH1 domain variant polypeptide comprise(s) or consist(s) of an amino acid substitution(s) at:
(I) position(s) 185 and/or 187;
(II) position(s) 145, 147, and/or 148;
(III) position(s) 147 or 148;
(IV) position 145;
(V) position(s) 166 and/or 187;
(VI) position(s) 145 and/or 147; or
(VII) position(s) 124 and/or 147,
optionally wherein the CH1 domain variant polypeptide preferentially pairs with a CLκ domain variant polypeptide and:
in (I), the amino acid substitution(s) in the CLκ domain variant polypeptide comprise(s) or consist(s) of an amino acid substitution at position 135;
in (II), the amino acid substitution(s) in the CLκ domain variant polypeptide comprise(s) or consist(s) of an amino acid substitution at position 124;
in (III), the amino acid substitution(s) in the CLκ domain variant polypeptide comprise(s) or consist(s) of an amino acid substitution at position 129;
in (IV), the amino acid substitution(s) in the CLκ domain variant polypeptide comprise(s) or consist(s) of an amino acid substitution at position 133;
in (V), the amino acid substitution(s) in the CLκ domain variant polypeptide comprise(s) or consist(s) of an amino acid substitution at position(s) 137 and/or 138;
in (VI), the amino acid substitution(s) in the CLκ domain variant polypeptide comprise(s) or consist(s) of an amino acid substitution at position(s) 178 and/or 180; or
in (VII), the amino acid substitution(s) in the CLκ domain variant polypeptide comprise(s) or consist(s) of an amino acid substitution at position 127.

Embodiment 3. The CH1 domain variant polypeptide of Embodiment 1, wherein the one or more amino acid substitution(s) of the CH1 domain variant polypeptide comprise or consist of an amino acid substitution(s) at:
(i) positions 168, 185, and 187; or (ii) positions 128 and 147; or (iii) positions 145, 147, and 181; or (iv) positions 147 and 185; or (v) position 148; or (vi) positions 139, 141, and 187; or (vii) positions 166 and 187; or (viii) positions 168 and 185; or (ix) positions 124 and 147; or (x) positions 147 and 148; or (xi) position 145; or (xii) positions 145 and 181; or (xii) positions 124, 145, and 147; or (xiv) positions 166 and 187; or (xv) positions 147 and 175; or (xvi) positions 147, 175, and 181; or (xvii) positions 145 and 147; or (xviii) positions 147 and 185, optionally wherein the CH1 domain variant polypeptide preferentially pairs with a CLκ domain variant polypeptide and.
in (i), the amino acid substitution(s) in the CLκ domain variant polypeptide comprise(s) or consist(s) of an amino acid substitution(s) at position 135;
in (ii), the amino acid substitution(s) in the CLκ domain variant polypeptide comprise(s) or consist(s) of an amino acid substitution(s) at positions 124, 133, and 178;
in (iii), the amino acid substitution(s) in the CLκ domain variant polypeptide comprise(s) or consist(s) of an amino acid substitution(s) at positions 129, 178, and 180; or
in (iv), the amino acid substitution(s) in the CLκ domain variant polypeptide comprise(s) or consist(s) of an amino acid substitution(s) at positions 135 and 178;
in (v), the amino acid substitution(s) in the CLκ domain variant polypeptide comprise(s) or consist(s) of an amino acid substitution(s) at positions 124 and 129;
in (vi), the amino acid substitution(s) in the CLκ domain variant polypeptide comprise(s) or consist(s) of an amino acid substitution(s) at positions 114, 135, and 138;
in (vii), the amino acid substitution(s) in the CLκ domain variant polypeptide comprise(s) or consist(s) of an amino acid substitution(s) at positions 137 and 138;
in (viii), the amino acid substitution(s) in the CLκ domain variant polypeptide comprise(s) or consist(s) of an amino acid substitution(s) at position 135;
in (ix), the amino acid substitution(s) in the CLκ domain variant polypeptide comprise(s) or consist(s) of an amino acid substitution(s) at positions 127 and 129;
in (x), the amino acid substitution(s) in the CLκ domain variant polypeptide comprise(s) or consist(s) of an amino acid substitution(s) at positions 127 and 129;
in (xi), the amino acid substitution(s) in the CLκ domain variant polypeptide comprise(s) or consist(s) of an amino acid substitution(s) at position 133 or an amino acid substitution(s) at positions 124 and 133;
in (xii), the amino acid substitution(s) in the CLκ domain variant polypeptide comprise(s) or consist(s) of an amino acid substitution(s) at position 133 or an amino acid substitution(s) at positions 120, 178, and 180;
in (xiii), the amino acid substitution(s) in the CLκ domain variant polypeptide comprise(s) or consist(s) of an amino acid substitution(s) at positions 127, 129, and 178;
in (xiv), the amino acid substitution(s) in the CLκ domain variant polypeptide comprise(s) or consist(s) of an amino acid substitution(s) at positions 114, 137, and 138;
in (xv), the amino acid substitution(s) in the CLκ domain variant polypeptide comprise(s) or consist(s) of an amino acid substitution(s) at positions 129, 178, and 180;
in (xvi), the amino acid substitution(s) in the CLκ domain variant polypeptide comprise(s) or consist(s) of an amino acid substitution(s) at positions 129 and 180;
in (xvii), the amino acid substitution(s) in the CLκ domain variant polypeptide comprise(s) or consist(s) of an amino acid substitution(s) at positions 133 and 180; or
in (xviii), the amino acid substitution(s) in the CLκ domain variant polypeptide comprise(s) or consist(s) of an amino acid substitution(s) at positions 129 and 180.

Embodiment 4. The CH1 domain variant polypeptide of any one of Embodiments 1-3, wherein the amino acid substitution(s) in the CH1 domain variant polypeptide comprise(s) or consist(s) of: 124R, 128R, 139R, 141Q, 145Q, 145S, 147E, 147H, 147N, 147Q, 147R, 147T, 148E, 148R, 166K, 168R, 168S, 175D, 175E, 181E, 181Q, 185E, 185Q, 185S, 185Y, 187D, 187K, and/or 187Q.

Embodiment 5. The CH1 domain variant polypeptide of any one of Embodiments 1-4, wherein the amino acid substitution(s) of the CH1 domain variant comprise(s) or consist(s) of:
(i) 168S, 185S, and 187D; (ii) 128R and 147R; (iii) 145Q, 147E, and 181E; (iv) 147T and 185Q; (v) 148R; (vi) 139R, 141Q, and 187Q; (vii) 166K and 187K; (viii) 168R and 185E; (ix) 124R and 147R; (x) 147H and 148E; (xi) 145S; (xii) 145S and 181Q; (xiii) 145Q and 181E; (xiv) 124R, 145S, and 147Q; (xv) 166K and 187K; (xvi) 147R and 175D; (xvii) 147R, 175E, and 181Q; (xiii) 145S and 147N; or (xix) 147N and 185Y,
optionally wherein the CH1 domain variant polypeptide preferentially pairs with a CLκ domain variant polypeptide and:
   in (i), the amino acid substitution(s) in the CLκ domain variant polypeptide comprise(s) or consist(s) of 135R;
   in (ii), the amino acid substitution(s) in the CLκ domain variant polypeptide comprise(s) or consist(s) of 124E, 133Q, and 178E;
   in (iii), the amino acid substitution(s) in the CLκ domain variant polypeptide comprise(s) or consist(s) of 129R, 178R, and 180Q;
   in (iv), the amino acid substitution(s) in the CLκ domain variant polypeptide comprise(s) or consist(s) of 135S and 178R;
   in (v), the amino acid substitution(s) in the CLκ domain variant polypeptide comprise(s) or consist(s) of 124S and 129E;
   in (vi), the amino acid substitution(s) in the CLκ domain variant polypeptide comprise(s) or consist(s) of 114D, 135S, and 138R;
   in (vii), the amino acid substitution(s) in the CLκ domain variant polypeptide comprise(s) or consist(s) of 137S and 138E;
   in (viii), the amino acid substitution(s) in the CLκ domain variant polypeptide comprise(s) or consist(s) of 135S;
   in (ix), the amino acid substitution(s) in the CLκ domain variant polypeptide comprise(s) or consist(s) of 127D and 129E;
   in (x), the amino acid substitution(s) in the CLκ domain variant polypeptide comprise(s) or consist(s) of 127R and 129R;
   in (xi), the amino acid substitution(s) in the CLκ domain variant polypeptide comprise(s) or consist(s) of 133Y or 124E and 133Y;
   in (xii), the amino acid substitution(s) in the CLκ domain variant polypeptide comprise(s) or consist(s) of 133Y;
   in (xiii), the amino acid substitution(s) in the CLκ domain variant polypeptide comprise(s) or consist(s) of 120S, 178H, and 180Q;
   in (xiv), the amino acid substitution(s) in the CLκ domain variant polypeptide comprise(s) or consist(s) of 127T, 129D, and 178R;
   in (xv), the amino acid substitution(s) in the CLκ domain variant polypeptide comprise(s) or consist(s) of 114Q, 137T, and 138E;
   in (xvi), the amino acid substitution(s) in the CLκ domain variant polypeptide comprise(s) or consist(s) of 129D, 178R, and 180H;
   in (xvii), the amino acid substitution(s) in the CLκ domain variant polypeptide comprise(s) or consist(s) of 129D and 180Q;
   in (xiii), the amino acid substitution(s) in the CLκ domain variant polypeptide comprise(s) or consist(s) of 133Y and 180R; or
   in (xix), the amino acid substitution(s) in the CLκ domain variant polypeptide comprise(s) or consist(s) of 129R and 180S.

Embodiment 6. The CH1 domain variant polypeptide of any one of Embodiments 1-4, wherein the amino acid substitution(s) in the CH1 domain variant polypeptide consist(s) of
(i) 168S, 185S, and 187D;
(ii) 128R and 147R;
(iii) 145Q, 147E, and 181E; or
(iv) 147T and 185Q,
optionally wherein the CH1 domain variant polypeptide preferentially pairs with a CLκ domain variant polypeptide and:
in (i), the amino acid substitution(s) in the CLκ domain variant polypeptide comprise(s) or consist(s) of 135R;
in (ii), the amino acid substitution(s) in the CLκ domain variant polypeptide comprise(s) or consist(s) of 124E, 133Q, and 178E;
in (iii), the amino acid substitution(s) in the CLκ domain variant polypeptide comprise(s) or consist(s) of 129R, 178R, and 180Q; or
in (iv), the amino acid substitution(s) in the CLκ domain variant polypeptide comprise(s) or consist(s) of 135S and 178R.

Embodiment 7. The CH1 domain variant polypeptide of one of Embodiments 1-6, comprising the amino acid sequence according to any one of SEQ ID NOS: 11, 21, 31, 41, 51, 61, 71, 81, 91, 101, 111, 121, 131, 141, 151, 161, 171, 181, 191, or 201.

Embodiment 8. The CH1 domain variant polypeptide of any one of Embodiments 1-6, comprising the amino acid sequence according to any one of SEQ ID NOS: 11, 21, 31, or 41.

Embodiment 9. A CLκ domain variant polypeptide comprising an amino acid substitution(s), wherein the amino acid substitution(s) comprise(s) or consist(s) of an amino acid substitution(s) at one or more of the following amino acid positions: 114, 120, 124, 127, 129, 133, 135, 137, 138, 178, and/or 180, according to EU numbering,
optionally such that the CLκ domain variant polypeptide preferentially pairs with a CH1 domain variant polypeptide comprising an amino acid substitution(s), wherein the amino acid substitution(s) in the CH1 domain variant polypeptide comprise(s) or consist(s) of an amino acid substitution(s) at one or more of the following positions: 124, 128, 139, 141, 145, 147, 148, 166, 168, 175, 181, 185, and 187, according to EU numbering,
and optionally wherein:
   (i) when the amino acid substitution(s) in the CLκ domain variant polypeptide consists of N137K and S114A and preferentially pairs with a CH1 domain variant polypeptide, the amino acid substitution(s) in the CH1 domain variant polypeptide do(es) not consist of T187E;
   (ii) when the amino acid substitution(s) in the CLκ domain variant polypeptide consists of V133T; S176V and preferentially pairs with a CH1 domain variant polypeptide, the amino acid substitution(s) in the CH1 domain variant polypeptide do(es) not consist of L145Q and S183V;
   (iii) when the amino acid substitution(s) in the CLκ domain variant polypeptide consists of V133E and preferentially pairs with a CH1 domain variant polypeptide, the amino acid substitution(s) in the CH1 domain variant polypeptide do(es) not consist of S183K;
   (iv) when the amino acid substitution(s) in the CLκ domain variant polypeptide consists of F116A, L135V, S174A, S176F, and T178V and preferentially pairs with a CH1 domain variant polypeptide, the amino acid substitution(s) in the CH1 domain variant polypeptide do(es) not consist of A141I, F170S, S181M, S183A, and V185A;
   (v) when the amino acid substitution(s) in the CLκ domain variant polypeptide consists of T129R and preferentially pairs with a CH1 domain variant polypeptide, the amino acid substitution(s) in the CH1 domain variant polypeptide do(es) not consist of K147D;
   (vi) when the amino acid substitution(s) in the CLκ domain variant polypeptide consists of S176 and T178 and preferentially pairs with a CH1 domain variant polypeptide, the amino acid substitution(s) in the CH1 domain variant polypeptide do(es) not consist of S181E and S183V; or
   (vii) when the amino acid substitution(s) in the CLκ domain variant polypeptide consists of V133S and preferentially pairs with a CH1 domain variant polypeptide, the amino acid substitution(s) in the CH1 domain variant polypeptide do(es) not consist of S183L and V185Y.

Embodiment 10. The CLκ domain variant polypeptide of Embodiment 9, wherein the amino acid substitution(s) of the CLκ domain variant polypeptide comprise(s) or consist(s) of an amino acid substitution(s) at:
(I) position 135; (II) position 124; (III) position 129; (IV) position 133; (V) position(s) 137 and/or 138; (VI) position(s) 178 and/or 180; or (VII) position 127,
optionally wherein the CLκ domain variant polypeptide preferentially pairs with a CH1 domain variant polypeptide and:
in (I), the amino acid substitution(s) in the CH1 domain variant polypeptide comprise(s) or consist(s) of an amino acid position(s) 185 and/or 187;
in (II), the amino acid substitution(s) in the CH1 domain variant polypeptide comprise(s) or consist(s) of an amino acid substitution at position(s) 145, 147, and/or 148;
in (III), the amino acid substitution(s) in the CH1 domain variant polypeptide comprise(s) or consist(s) of an amino acid substitution at position(s) 147 or 148;
in (IV), the amino acid substitution(s) in the CH1 domain variant polypeptide comprise(s) or consist(s) of an amino acid substitution at position 145;
in (V), the amino acid substitution(s) in the CH1 domain variant polypeptide comprise(s) or consist(s) of an amino acid substitution at position(s) 166 and/or 187;
in (VI), the amino acid substitution(s) in the CH1 domain variant polypeptide comprise(s) or consist(s) of an amino acid substitution at position(s) 145 and/or 147; or
in (VII), the amino acid substitution(s) in the CH1 domain variant polypeptide comprise(s) or consist(s) of an amino acid substitution at position(s) 124 and/or 147.

Embodiment 11. The CLκ domain variant polypeptide of Embodiment 9, wherein the amino acid substitution(s) of the CLκ domain variant polypeptide comprises or consist of an amino acid substitution(s) at:
(i) position 135; (ii) positions 124, 133, and 178; (iii) positions 129, 178, and 180; (iv) positions 135 and 178; (v) positions 124 and 129; (vi) positions 114, 135, and 138; (vii) positions 137 and 138; (viii) positions 127 and 129; (ix) position 133; (x) positions 124 and 133; (xi) positions 120, 178, and 180; (xii) positions 127, 129, and 178; (xiii) positions 114, 137, and 138; (xiv) positions 129 and 180; or (xv) positions 133 and 180,
optionally wherein the CLκ domain variant polypeptide preferentially pairs with a CH1 domain variant polypeptide and:
in (i), the amino acid substitution(s) in the CH1 domain variant polypeptide comprise(s) or consist(s) of an amino acid substitution(s) at positions 168 and 185 or positions 168, 185, and 187;
in (ii), the amino acid substitution(s) in the CH1 domain variant polypeptide comprise(s) or consist(s) of an amino acid substitution(s) at positions 128 and 147;
in (iii), the amino acid substitution(s) in the CH1 domain variant polypeptide comprise(s) or consist(s) of an amino acid substitution(s) at positions 145, 147, and 181 or positions 147 and 175;
in (iv), the amino acid substitution(s) in the CH1 domain variant polypeptide comprise(s) or consist(s) of an amino acid substitution(s) at positions 147 and 185;
in (v), the amino acid substitution(s) in the CH1 domain variant polypeptide comprise(s) or consist(s) of an amino acid substitution(s) at position 148;
in (vi), the amino acid substitution(s) in the CH1 domain variant polypeptide comprise(s) or consist(s) of an amino acid substitution(s) at positions 139, 141, and 187;
in (vii), the amino acid substitution(s) in the CH1 domain variant polypeptide comprise(s) or consist(s) of an amino acid substitution(s) at positions 166 and 187;
in (viii), the amino acid substitution(s) in the CH1 domain variant polypeptide comprise(s) or consist(s) of an amino acid substitution(s) at positions 124 and 147 or positions 147 and 148;
in (ix), the amino acid substitution(s) in the CH1 domain variant polypeptide comprise(s) or consist(s) of an amino acid substitution(s) at position 145 or positions 145 and 181;
in (x), the amino acid substitution(s) in the CH1 domain variant polypeptide comprise(s) or consist(s) of an amino acid substitution(s) at position 145;
in (xi), the amino acid substitution(s) in the CH1 domain variant polypeptide comprise(s) or consist(s) of an amino acid substitution(s) at positions 145 and 181;
in (xii), the amino acid substitution(s) in the CH1 domain variant polypeptide comprise(s) or consist(s) of an amino acid substitution(s) at positions 124, 145, and 147;
in (xiii), the amino acid substitution(s) in the CH1 domain variant polypeptide comprise(s) or consist(s) of an amino acid substitution(s) at positions 166 and 187;
in (xiv), the amino acid substitution(s) in the CH1 domain variant polypeptide comprise(s) or consist(s) of an amino acid substitution(s) at positions 147 and 185 or positions 147, 175, and 181; or
in (xv), the amino acid substitution(s) in the CH1 domain variant polypeptide comprise(s) or consist(s) of an amino acid substitution(s) at positions 145 and 147.

Embodiment 12. The CLκ domain variant polypeptide of any one of Embodiments 9-11, wherein the amino acid substitution(s) in the CLκ domain variant polypeptide comprise(s) or consist(s) of: 114D, 114Q, 120S, 124E, 124S, 127D, 127R, 127T, 129D, 129E, 129R, 133Q, 133Y, 135R, 135S, 137S, 137T, 138E, 138R, 178E, 178H, 178R, and 180H, 180Q, 180R, and/or 180S.

Embodiment 13. The CLκ domain variant polypeptide of any one of Embodiments 9-12, wherein the amino acid substitution(s) of the CLκ domain variant polypeptide comprise(s) or consist(s) of:
(i) 135R; (ii) 124E, 133Q, and 178E; (iii) 129R, 178R, and 180Q; (iv) 135S and 178R; (v) 124S and 129E; (vi) 114D, 135S, and 138R; (vii) 137S and 138E; (viii) 135S; (ix) 127D and 129E; (x) 127R and 129R; (xi) 133Y; (xii) 133Y; (xiii) 124E and 133Y; (xiv) 120S, 178H, and 180Q; (xv) 127T, 129D, and 178R; (xvi) 114Q, 137T, and 138E; (xvii) 129D, 178R, and 180H; (xviii) 129D and 180Q; (xix) 133Y and 180R; or (xx) 129R and 180S,
optionally wherein the CLκ domain variant polypeptide preferentially pairs with a CH1 domain variant polypeptide and:
in (i), the amino acid substitution(s) in the CH1 domain variant polypeptide comprise(s) or consist(s) of 168S, 185S, and 187D;
in (ii), the amino acid substitution(s) in the CH1 domain variant polypeptide comprise(s) or consist(s) of 128R and 147R;
in (iii), the amino acid substitution(s) in the CH1 domain variant polypeptide comprise(s) or consist(s) of 145Q, 147E, and 181E;
in (iv), the amino acid substitution(s) in the CH1 domain variant polypeptide comprise(s) or consist(s) of 147T and 185Q;
in (v), the amino acid substitution(s) in the CH1 domain variant polypeptide comprise(s) or consist(s) of 148R;
in (vi), the amino acid substitution(s) in the CH1 domain variant polypeptide comprise(s) or consist(s) of 139R, 141Q, and 187Q;
in (vii), the amino acid substitution(s) in the CH1 domain variant polypeptide comprise(s) or consist(s) of 166K and 187K;
in (viii), the amino acid substitution(s) in the CH1 domain variant polypeptide comprise(s) or consist(s) of 168R and 185E;
in (ix), the amino acid substitution(s) in the CH1 domain variant polypeptide comprise(s) or consist(s) of 124R and 147R;
in (x), the amino acid substitution(s) in the CH1 domain variant polypeptide comprise(s) or consist(s) of 147H and 148E;
in (xi), the amino acid substitution(s) in the CH1 domain variant polypeptide comprise(s) or consist(s) of 145S;
in (xii), the amino acid substitution(s) in the CH1 domain variant polypeptide comprise(s) or consist(s) of 145S and 181Q;
in (xiii), the amino acid substitution(s) in the CH1 domain variant polypeptide comprise(s) or consist(s) of 145S;
in (xiv), the amino acid substitution(s) in the CH1 domain variant polypeptide comprise(s) or consist(s) of 145Q and 181E;
in (xv), the amino acid substitution(s) in the CH1 domain variant polypeptide comprise(s) or consist(s) of 124R, 145S, and 147Q;
in (xvi), the amino acid substitution(s) in the CH1 domain variant polypeptide comprise(s) or consist(s) of 166K and 187K;
in (xvii), the amino acid substitution(s) in the CH1 domain variant polypeptide comprise(s) or consist(s) of 147R and 175D;
in (xviii), the amino acid substitution(s) in the CH1 domain variant polypeptide comprise(s) or consist(s) of 147R, 175E, and 181Q;
in (xix), the amino acid substitution(s) in the CH1 domain variant polypeptide comprise(s) or consist(s) of 145S and 147N; or
in (xx), the amino acid substitution(s) in the CH1 domain variant polypeptide comprise(s) or consist(s) of 147N and 185Y.

Embodiment 14. The CLκ domain variant polypeptide of any one of Embodiments 9-12, wherein the amino acid substitution(s) in the CLκ domain variant polypeptide consist(s) of
(i) 135R;
(ii) 124E, 133Q, and 178E;
(iii) 129R, 178R, and 180Q; or
(iv) 135S and 178R,
optionally wherein the CLκ domain variant polypeptide preferentially pairs with a CH1 domain variant polypeptide and:
in (i), the amino acid substitution(s) in the CH1 domain variant polypeptide comprise(s) or consist(s) of 168S, 185S, and 187D;
in (ii), the amino acid substitution(s) in the CH1 domain variant polypeptide comprise(s) or consist(s) of 128R and 147R;
in (iii), the amino acid substitution(s) in the CH1 domain variant polypeptide comprise(s) or consist(s) of 145Q, 147E, and 181E; or
in (iv), the amino acid substitution(s) in the CH1 domain variant polypeptide comprise(s) or consist(s) of 147T and 185Q.

Embodiment 15. The CLκ domain variant polypeptide of any one of Embodiments 9-14, comprising the amino acid sequence according to any one of SEQ ID NOS: 12, 22, 32, 42, 52, 62, 72, 82, 92, 102, 112, 122, 132, 142, 152, 162, 172, 182, 192, or 202. Embodiment 16. The CLκ domain variant polypeptide of any one of Embodiments 9-14, comprising the amino acid sequence according to any one of SEQ ID NOS: 12, 22, 32, 42.

Embodiment 17. An immunoglobulin polypeptide comprising at least one CH1 domain variant polypeptide according to any one of Embodiments 1-8.

Embodiment 18. The polypeptide of Embodiment 17, further comprising:
(i) an antigen-binding domain;
(ii) a second CH1 domain or domain variant;
(iii) an immunoglobulin heavy chain constant region 2 (“CH2”) domain or domain variant;
(iv) an immunoglobulin heavy chain constant region 3 (“CH3”) domain or domain variant; and/or
(v) a CL domain or domain variant,
optionally wherein:
in (i), the antigen-binding domain comprises an immunoglobulin heavy chain variable region (“VH”) domain, an immunoglobulin light chain variable region (“VL”) domain, a single chain fragment variable (“scFv”), an antigen-binding fragment (Fab), a F(ab′), a F(ab′)2, F(ab′)2, or a combination thereof;
in (ii), the CH1 domain comprises a wild-type CH1 amino acid sequence or comprises one or more amino acid substitutions relative to a wild-type CH1 amino acid sequence;
in (iii), the CH2 domain comprises a wild-type CH2 amino acid sequence or comprises one or more amino acid substitutions relative to a wild-type CH2 amino acid sequence;
in (iv), the CH3 domain comprises a wild-type CH3 amino acid sequence or comprises one or more amino acid substitutions relative to a wild-type CH3 amino acid sequence; and/or
in (v), the CL domain comprises a wild-type CL amino acid sequence or comprises one or more amino acid substitutions relative to a wild-type CL amino acid sequence.

Embodiment 19. The polypeptide of Embodiment 17 or 18, which:
(I) comprises a VH domain and is bound to or paired with another polypeptide comprising a VL domain, wherein the VH domain and the VL domain form an antigen-binding site; or
(II) comprises a VL domain and is bound to or paired with another polypeptide comprising a VH domain, wherein the VL domain and the VH domain form an antigen-binding site.

Embodiment 20. An immunoglobulin polypeptide comprising at least one CLκ domain variant polypeptide according to any one of Embodiments 9-16.

Embodiment 21. The polypeptide of Embodiment 20, further comprising:
(i) an antigen-binding domain; (ii) a CH1 domain or domain variant; (iii) a CH2 domain or domain variant; (iv) a CH3 domain or domain variant; and/or (v) a second CL domain or domain variant, optionally wherein:
in (i), the antigen-binding domain comprises a VH domain, a VL domain, a scFv, a Fab, a F(ab′), a F(ab′)2, F(ab′)2, or a combination thereof;
in (ii), the CH1 domain comprises a wild-type CH1 amino acid sequence or comprises one or more amino acid substitutions relative to a wild-type CH1 amino acid sequence;
in (iii), the CH2 domain comprises a wild-type CH2 amino acid sequence or comprises one or more amino acid substitutions relative to a wild-type CH2 amino acid sequence;
in (iv), the CH3 domain comprises a wild-type CH3 amino acid sequence or comprises one or more amino acid substitutions relative to a wild-type CH3 amino acid sequence; and/or
in (v), the CL domain comprises a wild-type CL amino acid sequence or comprises one or more amino acid substitutions relative to a wild-type CL amino acid sequence.

Embodiment 22. The polypeptide of Embodiment 20 or 21, which:
(I) comprises a VH domain and is bound to or paired with another polypeptide comprising a VL domain, wherein the VH domain and the VL domain form an antigen-binding site; or
(II) comprises a VL domain and is bound to or paired with another polypeptide comprising a VH domain, wherein the VL domain and the VH domain form an antigen-binding site.

Embodiment 23. A molecule comprising at least a first polypeptide and a second polypeptide, wherein:
(A) the first polypeptide comprises the CH1 domain variant polypeptide of Embodiments 1-8; and
(B) the second polypeptide comprises the CLκ domain variant polypeptide of any one of Embodiments 9-16,
and wherein the first polypeptide and the second polypeptide are bound to or paired with each other, optionally via a disulfide bond(s).

Embodiment 24. The molecule of Embodiment 23, wherein:
(A) the first polypeptide is the polypeptide according to any one of Embodiments 17-19; and/or
(B) the second polypeptide is the polypeptide according to any one of Embodiments 20-22.

Embodiment 25. The molecule of Embodiment 23 or 24, wherein:
(A) the first polypeptide comprises an antigen-binding domain; and/or
(B) the second polypeptide comprises an antigen-binding domain,
optionally wherein the antigen-binding domain of the first polypeptide and the antigen-binding domain of the second polypeptide:
(I) comprise a VH and a VL, respectively, or a VL and a VH, respectively, further optionally forming an antigen binding site specific for a first epitope; or
(II) comprises a scFv specific for a first epitope and a scFv specific for a second epitope, respectively, further optionally wherein the first epitope is the same as or is different than the second epitope.

Embodiment 26. The molecule of any one of Embodiments 23-25, further comprising:
(C) a third polypeptide comprising the CH1 domain variant polypeptide of Embodiments 1-8; and
(D) a fourth polypeptide comprising the CLκ domain variant polypeptide of any one of Embodiments 9-16,
wherein the third polypeptide and the fourth polypeptide are bound to or paired with each other, optionally via a disulfide bond(s),
optionally wherein:
(C) the CH1 domain variant polypeptide of the third polypeptide is the same as or is different than the CH1 domain variant polypeptide of the first polypeptide; and/or
(D) the CLκ domain variant polypeptide of the fourth polypeptide is same as or different from the CLκ domain variant polypeptide of the second polypeptide.

Embodiment 27. The molecule of Embodiment 26, wherein:
(C) the third polypeptide is the polypeptide according to any one of Embodiments 17-19; and/or
(D) the fourth polypeptide is the polypeptide according to any one of Embodiments 20-22.

Embodiment 28. The molecule of Embodiment 26 or 27, wherein:
(C) the third polypeptide comprises an antigen-binding domain; and
(D) the fourth polypeptide comprises an antigen-binding domain,
optionally wherein the antigen-binding domain of the third polypeptide and the antigen-binding domain of the fourth polypeptide:
(I) comprise a VH and a VL, respectively, or a VL and a VH, respectively, optionally forming an antigen-binding site specific for a third epitope, further optionally wherein the third epitope is same as or different from the first and/or second epitope; or
(II) comprises a scFv specific for a third epitope and a scFv specific for a fourth epitope, respectively, optionally wherein the third epitope is same as or different from the fourth epitope, further optionally wherein the third and/or fourth epitopes are same as or different from the first and/or second epitope.

Embodiment 29. The molecule of any one of Embodiments 26-28, which is a multi-specific antibody or antigen-binding antibody fragment, optionally a bispecific, tri-specific, tetra-specific, penta-specific, or hexa-specific antibody or antigen-binding antibody fragment, further optionally comprising a structure as depicted in any one of FIGS. 2-7.

Embodiment 30. The molecule of any one of Embodiments 26-29, wherein:
(A) the amino acid substitutions in the CH1 domain of the first polypeptide comprise or consist of 145Q, 147E, and 181E, the amino acid substitutions in the CLκ domain of the second polypeptide comprise or consist of 129R, 178R, and 180Q, and the amino acid substitutions in the CH1 domain of the third polypeptide comprise or consist of 128R and 147R, and the amino acid substitutions in the CLκ domain of the fourth polypeptide comprise or consist of 124E, 133Q, and 178E; or
(B) the amino acid substitutions in the CH1 domain of the first polypeptide comprise or consist of 128R and 147R, the amino acid substitutions in the CLκ domain of the second polypeptide comprise or consist of 124E, 133Q, and 178E, the amino acid substitutions in the CH1 domain of the third polypeptide comprise or consist of 145Q, 147E, and 181E, and the amino acid substitutions in the CLκ domain of the fourth polypeptide comprise or consist of 129R, 178R, and 180Q.

Embodiment 31. The molecule of any one of Embodiments 26-30, wherein the CH1 domain of the first polypeptide, the CLκ domain of the second polypeptide, the CH1 domain of the third polypeptide, and the CLκ domain of the fourth polypeptide comprise the amino acid sequence of
(A) SEQ ID NOS: 31, 32, 21, and 22, respectively; or
(B) SEQ ID NOS: 21, 22, 31, and 32, respectively.

Embodiment 32. A polynucleotide or polynucleotides encoding:
(i) the CH1 domain variant polypeptide of any one of Embodiments 1-8,
(ii) the CLκ domain variant polypeptide of any one of Embodiments 9-16;
(iii) the polypeptide of any one of Embodiments 17-22; and/or
(iv) the molecule of any one of Embodiments 23-31.

Embodiment 33. A vector or vectors comprising the polynucleotide or polynucleotides according to Embodiment 32.

Embodiment 34. A cell, which comprises:
(i) the CH1 domain variant polypeptide of any one of Embodiments 1-8,
(ii) the CLκ domain variant polypeptide of any one of Embodiments 9-16;
(iii) the polypeptide of any one of Embodiments 17-22;
(iv) the molecule of any one of Embodiments 23-31;
(v) the polynucleotide or polynucleotides according to Embodiment 32; and/or
(vi) the vector or vectors according to Embodiment 33,
optionally wherein the cell is a mammalian cell.

Embodiment 35. A composition, comprising:
(I) (i) the CH1 domain variant polypeptide of any one of Embodiments 1-8,
(ii) the CLκ domain variant polypeptide of any one of Embodiments 9-16;
(iii) the polypeptide of any one of Embodiments 17-22;
(iv) the molecule of any one of Embodiments 23-31;
(v) the polynucleotide or polynucleotides according to Embodiment 32; and/or
(vi) the vector or vectors according to Embodiment 33; and/or
(vii) the cell of Embodiment 34; and
(II) a pharmaceutically acceptable carrier.

Embodiment 36. A method of generating a CH1 domain variant library, comprising incorporating a mutation at or randomizing the nucleic acid at one or more pre-determined nucleotide positions, wherein at least one of the one or more pre-determined nucleotide positions is within the codon(s) encoding the amino acid at one or more of pre-determined CH1 domain amino acid positions selected from positions 124, 128, 139, 141, 145, 147, 148, 166, 168, 175, 181, 185, and 187, according to EU numbering,
optionally wherein the one or more mutations are generated via a degenerate codon, optionally a degenerate RMW codon representing six naturally occurring amino acids (D, T, A, E, K, and N) or a degenerate NNK codon representing all 20 naturally occurring amino acid residues,
further optionally wherein the library is for identifying one or more CH1 domain variant polypeptides which preferentially pairs with a CLκ domain or a CLκ domain variant polypeptide with a wild-type CLκ, another CLκ domain variant polypeptide, a lambda light chain constant region (CLλ) domain, or a CLλ domain variant.

Embodiment 37. A method of generating a CLκ domain variant library, comprising incorporating a mutation at or randomizing the nucleic acid at one or more pre-determined nucleotide positions, wherein at least one of the one or more pre-determined nucleotide positions is within the codon(s) encoding the amino acid at one or more of pre-determined CLκ domain amino acid positions selected from positions 114, 120, 124, 127, 129, 133, 135, 137, 138, 178, and 180, according to EU numbering,
optionally wherein the one or more mutations are generated via a degenerate codon, optionally a degenerate RMW codon representing six naturally occurring amino acids (D, T, A, E, K, and N) or a degenerate NNK codon representing all 20 naturally occurring amino acid residues,
further optionally wherein the library is for identifying one or more CLκ domain variant polypeptides which preferentially pairs with a CH1 domain variant with a wild-type CH1 or another CH1 domain variant polypeptide.

Embodiment 38. A CH1 domain variant library produced according to Embodiment 36.

Embodiment 39. A CLκ domain variant library produced according to Embodiment 37.

Embodiment 40. A method of identifying one or more sets of a CH1 domain variant polypeptide and a CLκ domain variant polypeptide, wherein the CH1 domain variant polypeptide preferentially pairs with the CLκ domain variant polypeptide, the method comprising:
(a) computationally or recombinantly co-expressing or combining (a-1) a first polypeptide or a first set of polypeptides each comprising a wild-type CH1 domain polypeptide or a CH1 domain variant polypeptide expressed from the CH1 domain variant library according to Embodiment 38 and (a-2) a second polypeptide or a second set of polypeptides each comprising a wild-type CLκ domain polypeptide or a CLκ domain variant polypeptide expressed from a CLκ domain variant library according to Embodiment 39;
(b) quantifying the binding preference between the CH1 domain variant polypeptide and the CLκ domain variant polypeptide; and
(c) selecting one or more sets of a CH1 domain variant polypeptide and a CLκ domain variant polypeptide which provide preferential CH1-CLκ paring, optionally equivalent or higher preferential pairing relative to a reference CH1-CLκ set, further optionally wherein the reference CH1-CLκ set comprises a wildtype CH1 domain, a wildtype CH1 domain, the CH1 domain variant polypeptide of any one of Embodiments 1-8, and/or the CLκ domain variant polypeptide of any one of Embodiments 9-16.

Embodiment 41. The method of Embodiment 40, wherein:
(i) the one or more predetermined CH1 domain amino acid positions of the CH1 domain library comprise or consist of positions 168, 185, and/or 187, and/or the one or more predetermined CLκ domain amino acid positions of the CLκ domain library comprise or consist of position 135;
(ii) the one or more predetermined CH1 domain amino acid positions of the CH1 domain library comprise or consist of positions 128 and/or 147, and/or the one or more predetermined CLκ domain amino acid positions of the CLκ domain library comprise or consist of positions 124, 133, and/or 178;
(iii) the one or more predetermined CH1 domain amino acid positions of the CH1 domain library comprise or consist of positions 145, 147, and/or 181, and/or the one or more predetermined CLκ domain amino acid positions of the CLκ domain library comprise or consist of positions 129, 178, and/or 180;
(iv) the one or more predetermined CH1 domain amino acid positions of the CH1 domain library comprise or consist of positions 147 and/or 185, and/or the one or more predetermined CLκ domain amino acid positions of the CLκ domain library comprise or consist of positions 135 and/or 178;
(v) the one or more predetermined CH1 domain amino acid positions of the CH1 domain library comprise or consist of position 148, and/or the one or more predetermined CLκ domain amino acid positions of the CLκ domain library comprise or consist of positions 124 and/or 129;
(vi) the one or more predetermined CH1 domain amino acid positions of the CH1 domain library comprise or consist of positions 139, 141, and/or 187, and/or the one or more predetermined CLκ domain amino acid positions of the CLκ domain library comprise or consist of positions 114, 135, and/or 138;
(vii) the one or more predetermined CH1 domain amino acid positions of the CH1 domain library comprise or consist of positions 166 and/or 187, and/or the one or more predetermined CLκ domain amino acid positions of the CLκ domain library comprise or consist of positions 137 and/or 138;
(viii) the one or more predetermined CH1 domain amino acid positions of the CH1 domain library comprise or consist of positions 168 and/or 185, and/or the one or more predetermined CLκ domain amino acid positions of the CLκ domain library comprise or consist of position 135;
(ix) the one or more predetermined CH1 domain amino acid positions of the CH1 domain library comprise or consist of positions 124 and/or 147, and/or the one or more predetermined CLκ domain amino acid positions of the CLκ domain library comprise or consist of positions 127 and/or 129;
(x) the one or more predetermined CH1 domain amino acid positions of the CH1 domain library comprise or consist of positions 147 and/or 148, and/or the one or more predetermined CLκ domain amino acid positions of the CLκ domain library comprise or consist of positions 127 and/or 129;
(xi) the one or more predetermined CH1 domain amino acid positions of the CH1 domain library comprise or consist of position 145, and/or the one or more predetermined CLκ domain amino acid positions of the CLκ domain library comprise or consist of position 133;
(xii) the one or more predetermined CH1 domain amino acid positions of the CH1 domain library comprise or consist of positions 145 and/or 181, and/or the one or more predetermined CLκ domain amino acid positions of the CLκ domain library comprise or consist of position 133;
(xiii) the one or more predetermined CH1 domain amino acid positions of the CH1 domain library comprise or consist of position 145, and/or the one or more predetermined CLκ domain amino acid positions of the CLκ domain library comprise or consist of positions 124 and/or 133;
(xiv) the one or more predetermined CH1 domain amino acid positions of the CH1 domain library comprise or consist of positions 145 and/or 181, and/or the one or more predetermined CLκ domain amino acid positions of the CLκ domain library comprise or consist of positions 120, 178, and/or 180;
(xv) the one or more predetermined CH1 domain amino acid positions of the CH1 domain library comprise or consist of positions 124, 145, and/or 147, and/or the one or more predetermined CLκ domain amino acid positions of the CLκ domain library comprise or consist of positions 127, 129, and/or 178;
(xvi) the one or more predetermined CH1 domain amino acid positions of the CH1 domain library comprise or consist of positions 166 and/or 187, and/or the one or more predetermined CLκ domain amino acid positions of the CLκ domain library comprise or consist of positions 114, 137, and/or 138;
(xvii) the one or more predetermined CH1 domain amino acid positions of the CH1 domain library comprise or consist of positions 147 and/or 175, and/or the one or more predetermined CLκ domain amino acid positions of the CLκ domain library comprise or consist of positions 129, 178, and/or 180;
(xviii) the one or more predetermined CH1 domain amino acid positions of the CH1 domain library comprise or consist of positions 147, 175, and/or 181, and/or the one or more predetermined CLκ domain amino acid positions of the CLκ domain library comprise or consist of positions 129 and/or 180;
(xix) the one or more predetermined CH1 domain amino acid positions of the CH1 domain library comprise or consist of positions 145 and/or 147, and/or the one or more predetermined CLκ domain amino acid positions of the CLκ domain library comprise or consist of positions 133 and/or 180; or
(xx) the one or more predetermined CH1 domain amino acid positions of the CH1 domain library comprise or consist of positions 147 and/or 185, and/or the one or more predetermined CLκ domain amino acid positions of the CLκ domain library comprise or consist of positions 129 and/or 180.

Embodiment 42. The method of Embodiment 40 or 41, wherein:
(a-1) the first polypeptide or each polypeptide of the first set of polypeptides comprises or linked to a first label; and/or
(a-2) the second polypeptide or each of the second set of polypeptides comprises or linked to a second label.

Embodiment 43. The method of Embodiment 42, wherein the quantifying step (b) comprises detecting the first label and/or the second label.

Embodiment 44. The method of any one of Embodiments 40-43, wherein:
in step (a), the first polypeptide or the first set of polypeptides and the second polypeptide or the second set of polypeptides are computationally co-expressed; and
in step (b), the quantifying comprises calculating a score, optionally selected from: ΔΔG; ΔΔGcognate total score; ΔΔGcognate hbond_all; RBPP; RBPPtotal score; RBPPhbond_all; and/or RBPPbond elec backrub 18k.

Embodiment 45. The method of any one of Embodiments 40-43, wherein:
in step (a), the first polypeptide or the first set of polypeptides and the second polypeptide or the second set of polypeptides are recombinantly co-expressed; and
in step (b), the quantifying comprises measuring the amounts of CH1-CLκ pairs via liquid chromatography-mass spectrometry (LC-MS), ion exchange chromatography (IEX), AlphaLISA®, and/or flow cytometry.

### APPENDIX

APPENDIX TABLE A
CH1 sequences

| CH1 | | SEQ |
|---|---|---|
| substitution(s) | Exemplary CH1 sequence | ID NO: |
| WT | ASTKGPSVFPLAPSSKSTSGGTAALGCLVKDYFPEPVTVSWNSGA | 1 |
| | LTSGVHTFPAVLQSSGLYSLSSVVTVPSSSLGTQTYICNVNHKPSN | |
| | TKVDKKVEPKSC | |
| 168S; 185S; | ASTKGPSVFPLAPSSKSTSGGTAALGCLVKDYFPEPVTVSWNSGA | 11 |
| 187D | LTSGVSTFPAVLQSSGLYSLSSSVDVPSSSLGTQTYICNVNHKPSN | |
| | TKVDKKVEPKSC | |
| 128R; 147R | ASTKGPSVFPRAPSSKSTSGGTAALGCLVRDYFPEPVTVSWNSGA | 21 |
| | LTSGVHTFPAVLQSSGLYSLSSVVTVPSSSLGTQTYICNVNHKPSN | |
| | TKVDKKVEPKSC | |
| 145Q; 147E; | ASTKGPSVFPLAPSSKSTSGGTAALGCQVEDYFPEPVTVSWNSGA | 31 |
| 181E | LTSGVHTFPAVLQSSGLYELSSVVTVPSSSLGTQTYICNVNHKPSN | |
| | TKVDKKVEPKSC | |
| 147T; 185Q | ASTKGPSVFPLAPSSKSTSGGTAALGCLVTDYFPEPVTVSWNSGA | 41 |
| | LTSGVHTFPAVLQSSGLYSLSSQVTVPSSSLGTQTYICNVNHKPSN | |
| | TKVDKKVEPKSC | |
| 148R | ASTKGPSVFPLAPSSKSTSGGTAALGCLVKRYFPEPVTVSWNSGA | 51 |
| | LTSGVHTFPAVLQSSGLYSLSSVVTVPSSSLGTQTYICNVNHKPSN | |
| | TKVDKKVEPKSC | |
| 139R; 141Q; | ASTKGPSVFPLAPSSKSTSGGRAQLGCLVKDYFPEPVTVSWNSGA | 61 |
| 187Q | LTSGVHTFPAVLQSSGLYSLSSVVQVPSSSLGTQTYICNVNHKPSN | |
| | TKVDKKVEPKSC | |
| 166K; 187K | ASTKGPSVFPLAPSSKSTSGGTAALGCLVKDYFPEPVTVSWNSGA | 71 |
| | LTSKVHTFPAVLQSSGLYSLSSVVKVPSSSLGTQTYICNVNHKPSN | |
| | TKVDKKVEPKSC | |
| 168R; 185E | ASTKGPSVFPLAPSSKSTSGGTAALGCLVKDYFPEPVTVSWNSGA | 81 |
| | LTSGVRTFPAVLQSSGLYSLSSEVTVPSSSLGTQTYICNVNHKPSN | |
| | TKVDKKVEPKSC | |
| 124R; 147R | ASTKGPRVFPLAPSSKSTSGGTAALGCLVRDYFPEPVTVSWNSGA | 91 |
| | LTSGVHTFPAVLQSSGLYSLSSVVTVPSSSLGTQTYICNVNHKPSN | |
| | TKVDKKVEPKSC | |
| 147H; 148E | ASTKGPSVFPLAPSSKSTSGGTAALGCLVHEYFPEPVTVSWNSGA | 101 |
| | LTSGVHTFPAVLQSSGLYSLSSVVTVPSSSLGTQTYICNVNHKPSN | |
| | TKVDKKVEPKSC | |
| 145S | ASTKGPSVFPLAPSSKSTSGGTAALGCSVKDYFPEPVTVSWNSGA | 111 |
| | LTSGVHTFPAVLQSSGLYSLSSVVTVPSSSLGTQTYICNVNHKPSN | |
| | TKVDKKVEPKSC | |
| 145S; 181Q | ASTKGPSVFPLAPSSKSTSGGTAALGCSVKDYFPEPVTVSWNSGA | 121 |
| | LTSGVHTFPAVLQSSGLYQLSSVVTVPSSSLGTQTYICNVNHKPSN | |
| | TKVDKKVEPKSC | |
| 145S | ASTKGPSVFPLAPSSKSTSGGTAALGCSVKDYFPEPVTVSWNSGA | 131 |
| | LTSGVHTFPAVLQSSGLYSLSSVVTVPSSSLGTQTYICNVNHKPSN | |
| | TKVDKKVEPKSC | |
| 145Q; 181E | ASTKGPSVFPLAPSSKSTSGGTAALGCQVKDYFPEPVTVSWNSGA | 141 |
| | LTSGVHTFPAVLQSSGLYELSSVVTVPSSSLGTQTYICNVNHKPSN | |
| | TKVDKKVEPKSC | |
| 124R; 145S; | ASTKGPRVFPLAPSSKSTSGGTAALGCSVQDYFPEPVTVSWNSGA | 151 |
| 147Q | LTSGVHTFPAVLQSSGLYSLSSVVTVPSSSLGTQTYICNVNHKPSN | |
| | TKVDKKVEPKSC | |
| 166K; 187K | ASTKGPSVFPLAPSSKSTSGGTAALGCLVKDYFPEPVTVSWNSGA | 161 |
| | LTSKVHTFPAVLQSSGLYSLSSVVKVPSSSLGTQTYICNVNHKPSN | |
| | TKVDKKVEPKSC | |
| 147R; 175D | ASTKGPSVFPLAPSSKSTSGGTAALGCLVRDYFPEPVTVSWNSGA | 171 |
| | LTSGVHTFPAVLDSSGLYSLSSVVTVPSSSLGTQTYICNVNHKPSN | |
| | TKVDKKVEPKSC | |
| 147R; 175E; | ASTKGPSVFPLAPSSKSTSGGTAALGCLVRDYFPEPVTVSWNSGA | 181 |
| 181Q | LTSGVHTFPAVLESSGLYQLSSVVTVPSSSLGTQTYICNVNHKPSN | |
| | TKVDKKVEPKSC | |
| 145S; 147N | ASTKGPSVFPLAPSSKSTSGGTAALGCSVNDYFPEPVTVSWNSGA | 191 |
| | LTSGVHTFPAVLQSSGLYSLSSVVTVPSSSLGTQTYICNVNHKPSN | |
| | TKVDKKVEPKSC | |
| 147N; 185Y | ASTKGPSVFPLAPSSKSTSGGTAALGCLVNDYFPEPVTVSWNSGA | 201 |
| | LTSGVHTFPAVLQSSGLYSLSSYVTVPSSSLGTQTYICNVNHKPSN | |
| | TKVDKKVEPKSC | |

* underlined: amino acid substitution relative to WT

APPENDIX TABLE B
CLκ sequences

| CLκ | | SEQ |
|---|---|---|
| substitution(s) | Exemplary CLκ sequence | ID NO: |
| WT | RTVAAPSVFIFPPSDEQLKSGTASVVCLLNNFYPREAKVQWKVD | 2 |
| | NALQSGNSQESVTEQDSKDSTYSLSSTLTLSKADYEKHKVYACE | |
| | VTHQGLSSPVTKSFNRGEC | |
| 135R | RTVAAPSVFIFPPSDEQLKSGTASVVCRLNNFYPREAKVQWKVD | 12 |
| | NALQSGNSQESVTEQDSKDSTYSLSSTLTLSKADYEKHKVYACE | |
| | VTHQGLSSPVTKSFNRGEC | |
| 124E; 133Q; | RTVAAPSVFIFPPSDEELKSGTASVQCLLNNFYPREAKVQWKVD | 22 |
| 178E | NALQSGNSQESVTEQDSKDSTYSLSSELTLSKADYEKHKVYACE | |
| | VTHQGLSSPVTKSFNRGEC | |
| 129R; 178R; | RTVAAPSVFIFPPSDEQLKSGRASVVCLLNNFYPREAKVQWKVD | 32 |
| 180Q | NALQSGNSQESVTEQDSKDSTYSLSSRLQLSKADYEKHKVYACE | |
| | VTHQGLSSPVTKSFNRGEC | |
| 135S; 178R | RTVAAPSVFIFPPSDEQLKSGTASVVCSLNNFYPREAKVQWKVD | 42 |
| | NALQSGNSQESVTEQDSKDSTYSLSSRLTLSKADYEKHKVYACE | |
| | VTHQGLSSPVTKSFNRGEC | |
| 124S; 129E | RTVAAPSVFIFPPSDESLKSGEASVVCLLNNFYPREAKVQWKVD | 52 |
| | NALQSGNSQESVTEQDSKDSTYSLSSTLTLSKADYEKHKVYACE | |
| | VTHQGLSSPVTKSFNRGEC | |
| 114D; 135S; | RTVAAPDVFIFPPSDEQLKSGTASVVCSLNRFYPREAKVQWKVD | 62 |
| 138R | NALQSGNSQESVTEQDSKDSTYSLSSTLTLSKADYEKHKVYACE | |
| | VTHQGLSSPVTKSFNRGEC | |
| 137S; 138E | RTVAAPSVFIFPPSDEQLKSGTASVVCLLSEFYPREAKVQWKVD | 72 |
| | NALQSGNSQESVTEQDSKDSTYSLSSTLTLSKADYEKHKVYACE | |
| | VTHQGLSSPVTKSFNRGEC | |
| 135S | RTVAAPSVFIFPPSDEQLKSGTASVVCSLNNFYPREAKVQWKVD | 82 |
| | NALQSGNSQESVTEQDSKDSTYSLSSTLTLSKADYEKHKVYACE | |
| | VTHQGLSSPVTKSFNRGEC | |
| 127D; 129E | RTVAAPSVFIFPPSDEQLKDGEASVVCLLNNFYPREAKVQWKVD | 92 |
| | NALQSGNSQESVTEQDSKDSTYSLSSTLTLSKADYEKHKVYACE | |
| | VTHQGLSSPVTKSFNRGEC | |
| 127R; 129R | RTVAAPSVFIFPPSDEQLKRGRASVVCLLNNFYPREAKVQWKVD | 102 |
| | NALQSGNSQESVTEQDSKDSTYSLSSTLTLSKADYEKHKVYACE | |
| | VTHQGLSSPVTKSFNRGEC | |
| 133Y | RTVAAPSVFIFPPSDEQLKSGTASVYCLLNNFYPREAKVQWKVD | 112 |
| | NALQSGNSQESVTEQDSKDSTYSLSSTLTLSKADYEKHKVYACE | |
| | VTHQGLSSPVTKSFNRGEC | |
| 133Y | RTVAAPSVFIFPPSDEQLKSGTASVYCLLNNFYPREAKVQWKVD | 122 |
| | NALQSGNSQESVTEQDSKDSTYSLSSTLTLSKADYEKHKVYACE | |
| | VTHQGLSSPVTKSFNRGEC | |
| 124E; 133Y | RTVAAPSVFIFPPSDEELKSGTASVYCLLNNFYPREAKVQWKVD | 132 |
| | NALQSGNSQESVTEQDSKDSTYSLSSTLTLSKADYEKHKVYACE | |
| | VTHQGLSSPVTKSFNRGEC | |
| 120S; 178H; | RTVAAPSVFIFPSSDEQLKSGTASVVCLLNNFYPREAKVQWKVD | 142 |
| 180Q | NALQSGNSQESVTEQDSKDSTYSLSSHLQLSKADYEKHKVYACE | |
| | VTHQGLSSPVTKSFNRGEC | |
| 127T; 129D; | RTVAAPSVFIFPPSDEQLKTGDASVVCLLNNFYPREAKVQWKVD | 152 |
| 178R | NALQSGNSQESVTEQDSKDSTYSLSSRLTLSKADYEKHKVYACE | |
| | VTHQGLSSPVTKSFNRGEC | |
| 114Q; 137T; | RTVAAPQVFIFPPSDEQLKSGTASVVCLLTEFYPREAKVQWKVD | 162 |
| 138E | NALQSGNSQESVTEQDSKDSTYSLSSTLTLSKADYEKHKVYACE | |
| | VTHQGLSSPVTKSFNRGEC | |
| 129D; 178R; | RTVAAPSVFIFPPSDEQLKSGDASVVCLLNNFYPREAKVQWKVD | 172 |
| 180H | NALQSGNSQESVTEQDSKDSTYSLSSRLHLSKADYEKHKVYACE | |
| | VTHQGLSSPVTKSFNRGEC | |
| 129D; 180Q | RTVAAPSVFIFPPSDEQLKSGDASVVCLLNNFYPREAKVQWKVD | 182 |
| | NALQSGNSQESVTEQDSKDSTYSLSSTLQLSKADYEKHKVYACE | |
| | VTHQGLSSPVTKSFNRGEC | |
| 133Y; 180R | RTVAAPSVFIFPPSDEQLKSGTASVYCLLNNFYPREAKVQWKVD | 192 |
| | NALQSGNSQESVTEQDSKDSTYSLSSTLRLSKADYEKHKVYACE | |
| | VTHQGLSSPVTKSFNRGEC | |
| 129R; 180S | RTVAAPSVFIFPPSDEQLKSGRASVVCLLNNFYPREAKVQWKVD | 202 |
| | NALQSGNSQESVTEQDSKDSTYSLSSTLSLSKADYEKHKVYACE | |
| | VTHQGLSSPVTKSFNRGEC | |

* underlined: amino acid substitution relative to WT

APPENDIX TABLE C
CLλ sequences

| CLλ | | SEQ |
|---|---|---|
| substitution(s) | Exemplary CLλ sequence | ID NO: |
| WT | GQPKAAPSVTLFPPSSEELQANKATLVCLISDFYPGAVTVAWKAD | 9 |
| | SSPVKAGVETTTPSKQSNNKYAASSYLSLTPEQWKSHRSYSCQV | |
| | THEGSTVEKTVAPTECS | |
| 135R | GQPKAAPSVTLFPPSSEELQANKATLVCRISDFYPGAVTVAWKAD | 19 |
| | SSPVKAGVETTTPSKQSNNKYAASSYLSLTPEQWKSHRSYSCQV | |
| | THEGSTVEKTVAPTECS | |
| 133Q; 178E | GQPKAAPSVTLFPPSSEELQANKATLQCLISDFYPGAVTVAWKAD | 29 |
| | SSPVKAGVETTTPSKQSNNKYAASSELSLTPEQWKSHRSYSCQV | |
| | THEGSTVEKTVAPTECS | |
| 129R; 178R; | GQPKAAPSVTLFPPSSEELQANRATLVCLISDFYPGAVTVAWKAD | 39 |
| 180Q | SSPVKAGVETTTPSKQSNNKYAASSRLQLTPEQWKSHRSYSCQV | |
| | THEGSTVEKTVAPTECS | |
| 135S; 178R | GQPKAAPSVTLFPPSSEELQANKATLVCSISDFYPGAVTVAWKAD | 49 |
| | SSPVKAGVETTTPSKQSNNKYAASSRLSLTPEQWKSHRSYSCQV | |
| | THEGSTVEKTVAPTECS | |
| 124S; 129E | GQPKAAPSVTLFPPSSESLQANEATLVCLISDFYPGAVTVAWKAD | 59 |
| | SSPVKAGVETTTPSKQSNNKYAASSYLSLTPEQWKSHRSYSCQV | |
| | THEGSTVEKTVAPTECS | |
| 114D; 135S; | GQPKAAPDVTLFPPSSEELQANKATLVCSISRFYPGAVTVAWKAD | 69 |
| 138R | SSPVKAGVETTTPSKQSNNKYAASSYLSLTPEQWKSHRSYSCQV | |
| | THEGSTVEKTVAPTECS | |
| 138E | GQPKAAPSVTLFPPSSEELQANKATLVCLISEFYPGAVTVAWKAD | 79 |
| | SSPVKAGVETTTPSKQSNNKYAASSYLSLTPEQWKSHRSYSCQV | |
| | THEGSTVEKTVAPTECS | |
| 135S | GQPKAAPSVTLFPPSSEELQANKATLVCSISDFYPGAVTVAWKAD | 89 |
| | SSPVKAGVETTTPSKQSNNKYAASSYLSLTPEQWKSHRSYSCQV | |
| | THEGSTVEKTVAPTECS | |
| 127D; 129E | GQPKAAPSVTLFPPSSEELQDNEATLVCLISDFYPGAVTVAWKAD | 99 |
| | SSPVKAGVETTTPSKQSNNKYAASSYLSLTPEQWKSHRSYSCQV | |
| | THEGSTVEKTVAPTECS | |
| 127R; 129R | GQPKAAPSVTLFPPSSEELQRNRATLVCLISDFYPGAVTVAWKAD | 109 |
| | SSPVKAGVETTTPSKQSNNKYAASSYLSLTPEQWKSHRSYSCQV | |
| | THEGSTVEKTVAPTECS | |
| 133Y | GQPKAAPSVTLFPPSSEELQANKATLYCLISDFYPGAVTVAWKAD | 119 |
| | SSPVKAGVETTTPSKQSNNKYAASSYLSLTPEQWKSHRSYSCQV | |
| | THEGSTVEKTVAPTECS | |
| 133Y | GQPKAAPSVTLFPPSSEELQANKATLYCLISDFYPGAVTVAWKAD | 129 |
| | SSPVKAGVETTTPSKQSNNKYAASSYLSLTPEQWKSHRSYSCQV | |
| | THEGSTVEKTVAPTECS | |
| 133Y | GQPKAAPSVTLFPPSSEELQANKATLYCLISDFYPGAVTVAWKAD | 139 |
| | SSPVKAGVETTTPSKQSNNKYAASSYLSLTPEQWKSHRSYSCQV | |
| | THEGSTVEKTVAPTECS | |
| 120S; 178H; | GQPKAAPSVTLFPSSSEELQANKATLVCLISDFYPGAVTVAWKAD | 149 |
| 180Q | SSPVKAGVETTTPSKQSNNKYAASSHLQLTPEQWKSHRSYSCQV | |
| | THEGSTVEKTVAPTECS | |
| 127T; 129D; | GQPKAAPSVTLFPPSSEELQTNDATLVCLISDFYPGAVTVAWKAD | 159 |
| 178R | SSPVKAGVETTTPSKQSNNKYAASSRLSLTPEQWKSHRSYSCQV | |
| | THEGSTVEKTVAPTECS | |
| 114Q; 137T; | GQPKAAPQVTLFPPSSEELQANKATLVCLITEFYPGAVTVAWKAD | 169 |
| 138E | SSPVKAGVETTTPSKQSNNKYAASSYLSLTPEQWKSHRSYSCQV | |
| | THEGSTVEKTVAPTECS | |
| 129D; 178R; | GQPKAAPSVTLFPPSSEELQANDATLVCLISDFYPGAVTVAWKAD | 179 |
| 180H | SSPVKAGVETTTPSKQSNNKYAASSRLHLTPEQWKSHRSYSCQV | |
| | THEGSTVEKTVAPTECS | |
| 129D; 180Q | GQPKAAPSVTLFPPSSEELQANDATLVCLISDFYPGAVTVAWKAD | 189 |
| | SSPVKAGVETTTPSKQSNNKYAASSYLQLTPEQWKSHRSYSCQV | |
| | THEGSTVEKTVAPTECS | |
| 133Y; 180R | GQPKAAPSVTLFPPSSEELQANKATLYCLISDFYPGAVTVAWKAD | 199 |
| | SSPVKAGVETTTPSKQSNNKYAASSYLRLTPEQWKSHRSYSCQV | |
| | THEGSTVEKTVAPTECS | |
| 129R | GQPKAAPSVTLFPPSSEELQANRATLVCLISDFYPGAVTVAWKAD | 209 |
| | SSPVKAGVETTTPSKQSNNKYAASSYLSLTPEQWKSHRSYSCQV | |
| | THEGSTVEKTVAPTECS | |

* underlined: amino acid substitution relative to WT

What is claimed is:

1-66. (canceled)

67. A variant immunoglobulin heavy chain constant region 1 (“CH1”) domain polypeptide, or heavy chain polypeptide comprising said variant CH1 domain polypeptide, which variant CH1 domain polypeptide comprises at least one amino acid substitution, which comprise(s) or consist(s) of an amino acid substitution(s) at one or more of the following CH1 amino acid positions: 145, 147, 181, 128, 124, 139, 141, 148, 166, 168, 175, 185, and 187 according to EU numbering, optionally wherein:
(A) the variant CH1 domain polypeptide or heavy chain polypeptide comprising said variant CH1 domain polypeptide preferentially pairs with a variant immunoglobulin kappa light chain constant region (CLκ) or variant lambda light chain constant region (CLλ) domain polypeptide, or light chain polypeptide comprising said variant CLκ or CLλ domain polypeptide, which variant CLκ or CLλ domain polypeptide comprises at least one amino acid substitution, which substitution(s) optionally comprise(s) or consist(s) of an amino acid substitution(s) at one or more of the following CLκ or CLλ positions: 129, 178, 180, 124, 133, 114, 120, 127, 135, 137, and 138, according to EU numbering;
(B) the variant CH1 domain polypeptide is a variant of a CH1 domain of a human IgG, optionally a human IgG1, human IgG2, or human IgG4;
(C) the one or more amino acid substitution(s) of the variant CH1 domain polypeptide comprise or consist of an amino acid substitution(s) at:
(i) positions 145, 147, and 181; or
(ii) positions 128 and 147; or
(iii) positions 168, 185, and 187; or
(iv) positions 147 and 185; or
(v) position 148; or
(vi) positions 139, 141, and 187; or
(vii) positions 166 and 187; or
(viii) positions 168 and 185; or
(ix) positions 124 and 147; or
(x) positions 147 and 148; or
(xi) position 145; or
(xii) positions 145 and 181; or
(xiii) positions 124, 145, and 147; or
(xiv) positions 166 and 187; or
(xv) positions 147 and 175; or
(xvi) positions 147, 175, and 181; or
(xvii) positions 145 and 147; or
(xviii) positions 147 and 185,
optionally wherein the variant CH1 domain polypeptide preferentially pairs with a variant CLκ or CLλ domain polypeptide, further optionally wherein:
in (i), the amino acid substitution(s) in the variant CLκ or CLλ domain polypeptide comprise(s) or consist(s) of an amino acid substitution(s) at positions 129, 178, and 180;
in (ii), the amino acid substitution(s) in the variant CLκ domain polypeptide comprise(s) or consist(s) of an amino acid substitution(s) at positions 124, 133, and 178 or the amino acid substitution(s) in the variant CLλ domain polypeptide comprise(s) or consist(s) of an amino acid substitution(s) at positions 133 and 178;
in (iii), the amino acid substitution(s) in the variant CLκ or CLλ domain polypeptide comprise(s) or consist(s) of an amino acid substitution(s) at position 135;
in (iv), the amino acid substitution(s) in the variant CLκ or CLλ domain polypeptide comprise(s) or consist(s) of an amino acid substitution(s) at positions 135 and 178;
in (v), the amino acid substitution(s) in the variant CLκ or CLλ domain polypeptide comprise(s) or consist(s) of an amino acid substitution(s) at positions 124 and 129;
in (vi), the amino acid substitution(s) in the variant CLκ or CLλ domain polypeptide comprise(s) or consist(s) of an amino acid substitution(s) at positions 114, 135, and 138;
in (vii), the amino acid substitution(s) in the variant CLκ domain polypeptide comprise(s) or consist(s) of an amino acid substitution(s) at positions 137 and 138 or the amino acid substitution(s) in the variant CLλ domain polypeptide comprise(s) or consist(s) of an amino acid substitution(s) at position 138;
in (viii), the amino acid substitution(s) in the variant CLκ or CLλ domain polypeptide comprise(s) or consist(s) of an amino acid substitution(s) at position 135;
in (ix), the amino acid substitution(s) in the variant CLκ or CLλ domain polypeptide comprise(s) or consist(s) of an amino acid substitution(s) at positions 127 and 129;
in (x), the amino acid substitution(s) in the variant CLκ or CLλ domain polypeptide comprise(s) or consist(s) of an amino acid substitution(s) at positions 127 and 129;
in (xi), the amino acid substitution(s) in the variant CLκ domain polypeptide comprise(s) or consist(s) of an amino acid substitution(s) at position 133 or an amino acid substitution(s) at positions 124 and 133 or the amino acid substitution(s) in the variant CLλ domain polypeptide comprise(s) or consist(s) of an amino acid substitution(s) at position 133;
in (xii), the amino acid substitution(s) in the variant CLκ or CLλ domain polypeptide comprise(s) or consist(s) of an amino acid substitution(s) at position 133 or an amino acid substitution(s) at positions 120, 178, and 180;
in (xiii), the amino acid substitution(s) in the variant CLκ or CLλ domain polypeptide comprise(s) or consist(s) of an amino acid substitution(s) at positions 127, 129, and 178;
in (xiv), the amino acid substitution(s) in the variant CLκ or CLλ domain polypeptide comprise(s) or consist(s) of an amino acid substitution(s) at positions 114, 137, and 138;
in (xv), the amino acid substitution(s) in the variant CLκ or CLλ domain polypeptide comprise(s) or consist(s) of an amino acid substitution(s) at positions 129, 178, and 180;
in (xvi), the amino acid substitution(s) in the variant CLκ or CLλ domain polypeptide comprise(s) or consist(s) of an amino acid substitution(s) at positions 129 and 180;
in (xvii), the amino acid substitution(s) in the variant CLκ or CLλ domain polypeptide comprise(s) or consist(s) of an amino acid substitution(s) at positions 133 and 180; or
in (xviii), the amino acid substitution(s) in the variant CLκ domain polypeptide comprise(s) or consist(s) of an amino acid substitution(s) at positions 129 and 180 or the amino acid substitution(s) in the variant CLλ domain polypeptide comprise(s) or consist(s) of an amino acid substitution(s) at position 129, wherein in each of the foregoing the substitution positions are according to EU numbering;
(D) the amino acid substitution(s) in the variant CH1 domain polypeptide comprise(s) or consist(s) of: 145Q, 147E, 181E, 128R, 147R, 124R, 139R, 141Q, 145S, 147H, 147N, 147Q, 147T, 148E, 148R, 166K, 168R, 168S, 175D, 175E, 181Q, 185E, 185Q, 185S, 185Y, 187D, 187K, and/or 187Q according to EU numbering;
(E) the amino acid substitution(s) of the variant CH1 domain comprise(s) or consist(s) of:
(i) 145Q, 147E, and 181E;
(ii) 128R and 147R;
(iii) 168S, 185S, and 187D;
(iv) 147T and 185Q;
(v) 148R;
(vi) 139R, 141Q, and 187Q;
(vii) 166K and 187K;
(viii) 168R and 185E;
(ix) 124R and 147R;
(x) 147H and 148E;
(xi) 145S;
(xii) 145S and 181Q;
(xiii) 145Q and 181E;
(xiv) 124R, 145S, and 147Q;
(xv) 166K and 187K;
(xvi) 147R and 175D;
(xvii) 147R, 175E, and 181Q;
(xiii) 145S and 147N; or
(xix) 147N and 185Y,
optionally wherein the variant CH1 domain polypeptide preferentially pairs with a variant CLκ or CLλ domain polypeptide, further optionally wherein:
in (i), the amino acid substitution(s) in the variant CLκ or CLλ domain polypeptide comprise(s) or consist(s) of 129R, 178R, and 180Q;
in (ii), the amino acid substitution(s) in the variant CLκ domain polypeptide comprise(s) or consist(s) of 124E, 133Q, and 178E or the amino acid substitution(s) in the variant CLλ domain polypeptide comprise(s) or consist(s) of 133Q and 178E;
in (iii), the amino acid substitution(s) in the variant CLκ or CLλ domain polypeptide comprise(s) or consist(s) of 135R;
in (iv), the amino acid substitution(s) in the variant CLκ or CLλ domain polypeptide comprise(s) or consist(s) of 135S and 178R;
in (v), the amino acid substitution(s) in the variant CLκ or CLλ domain polypeptide comprise(s) or consist(s) of 124S and 129E;
in (vi), the amino acid substitution(s) in the variant CLκ or CLλ domain polypeptide comprise(s) or consist(s) of 114D, 135S, and 138R;
in (vii), the amino acid substitution(s) in the variant CLκ domain polypeptide comprise(s) or consist(s) of 137S and 138E or the amino acid substitution(s) in the variant CLλ domain polypeptide comprise(s) or consist(s) of 138E;
in (viii), the amino acid substitution(s) in the variant CLκ or CLλ domain polypeptide comprise(s) or consist(s) of 135S;
in (ix), the amino acid substitution(s) in the variant CLκ or CLλ domain polypeptide comprise(s) or consist(s) of 127D and 129E;
in (x), the amino acid substitution(s) in the variant CLκ or CLλ domain polypeptide comprise(s) or consist(s) of 127R and 129R;
in (xi), the amino acid substitution(s) in the variant CLκ domain polypeptide comprise(s) or consist(s) of 133Y or 124E and 133Y or the amino acid substitution(s) in the variant CLλ domain polypeptide comprise(s) or consist(s) of 133Y;
in (xii), the amino acid substitution(s) in the variant CLκ or CLλ domain polypeptide comprise(s) or consist(s) of 133Y;
in (xiii), the amino acid substitution(s) in the variant CLκ or CLλ domain polypeptide comprise(s) or consist(s) of 120S, 178H, and 180Q;
in (xiv), the amino acid substitution(s) in the variant CLκ or CLλ domain polypeptide comprise(s) or consist(s) of 127T, 129D, and 178R;
in (xv), the amino acid substitution(s) in the variant CLκ or CLλ domain polypeptide comprise(s) or consist(s) of 114Q, 137T, and 138E;
in (xvi), the amino acid substitution(s) in the variant CLκ or CLλ domain polypeptide comprise(s) or consist(s) of 129D, 178R, and 180H;
in (xvii), the amino acid substitution(s) in the variant CLκ or CLλ domain polypeptide comprise(s) or consist(s) of 129D and 180Q;
in (xiii), the amino acid substitution(s) in the variant CLκ or CLλ domain polypeptide comprise(s) or consist(s) of 133Y and 180R; or
in (xix), the amino acid substitution(s) in the variant CLκ domain polypeptide comprise(s) or consist(s) of 129R and 180S or the amino acid substitution(s) in the variant CLλ domain polypeptide comprise(s) or consist(s) of 129R, wherein in each of the foregoing the substitution positions are according to EU numbering;
(F) the amino acid substitution(s) in the variant CH1 domain polypeptide consist(s) of
(i) 145Q, 147E, and 181E; or
(ii) 128R and 147R;
(iii) 168S, 185S, and 187D; or
(iv) 147T and 185Q,
optionally wherein the variant CH1 domain polypeptide preferentially pairs with a variant CLκ or CLλ domain polypeptide and:
in (i), the amino acid substitution(s) in the variant CLκ or CLλ domain polypeptide comprise(s) or consist(s) of 129R, 178R, and 180Q; or
in (ii), the amino acid substitution(s) in the variant CLκ domain polypeptide comprise(s) or consist(s) of 124E, 133Q, and 178E or the amino acid substitution(s) in the variant CLλ domain polypeptide comprise(s) or consist(s) of 133Q and 178E;
in (iii), the amino acid substitution(s) in the variant CLκ or CLλ domain polypeptide comprise(s) or consist(s) of 135R; or
in (iv), the amino acid substitution(s) in the variant CLκ or CLλ domain polypeptide comprise(s) or consist(s) of 135S and 178R, wherein in each of the foregoing the substitution positions are according to EU numbering;
(G) the variant CH1 domain polypeptide or heavy chain polypeptide comprising said variant CH1 domain polypeptide comprises an amino acid sequence selected from one of SEQ ID NOS: 31, 21, 11, 41, 51, 61, 71, 81, 91, 101, 111, 121, 131, 141, 151, 161, 171, 181, 191 and 201;
(H) the variant CH1 domain polypeptide or heavy chain polypeptide comprising said variant CH1 domain polypeptide comprises an amino acid sequence selected from one of SEQ ID NOS: 31, 21, 11, and 41;
(I) the variant CH1 domain polypeptide or heavy chain polypeptide comprising said variant CH1 domain polypeptide comprises one or more of the following:
(i) the amino acid substitution(s) in the variant CH1 domain polypeptide consists of T187E and the variant CH1 domain polypeptide preferentially pairs with a variant CLκ domain polypeptide, wherein the amino acid substitution(s) in the variant CLκ domain polypeptide do(es) not consist of N137K and S114A;
(ii) the amino acid substitution(s) in the variant CH1 domain polypeptide consists of L145Q and S183V and the variant CH1 domain polypeptide preferentially pairs with a variant CLκ domain polypeptide, wherein the amino acid substitution(s) in the variant CLκ domain polypeptide do(es) not consist of V133T and S176V;
(iii) the amino acid substitution(s) in the variant CH1 domain polypeptide consists of K147A and K213E and the variant CH1 domain polypeptide preferentially pairs with a variant CLκ domain polypeptide, wherein the amino acid substitution(s) in the variant CLκ domain polypeptide do(es) not consist of S131R and E123K;
(iv) the amino acid substitution(s) in the variant CH1 domain polypeptide consists of S183A and K147A and the variant CH1 domain polypeptide preferentially pairs with a variant CLκ domain polypeptide, wherein the amino acid substitution(s) in the variant CLκ domain polypeptide do(es) not consist of S176I and S131R;
(v) the amino acid substitution(s) in the variant CH1 domain polypeptide consists of S183G and K147A and the variant CH1 domain polypeptide preferentially pairs with a variant CLκ domain polypeptide, wherein the amino acid substitution(s) in the variant CLκ domain polypeptide do(es) not consist of S176I and S131R;
(vi) the amino acid substitution(s) in the variant CH1 domain polypeptide consists of K147A, K213E, and S183A and the variant CH1 domain polypeptide preferentially pairs with a variant CLκ domain polypeptide, wherein the amino acid substitution(s) in the variant CLκ domain polypeptide do(es) not consist of S131R, E123K, and S176I;
(vii) the amino acid substitution(s) in the variant CH1 domain polypeptide consists of K147A, K213E, and S183G and the variant CH1 domain polypeptide preferentially pairs with a variant CLκ domain polypeptide, wherein the amino acid substitution(s) in the variant CLκ domain polypeptide do(es) not consist of S131R, E123K, and S176I;
(viii) the amino acid substitution(s) in the variant CH1 domain polypeptide consists of A141I, F170S, S181M, S183A, and V185A and the variant CH1 domain polypeptide preferentially pairs with a variant CLκ domain polypeptide, wherein the amino acid substitution(s) in the variant CLκ domain polypeptide do(es) not consist of F116A, L135V, S174A, S176F, and T178V;
(ix) the amino acid substitution(s) in the variant CH1 domain polypeptide consists of A141L and the variant CH1 domain polypeptide preferentially pairs with a variant CLκ domain polypeptide, wherein the amino acid substitution(s) in the variant CLκ domain polypeptide do(es) not consist of F118S, F118A, or F118V;
(x) the amino acid substitution(s) in the variant CH1 domain polypeptide consists of K147D and the variant CH1 domain polypeptide preferentially pairs with a variant CLκ domain polypeptide, wherein the amino acid substitution(s) in the variant CLκ domain polypeptide do(es) not consist of T129R;
(xi) the amino acid substitution(s) in the variant CH1 domain polypeptide consists of S181E and S183V and the variant CH1 domain polypeptide preferentially pairs with a variant CLκ domain polypeptide, wherein the amino acid substitution(s) in the variant CLκ domain polypeptide do(es) not consist of S176 and T178;
(xii) the amino acid substitution(s) in the variant CH1 domain polypeptide consists of S183L and V185Y and the variant CH1 domain polypeptide preferentially pairs with a variant CLκ domain polypeptide, wherein the amino acid substitution(s) in the variant CLκ domain polypeptide do(es) not consist of V133S;
(xiii) the amino acid substitution(s) in the variant CH1 domain polypeptide consists of S183K and K214R and the variant CH1 domain polypeptide preferentially pairs with a variant CLλ domain polypeptide, wherein the amino acid substitution(s) in the variant CLλ domain polypeptide do(es) not consist of S176E, Y178E, and T212A; or
(xiv) the amino acid substitution(s) in the variant CH1 domain polypeptide consists of L128E, K147T, Q175E, S183W, and K214R and the variant CH1 domain polypeptide preferentially pairs with a variant CLκ domain polypeptide, wherein the amino acid substitution(s) in the variant CLκ domain polypeptide do(es) not consist of S131R, V133G, S176R, and T178A, wherein in each of the foregoing the substitution positions are according to EU numbering; and/or
(J) the amino acid substitution(s) of the variant CH1 domain polypeptide comprise(s) or consist(s) of an amino acid substitution(s) at:
(I) position(s) 185 and/or 187;
(II) position(s) 145, 147, and/or 148;
(III) position(s) 147 or 148;
(IV) position 145;
(V) position(s) 166 and/or 187;
(VI) position(s) 145 and/or 147; or
(VII) position(s) 124 and/or 147,
optionally wherein the variant CH1 domain polypeptide preferentially pairs with a variant CLκ or CLλ domain polypeptide and further optionally wherein:
in (I), the amino acid substitution(s) in the variant CLκ or CLλ domain polypeptide comprise(s) or consist(s) of an amino acid substitution at position 135;
in (II), the amino acid substitution(s) in the variant CLκ or CLλ domain polypeptide comprise(s) or consist(s) of an amino acid substitution at position 124;
in (III), the amino acid substitution(s) in the variant CLκ or CLλ domain polypeptide comprise(s) or consist(s) of an amino acid substitution at position 129;
in (IV), the amino acid substitution(s) in the variant CLκ or CLλ domain polypeptide comprise(s) or consist(s) of an amino acid substitution at position 133;
in (V), the amino acid substitution(s) in the variant CLκ or CLλ domain polypeptide comprise(s) or consist(s) of an amino acid substitution at position(s) 137 and/or 138;
in (VI), the amino acid substitution(s) in the variant CLκ or CLλ domain polypeptide comprise(s) or consist(s) of an amino acid substitution at position(s) 178 and/or 180; or
in (VII), the amino acid substitution(s) in the variant CLκ or CLλ domain polypeptide comprise(s) or consist(s) of an amino acid substitution at position 127, wherein in each of the foregoing the substitution positions are according to EU numbering.

68. A variant CLκ or CLλ domain polypeptide or light chain polypeptide comprising said variant CLκ or CLλ domain polypeptide, which comprises at least one amino acid substitution(s), which comprise(s) or consist(s) of an amino acid substitution(s) at one or more of the following amino acid positions: 129, 178, 180, 124, 133, 114, 120, 127, 135, 137 and 138, according to EU numbering, optionally wherein:
(A) the variant CLκ or CLλ domain polypeptide or light chain polypeptide comprising said variant CLκ or CLλ domain polypeptide preferentially pairs with a variant CH1 domain polypeptide comprising at least one amino acid substitution(s), wherein the amino acid substitution(s) in the variant CH1 domain polypeptide optionally comprise(s) or consist(s) of an amino acid substitution(s) at one or more of the following positions: 145, 147, 181, 128, 124, 139, 141, 148, 166, 168, 175, 185, and 187, according to EU numbering;
(B) the amino acid substitution(s) of the variant CLκ or CLλ domain polypeptide comprise or consist of amino acid substitution(s) selected from the following:
(i) positions 129, 178, and 180;
(ii) positions 124, 133, and 178; or positions 133 and 178;
(iii) position 135;
(iv) positions 135 and 178;
(v) positions 124 and 129;
(vi) positions 114, 135, and 138;
(vii) positions 137 and 138; or position 138;
(viii) positions 127 and 129;
(ix) position 133;
(x) positions 124 and 133;
(xi) positions 120, 178, and 180;
(xii) positions 127, 129, and 178;
(xiii) positions 114, 137, and 138;
(xiv) positions 129 and 180;
(xv) positions 133 and 180; and
(xvi) position 129,
optionally wherein the variant CLκ or CLλ domain polypeptide preferentially pairs with a variant CH1 domain polypeptide and further optionally wherein:
in (i), the amino acid substitution(s) in the variant CH1 domain polypeptide comprise(s) or consist(s) of an amino acid substitution(s) at positions 145, 147, and 181 or positions 147 and 175;
in (ii), the amino acid substitution(s) in the variant CH1 domain polypeptide comprise(s) or consist(s) of an amino acid substitution(s) at positions 128 and 147;
in (iii), the amino acid substitution(s) in the variant CH1 domain polypeptide comprise(s) or consist(s) of an amino acid substitution(s) at positions 168 and 185 or positions 168, 185, and 187;
in (iv), the amino acid substitution(s) in the variant CH1 domain polypeptide comprise(s) or consist(s) of an amino acid substitution(s) at positions 147 and 185;
in (v), the amino acid substitution(s) in the variant CH1 domain polypeptide comprise(s) or consist(s) of an amino acid substitution(s) at position 148;
in (vi), the amino acid substitution(s) in the variant CH1 domain polypeptide comprise(s) or consist(s) of an amino acid substitution(s) at positions 139, 141, and 187;
in (vii), the amino acid substitution(s) in the variant CH1 domain polypeptide comprise(s) or consist(s) of an amino acid substitution(s) at positions 166 and 187;
in (viii), the amino acid substitution(s) in the variant CH1 domain polypeptide comprise(s) or consist(s) of an amino acid substitution(s) at positions 124 and 147 or positions 147 and 148;
in (ix), the amino acid substitution(s) in the variant CH1 domain polypeptide comprise(s) or consist(s) of an amino acid substitution(s) at position 145 or positions 145 and 181;
in (x), the amino acid substitution(s) in the variant CH1 domain polypeptide comprise(s) or consist(s) of an amino acid substitution(s) at position 145;
in (xi), the amino acid substitution(s) in the variant CH1 domain polypeptide comprise(s) or consist(s) of an amino acid substitution(s) at positions 145 and 181;
in (xii), the amino acid substitution(s) in the variant CH1 domain polypeptide comprise(s) or consist(s) of an amino acid substitution(s) at positions 124, 145, and 147;
in (xiii), the amino acid substitution(s) in the variant CH1 domain polypeptide comprise(s) or consist(s) of an amino acid substitution(s) at positions 166 and 187;
in (xiv), the amino acid substitution(s) in the variant CH1 domain polypeptide comprise(s) or consist(s) of an amino acid substitution(s) at positions 147 and 185 or positions 147, 175, and 181;
in (xv), the amino acid substitution(s) in the variant CH1 domain polypeptide comprise(s) or consist(s) of an amino acid substitution(s) at positions 145 and 147; or
in (xvi), the amino acid substitution(s) in the variant CH1 domain polypeptide comprise(s) or consist(s) of an amino acid substitution(s) at positions 147 and 185, wherein in each of the foregoing the substitution positions are according to EU numbering;
(C) the amino acid substitution(s) in the variant CLκ or CLλ domain polypeptide comprise(s) or consist(s) of: 129R, 178R, 180Q, 124E, 133Q, 178E, 114D, 114Q, 120S, 124S, 127D, 127R, 127T, 129D, 129E, 133Y, 135R, 135S, 137S, 137T, 138E, 138R, 178H, and 180H, 180R, and/or 180S according to EU numbering;
(D) the amino acid substitution(s) of the variant CLκ domain polypeptide comprise(s) or consist(s) of:
(i) 129R, 178R, and 180Q;
(ii) 124E, 133Q, and 178E; or 133Q and 178E;
(iii) 135R;
(iv) 135S and 178R;
(v) 124S and 129E;
(vi) 114D, 135S, and 138R;
(vii) 137S and 138E; or 138E;
(viii) 135S;
(ix) 127D and 129E;
(x) 127R and 129R;
(xi) 133Y;
(xii) 133Y;
(xiii) 124E and 133Y; or 133Y;
(xiv) 120S, 178H, and 180Q;
(xv) 127T, 129D, and 178R;
(xvi) 114Q, 137T, and 138E;
(xvii) 129D, 178R, and 180H;
(xviii) 129D and 180Q;
(xix) 133Y and 180R; or
(xx) 129R and 180S; or 129R,
optionally wherein the variant CLκ or CLλ domain polypeptide preferentially pairs with a variant CH1 domain polypeptide and further optionally wherein:
in (i), the amino acid substitution(s) in the variant CH1 domain polypeptide comprise(s) or consist(s) of 145Q, 147E, and 181E;
in (ii), the amino acid substitution(s) in the variant CH1 domain polypeptide comprise(s) or consist(s) of 128R and 147R;
in (iii), the amino acid substitution(s) in the variant CH1 domain polypeptide comprise(s) or consist(s) of 168S, 185S, and 187D;
in (iv), the amino acid substitution(s) in the variant CH1 domain polypeptide comprise(s) or consist(s) of 147T and 185Q;
in (v), the amino acid substitution(s) in the variant CH1 domain polypeptide comprise(s) or consist(s) of 148R;
in (vi), the amino acid substitution(s) in the variant CH1 domain polypeptide comprise(s) or consist(s) of 139R, 141Q, and 187Q;
in (vii), the amino acid substitution(s) in the variant CH1 domain polypeptide comprise(s) or consist(s) of 166K and 187K;
in (viii), the amino acid substitution(s) in the variant CH1 domain polypeptide comprise(s) or consist(s) of 168R and 185E;
in (ix), the amino acid substitution(s) in the variant CH1 domain polypeptide comprise(s) or consist(s) of 124R and 147R;
in (x), the amino acid substitution(s) in the variant CH1 domain polypeptide comprise(s) or consist(s) of 147H and 148E;
in (xi), the amino acid substitution(s) in the variant CH1 domain polypeptide comprise(s) or consist(s) of 145S;
in (xii), the amino acid substitution(s) in the variant CH1 domain polypeptide comprise(s) or consist(s) of 145S and 181Q;
in (xiii), the amino acid substitution(s) in the variant CH1 domain polypeptide comprise(s) or consist(s) of 145S;
in (xiv), the amino acid substitution(s) in the variant CH1 domain polypeptide comprise(s) or consist(s) of 145Q and 181E;
in (xv), the amino acid substitution(s) in the variant CH1 domain polypeptide comprise(s) or consist(s) of 124R, 145S, and 147Q;
in (xvi), the amino acid substitution(s) in the variant CH1 domain polypeptide comprise(s) or consist(s) of 166K and 187K;
in (xvii), the amino acid substitution(s) in the variant CH1 domain polypeptide comprise(s) or consist(s) of 147R and 175D;
in (xviii), the amino acid substitution(s) in the variant CH1 domain polypeptide comprise(s) or consist(s) of 147R, 175E, and 181Q;
in (xix), the amino acid substitution(s) in the variant CH1 domain polypeptide comprise(s) or consist(s) of 145S and 147N; or
in (xx), the amino acid substitution(s) in the variant CH1 domain polypeptide comprise(s) or consist(s) of 147N and 185Y, wherein in each of the foregoing the substitution positions are according to EU numbering;
(E) the amino acid substitution(s) in the variant CLκ or CLλ domain polypeptide consist(s) of
(i) 129R, 178R, and 180Q; or
(ii) 124E, 133Q, and 178E; or 133Q and 178E;
(iii) 135R;
(iv) 135S and 178R,
optionally wherein the variant CLκ or CLλ domain polypeptide preferentially pairs with a variant CH1 domain polypeptide and:
in (i), the amino acid substitution(s) in the variant CH1 domain polypeptide comprise(s) or consist(s) of 145Q, 147E, and 181E; or
in (ii), the amino acid substitution(s) in the variant CH1 domain polypeptide comprise(s) or consist(s) of 128R and 147R;
in (iii), the amino acid substitution(s) in the variant CH1 domain polypeptide comprise(s) or consist(s) of 168S, 185S, and 187D;
in (iv), the amino acid substitution(s) in the variant CH1 domain polypeptide comprise(s) or consist(s) of 147T and 185Q, wherein in each of the foregoing the substitution positions are according to EU numbering.
(F) the variant CLκ or CLλ domain polypeptide or light chain polypeptide comprising said variant CLκ or CL, domain polypeptide comprises an amino acid sequence selected from one of SEQ ID NOS: 32, 22, 12, 42, 52, 62, 72, 82, 92, 102, 112, 122, 132, 142, 152, 162, 172, 182, 192, or 202 or any one of SEQ ID NOS: 59, 99, 39, 199, 89, 49, 29, 19, 69, 79, 109, 119, 129, 139, 149, 159, 169, 179, 189, or 209; and/or
(G) the variant CLκ or CLλ domain polypeptide or light chain polypeptide comprising said variant CLκ or CL, domain polypeptide comprises an amino acid sequence selected from one of SEQ ID NOS: 32, 22, 12, 42 or any one of SEQ ID NOS: 59, 99, 39, 199, 89, 49, or 29;
(H) (i) when the amino acid substitution(s) in the variant CLκ domain polypeptide consists of N137K and S114A and preferentially pairs with a variant CH1 domain polypeptide, the amino acid substitution(s) in the variant CH1 domain polypeptide do(es) not consist of T187E;
(ii) when the amino acid substitution(s) in the variant CLκ domain polypeptide consists of V133T; S176V and preferentially pairs with a variant CH1 domain polypeptide, the amino acid substitution(s) in the variant CH1 domain polypeptide do(es) not consist of L145Q and S183V;
(iii) when the amino acid substitution(s) in the variant CLκ domain polypeptide consists of V133E and preferentially pairs with a variant CH1 domain polypeptide, the amino acid substitution(s) in the variant CH1 domain polypeptide do(es) not consist of S183K;
(iv) when the amino acid substitution(s) in the variant CLκ domain polypeptide consists of F116A, L135V, S174A, S176F, and T178V and preferentially pairs with a variant CH1 domain polypeptide, the amino acid substitution(s) in the variant CH1 domain polypeptide do(es) not consist of A141I, F170S, S181M, S183A, and V185A;
(v) when the amino acid substitution(s) in the variant CLκ domain polypeptide consists of T129R and preferentially pairs with a variant CH1 domain polypeptide, the amino acid substitution(s) in the variant CH1 domain polypeptide do(es) not consist of K147D;
(vi) when the amino acid substitution(s) in the variant CLκ domain polypeptide consists of S176 and T178 and preferentially pairs with a variant CH1 domain polypeptide, the amino acid substitution(s) in the variant CH1 domain polypeptide do(es) not consist of S181E and S183V;
(vii) when the amino acid substitution(s) in the variant CLκ domain polypeptide consists of V133S and preferentially pairs with a variant CH1 domain polypeptide, the amino acid substitution(s) in the variant CH1 domain polypeptide do(es) not consist of S183L and V185Y;
(viii) when the amino acid substitution(s) in the variant CLλ domain polypeptide consists of S176E, Y178E, and T212A and preferentially pairs with a variant CH1 domain polypeptide, the amino acid substitution(s) in the variant CH1 domain polypeptide do(es) not consist of S183K and K214R; or
(ix) when the amino acid substitution(s) in the variant CLκ domain polypeptide consists of S131R, V133G, S176R, and T178A and preferentially pairs with a variant CH1 domain polypeptide, the amino acid substitution(s) in the variant CH1 domain polypeptide do(es) not consist of L128E, K147T, Q175E, S183W and K214R, wherein in each of the foregoing the substitution positions are according to EU numbering; and/or
(I) the amino acid substitution(s) of the variant CLκ domain polypeptide comprise(s) or consist(s) of an amino acid substitution(s) at:
(I) position 135;
(II) position 124;
(III) position 129;
(IV) position 133;
(V) position(s) 137 and/or 138;
(VI) position(s) 178 and/or 180; or
(VII) position 127,
optionally wherein the variant CLκ or CLλ domain polypeptide preferentially pairs with a variant CH1 domain polypeptide and further optionally wherein:
in (I), the amino acid substitution(s) in the variant CH1 domain polypeptide comprise(s) or consist(s) of an amino acid position(s) 185 and/or 187;
in (II), the amino acid substitution(s) in the variant CH1 domain polypeptide comprise(s) or consist(s) of an amino acid substitution at position(s) 145, 147, and/or 148;
in (III), the amino acid substitution(s) in the variant CH1 domain polypeptide comprise(s) or consist(s) of an amino acid substitution at position(s) 147 or 148;
in (IV), the amino acid substitution(s) in the variant CH1 domain polypeptide comprise(s) or consist(s) of an amino acid substitution at position 145;
in (V), the amino acid substitution(s) in the variant CH1 domain polypeptide comprise(s) or consist(s) of an amino acid substitution at position(s) 166 and/or 187;
in (VI), the amino acid substitution(s) in the variant CH1 domain polypeptide comprise(s) or consist(s) of an amino acid substitution at position(s) 145 and/or 147; or
in (VII), the amino acid substitution(s) in the variant CH1 domain polypeptide comprise(s) or consist(s) of an amino acid substitution at position(s) 124 and/or 147, wherein in each of the foregoing the substitution positions are according to EU numbering.

69. An immunoglobulin polypeptide, which comprises at least one variant immunoglobulin heavy chain constant region 1 (“CH1”) domain polypeptide or heavy chain polypeptide comprising said variant CH1 domain polypeptide according to claim 67, optionally wherein:
(A) the immunoglobulin polypeptide comprises one or more of the following:
(i) an antigen-binding domain;
(ii) a second CH1 domain or variant CH1 domain;
(iii) an immunoglobulin heavy chain constant region 2 (“CH2”) domain or variant CH2 domain;
(iv) an immunoglobulin heavy chain constant region 3 (“CH3”) domain or variant CH3 domain; and/or
(v) a light chain constant region (CL) domain or variant CL domain, optionally a variant CLκ or CLλ domain,
optionally wherein:
in (i), the antigen-binding domain comprises an immunoglobulin heavy chain variable region (“VH”) domain, an immunoglobulin light chain variable region (“VL”) domain, a single chain fragment variable (“scFv”), an antigen-binding fragment (Fab), a F(ab′), a F(ab′)2, F(ab′)2, or a combination thereof,
in (ii), the CH1 domain comprises a wild-type CH1 amino acid sequence or comprises one or more amino acid substitutions relative to a wild-type CH1 amino acid sequence;
in (iii), the CH2 domain comprises a wild-type CH2 amino acid sequence or comprises one or more amino acid substitutions relative to a wild-type CH2 amino acid sequence;
in (iv), the CH3 domain comprises a wild-type CH3 amino acid sequence or comprises one or more amino acid substitutions relative to a wild-type CH3 amino acid sequence; and/or
in (v), the CL domain comprises a wild-type CL amino acid sequence or comprises one or more amino acid substitutions relative to a wild-type CL amino acid sequence;
and/or
(B) the immunoglobulin polypeptide:
(I) comprises a VH domain and is bound to or paired with another polypeptide comprising a VL domain, wherein the VH domain and the VL domain form an antigen-binding site; or
(II) comprises a VL domain and is bound to or paired with another polypeptide comprising a VH domain, wherein the VL domain and the VH domain form an antigen-binding site.

70. An immunoglobulin polypeptide, which comprises at least one variant CLκ or CLλ domain polypeptide or light chain polypeptide respectively comprising a variant CLκ or CLλ domain polypeptide according to claim 68, optionally wherein:
(A) the immunoglobulin polypeptide comprises one or more of the following:
(i) an antigen-binding domain;
(ii) a CH1 domain or variant CH1 domain;
(iii) a CH2 domain or variant CH2 domain;
(iv) a CH3 domain or variant CH3 domain; and/or
(v) a second CL domain or variant CL domain,
optionally wherein:
in (i), the antigen-binding domain comprises a VH domain, a VL domain, a scFv, a Fab, a F(ab′), a F(ab′)2, F(ab′)2, or a combination thereof
in (ii), the CH1 domain comprises a wild-type CH1 amino acid sequence or comprises one or more amino acid substitutions relative to a wild-type CH1 amino acid sequence;
in (iii), the CH2 domain comprises a wild-type CH2 amino acid sequence or comprises one or more amino acid substitutions relative to a wild-type CH2 amino acid sequence;
in (iv), the CH3 domain comprises a wild-type CH3 amino acid sequence or comprises one or more amino acid substitutions relative to a wild-type CH3 amino acid sequence; and/or
in (v), the CL domain comprises a wild-type CL amino acid sequence or comprises one or more amino acid substitutions relative to a wild-type CL amino acid sequence; and/or
(B) the immunoglobulin polypeptide:
(I) comprises a VH domain and is bound to or paired with another polypeptide comprising a VL domain, wherein the VH domain and the VL domain form an antigen-binding site; or
(II) comprises a VL domain and is bound to or paired with another polypeptide comprising a VH domain, wherein the VL domain and the VH domain form an antigen-binding site.

71. A molecule comprising at least a first polypeptide and a second polypeptide, wherein:
(A) the first polypeptide comprises a variant CH1 domain polypeptide or heavy chain polypeptide comprising a variant CH1 domain polypeptide according to claim 67; and
(B) the second polypeptide comprises a variant CLκ or CLλ domain polypeptide or light chain polypeptide comprising a variant CLκ or CLλ domain polypeptide,
optionally wherein:
(1) the first polypeptide comprises an antigen-binding domain and/or the second polypeptide comprises an antigen-binding domain, optionally wherein:
the antigen-binding domain of the first polypeptide and the antigen-binding domain of the second polypeptide comprise a VH and a VL, respectively, or a VL and a VH, respectively, further optionally forming an antigen binding site specific for a first epitope;
or
the antigen-binding domain of the first polypeptide comprises a scFv or nanobody specific for a first epitope and/or the antigen-binding domain of the second polypeptide comprises a scFv or nanobody specific for a second epitope, further optionally wherein the first epitope is the same as or is different than the second epitope;
(2) the molecule further comprises a third polypeptide, which comprises at least one variant CH1 domain polypeptide or heavy chain polypeptide according to claim 67 and further optionally comprises a fourth polypeptide, which comprises at least one variant CLκ or CLλ domain polypeptide or light chain polypeptide; and/or
(3) the molecule is a multi-specific antibody or antigen-binding antibody fragment.

72. A polynucleotide or polynucleotides encoding a variant CH1 domain polypeptide according to claim 67 or a heavy chain polypeptide comprising said variant CH1 domain polypeptide or an immunoglobulin polypeptide, which comprises at least one variant CH1 domain polypeptide or heavy chain polypeptide comprising said variant CH1 domain polypeptide according to claim 67, or a vector or vectors containing said polynucleotide or polynucleotides.

73. A polynucleotide or polynucleotides encoding a variant CLκ or CLλ domain polypeptide according to claim 68 or a light chain polypeptide comprising said variant CLκ or CLλ domain polypeptide or an or an immunoglobulin polypeptide, which comprises at least one variant CLκ or CLλ domain polypeptide or a light chain polypeptide comprising said variant CLκ or CLλ domain polypeptide according to claim 68, or a vector or vectors containing said polynucleotide or polynucleotides.

74. A recombinant cell, optionally a mammalian or yeast cell, which expresses a variant CH1 domain polypeptide according to claim 67 or a heavy chain polypeptide comprising said variant CH1 domain polypeptide or an immunoglobulin containing said variant CH1 domain polypeptide.

75. A recombinant cell, optionally a mammalian or yeast cell, which expresses a variant CLκ or CLλ domain polypeptide according to claim 68 or a light chain polypeptide comprising said variant CLκ or CLλ domain polypeptide or an immunoglobulin polypeptide containing said variant CLκ or CLλ domain polypeptide.

76. A method of generating a CH1 domain-encoding polynucleotide library optionally comprising a polynucleotide encoding a variant CH1 domain polypeptide according to claim 67, or a CH1 domain-encoding polynucleotide library generated using said method, wherein the method comprises in silico or in vitro incorporating a mutation at or randomizing the nucleic acid at one or more pre-determined nucleotide positions in a plurality of CH1 domain-encoding polynucleotides, wherein at least one of the one or more pre-determined nucleotide positions is within the codon(s) encoding the amino acid at one or more of pre-determined CH1 domain amino acid positions which is/are:
(i) present in or proximate to the interface of a CH1 domain and a CL domain;
(ii) predicted to affect CH1-CL interdomain interaction, optionally hydrogen bond-mediated interaction, optionally wherein the prediction is performed in silico or in vitro, further optionally wherein the prediction is performed in silico using Rosetta Monte Carlo (MC) Hydrogen Bond Network (HBNet); and/or
(iii) selected from positions 145, 147, 181, 128, 124, 139, 141, 148, 166, 168, 175, 185, and 187, according to EU numbering,
optionally wherein the one or more mutations are generated via a degenerate codon, optionally a degenerate RMW codon representing six naturally occurring amino acids (D, T, A, E, K, and N) or a degenerate NNK codon representing all 20 naturally occurring amino acid residues, further optionally wherein the library is for identifying one or more variant CH1 domain polypeptides which preferentially pairs with a given or variant CL domain polypeptide rather than with a wild-type or another given variant CL domain polypeptide;

77. A method of generating a CH1 domain polypeptide library, or a CH1 domain polypeptide library generated using said method, wherein the method comprises:
(I) in silico or in vitro obtaining a plurality of CH1 domain polypeptides corresponding to a plurality of CH1 domain-encoding polynucleotides contained in the CH1 domain-encoding polynucleotide library according to claim 76; or
(II) in silico or in vitro incorporating a substitution at one or more pre-determined CH1 domain amino acid positions in a plurality of CH1 domain polypeptides, wherein one or more of the one or more pre-determined CH1 domain amino acid position(s) is/are:
(i) present in or proximate to the interface of a CH1 domain and a CL domain;
(ii) predicted to affect CH1-CL interdomain interaction, optionally hydrogen bond-mediated interaction, optionally wherein the prediction is performed in silico or in vitro, further optionally wherein the prediction is performed in silico using Rosetta MC HBNet; and/or
(iii) selected from positions 145, 147, 181, 128, 124, 139, 141, 148, 166, 168, 175, 185, and 187, according to EU numbering,
optionally wherein the library is for identifying one or more variant CH1 domain polypeptides which preferentially pairs with a given or variant CL domain polypeptide rather than with a wild-type or another given variant CL domain polypeptide,
further optionally wherein the CH1 domain polypeptides of the library comprises a pre-determined number of CH1 substitution positions, optionally wherein the pre-determined number is 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

78. A method of generating a CLκ and/or CLλ domain-encoding polynucleotide library, optionally comprising a polynucleotide encoding a variant CLκ or CLλ domain polypeptide or light chain polypeptide according to claim 68, or a CLκ and/or CLλ domain-encoding polynucleotide library generated using said method, wherein the method comprises in silico or in vitro incorporating a mutation at or randomizing the nucleic acid at one or more pre-determined nucleotide positions in a plurality of CLκ and/or CLλ domain-encoding polynucleotides, wherein at least one of the one or more pre-determined nucleotide positions is within the codon(s) encoding the amino acid at one or more of pre-determined CLκ and/or CLλ domain amino acid positions which is/are:
(i) present in or proximate to the interface of a CH1 domain and a CLκ and/or CLλ domain;
(ii) predicted to affect CH1-CL interdomain interaction, optionally hydrogen bond-mediated interaction, optionally wherein the prediction is performed in silico or in vitro, further optionally wherein the prediction is performed in silico using Rosetta Monte Carlo (MC) Hydrogen Bond Network (HBNet);
and/or
(iii) selected from positions 129, 178, 180, 124, 133, 114, 120, 127, 135, 137, and 138, according to EU numbering,
optionally wherein the one or more mutations are generated via a degenerate codon, optionally a degenerate RMW codon representing six naturally occurring amino acids (D, T, A, E, K, and N) or a degenerate NNK codon representing all 20 naturally occurring amino acid residues, further optionally wherein the library is for identifying one or more variant CLκ domain polypeptides which preferentially pairs with a given or variant CH1 domain rather than with a wild-type CH1 or another given variant CH1 domain polypeptide,
and further optionally wherein the variant CLκ and/or CLλ domain library comprises CL domains of κ isotype only, CL domains of λ isotype only, or at least one CL domain of κ isotype and at least one CL domain of λ isotype.

79. A method of generating a CLκ and/or CLλ domain polypeptide library, or a CLκ and/or CLλ domain polypeptide library generated using the method, wherein the method comprises:
(I) in silico or in vitro obtaining a plurality of CLκ and/or CLλ domain polypeptides corresponding to a plurality of CLκ and/or CLλ domain-encoding polynucleotides contained in the CLκ and/or CLλ domain-encoding polynucleotide library according to claim 78; or
(II) in silico or in vitro incorporating a substitution at one or more pre-determined CLκ and/or CLλ domain amino acid positions in a plurality of CLκ and/or CLλ domain polypeptides, wherein one or more of the one or more pre-determined CLκ and/or CLλ domain amino acid position(s) is/are:
(i) present in or proximate to the interface of a CH1 domain and a CL domain;
(ii) predicted to affect CH1-CL interdomain interaction, optionally hydrogen bond-mediated interaction, optionally wherein the prediction is performed in silico or in vitro, further optionally wherein the prediction is performed in silico using Rosetta MC HBNet; and/or
(iii) selected from positions 129, 178, 180, 124, 133, 114, 120, 127, 135, 137, and 138, according to EU numbering,
optionally wherein the library is for identifying one or more variant CLκ and/or CLλ domain polypeptides which preferentially pairs with a given or variant CH1 domain polypeptide rather than with a wild-type or another given variant CH1 domain polypeptide,
further optionally wherein the CLκ and/or CLλ domain polypeptides of the library comprises a pre-determined number of CLκ and/or CLλ substitution positions, optionally wherein the pre-determined number is 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

80. A method of generating a CH1-CL domain-encoding polynucleotide set library optionally comprising a variant CH1 domain polypeptide according to claim 67, or a CH1-CL domain-encoding polynucleotide set library generated using the method, wherein the method comprises in silico or in vitro incorporating a mutation at or randomizing the nucleic acid at one or more pre-determined nucleotide positions in a plurality of CH1-CL domain-encoding polynucleotide sets, wherein at least one of the one or more pre-determined nucleotide positions is within the codon(s) encoding the amino acid at one or more of pre-determined CH1 and/or CL domain amino acid positions which is/are:
(i) present in or proximate to the interface of a CH1 domain and a CL domain;
(ii) predicted to affect CH1-CL interdomain interaction, optionally hydrogen bond-mediated interaction, optionally wherein the prediction is performed in silico or in vitro, further optionally wherein the prediction is performed in silico using Rosetta Monte Carlo (MC) Hydrogen Bond Network (HBNet);
(iii) selected from CH1 domain amino acid positions 145, 147, 181, 128, 124, 139, 141, 148, 166, 168, 175, 185, and 187, according to EU numbering; and/or
(iv) selected from CL domain amino acid positions 129, 178, 180, 124, 133, 114, 120, 127, 135, 137, and 138, according to EU numbering,
optionally wherein the one or more mutations are generated via a degenerate codon, optionally a degenerate RMW codon representing six naturally occurring amino acids (D, T, A, E, K, and N) or a degenerate NNK codon representing all 20 naturally occurring amino acid residues, further optionally wherein the library is for identifying one or more variant CL domain polypeptides which preferentially pairs with a given or variant CH1 domain rather than with a wild-type or another given variant CH1 domain polypeptide and/or for identifying one or more variant CH1 domain polypeptides which preferentially pairs with a given or variant CL domain rather than with a wild-type or another given variant CL domain polypeptide,
further optionally wherein the CL domains encoded in the CH1-CL domain-encoding polynucleotide set library comprise a CLκ domain(s) and/or a CLλ domain(s).

81. A method of generating a CH1-CL domain polypeptide set library, or a CH1-CL domain polypeptide set library generated using the method, wherein the method comprises:
(1) (I) in silico or in vitro obtaining a plurality of CH1-CL domain polypeptide sets corresponding to a plurality of CH1-CL domain-encoding polynucleotide sets contained in the CH1-CL domain-encoding polynucleotide set library according to claim 80; or
(II) in silico or in vitro incorporating a substitution at one or more pre-determined CH1 and/or CL domain amino acid positions in a plurality of CH1-CL domain polypeptide sets, wherein one or more of the one or more pre-determined CH1 and/or CL domain amino acid position(s) is/are:
(i) present in or proximate to the interface of a CH1 domain and a CL domain;
(ii) predicted to affect CH1-CL interdomain interaction, optionally hydrogen bond-mediated interaction, optionally wherein the prediction is performed in silico or in vitro, further optionally wherein the prediction is performed in silico using Rosetta Monte Carlo (MC) Hydrogen Bond Network (HBNet);
(iii) selected from CH1 domain amino acid positions 145, 147, 181, 128, 124, 139, 141, 148, 166, 168, 175, 185, and 187, according to EU numbering; and/or
(iv) selected from CL domain amino acid positions 129, 178, 180, 124, 133, 114, 120, 127, 135, 137, and 138, according to EU numbering,
optionally wherein the library is for identifying one or more variant CL domain polypeptides which preferentially pairs with a given or variant CH1 domain rather than with a wild-type or another given variant CH1 domain polypeptide and/or for identifying one or more variant CH1 domain polypeptides which preferentially pairs with a given or variant CL domain rather than with a wild-type or another given variant CL domain polypeptide; or
(2) (i) providing a plurality of CH1-CL domain polypeptide sets;
(ii) calculating the CH1-CL interdomain interaction strength for one or more of the plurality of CH1-CL domain polypeptide sets, optionally wherein the calculating is:
(a) in silico or in vitro, optionally in silico using Rosetta Monte Carlo (MC) Hydrogen Bond Network (HBNet); and/or
(b) based on the strength of CH1-CL interdomain hydrogen bond(s) and/or of CH1-CL interdomain binding energy;
(iii) selecting one or more CH1-CL domain polypeptide sets calculated to have stronger CH1-CL interdomain interaction compared to:
(a) a reference CH1-CL domain polypeptide set, which is optionally a WT CH1-CL domain polypeptide set or a known CH1-CL domain polypeptide set; or
(b) a reference CH1-CL interdomain interaction strength, which is optionally a the CH1-CL interdomain interaction strength of a WT CH1-CL domain polypeptide set or of a known CH1-CL polypeptide domain set,
optionally wherein, in (1) and/or (2),
the CL domain polypeptides in the CH1-CL domain polypeptide set library comprise a CLκ domain polypeptide(s) and/or a CLλ domain polypeptide(s); and/or
the CH1-CL domain polypeptide set library is a CH1-CLκ domain polypeptide set library, CH1-CLλ domain polypeptide set library, or a CH1-CL domain polypeptide set library in which the CL domain polypeptides of the library comprise one or more CLκ domain polypeptides and one or more CLλ domain polypeptides,
further optionally wherein, in (1) and/or (2):
(A) the CH1 domain polypeptides of the CH1-CL domain polypeptide set library comprises a pre-determined number of CH1 substitution positions, optionally wherein the pre-determined number is 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5; and/or
(B) the CL domain polypeptides of the CH1-CL domain polypeptide set library comprises a pre-determined number of CL substitution positions, optionally wherein the pre-determined number is 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

82. A method of identifying one or more sets of a variant CH1 domain polypeptide and a variant CLκ or CLλ domain polypeptide, optionally comprising a variant CH1 domain polypeptide according to claim 67, wherein the variant CH1 domain polypeptide and the variant CLκ or CLλ domain polypeptide preferentially pair with each other, and wherein the method comprising:
(a) providing multiple sets of (a-1) a first polypeptide comprising a wild-type or variant CH1 domain polypeptide and (a-2) a second polypeptide comprising a wild-type or a variant, CLκ or CLλ domain polypeptide, optionally wherein the multiple sets of (a-1) and (a-2) are provided in silico or in vitro;
(b) quantifying the binding preference between the variant CH1 domain polypeptide and the variant CLκ or CLλ domain polypeptide, optionally wherein the binding preference is based on the strength of CH1-CL interdomain hydrogen bond(s) and/or of CH1-CL interdomain binding energy, further optionally wherein the quantifying is performed in silico or in vitro; and
(c) selecting one or more sets of a variant CH1 domain polypeptide and a variant CLκ or CLλ domain polypeptide which provide preferential CH1-CL paring, optionally equivalent or higher preferential pairing relative to a reference CH1-CL domain polypeptide set.

83. A method of screening for a combination of (i) a first set of a first variant CH1 domain polypeptide and a first variant CL domain polypeptide (“first CH1-CL domain polypeptide set”) and (ii) a second set of a second variant CH1 domain polypeptide and a second variant CL domain polypeptide (“second CH1-CL domain polypeptide set”), which optionally comprises a variant CH1 domain polypeptide according to claim 67, wherein the combination is suited for a multi-specific antibody or antigen-binding antibody fragment of interest having an antibody or antibody fragment structure of interest and/or antigen specificities of interest, optionally having variable region sequences of interest, the method comprising:
(a) expressing a plurality of multi-specific antibodies and/or antigen-binding antibody fragments, comprising different combinations of (i) a first CH1-CL domain polypeptide set candidate and (ii) a second CH1-CL domain polypeptide set candidate; and
(b) selecting one or more combinations of (i) a first CH1-CL domain polypeptide set and (ii) a second CH1-CL domain polypeptide set based on one or more characteristics of the plurality of multi-specific antibodies and/or antigen-binding antibody fragments expressed in step (a), optionally wherein at least one of the one or more characteristics is selected from the following:
(i) (i-1) production yield, optionally assessed in one or more cell types, optionally mammalian cells such as CHO cells and HEK cells, yeast cells, insect cells, and/or plant cells and/or (i-2) compatibility to one or more antibody purification methods, optionally comprising protein A affinity purification;
(ii) degree of aggregation, optionally presence of multimers of a full-size antibody, optionally quantified using chromatography, optionally size exclusion chromatography (SEC) or electrophoresis, optionally SDS-PAGE;
(iii) the rate of correct pairing, optionally correct paring between CH1 domains and/or between CH1 and CL domains, optionally assessed using LC-MS;
(iv) melting temperature (Tm) and/or aggregation temperature (Tagg), optionally Tagg266, optionally measured using Differential scanning fluorimetry (DSF) and/or Differential scanning calorimetry (DSC) and/or using an instrument, optionally Uncle®;
(v) isoelectric point (“pI”);
(vi) the level of interaction with polyspecificity reagent (“PSR”), optionally measured the method described in in WO2014/179363;
(vii) hydrophobic interaction of the antibody optionally measured using hydrophobic interaction chromatography (“HIC”), optionally as described in Estep P, et al. MAbs. 2015 May-June; 7(3): 553-561;
(viii) self-interaction, optionally measured by (viii-1) affinity-capture self-interaction nanoparticle spectroscopy (AC-SINS), optionally as described in Liu Y et al., MAbs. March-April 2014; 6(2):483-92 or (viii-2) dynamic light scattering (DLS);
(ix) stability to high or low pH stress;
(x) solubility;
(xi) production costs and/or time;
(xii) other stability parameters;
(xiii) shelf life;
(xiv) in vivo half-life; and/or
(xv) immunogenicity,
optionally wherein the plurality of multi-specific antibodies and/or antigen-binding antibody fragments comprise:
(I) a first polypeptide comprising a first variant CH1 domain polypeptide and a first antigen-binding domain polypeptide;
(II) a second polypeptide comprising a second variant CH1 domain polypeptide and a second antigen-binding domain polypeptide;
(III) a third polypeptide comprising a first variant CL domain polypeptide and a third antigen-binding domain polypeptide; and
(IV) a fourth polypeptide comprising a second variant CL domain polypeptide and a fourth antigen-binding domain polypeptide,
optionally wherein the first and third polypeptide preferentially pair with each other and the second and fourth polypeptide preferentially pair with each other,
and optionally the plurality of multi-specific antibodies and/or antigen-binding antibody fragments comprise a structure depicted in any of FIGS. 2-7, further optionally an IgG, still further optionally an IgG1, IgG2, IgG3 or IgG4;

84. A method of generating a library of sets of a first candidate polypeptide-encoding polynucleotide and a second candidate polypeptide-encoding polynucleotide, or a library of sets of a first candidate polypeptide-encoding polynucleotide and a second candidate polypeptide-encoding polynucleotide generated using said method, wherein:
(i) the first candidate polypeptide is the same as or is a variant of a first parent polypeptide; and
(ii) the second candidate polypeptide is the same as or is a variant of a second parent polypeptide,
the method comprising:
(a) providing a set of a polynucleotide encoding the first parent polypeptide and a polynucleotide encoding the second parent polypeptide; and
(b) in silico or in vitro incorporating a mutation at or randomizing the nucleic acid at one or more pre-determined nucleotide positions in the polynucleotide set of step (a), wherein at least one of the one or more pre-determined nucleotide positions is within the codon(s) encoding the amino acid at one or more of pre-determined amino acid positions of the first and/or second parent polypeptides which is/are:
(i) present in or proximate to the interface of the first parent polypeptide and the second parent polypeptide, optionally wherein the amino acid position(s) present in or proximate to the interface is predicted in silico or in vitro; and/or
(ii) predicted to affect interaction between the first parent polypeptide and the second parent polypeptide, optionally inter-polypeptide hydrogen bond-mediated interaction and/or inter-polypeptide binding energy, optionally wherein the prediction is performed in silico or in vitro, further optionally wherein the prediction is performed in silico using Rosetta Monte Carlo (MC) Hydrogen Bond Network (HBNet),
optionally wherein the one or more mutations are generated via a degenerate codon, optionally a degenerate RMW codon representing six naturally occurring amino acids (D, T, A, E, K, and N) or a degenerate NNK codon representing all 20 naturally occurring amino acid residues, and optionally wherein the library is for identifying a first polypeptide and a second polypeptide which preferentially pair with each other, optionally relative to a set of the first parent polypeptide and the second parent polypeptide.

85. A method of generating a library of sets of a first candidate polypeptide and a second candidate polypeptide, or a library of sets of a first candidate polypeptide and a second candidate polypeptide generated using said method, wherein:
(i) the first candidate polypeptide is the same as or is a variant of a first parent polypeptide; and
(ii) the second candidate polypeptide the same as or is a variant of a second parent polypeptide,
the method comprising:
(I) in silico or in vitro obtaining multiple sets of a first candidate polypeptide and a second candidate polypeptide corresponding to the first candidate polypeptide-encoding polynucleotides and the second candidate polypeptide-encoding polynucleotides contained in the polynucleotide library according to claim 84; or
(II) in silico or in vitro incorporating a substitution at one or more pre-determined amino acid positions of the first and/or second parent polypeptide(s), wherein one or more of the one or more pre-determined amino acid position(s) is/are:
(i) present in or proximate to the interface of the first parent polypeptide and the second parent polypeptide, optionally wherein the amino acid position(s) present in or proximate to the interface is predicted in silico or in vitro; and/or
(ii) predicted to affect interaction between the first parent polypeptide and the second parent polypeptide, optionally inter-polypeptide hydrogen bond-mediated interaction and/or inter-polypeptide binding energy, optionally wherein the prediction is performed in silico or in vitro, further optionally wherein the prediction is performed in silico using Rosetta MC HBNet,
optionally wherein the library is for identifying a first polypeptide and a second polypeptide which preferentially pair with each other, optionally relative to a set of the first parent polypeptide and the second parent polypeptide,
further optionally wherein:
(A) the first candidate polypeptides in the library comprise a pre-determined number(s) of substitutions relative to the first parent polypeptide, optionally wherein the pre-determined number(s) is/are 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5; and/or
(B) the second candidate polypeptides in the library comprise a pre-determined number(s) of substitutions relative to the second parent polypeptide, optionally wherein the pre-determined number(s) is/are 1 or more, 2 or more, 3 or more, 4 or more, 5 or more; 10 or below, 9 or below, 8 or below, 7 or below, 6 or below, 5 or below, 4 or below, 3 or below, or 2 or below; between 1-10, between 1-9, between 1-8, between 1-7, between 1-6, between 1-5, between 1-4; between 1-3; between 1-2; and/or 1, 2, 3, 4, or 5.

86. A method of identifying one or more sets of a first polypeptide and a second polypeptide, wherein:
(i) the first polypeptide is the same as or is a variant of a first parent polypeptide;
(ii) the second polypeptide is the same as or is a variant of a second parent polypeptide;
(iii) the first polypeptide is a variant of the first parent polypeptide and/or the second polypeptide is a variant of the second parent polypeptide; and
(iv) the first and second polypeptides preferentially pair with each other, optionally more preferentially compared to the first and second parent polypeptides,
the method comprising:
(a) providing multiple sets of a first candidate polypeptide and a second candidate polypeptide, optionally wherein the providing is performed in silico or in vitro and optionally wherein at least one set of the first candidate polypeptide and the second candidate polypeptide is derived from the library according to claim 85;
(b) quantifying the binding preference between the first candidate polypeptide and the second candidate polypeptide, optionally wherein the binding preference is based on the strength of inter-polypeptide hydrogen bond(s) and/or of inter-polypeptide binding energy, further optionally wherein the quantifying is performed in silico or in vitro; and
(c) selecting one or more sets of a first polypeptide and a second polypeptide which provide preferential inter-polypeptide paring, optionally equivalent or higher preferential pairing relative to a reference polypeptide set, further optionally wherein the reference polypeptide set is a set of (I) a first parent polypeptide or a variant thereof and (II) a second parent polypeptide or a variant thereof.


SEQUENCE LISTING (209 sequences)

SEQ ID NO: 1 - protein, 103 aa, Artificial Sequence
  Exemplary CH1 sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 107 aa, Artificial Sequence [sequence omitted]

SEQ ID NO: 3-8 - skipped (6 entries)

SEQ ID NO: 9 - protein, 106 aa, Artificial Sequence
  CL Lambda sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - skipped

SEQ ID NO: 11 - protein, 103 aa, Artificial Sequence
  Exemplary CH1 sequence
ASTKGPSVFPLAPSSKSTSGGTAALGCLVKDYFPEPVTVSWNSGALTSGVSTFPAVLQSSGLYSLSSSVDVPSSSLGTQTYICNVNHKPSNTKVDKKVEPKSC

SEQ ID NO: 12 - protein, 107 aa, Artificial Sequence
  Exemplary CLK sequence
RTVAAPSVFIFPPSDEQLKSGTASVVCRLNNFYPREAKVQWKVDNALQSGNSQESVTEQDSKDSTYSLSSTLTLSKADYEKHKVYACEVTHQGLSSPVTKSFNRGEC

SEQ ID NO: 13-18 - skipped (6 entries)

SEQ ID NO: 19 - protein, 106 aa, Artificial Sequence
  CL Lambda sequence
GQPKAAPSVTLFPPSSEELQANKATLVCRISDFYPGAVTVAWKADSSPVKAGVETTTPSKQSNNKYAASSYLSLTPEQWKSHRSYSCQVTHEGSTVEKTVAPTECS

SEQ ID NO: 20 - skipped

SEQ ID NO: 21 - protein, 103 aa, Artificial Sequence
  Exemplary CH1 sequence
ASTKGPSVFPRAPSSKSTSGGTAALGCLVRDYFPEPVTVSWNSGALTSGVHTFPAVLQSSGLYSLSSVVTVPSSSLGTQTYICNVNHKPSNTKVDKKVEPKSC

SEQ ID NO: 22 - protein, 107 aa, Artificial Sequence
  Exemplary CLK sequence
RTVAAPSVFIFPPSDEELKSGTASVQCLLNNFYPREAKVQWKVDNALQSGNSQESVTEQDSKDSTYSLSSELTLSKADYEKHKVYACEVTHQGLSSPVTKSFNRGEC

SEQ ID NO: 23-28 - skipped (6 entries)

SEQ ID NO: 29 - protein, 106 aa, Artificial Sequence
  CL Lambda sequence
GQPKAAPSVTLFPPSSEELQANKATLQCLISDFYPGAVTVAWKADSSPVKAGVETTTPSKQSNNKYAASSELSLTPEQWKSHRSYSCQVTHEGSTVEKTVAPTECS

SEQ ID NO: 30 - skipped

SEQ ID NO: 31 - protein, 103 aa, Artificial Sequence
  Exemplary CH1 sequence
ASTKGPSVFPLAPSSKSTSGGTAALGCQVEDYFPEPVTVSWNSGALTSGVHTFPAVLQSSGLYELSSVVTVPSSSLGTQTYICNVNHKPSNTKVDKKVEPKSC

SEQ ID NO: 32 - protein, 107 aa, Artificial Sequence
  Exemplary CLK sequence
RTVAAPSVFIFPPSDEQLKSGRASVVCLLNNFYPREAKVQWKVDNALQSGNSQESVTEQDSKDSTYSLSSRLQLSKADYEKHKVYACEVTHQGLSSPVTKSFNRGEC

SEQ ID NO: 33-38 - skipped (6 entries)

SEQ ID NO: 39 - protein, 106 aa, Artificial Sequence
  CL Lambda sequence
GQPKAAPSVTLFPPSSEELQANRATLVCLISDFYPGAVTVAWKADSSPVKAGVETTTPSKQSNNKYAASSRLQLTPEQWKSHRSYSCQVTHEGSTVEKTVAPTECS

SEQ ID NO: 40 - skipped

SEQ ID NO: 41 - protein, 103 aa, Artificial Sequence
  Exemplary CH1 sequence
ASTKGPSVFPLAPSSKSTSGGTAALGCLVTDYFPEPVTVSWNSGALTSGVHTFPAVLQSSGLYSLSSQVTVPSSSLGTQTYICNVNHKPSNTKVDKKVEPKSC

SEQ ID NO: 42 - protein, 107 aa, Artificial Sequence
  Exemplary CLK sequence
RTVAAPSVFIFPPSDEQLKSGTASVVCSLNNFYPREAKVQWKVDNALQSGNSQESVTEQDSKDSTYSLSSRLTLSKADYEKHKVYACEVTHQGLSSPVTKSFNRGEC

SEQ ID NO: 43-48 - skipped (6 entries)

SEQ ID NO: 49 - protein, 106 aa, Artificial Sequence
  CL Lambda sequence
GQPKAAPSVTLFPPSSEELQANKATLVCSISDFYPGAVTVAWKADSSPVKAGVETTTPSKQSNNKYAASSRLSLTPEQWKSHRSYSCQVTHEGSTVEKTVAPTECS

SEQ ID NO: 50 - skipped

SEQ ID NO: 51 - protein, 103 aa, Artificial Sequence
  Exemplary CH1 sequence
ASTKGPSVFPLAPSSKSTSGGTAALGCLVKRYFPEPVTVSWNSGALTSGVHTFPAVLQSSGLYSLSSVVTVPSSSLGTQTYICNVNHKPSNTKVDKKVEPKSC

SEQ ID NO: 52 - protein, 107 aa, Artificial Sequence
  Exemplary CLK sequence
RTVAAPSVFIFPPSDESLKSGEASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53-58 - skipped (6 entries)

SEQ ID NO: 59 - protein, 106 aa, Artificial Sequence
  CL Lambda sequence
GQPKAAPSVTLFPPSSESLQANEATLVCLISDFYPGAVTVAWKADSSPVKAGVETTTPSKQSNNKYAASSYLSLTPEQWKSHRSYSCQVTHEGSTVEKTVAPTECS

SEQ ID NO: 60 - skipped

SEQ ID NO: 61 - protein, 103 aa, Artificial Sequence
  Exemplary CH1 sequence
ASTKGPSVFPLAPSSKSTSGGRAQLGCLVKDYFPEPVTVSWNSGALTSGVHTFPAVLQSSGLYSLSSVVQVPSSSLGTQTYICNVNHKPSNTKVDKKVEPKSC

SEQ ID NO: 62 - protein, 107 aa, Artificial Sequence
  Exemplary CLK sequence
RTVAAPDVFIFPPSDEQLKSGTASVVCSLNRFYPREAKVQWKVDNALQSGNSQESVTEQDSKDSTYSLSSTLTLSKADYEKHKVYACEVTHQGLSSPVTKSFNRGEC

SEQ ID NO: 63-68 - skipped (6 entries)

SEQ ID NO: 69 - protein, 106 aa, Artificial Sequence
  CL Lambda sequence
GQPKAAPDVTLFPPSSEELQANKATLVCSISRFYPGAVTVAWKADSSPVKAGVETTTPSKQSNNKYAASSYLSLTPEQWKSHRSYSCQVTHEGSTVEKTVAPTECS

SEQ ID NO: 70 - skipped

SEQ ID NO: 71 - protein, 103 aa, Artificial Sequence
  Exemplary CH1 sequence
ASTKGPSVFPLAPSSKSTSGGTAALGCLVKDYFPEPVTVSWNSGALTSKVHTFPAVLQSSGLYSLSSVVKVPSSSLGTQTYICNVNHKPSNTKVDKKVEPKSC

SEQ ID NO: 72 - protein, 107 aa, Artificial Sequence
  Exemplary CLK sequence
RTVAAPSVFIFPPSDEQLKSGTASVVCLLSEFYPREAKVQWKVDNALQSGNSQESVTEQDSKDSTYSLSSTLTLSKADYEKHKVYACEVTHQGLSSPVTKSFNRGEC

SEQ ID NO: 73-78 - skipped (6 entries)

SEQ ID NO: 79 - protein, 106 aa, Artificial Sequence
  CL Lambda sequence
GQPKAAPSVTLFPPSSEELQANKATLVCLISEFYPGAVTVAWKADSSPVKAGVETTTPSKQSNNKYAASSYLSLTPEQWKSHRSYSCQVTHEGSTVEKTVAPTECS

SEQ ID NO: 80 - skipped

SEQ ID NO: 81 - protein, 103 aa, Artificial Sequence
  Exemplary CH1 sequence
ASTKGPSVFPLAPSSKSTSGGTAALGCLVKDYFPEPVTVSWNSGALTSGVRTFPAVLQSSGLYSLSSEVTVPSSSLGTQTYICNVNHKPSNTKVDKKVEPKSC

SEQ ID NO: 82 - protein, 107 aa, Artificial Sequence
  Exemplary CLK sequence
RTVAAPSVFIFPPSDEQLKSGTASVVCSLNNFYPREAKVQWKVDNALQSGNSQESVTEQDSKDSTYSLSSTLTLSKADYEKHKVYACEVTHQGLSSPVTKSFNRGEC

SEQ ID NO: 83-88 - skipped (6 entries)

SEQ ID NO: 89 - protein, 106 aa, Artificial Sequence
  CL Lambda sequence
GQPKAAPSVTLFPPSSEELQANKATLVCSISDFYPGAVTVAWKADSSPVKAGVETTTPSKQSNNKYAASSYLSLTPEQWKSHRSYSCQVTHEGSTVEKTVAPTECS

SEQ ID NO: 90 - skipped

SEQ ID NO: 91 - protein, 103 aa, Artificial Sequence
  Exemplary CH1 sequence
ASTKGPRVFPLAPSSKSTSGGTAALGCLVRDYFPEPVTVSWNSGALTSGVHTFPAVLQSSGLYSLSSVVTVPSSSLGTQTYICNVNHKPSNTKVDKKVEPKSC

SEQ ID NO: 92 - protein, 107 aa, Artificial Sequence
  Exemplary CLK sequence
RTVAAPSVFIFPPSDEQLKDGEASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93-98 - skipped (6 entries)

SEQ ID NO: 99 - protein, 106 aa, Artificial Sequence
  CL Lambda sequence
GQPKAAPSVTLFPPSSEELQDNEATLVCLISDFYPGAVTVAWKADSSPVKAGVETTTPSKQSNNKYAASSYLSLTPEQWKSHRSYSCQVTHEGSTVEKTVAPTECS

SEQ ID NO: 100 - skipped

SEQ ID NO: 101 - protein, 103 aa, Artificial Sequence
  Exemplary CH1 sequence
ASTKGPSVFPLAPSSKSTSGGTAALGCLVHEYFPEPVTVSWNSGALTSGVHTFPAVLQSSGLYSLSSVVTVPSSSLGTQTYICNVNHKPSNTKVDKKVEPKSC

SEQ ID NO: 102 - protein, 107 aa, Artificial Sequence
  Exemplary CLK sequence
RTVAAPSVFIFPPSDEQLKRGRASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103-108 - skipped (6 entries)

SEQ ID NO: 109 - protein, 106 aa, Artificial Sequence
  CL Lambda sequence
GQPKAAPSVTLFPPSSEELQRNRATLVCLISDFYPGAVTVAWKADSSPVKAGVETTTPSKQSNNKYAASSYLSLTPEQWKSHRSYSCQVTHEGSTVEKTVAPTECS

SEQ ID NO: 110 - skipped

SEQ ID NO: 111 - protein, 103 aa, Artificial Sequence
  Exemplary CH1 sequence
ASTKGPSVFPLAPSSKSTSGGTAALGCSVKDYFPEPVTVSWNSGALTSGVHTFPAVLQSSGLYSLSSVVTVPSSSLGTQTYICNVNHKPSNTKVDKKVEPKSC

SEQ ID NO: 112 - protein, 107 aa, Artificial Sequence
  Exemplary CLK sequence
RTVAAPSVFIFPPSDEQLKSGTASVYCLLNNFYPREAKVQWKVDNALQSGNSQESVTEQDSKDSTYSLSSTLTLSKADYEKHKVYACEVTHQGLSSPVTKSFNRGEC

SEQ ID NO: 113-118 - skipped (6 entries)

SEQ ID NO: 119 - protein, 106 aa, Artificial Sequence
  CL Lambda sequence
GQPKAAPSVTLFPPSSEELQANKATLYCLISDFYPGAVTVAWKADSSPVKAGVETTTPSKQSNNKYAASSYLSLTPEQWKSHRSYSCQVTHEGSTVEKTVAPTECS

SEQ ID NO: 120 - skipped

SEQ ID NO: 121 - protein, 103 aa, Artificial Sequence
  Exemplary CH1 sequence
ASTKGPSVFPLAPSSKSTSGGTAALGCSVKDYFPEPVTVSWNSGALTSGVHTFPAVLQSSGLYQLSSVVTVPSSSLGTQTYICNVNHKPSNTKVDKKVEPKSC

SEQ ID NO: 122 - protein, 107 aa, Artificial Sequence; identical to SEQ ID NO: 112
  Exemplary CLK sequence

SEQ ID NO: 123-128 - skipped (6 entries)

SEQ ID NO: 129 - protein, 106 aa, Artificial Sequence; identical to SEQ ID NO: 119
  CL Lambda sequence

SEQ ID NO: 130 - skipped

SEQ ID NO: 131 - protein, 103 aa, Artificial Sequence; identical to SEQ ID NO: 111
  Exemplary CH1 sequence

SEQ ID NO: 132 - protein, 107 aa, Artificial Sequence
  Exemplary CLK sequence
RTVAAPSVFIFPPSDEELKSGTASVYCLLNNFYPREAKVQWKVDNALQSGNSQESVTEQDSKDSTYSLSSTLTLSKADYEKHKVYACEVTHQGLSSPVTKSFNRGEC

SEQ ID NO: 133-138 - skipped (6 entries)

SEQ ID NO: 139 - protein, 106 aa, Artificial Sequence; identical to SEQ ID NO: 119
  CL Lambda sequence

SEQ ID NO: 140 - skipped

SEQ ID NO: 141 - protein, 103 aa, Artificial Sequence
  Exemplary CH1 sequence
ASTKGPSVFPLAPSSKSTSGGTAALGCQVKDYFPEPVTVSWNSGALTSGVHTFPAVLQSSGLYELSSVVTVPSSSLGTQTYICNVNHKPSNTKVDKKVEPKSC

SEQ ID NO: 142 - protein, 107 aa, Artificial Sequence
  Exemplary CLK sequence
RTVAAPSVFIFPSSDEQLKSGTASVVCLLNNFYPREAKVQWKVDNALQSGNSQESVTEQDSKDSTYSLSSHLQLSKADYEKHKVYACEVTHQGLSSPVTKSFNRGEC

SEQ ID NO: 143-148 - skipped (6 entries)

SEQ ID NO: 149 - protein, 106 aa, Artificial Sequence
  CL Lambda sequence
GQPKAAPSVTLFPSSSEELQANKATLVCLISDFYPGAVTVAWKADSSPVKAGVETTTPSKQSNNKYAASSHLQLTPEQWKSHRSYSCQVTHEGSTVEKTVAPTECS

SEQ ID NO: 150 - skipped

SEQ ID NO: 151 - protein, 103 aa, Artificial Sequence
  Exemplary CH1 sequence
ASTKGPRVFPLAPSSKSTSGGTAALGCSVQDYFPEPVTVSWNSGALTSGVHTFPAVLQSSGLYSLSSVVTVPSSSLGTQTYICNVNHKPSNTKVDKKVEPKSC

SEQ ID NO: 152 - protein, 107 aa, Artificial Sequence
  Exemplary CLK sequence
RTVAAPSVFIFPPSDEQLKTGDASVVCLLNNFYPREAKVQWKVDNALQSGNSQESVTEQDSKDSTYSLSSRLTLSKADYEKHKVYACEVTHQGLSSPVTKSFNRGEC

SEQ ID NO: 153-158 - skipped (6 entries)

SEQ ID NO: 159 - protein, 106 aa, Artificial Sequence
  CL Lambda sequence
GQPKAAPSVTLFPPSSEELQTNDATLVCLISDFYPGAVTVAWKADSSPVKAGVETTTPSKQSNNKYAASSRLSLTPEQWKSHRSYSCQVTHEGSTVEKTVAPTECS

SEQ ID NO: 160 - skipped

SEQ ID NO: 161 - protein, 103 aa, Artificial Sequence; identical to SEQ ID NO: 71
  Exemplary CH1 sequence

SEQ ID NO: 162 - protein, 107 aa, Artificial Sequence
  Exemplary CLK sequence
RTVAAPQVFIFPPSDEQLKSGTASVVCLLTEFYPREAKVQWKVDNALQSGNSQESVTEQDSKDSTYSLSSTLTLSKADYEKHKVYACEVTHQGLSSPVTKSFNRGEC

SEQ ID NO: 163-168 - skipped (6 entries)

SEQ ID NO: 169 - protein, 106 aa, Artificial Sequence
  CL Lambda sequence
GQPKAAPQVTLFPPSSEELQANKATLVCLITEFYPGAVTVAWKADSSPVKAGVETTTPSKQSNNKYAASSYLSLTPEQWKSHRSYSCQVTHEGSTVEKTVAPTECS

SEQ ID NO: 170 - skipped

SEQ ID NO: 171 - protein, 103 aa, Artificial Sequence
  Exemplary CH1 sequence
ASTKGPSVFPLAPSSKSTSGGTAALGCLVRDYFPEPVTVSWNSGALTSGVHTFPAVLDSSGLYSLSSVVTVPSSSLGTQTYICNVNHKPSNTKVDKKVEPKSC

SEQ ID NO: 172 - protein, 107 aa, Artificial Sequence
  Exemplary CLK sequence
RTVAAPSVFIFPPSDEQLKSGDASVVCLLNNFYPREAKVQWKVDNALQSGNSQESVTEQDSKDSTYSLSSRLHLSKADYEKHKVYACEVTHQGLSSPVTKSFNRGEC

SEQ ID NO: 173-178 - skipped (6 entries)

SEQ ID NO: 179 - protein, 106 aa, Artificial Sequence
  CL Lambda sequence
GQPKAAPSVTLFPPSSEELQANDATLVCLISDFYPGAVTVAWKADSSPVKAGVETTTPSKQSNNKYAASSRLHLTPEQWKSHRSYSCQVTHEGSTVEKTVAPTECS

SEQ ID NO: 180 - skipped

SEQ ID NO: 181 - protein, 103 aa, Artificial Sequence
  Exemplary CH1 sequence
ASTKGPSVFPLAPSSKSTSGGTAALGCLVRDYFPEPVTVSWNSGALTSGVHTFPAVLESSGLYQLSSVVTVPSSSLGTQTYICNVNHKPSNTKVDKKVEPKSC

SEQ ID NO: 182 - protein, 107 aa, Artificial Sequence
  Exemplary CLK sequence
RTVAAPSVFIFPPSDEQLKSGDASVVCLLNNFYPREAKVQWKVDNALQSGNSQESVTEQDSKDSTYSLSSTLQLSKADYEKHKVYACEVTHQGLSSPVTKSFNRGEC

SEQ ID NO: 183-188 - skipped (6 entries)

SEQ ID NO: 189 - protein, 106 aa, Artificial Sequence
  CL Lambda sequence
GQPKAAPSVTLFPPSSEELQANDATLVCLISDFYPGAVTVAWKADSSPVKAGVETTTPSKQSNNKYAASSYLQLTPEQWKSHRSYSCQVTHEGSTVEKTVAPTECS

SEQ ID NO: 190 - skipped

SEQ ID NO: 191 - protein, 103 aa, Artificial Sequence
  Exemplary CH1 sequence
ASTKGPSVFPLAPSSKSTSGGTAALGCSVNDYFPEPVTVSWNSGALTSGVHTFPAVLQSSGLYSLSSVVTVPSSSLGTQTYICNVNHKPSNTKVDKKVEPKSC

SEQ ID NO: 192 - protein, 107 aa, Artificial Sequence
  Exemplary CLK sequence
RTVAAPSVFIFPPSDEQLKSGTASVYCLLNNFYPREAKVQWKVDNALQSGNSQESVTEQDSKDSTYSLSSTLRLSKADYEKHKVYACEVTHQGLSSPVTKSFNRGEC

SEQ ID NO: 193-198 - skipped (6 entries)

SEQ ID NO: 199 - protein, 106 aa, Artificial Sequence
  CL Lambda sequence
GQPKAAPSVTLFPPSSEELQANKATLYCLISDFYPGAVTVAWKADSSPVKAGVETTTPSKQSNNKYAASSYLRLTPEQWKSHRSYSCQVTHEGSTVEKTVAPTECS

SEQ ID NO: 200 - skipped

SEQ ID NO: 201 - protein, 103 aa, Artificial Sequence
  Exemplary CH1 sequence
ASTKGPSVFPLAPSSKSTSGGTAALGCLVNDYFPEPVTVSWNSGALTSGVHTFPAVLQSSGLYSLSSYVTVPSSSLGTQTYICNVNHKPSNTKVDKKVEPKSC

SEQ ID NO: 202 - protein, 107 aa, Artificial Sequence
  Exemplary CLK sequence
RTVAAPSVFIFPPSDEQLKSGRASVVCLLNNFYPREAKVQWKVDNALQSGNSQESVTEQDSKDSTYSLSSTLSLSKADYEKHKVYACEVTHQGLSSPVTKSFNRGEC

SEQ ID NO: 203-208 - skipped (6 entries)

SEQ ID NO: 209 - protein, 106 aa, Artificial Sequence
  CL Lambda sequence
GQPKAAPSVTLFPPSSEELQANRATLVCLISDFYPGAVTVAWKADSSPVKAGVETTTPSKQSNNKYAASSYLSLTPEQWKSHRSYSCQVTHEGSTVEKTVAPTECS